(19) United States
(12) Patent Application Publication
(10) Pub. No.: US 2026/0356933 A1
(43) Pub. Date: Nov. 12, 2026

(54) ANTI-INTERLEUKIN-6 SINGLE-DOMAIN ANTIBODIES

(71) Applicants: Christopher REES, Cobham (GB); SEERAVE VENTURES LIMITED, St Helier (GB)

(72) Inventor: Manuel FANKHAUSER, St Helier (GB)

(73) Assignees: Christopher REES, Cobham (GB); SEERAVE VENTURES LIMITED, St Helier (GB)

(21) Appl. No.: 19/483,601

(22) PCT Filed: May 10, 2024
(86) PCT No.: PCT/EP2024/062988

§ 371 Date: Nov. 11, 2025

(30) Foreign Application Priority Data
May 12, 2023 (EP) 23173244.7

(51) Int. Cl. C07K 16/24; G01N 33/68

(52) U.S. Cl. CPC C07K 16/248; G01N 33/6893; C07K 2317/565; C07K 2317/567; C07K 2317/569; C07K 2317/76; C07K 2317/92; G01N 2333/5412

(57) ABSTRACT

The present invention relates to single-domain antibodies (sdAbs) binding interleukin 6 (IL6). In particular, the invention relates to sdAbs that bind IL6 within a specific range of an on-rate constant and off-rate constant. The sdAbs of the present invention can associate and dissociate with IL6 in a continuous manner. The invention further relates to nucleic acids encoding anti-IL6 sdAbs, methods for detecting IL6, methods of treatment and diagnosis making use of the anti-IL6 sdAbs, methods for monitoring IL6 levels and apparatuses comprising the anti-IL6 sdAbs.

The present invention relates to single-domain antibodies (sdAbs) binding interleukin 6 (IL6). In particular, the invention relates to sdAbs that bind IL6 within a specific range of an on-rate constant and off-rate constant. The sdAbs of the present invention can associate and dissociate with IL6 in a continuous manner. The invention further relates to nucleic acids encoding anti-IL6 sdAbs, methods for detecting IL6, treatments and diagnoses making use of the anti-IL6 sdAbs, methods for monitoring IL6 levels and apparatuses comprising the anti-IL6 sdAbs.

Single-domain antibodies (sdAb) are a type of antibody fragment consisting of a single monomeric variable antibody domain only. Similarly, to their larger counterparts they are able to selectively bind to a specific antigen. sdAb can, e.g. be engineered from heavy-chain antibodies in camelids, referred to as V_{H}H fragments. An advantage when using sdAbs is that they can be designed to not affect the biological activity or structure of a bound molecule. However, they may also be specifically designed to block biological activity. Further, sdAbs are particularly robust which is advantageous for e.g. application as biosensors or as components of biosensors. Thus, sdAbs are advantageous to specifically detect a molecule of interest with or without interfering with biological functions, e.g. as or in a biosensor. Interleukin 6 (IL6) is a cytokine, e.g. secreted by macrophages in response to specific microbial molecules, such as pathogen-associated molecular patterns (PAMPs). Secretion of IL6 can induce an inflammatory reaction. The pathological significance of IL6 for various diseases has been indicated in numerous studies, including chronic inflammatory diseases, autoimmune diseases (e.g., rheumatoid arthritis, Crohn's disease, Castleman's disease and psoriasis), cancer (e.g., multiple myeloma, leukaemia, breast cancer, pancreatic cancer, prostate cancer and various cancers), and cachexia and coronary heart disease. The treatment of such diseases with drugs preferably results in reduced inflammation, i.e. a reduced level of IL6 in a subject, for example reduced to the upper limit of normal (ULN) in a healthy non-inflammatory subject avoiding excessive tissue damage. To achieve this and to avoid overdosing or underdosing of drugs that may invoke adverse effects or result in an ineffective therapy, precise monitoring of IL6 levels in a subject is desired. In addition, monitoring of IL6 levels is useful to assess the therapeutic outcome of a potential treatment and whether a subject is for example incompatible with a treatment resulting in inflammation after drug administration. To achieve the above, precise measurement of IL6 levels, preferably over an extended period of time, i.e. continuous monitoring of IL6 levels is needed. However, the current means for detecting IL6 are limited to antibodies with exceptionally high affinities to IL6 which do however not consider the dynamic range of IL6 levels in a subject and are not feasible for use in a biosensor. For example, US 2011/0059101 A9 discloses anti-IL6 nanobodies. A very high affinity IL6 antibody results in a permanent binding of IL6 which would saturate a biosensor. A saturated biosensor is unable to continuously monitor levels of IL6 thereby rendering the same useless for measurements over time. When using a very high affinity IL6 antibody, detection of IL6 is limited to a yes, no type of spot measurement of IL6 which does not allow for continuously monitoring dynamic levels of IL6 that may be specific for certain diseases and therapies.

Accordingly, there is a need for continuous monitoring of dynamic IL6 levels, e.g. in a subject undergoing therapy or in a subject suffering from a disease. The technical problem underlying the present invention is the provision of means and methods for the detection, quantification and/or the monitoring of IL6, particularly continuous detection, quantification and/or monitoring. The technical problem is solved by provision of the embodiments characterized in the claims and as provided herein below. Specifically, the technical problem is solved, and the above-mentioned difficulties are overcome by the provision of an anti-IL6 sdAb.

The inventors have predicted the dynamic range of IL6 levels in the context of disease and homeostasis, i.e. in a healthy context. They have shown that means for detecting, quantifying and monitoring IL6 should cover said dynamic range to allow for an unambiguous conclusion about the inflammation status in a subject. The inventors have used the dynamic range of IL6 to specifically generate sdAb that are capable of detecting IL6 throughout the entire range, which allows detection and monitoring of the IL6 level and thus the inflammation status in a subject or sample. In addition, the sdAb of the present invention embody a specific on-rate constant and off-rate constant, allowing for continuous association and dissociation with IL6, thereby enabling continuous measurement of IL6 levels over time.

The invention, inter alia, provides the following advantages:
Anti-IL6 sdAb specifically associate with IL6 over the entire dynamic range of IL6 allowing for precise detection and quantification of the IL6 level and thus inflammation status that is relevant in therapy.
The anti-IL6 sdAbs can detect IL6 from baseline IL6 levels to IL6 levels being present in severe inflammation.
The anti-IL6 sdAbs can continuously and repeatedly bind IL6 allowing for continuous detection, quantification and monitoring of IL6 levels, e.g. when applied as a biosensor or as an implanted biosensor in a sample or subject, thereby overcoming extensive and laborious IL6 spot measurements.
The dynamic detection of IL6 levels contributes to the assessment of the therapeutic outcome of a treatment, or a possible adverse effect invoked by an intolerance to a drug.
The detection and quantification of IL6 levels over time allow for targeted therapy of inflammatory diseases, thereby reducing the amount of medication and potential side effects of said medication
The anti-IL6 sdAbs can be used as a biosensor in a device that allows for continuous monitoring of IL6 levels in a subject.

The invention provides the following items:
1. An anti-interleukin 6 (IL6) single-domain antibody (sdAb) comprising an on-rate constant (kₒₙ) of about 6.3×10\hat{ }4-1.3×10\hat{ }7 M⁻¹ s⁻¹ and an off-rate constant (k_{off}) of about 1×10\hat{ }−6 s⁻¹-1 s⁻¹ (preferably an off-rate constant (k_{off}) of about 1.2×10\hat{ }−6 s⁻¹-0.31 s⁻¹), preferably wherein said sdAb comprises an IC50 of about 1 nM or more than about 1 nM, and preferably wherein said sdAb is capable of detecting and/or quantifying IL6 at a concentration of about 45 fM or less to about 4.5 pM or more.
2. The anti-IL6 sdAb of item 1, wherein the sdAb comprises a first complementary determining region (CDR1), a second CDR (CDR2) and third CDR (CDR3), wherein:
   (i) CDR1 comprises SEQ ID NO: 3 or a variant thereof having up to two amino acid substitutions, CDR2 comprises SEQ ID NO: 5 or a variant thereof having up to two amino acid substitutions, CDR3 comprises SEQ ID NO: 7 or a variant thereof having up to five amino acid substitutions; or
   (ii) CDR1 comprises SEQ ID NO: 11 or a variant thereof having up to two amino acid substitutions, CDR2 comprises SEQ ID NO: 13 or a variant thereof having up to two amino acid substitutions, CDR3 comprises SEQ ID NO: 15 or a variant thereof having up to five amino acid substitutions; or
   (iii) CDR1 comprises SEQ ID NO: 19 or a variant thereof having up to two amino acid substitutions, CDR2 comprises SEQ ID NO: 21 or a variant thereof having up to two amino acid substitutions, CDR3 comprises SEQ ID NO: 23 or a variant thereof having up to five amino acid substitutions; or
   (iv) CDR1 comprises SEQ ID NO: 27 or a variant thereof having up to two amino acid substitutions, CDR2 comprises SEQ ID NO: 29 or a variant thereof having up to two amino acid substitutions, CDR3 comprises SEQ ID NO: 31 or a variant thereof having up to five amino acid substitutions; or
   (v) CDR1 comprises SEQ ID NO: 35 or a variant thereof having up to two amino acid substitutions, CDR2 comprises SEQ ID NO: 37 or a variant thereof having up to two amino acid substitutions, CDR3 comprises SEQ ID NO: 39 or a variant thereof having up to five amino acid substitutions; or
   (vi) CDR1 comprises SEQ ID NO: 43 or a variant thereof having up to two amino acid substitutions, CDR2 comprises SEQ ID NO: 45 or a variant thereof having up to two amino acid substitutions, CDR3 comprises SEQ ID NO: 47 or a variant thereof having up to five amino acid substitutions; or
   (vii) CDR1 comprises SEQ ID NO: 51 or a variant thereof having up to two amino acid substitutions, CDR2 comprises SEQ ID NO: 53 or a variant thereof having up to two amino acid substitutions, CDR3 comprises SEQ ID NO: 55 or a variant thereof having up to five amino acid substitutions; or
   (viii) CDR1 comprises SEQ ID NO: 59 or a variant thereof having up to two amino acid substitutions, CDR2 comprises SEQ ID NO: 61 or a variant thereof having up to two amino acid substitutions, CDR3 comprises SEQ ID NO: 63 or a variant thereof having up to five amino acid substitutions.
3. The anti-IL6 sdAb of item 1 or 2, wherein the sdAb comprises a first framework region (FR1), a second framework region (FR2) and third framework region (FR3), wherein:
   (i) FR1 comprises SEQ ID NO: 2 or a variant thereof having up to six amino acid substitutions, FR2 comprises SEQ ID NO: 4 or a variant thereof having up to four amino acid substitutions, FR3 comprises SEQ ID NO: 6 or a variant thereof having up to ten amino acid substitutions, and FR4 comprises SEQ ID NO: 8 or a variant thereof having up to three amino acid substitutions; or
   (ii) FR1 comprises SEQ ID NO: 10 or a variant thereof having up to six amino acid substitutions, FR2 comprises SEQ ID NO: 12 or a variant thereof having up to four amino acid substitutions, FR3 comprises SEQ ID NO: 14 or a variant thereof having up to ten amino acid substitutions, and FR4 comprises SEQ ID NO: 16 or a variant thereof having up to three amino acid substitutions; or
   (iii) FR1 comprises SEQ ID NO: 18 or a variant thereof having up to six amino acid substitutions, FR2 comprises SEQ ID NO: 20 or a variant thereof having up to four amino acid substitutions, FR3 comprises SEQ ID NO: 22 or a variant thereof having up to ten amino acid substitutions, and FR4 comprises SEQ ID NO: 24 or a variant thereof having up to three amino acid substitutions; or
   (iv) FR1 comprises SEQ ID NO: 26 or a variant thereof having up to six amino acid substitutions, FR2 comprises SEQ ID NO: 28 or a variant thereof having up to four amino acid substitutions, FR3 comprises SEQ ID NO: 30 or a variant thereof having up to ten amino acid substitutions, and FR4 comprises SEQ ID NO: 32 or a variant thereof having up to three amino acid substitutions; or
   (v) FR1 comprises SEQ ID NO: 34 or a variant thereof having up to six amino acid substitutions, FR2 comprises SEQ ID NO: 36 or a variant thereof having up to four amino acid substitutions, FR3 comprises SEQ ID NO: 38 or a variant thereof having up to ten amino acid substitutions, and FR4 comprises SEQ ID NO: 40 or a variant thereof having up to three amino acid substitutions; or
   (vi) FR1 comprises SEQ ID NO: 42 or a variant thereof having up to six amino acid substitutions, FR2 comprises SEQ ID NO: 44 or a variant thereof having up to four amino acid substitutions, FR3 comprises SEQ ID NO: 46 or a variant thereof having up to ten amino acid substitutions, and FR4 comprises SEQ ID NO: 48 or a variant thereof having up to three amino acid substitutions; or
   (vii) FR1 comprises SEQ ID NO: 50 or a variant thereof having up to six amino acid substitutions, FR2 comprises SEQ ID NO: 52 or a variant thereof having up to four amino acid substitutions, FR3 comprises SEQ ID NO: 54 or a variant thereof having up to ten amino acid substitutions, and FR4 comprises SEQ ID NO: 56 or a variant thereof having up to three amino acid substitutions; or
   (viii) FR1 comprises SEQ ID NO: 58 or a variant thereof having up to six amino acid substitutions, FR2 comprises SEQ ID NO: 60 or a variant thereof having up to four amino acid substitutions, FR3 comprises SEQ ID NO: 62 or a variant thereof having up to ten amino acid substitutions, and FR4 comprises SEQ ID NO: 64 or a variant thereof having up to three amino acid substitutions.
4. The anti-IL6 sdAb of any one of items 1 to 3, wherein the sdAb comprises any one of
   (i) SEQ ID NO: 1 or a variant thereof having at least about 90% sequence identity to SEQ ID NO: 1; or
   (ii) SEQ ID NO: 9 or a variant thereof having at least about 90% sequence identity to SEQ ID NO: 9; or
   (iii) SEQ ID NO: 17 or a variant thereof having at least about 90% sequence identity to SEQ ID NO: 17; or
   (iv) SEQ ID NO: 25 or a variant thereof having at least about 90% sequence identity to SEQ ID NO: 25; or
   (v) SEQ ID NO: 33 or a variant thereof having at least about 90% sequence identity to SEQ ID NO: 33; or
   (vi) SEQ ID NO: 41 or a variant thereof having at least about 90% sequence identity to SEQ ID NO: 41; or
   (vii) SEQ ID NO: 49 or a variant thereof having at least about 90% sequence identity to SEQ ID NO: 49; or
   (viii) SEQ ID NO: 57 or a variant thereof having at least about 90% sequence identity to SEQ ID NO: 57.
5. The anti-IL6 sdAb of any one of items 1-4, wherein the anti-IL6 sdAb comprises an on-rate constant (kₒₙ) of about 0.15×10\hat{ }6 M⁻¹ s⁻¹-2.25×10\hat{ }6 M⁻¹ s⁻¹ and an off-rate constant (k_{off}) of about 0.0002 s⁻¹-0.002 s⁻¹.
6. The anti-IL6 sdAb of any one of items 1-4, wherein the anti-IL6 sdAb comprises an on-rate constant (kₒₙ) of about 1.86×10\hat{ }5 M⁻¹ s⁻¹-1.82×10\hat{ }6 M⁻¹ s⁻¹ (optionally 2.21×10\hat{ }6 M⁻¹ s⁻¹) and an off-rate constant (k_{off}) of about 0.000308 s⁻¹-0.00161 s⁻¹ and optionally a K_{D} of about 3×10\hat{ }−10 M-6.8×10\hat{ }−9 M.
7. The anti-IL6 sdAb of any one of items 1-4, wherein the anti-IL6 sdAb comprises an on-rate constant (kₒₙ) of about 1.20×10\hat{ }6 M⁻¹ s⁻¹-1.30×10\hat{ }7 M⁻¹ s⁻¹ and an off-rate constant (k_{off}) of about 1.20×10\hat{ }−6 s⁻¹-7.40×10\hat{ }−2 s⁻¹ and optionally a K_{D} of about 2.80×10\hat{ }−13 M-1.40×10\hat{ }−8 M.
8. The anti-IL6 sdAb of any one of items 1-4, wherein the anti-IL6 sdAb comprises an on-rate constant (kₒₙ) of about 1.86×10\hat{ }5 M⁻¹ s⁻¹-2.21×10\hat{ }6 M⁻¹ s⁻¹±1.12×10\hat{ }3 M⁻¹ s⁻¹-1.69×10\hat{ }4 M⁻¹ s⁻¹ and an off-rate constant (k_{off}) of about 3.08×10\hat{ }−4 s⁻¹-1.61×10\hat{ }−3 s⁻¹±1.86×10\hat{ }−6 s⁻¹-2.94×10\hat{ }−5 s⁻¹ and optionally a K_{D} of about 0.30 M-6.80 M, optionally wherein the values are obtained by a SPR Biacore binding kinetic measurement, preferably wherein the sdAb is defined in any one of SEQ ID NOs: 1, 9, 17, 33, 41, 49, 57.
9. The anti-IL6 sdAb of any one of items 1-4, wherein the anti-IL6 sdAb comprises an on-rate constant (kₒₙ) of about 6.11×10\hat{ }5 M⁻¹ s⁻¹-2.33×10\hat{ }6 M⁻¹ s⁻¹±2.60×10\hat{ }4 M⁻¹ s⁻¹-3.10×10\hat{ }6 M⁻¹ s⁻¹ and an off-rate constant (k_{off}) of about 1.25×10\hat{ }−4 s⁻¹-3.57×10\hat{ }−2 s⁻¹±6.11×10\hat{ }−5 s⁻¹-2.32×10\hat{ }−2 s⁻¹ and optionally a K_{D} of about 2.81×10\hat{ }−1 M-2.66×10\hat{ }1 M, optionally wherein the values are obtained by an Octet BLI binding kinetic measurement, preferably wherein the sdAb is defined in any one of SEQ ID NOs: 1, 9, 17, 25, 33, 41, 49, 57.
10. The anti-IL6 sdAb of any one of items 1-9, wherein the anti-IL6 sdAb continuously and reversibly binds IL-6.
11. The anti-IL6 sdAb of any one of items 1-10, wherein the anti-IL6 sdAb binds a second or further IL6 after dissociating from a first IL6.
12. The anti-IL6 sdAb of any one of items 1-11, wherein the anti-IL6 sdAb inhibits the biological function of IL6.
13. The anti-IL6 sdAb of any one of items 1-11, wherein the anti-IL6 sdAb does not inhibit the biological function of IL6.
14. The anti-IL6 sdAb of item 12 or 13, wherein the biological function of IL6 is IL6 signalling or the IL6 signalling pathway.
15. The anti-IL6 sdAb of any one of items 1-14, wherein the anti-IL6 sdAb is conjugated to a biosensor electrode, such as a working electrode.
16. The anti-IL6 sdAb of item 15, wherein the anti-IL6 sdAb is conjugated to a biosensor electrode via a linker, such as a nucleic acid.
17. The anti-IL6 sdAb of item 15 or 16, wherein the biosensor comprises one or more electrode, such as a working electrode and/or a counter electrode and/or a reference electrode, and a substrate.
18. The anti-IL6 sdAb of any one of items 15-17, wherein the biosensor comprises at least a working electrode and a substrate.
19. The anti-IL6 sdAb of any one of items 15-18, wherein the biosensor comprises an electrode comprising a conductive material such as carbon, metal, or metallic compound.
20. The anti-IL6 sdAb of any one of items 1-19, wherein the anti-IL6 sdAb further comprises a detection label or tag.
21. The anti-IL6 sdAb of any one of items 1-19, wherein the anti-IL6 sdAb does not comprise a detection label or tag.
22. A nucleic acid encoding the anti-IL6 sdAb of any one of items 1-21.
23. A vector comprising the nucleic acid of item 22.
24. The vector of item 23, wherein the vector is an expression vector.
25. A method for detecting and/or quantifying IL6 in a sample, the method comprising:
   (i) contacting a sample with the anti-IL6 sdAb of any one of items 1-21, and
   (ii) detecting binding of the anti-IL6 sdAb.
26. An in situ method for detecting and/or quantifying IL6 in a subject, the method comprising
   (i) contacting a subject's biological fluid with the anti-IL6 sdAb of any one of items 1-21; and
   (ii) detecting binding of the anti-IL6 sdAb.
27. The method of item 25 or 26, further comprising detecting the binding of the anti-IL6 sdAb over time.
28. The method of any one of items 25-27, further comprising detecting the repeated binding of the anti-IL6 sdAb over time.
29. The method of any one of items 25-28, wherein binding of the anti-IL6 sdAb is detected by a biosensor.
30. The method of any one of items 25-29, wherein the sample comprises a biological fluid, such as a bodily fluid, for example interstitial fluid (ISF), blood, urine, stool, serum, tear fluid, sweat or saliva; or wherein the biological fluid is a bodily fluid, for example interstitial fluid (ISF), blood, urine, stool, serum, tear fluid, sweat or saliva.
31. The method of any one of items 25-30, wherein the sample comprises human interstitial fluid (ISF), blood, urine, stool, serum, tear, sweat or saliva, or wherein the biological fluid is a human bodily fluid, for example human interstitial fluid (ISF), blood, urine, stool, serum, tear fluid, sweat or saliva.
32. The method of any one of items 25 and 27-29, wherein the sample comprises a sample from a biological production system such as a culture sample.
33. The anti-IL6 sdAb of any one of items 1-21 for use as a medicament.
34. The anti-IL6 sdAb of any one of items 1-21 for use in in vivo diagnosis.
35. The anti-IL6 sdAb of any one of items 1-21 for use in the treatment of a disease associated with an inflammation, optionally a systemic inflammation.
36. A method of treatment a disease associated with an inflammation, optionally a systemic inflammation comprising a step of administering the anti-IL6 sdAb of any one of items 1-21 to a subject in need thereof.
37. The anti-IL6 sdAb for use of item 35, or the method of item 36, wherein the disease is associated with an increased production or release of inflammatory markers.
38. The anti-IL6 sdAb for use of item 35 or 37, or the method of item 36 or 37, wherein the disease is associated with an increased production or release of IL6.
39. The anti-IL6 sdAb for use of any one of items 35 or 37-38, or the method of any one of items 36-38, wherein the disease is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.
40. The anti-IL6 sdAb of any one of items 1-21 for use in a method of in vivo diagnosis of a disease associated with an inflammation, optionally a systemic inflammation, the method comprising:
   (i) contacting a subject with the anti-IL6 sdAb of any one of items 1-21, and
   (ii) detecting the binding of the anti-IL6 sdAb.
41. A method of in vivo diagnosis of a disease associated with an inflammation, optionally a systemic inflammation, the method comprising:
   (i) contacting a subject with the anti-IL6 sdAb of any one of items 1-21, and
   (ii) detecting binding of the anti-IL6 sdAb.
42. An ex vivo or in vitro method of monitoring the level of IL6 comprising:
   (i) contacting a sample of a subject with the anti-IL6 sdAb of any one of items 1-21, and
   (ii) detecting binding of the anti-IL6 sdAb.
43. A method of monitoring the level of IL6 in a subject comprising:
   (i) contacting the subject with the anti-IL6 sdAb of any one of items 1-21, and
   (ii) detecting binding of the anti-IL6 sdAb.
44. The anti-IL6 sdAb for use of any one of items 35 or 37-40, or the method of any one of items 36-39 or 41-43, wherein binding of the anti-IL6 sdAb is repeatedly detected over time.
45. An apparatus comprising:
   (i) the anti-IL6 sdAb of any one of items 1-21, and
   (ii) a contacting unit for contacting a sample or a subject with the anti-IL6 sdAb, and
   (iii) a detecting unit for detecting and/or quantifying binding of the anti-IL6 sdAb, and optionally
   (iv) a dispensing unit for dispensing a drug, which optionally is the anti-IL6 sdAb of any one of items 1-21.
46. The apparatus of item 45 for use in the treatment or in vivo diagnosis of a disease associated with an inflammation, optionally a systemic inflammation.
47. The apparatus of item 45 or the apparatus for use of item 46, wherein the detecting unit for detecting and/or quantifying binding of the anti-IL6 sdAb comprises a biosensor.
48. The apparatus or the apparatus for use of item 47, wherein the biosensor comprises one or more electrode, such as a working electrode and/or a counter electrode and/or a reference electrode, and a substrate, optionally wherein the sdAb is conjugated to the working electrode.
49. The apparatus or the apparatus for use of item 47 or 48, wherein the biosensor comprises at least a working electrode and a substrate.
50. The apparatus or the apparatus for use of any one of items 47-49, wherein the biosensor comprises an electrode comprising a conductive material such as carbon, a metal or metallic compound.
51. The apparatus or the apparatus for use of any one of items 48-50, wherein the anti-IL6 sdAb is layered on the one or more electrode or conjugated to the one or more electrode, preferably the working electrode and optionally, wherein the anti-IL6 sdAb is conjugated to said electrode via a linker.
52. The anti-IL6 sdAb for use of item 35 or 40, or the method of item 36 or 41, or the apparatus for use of any one of items 46-51, wherein the disease is associated with an increased production or release of inflammatory markers.
53. The anti-IL6 sdAb for use of any one of items 35, 40 or 52, or the method of any one of items 36, 41 or 52, or the apparatus for use of any one of items 46-52, wherein the disease is associated with an increased production or release of IL6.
54. The anti-IL6 sdAb for use of any one of items 35, 40 or 52-53, or the method of any one of items 36, 41 or 52-53, or the apparatus of any one of items 46-53, wherein the disease is any one of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.
55. A method of producing an anti-IL6 sdAb comprising:
   (i) contacting a subject with a recombinant human IL6, and
   (ii) isolating a sdAb which successfully binds to IL6, and
   (iii) selecting for an on-rate constant (kₒₙ) of about 6.3×10\hat{ }4 M⁻¹ s⁻¹-1.3×10\hat{ }7 M⁻¹ s⁻¹ and an off-rate constant (k_{off}) of about 1.2×10\hat{ }−6 s⁻¹-0.31 s⁻¹.
56. The method of item 55, wherein the method comprises selecting for an on-rate constant (kₒₙ) of about 0.15×10\hat{ }6 M⁻¹ s⁻¹-2.25×10\hat{ }6 M⁻¹ s⁻¹ and an off-rate constant (k_{off}) of about 0.0002 s⁻¹-0.002 s⁻¹.
57. The method of item 55 or 56, wherein the method further comprises a step of selecting for continuous and reversible binding to IL6.
58. The method of any one of items 55-57, wherein the method further comprises a step of selecting for binding to a second or further IL6 after dissociation from a first IL6.
59. The method of any one of items 55-58, wherein the method further comprises a step of selecting for inhibiting the biological function of IL6 or not inhibiting the biological function of IL6.
60. The method of item 59, wherein the biological function of IL6 is IL6 signalling or the IL6 signalling pathway.
61. An anti-IL6 sdAb produced by the method of any one of items 55-60.
62. A biosensor comprising the anti-IL6 sdAb of any one of items 1-21.
63. The biosensor of item 62, wherein the biosensor comprises one or more biosensor electrodes.
64. The biosensor of item 62 or 63, wherein the biosensor comprises a working electrode and a substrate
65. The biosensor of item 64, wherein the biosensor further comprises a counter electrode and/or a reference electrode.
66. The biosensor of any one of items 62-65, wherein the anti-IL6 sdAb is conjugated to a biosensor electrode, optionally to a working electrode.
67. The biosensor of item 66, wherein the anti-IL6 sdAb is conjugated to a biosensor electrode, optionally to a working electrode, via a linker, such as a nucleic acid linker, amino acid/peptide linker, peptide nucleic acid linker, polymer linker, polyethylenglycol (PEG) linker, protein-tag linker, or conventional chemistry technique linker.
68. The biosensor of any one of items 64-67, wherein the working electrode comprises a conductive material, such as carbon, metal or metallic compound.
69. The biosensor of any one of items 62-68, wherein the biosensor, the anti-IL6 sdAb or the linker comprises a detection label, a tag, or red-ox reporters/indicators.
70. The method of any one of items 25-32, 36-39 or 41-44, or the anti-IL6 sdAb for use of any one of items 35, 37-40 or 44, wherein binding of the anti-IL6 sdAb to IL6 is detected and/or quantified by electrochemical ELISA.

The present invention provides single-domain antibodies (sdAb) that specifically bind to interleukin 6. The sdAb specifically binding IL6 of the present invention may be referred to anti-IL6 sdAb or anti-IL6 VHH. Single-domain antibodies are also known as nanobodies. In the sense of the present invention a sdAb is the heavy chain variable domain of a single chain of a heavy-chain antibody. A heavy-chain antibody is an antibody which consists only of two heavy chains, each of which contain a heavy chain variable domain (V_{H}). The sdAb of the present invention is preferably engineered from the heavy chain variable domain of a camelid heavy-chain antibody.

The sdAbs of the present invention are particularly useful for detecting and monitoring the IL6 level over time since they embody a kinetic binding range and allow for continuous association and dissociation within the dynamic range of IL6 levels. The sdAb of the present invention have specifically been developed to be capable of detecting IL6 throughout the relevant inflammation ranges. Thus, the inventors have found sdAb that can detect IL6 levels from a baseline, e.g. in a healthy subject, to IL6 levels present during severe inflammation. Consequently, the sdAb of the present invention allow for precise single or continuous, quantitative detection of IL6 levels in all clinically relevant ranges of IL6 levels.

The sdAbs of the present invention can embody a specific on-rate constant (kₒₙ) and off-rate constant (k_{off}). The kₒₙ (kₐ) describes the rate of complex formation, i.e. the number of sdAb-IL6 complexes formed per second in a one molar solution of sdAb and IL6. The k_{off} (k_{d}) describes the rate or speed at which e.g. a ligand dissociates from a protein, such as a sdAb from IL6. The binding constant (also affinity constant or association constant) Kₐ is defined as kₒₙ/k_{off}. The dissociation constant K_{D} is defined as 1/Kₐ.

Unless otherwise defined, the herein provided kₒₙ(kₐ) values are displayed/provided in the unit [s⁻¹M⁻¹]. Unless otherwise defined, the herein provided k_{off} (k_{d}) values are displayed/provided in the unit [s⁻¹]. Unless otherwise defined, the herein provided dissociation constant K_{D} values are displayed/provided in the unit [nM].

In the sense of the present invention, a sdAb can have a kₒₙ and k_{off} in a range that allows for continuous association and dissociation to IL6. The invention is not particularly limited to any sdAb having specific kₒₙ or k_{off} values, rather any sdAb having kₒₙ and k_{off} values that allow for association and dissociation to IL6 can be used.

Accordingly, the present invention provides an anti-interleukin 6 (IL6) single-domain antibody (sdAb) comprising an on-rate constant (kₒₙ) of about 6.3×10\hat{ }4-1.3×10\hat{ }7 M⁻¹ s⁻¹ and an off-rate constant (k_{off}) of about 1×10\hat{ }−6 s⁻¹-1 s⁻¹ (preferably an off-rate constant (k_{off}) of about 1.2×10\hat{ }−6 s⁻¹-0.31 s⁻¹), preferably wherein the values are obtained by a Octet BLI binding kinetic measurement, more preferably wherein said sdAb comprises an IC50 of about 1 nM or more than about 1 nM, and preferably wherein said sdAb is capable of detecting and/or quantifying IL6 at a concentration of about 45 fM or less to about 4.5 pM or more.

In the context of the present invention, an anti-interleukin 6 (IL6) single-domain antibody (sdAb) can comprise an on-rate constant (kₒₙ) of about 1.1×10\hat{ }5 s⁻¹M⁻¹-3×10\hat{ }6 s⁻¹M⁻¹ and/or an off-rate constant (k_{off}) of about 2.5×10\hat{ }−4 s⁻¹-2×10\hat{ }−3, preferably wherein the values are obtained by a SPR Biacore binding kinetic measurement (preferably on samples comprising purified sdAbs).

Accordingly, the present invention relates to an anti-interleukin 6 (IL6) single-domain antibody (sdAb) preferably comprising an on-rate constant (kₒₙ) of about 1.1×10\hat{ }5 s⁻¹M⁻¹-3×10\hat{ }6 s⁻¹M⁻¹.

Preferably the lower limit is about 1.2×10\hat{ }5 s⁻¹M⁻¹, 1.3×10\hat{ }5 s⁻¹M⁻¹, 1.4×10\hat{ }5 s⁻¹M⁻¹, more preferably about 1.5×10\hat{ }5 s⁻¹M⁻¹, 1.6×10\hat{ }5 s⁻¹M⁻¹, 1.7×10\hat{ }5 s⁻¹M⁻¹, 1.8×10\hat{ }5 s⁻¹M⁻¹, particularly preferred about 1.81×10\hat{ }5 s⁻¹M⁻¹, 1.82×10\hat{ }5 s⁻¹M⁻¹, 1.83×10\hat{ }5 s⁻¹M⁻¹, 1.84×10\hat{ }5 s⁻¹M⁻¹, or 1.85×10\hat{ }5 s⁻¹M⁻¹, and most preferred about 1.86×10\hat{ }5 s⁻¹M⁻¹.

The lower limit may be about 2×10\hat{ }5 s⁻¹M⁻¹, 3×10\hat{ }5 s⁻¹M⁻¹, 4×10\hat{ }5 s⁻¹M⁻¹, 5×10\hat{ }5 s⁻¹M⁻¹, 6×10\hat{ }5 s⁻¹M⁻¹, 7×10\hat{ }5 s⁻¹M⁻¹ (e.g. about 7.3 10\hat{ }5 s⁻¹M⁻¹, specifically about 7.33 10\hat{ }5 s⁻¹M⁻¹) or 8×10\hat{ }5 s⁻¹M⁻¹ (e.g. about 8.1×10\hat{ }5 s⁻¹M⁻¹, 8.2×10\hat{ }5 s⁻¹M⁻¹, 8.3×10\hat{ }5 s⁻¹M⁻¹, 8.4×10\hat{ }5 s⁻¹M⁻¹, 8.5×10\hat{ }5 s⁻¹M⁻¹, or 8.6×10\hat{ }5 s⁻¹M⁻¹, specifically about 8.37 10\hat{ }5 s⁻¹M⁻¹, 8.45 10\hat{ }5 s⁻¹M⁻¹, or 8.64 10\hat{ }5 s⁻¹M⁻¹).

The upper limit may also be 9×10\hat{ }6 s⁻¹M⁻¹, 8×10\hat{ }6 s⁻¹M⁻¹, 7×10\hat{ }6 s⁻¹M⁻¹, 6×10\hat{ }6 s⁻¹M⁻¹, 5×10\hat{ }6 s⁻¹M⁻¹, 4×10\hat{ }6 s⁻¹M⁻¹.

More preferably, the upper limit is about 3×10\hat{ }6 s⁻¹M⁻¹, 2.9×10\hat{ }6 s⁻¹M⁻¹, 2.8×10\hat{ }6 s⁻¹M⁻¹, 2.7×10\hat{ }6 s⁻¹M⁻¹, 2.6×10\hat{ }6 s⁻¹M⁻¹, 2.5×10\hat{ }6 s⁻¹M⁻¹, 2.4×10\hat{ }6 s⁻¹M⁻¹, or 2.3×10\hat{ }6 s⁻¹M⁻¹, even more preferably about 2.2×10\hat{ }6 s⁻¹M⁻¹, most preferred about 2.21×10\hat{ }6 s⁻¹M⁻¹.

The upper limit may be about 2.1×10\hat{ }6 s⁻¹M⁻¹, 2.0×10\hat{ }6 s⁻¹M⁻¹, 1.9×10\hat{ }6 s⁻¹M⁻¹, 1.8×10\hat{ }6 s⁻¹M⁻¹ (preferably 1.82×10\hat{ }6 s⁻¹M⁻¹), 1.7×10\hat{ }6 s⁻¹M⁻¹, 1.6×10\hat{ }6 s⁻¹M⁻¹, 1.5×10\hat{ }6 s⁻¹M⁻¹, 1.4×10\hat{ }6 s⁻¹M⁻¹, 1.3×10\hat{ }6 s⁻¹M⁻¹, 1.2×10\hat{ }6 s⁻¹M⁻¹, 1.1×10\hat{ }6 s⁻¹M⁻¹, 1.0×10\hat{ }6 s⁻¹M⁻¹, or 9.0×10\hat{ }5 s⁻¹M⁻¹.

In this context, the on-rate constant (kₒₙ) values are preferably determined using an SPR Biacore measurement (preferably on samples comprising purified sdAbs).

Accordingly, the present invention relates to an anti-interleukin 6 (IL6) single-domain antibody (sdAb) preferably comprising an off-rate constant (k_{off}) of about 2.5×10\hat{ }−4 s⁻¹-2×10\hat{ }−3 s⁻¹. Preferably the lower limit is about 2.5×10\hat{ }−4 s⁻¹, 2.6×10\hat{ }−4 s⁻¹, 2.7×10\hat{ }−4 s⁻¹, 2.8×10\hat{ }−4 s⁻¹, 2.9×10\hat{ }−4 s⁻¹, 3×10\hat{ }−4 s⁻¹, more preferably about 3.05×10\hat{ }−4 s⁻¹, 3.06×10\hat{ }−4 s⁻¹, 3.07×10\hat{ }−4 s⁻¹, most preferred about 3.08×10\hat{ }−4 s⁻¹.

The lower limit may be about 3.1×10\hat{ }−4 s⁻¹, 4×10\hat{ }−4 s⁻¹, 5×10\hat{ }−4 s⁻¹, 5.1×10\hat{ }−4 s⁻¹, 5.2×10\hat{ }−4 s⁻¹, 5.3×10\hat{ }−4 s⁻¹, (specifically 5.35×10\hat{ }−4 s⁻¹), 5.4×10\hat{ }−4 s⁻¹, 5.5×10\hat{ }−4 s⁻¹ (specifically 5.52×10\hat{ }−4 s⁻¹), 5.6×10\hat{ }−4 s⁻¹, 5.7×10\hat{ }−4 s⁻¹, 5.8×10\hat{ }−4 s⁻¹, 5.9×10\hat{ }−4 s⁻¹, 6.0×10\hat{ }−4 s⁻¹, 6.1×10\hat{ }−4 s⁻¹, 6.2×10\hat{ }−4 s⁻¹, 6.3×10\hat{ }−4 s⁻¹, 6.4×10\hat{ }−4 s⁻¹, 6.5×10\hat{ }−4 s⁻¹ (specifically 6.53×10\hat{ }−4 s⁻¹), 6.6×10\hat{ }−4 s⁻¹, or 6.7×10\hat{ }−4 s⁻¹ (specifically 6.71×10\hat{ }−4 s⁻¹). More preferably, the upper limit is about 4×10\hat{ }−3, 3×10\hat{ }−3, or 2×10\hat{ }−3, (preferably 1.61×10\hat{ }−3).

Even more preferably, the upper limit is about 1.9×10\hat{ }−3 s⁻¹, 1.8×10\hat{ }−3 s⁻¹, 1.7×10\hat{ }−3 s⁻¹, 1.6×10\hat{ }−3 s⁻¹, (specifically 1.61×10\hat{ }−3 s⁻¹) 1.5×10\hat{ }−3 s⁻¹, 1.4×10\hat{ }−3 s⁻¹, 1.3×10\hat{ }−3 s⁻¹, (specifically 1.27×10\hat{ }−3 s⁻¹) 1.2×10\hat{ }−3 s⁻¹, 1.1×10\hat{ }−3 s⁻¹, 9×10\hat{ }−4, 8×10\hat{ }−4, or 7×10\hat{ }−4.

In this context, the off-rate constant (k_{off}) values are preferably determined using an SPR Biacore measurement (preferably on samples comprising purified sdAbs).

Accordingly, the present invention relates to an anti-interleukin 6 (IL6) single-domain antibody (sdAb) preferably comprising an on-rate constant (kₒₙ) of about 1.1×10\hat{ }5 s⁻¹M⁻¹-3×10\hat{ }6 s⁻¹M⁻¹ and/or an off-rate constant (k_{off}) of about 2.5×10\hat{ }−4 s⁻¹-2×10\hat{ }−3 s⁻¹,

preferably wherein the lower limit of the on-rate constant (kₒₙ) is about 1.2×10\hat{ }5 s⁻¹M⁻¹, 1.3×10\hat{ }5 s⁻¹M⁻¹, 1.4×10\hat{ }5 s⁻¹M⁻¹, more preferably about 1.5×10\hat{ }5 s⁻¹M⁻¹, 1.6×10\hat{ }5 s⁻¹M⁻¹, 1.7×10\hat{ }5 s⁻¹M⁻¹, 1.8×10\hat{ }5 s⁻¹M⁻¹, particularly preferred about 1.81×10\hat{ }5 s⁻¹M⁻¹, 1.82×10\hat{ }5 s⁻¹M⁻¹, 1.83×10\hat{ }5 s⁻¹M⁻¹, 1.84×10\hat{ }5 s⁻¹M⁻¹, or 1.85×10\hat{ }5 s⁻¹M⁻¹, and most preferred about 1.86×10\hat{ }5 s⁻¹M⁻¹, and wherein the upper limit of the on-rate constant (kₒₙ) is about 3×10\hat{ }6 s⁻¹M⁻¹, 2.9×10\hat{ }6 s⁻¹M⁻¹, 2.8×10\hat{ }6 s⁻¹M⁻¹, 2.7×10\hat{ }6 s⁻¹M⁻¹, 2.6×10\hat{ }6 s⁻¹M⁻¹, 2.5×10\hat{ }6 s⁻¹M⁻¹, 2.4×10\hat{ }6 s⁻¹M⁻¹, or 2.3×10\hat{ }6 s⁻¹M⁻¹, preferably about 2.2×10\hat{ }6 s⁻¹M⁻¹, most preferred about 2.21×10\hat{ }6 s⁻¹M⁻¹, and/or wherein the lower limit of the off-rate constant (k_{off}) is about 2.5×10\hat{ }−4 s⁻¹, 2.6×10\hat{ }−4 s⁻¹, 2.7×10\hat{ }−4 s⁻¹, 2.8×10\hat{ }−4 s⁻¹, 2.9×10\hat{ }−4 s⁻¹, 3×10\hat{ }−4 s⁻¹ preferably about 3.05×10\hat{ }−4 s⁻¹, 3.06×10\hat{ }−4 s⁻¹, 3.07×10\hat{ }−4 s⁻¹, most preferred about 3.08×10\hat{ }−4 s⁻¹, and wherein the upper limit of the off-rate constant (k_{off}) is about 1.9×10\hat{ }−3 s⁻¹, 1.8×10\hat{ }−3 s⁻¹, 1.7×10\hat{ }−3 s⁻¹, 1.6×10\hat{ }−3 s⁻¹, (specifically 1.61×10\hat{ }−3 s⁻¹) 1.5×10\hat{ }−3 s⁻¹, 1.4×10\hat{ }−3 s⁻¹, 1.3×10\hat{ }−3 s⁻¹, (specifically 1.27×10\hat{ }−3 s⁻¹) 1.2×10\hat{ }−3 s⁻¹, 1.1×10\hat{ }−3 s⁻¹, 9×10\hat{ }−4, 8×10\hat{ }−4, or 7×10\hat{ }−4, preferably wherein the values are obtained by a SPR Biacore binding kinetic measurement (preferably on samples comprising purified sdAbs).

Accordingly, in the context of the present invention, an anti-IL6 sdAb can preferably comprise an on-rate constant (kₒₙ) of about 1.86×10\hat{ }5 s⁻¹M⁻¹-2.21×10\hat{ }6 s⁻¹M⁻¹ and preferably an off-rate constant (k_{off}) of about 3.08×10\hat{ }−4 s⁻¹-1.61×10\hat{ }−3 s⁻¹, preferably wherein the values are obtained by a SPR Biacore binding kinetic measurement (preferably on samples comprising purified sdAbs).

Accordingly, the present invention provides for an anti-interleukin 6 (IL6) single-domain antibody (sdAb) comprising an on-rate constant (kₒₙ) of about 1.1×10\hat{ }5 s⁻¹M⁻¹-3×10\hat{ }6 s⁻¹M⁻¹ and an off-rate constant (k_{off}) of about 2.5×10\hat{ }−4 s⁻¹-2×10\hat{ }−3 s⁻¹, preferably wherein said values are obtained by an SPR Biacore kinetic measurement, more preferably wherein said sdAb comprises an IC50 of about 1 nM or more than about 1 nM, and preferably wherein said sdAb is capable of detecting and/or quantifying IL6 at a concentration of about 45 fM or less to about 4.5 pM or more.

As is further detailed herein above and below, the present invention provides for anti-IL6 sdAbs that are, inter alia, particularly useful in in vitro or in vivo diagnostic applications or for continuous monitoring of IL6 levels. Such anti-IL6 sdAbs may be any of the herein provided sdAbs. Preferably, the anti-IL6 sdAbs (that are particularly useful in vitro or in vivo diagnostic applications or continuous monitoring of IL6 levels such as, for example biosensors which can be used either in vitro or in vivo, i.e. being partially or fully implanted into a subject) comprise an on-rate constant (kₒₙ) of about 2×10\hat{ }5 s⁻¹M⁻¹-1×10\hat{ }6 s⁻¹M⁻¹, more preferably of about 3×10\hat{ }5 s⁻¹M⁻¹-9×10\hat{ }5 s⁻¹M⁻¹, even more preferably of about 4×10\hat{ }5 s⁻¹M⁻¹-9×10\hat{ }5 s⁻¹M⁻¹, even more preferably of about 5×10\hat{ }5 s⁻¹M⁻¹-9×10\hat{ }5 s⁻¹M⁻¹, even more preferably of about 6×10\hat{ }5 s⁻¹M⁻¹-9×10\hat{ }5 s⁻¹M⁻¹, even more preferably of about 7×10\hat{ }5 s⁻¹M⁻¹-9×10\hat{ }5 s⁻¹M⁻¹, even more preferably of about 7.1×10\hat{ }5 s⁻¹M⁻¹-8.9×10\hat{ }5 s⁻¹M⁻¹, even more preferably of about 7.2×10\hat{ }5 s⁻¹M⁻¹-8.8×10\hat{ }5 s⁻¹M⁻¹, even more preferably of about 7.3×10\hat{ }5 s⁻¹M⁻¹-8.7×10\hat{ }5 s⁻¹M⁻¹, most preferably of about 7.33×10\hat{ }5 s⁻¹M⁻¹-8.64×10\hat{ }5 s⁻¹M⁻¹. In this context, the on-rate constant (kₒₙ) values are preferably determined using an SPR Biacore measurement (preferably on samples comprising purified sdAbs). As is further detailed in the enclosed examples, anti-IL6 sdAbs SIL-2D8, SIL-2F5, SIL-2D1, SIL-3B6, SIL-2E4, SIL-3E7 and SIL-3B7 are particularly useful for in vitro or in vivo diagnostic applications or continuous monitoring of IL6 levels (such as biosensors which can be used either in vitro or in vivo, i.e. being partially or fully implanted into a subject). In this context SIL-2D1 and SIL-3E7 may be especially useful as they cover a surprisingly broad inflammation range from baseline to severe IL6 concentrations, comprise a surprisingly high affinity towards IL6, and comprise a surprisingly high IC50 (see inter alia Table 13 and the respective examples).

As is further detailed herein below and above, the present invention further provides for anti-IL6 sdAbs that are, inter alia, particularly useful in in vitro or in vivo diagnostic applications or for continuous monitoring of IL6 levels. Such anti-IL6 sdAbs may be any of the herein provided sdAbs. Preferably, the anti-IL6 sdAbs (that are particularly useful in in vitro or in vivo diagnostic applications or for continuous monitoring of IL6 levels) comprise an off-rate constant (k_{off}) of about 4×10\hat{ }−4 s⁻¹-1×10\hat{ }−3 s⁻¹, more preferably of about 4×10\hat{ }−4 s⁻¹-9×10\hat{ }−4 s⁻¹, even more preferably of about 4×10\hat{ }−4 s⁻¹-8×10\hat{ }−4 s⁻¹, even more preferably of about 5×10\hat{ }−4 s⁻¹-7×10\hat{ }−4 s⁻¹, even more preferably of about 5.1×10\hat{ }−4 s⁻¹-6.9×10\hat{ }−4 s⁻¹, even more preferably of about 5.2×10\hat{ }−4 s⁻¹-6.8×10\hat{ }−4 s⁻¹, even more preferably of about 5.3×10\hat{ }−4 s⁻¹-6.7×10\hat{ }−4 s⁻¹, most preferably of about 5.35×10\hat{ }−4 s⁻¹-6.71×10\hat{ }−4 s⁻¹. In this context, the off-rate constant (k_{off}) values are preferably determined using an SPR Biacore measurement (preferably on samples comprising purified sdAbs).

As is further detailed in the enclosed examples, anti-IL6 sdAbs SIL-3E7, SIL-2D1, SIL-2D8, SIL-2F5, SIL-3B6, and SIL-2E4 are particularly useful for in vitro or in vivo diagnostic applications or for continuous monitoring of IL6 levels.

Anti-IL6 sdAbs SIL-2D8 and SIL-2F5 may be particularly useful for in vitro or in vivo diagnostic applications or for continuous monitoring of IL6 levels. Accordingly, the present invention provides for anti-IL6 sdAbs (that are particularly useful in in vitro or in vivo diagnostic applications or for continuous monitoring of IL6 levels) that preferably comprise an off-rate constant (k_{off}) of about 6×10\hat{ }−4 s⁻¹-7×10\hat{ }−4 s⁻¹, more preferably of about 6.1×10\hat{ }−4 s⁻¹-6.9×10\hat{ }−4 s⁻¹, even more preferably of about 6.2×10\hat{ }−4 s⁻¹-6.8×10\hat{ }−4 s⁻¹, even more preferably of about 6.3×10\hat{ }−4 s⁻¹-6.8×10\hat{ }−4 s⁻¹, even more preferably of about 6.4×10\hat{ }−4 s⁻¹-6.8×10\hat{ }−4 s⁻¹, even more preferably of about 6.5×10\hat{ }−4 s⁻¹-6.8×10\hat{ }−4 s⁻¹, most preferably of about 6.53×10\hat{ }−4 s⁻¹-6.71×10\hat{ }−4 s⁻¹.

Accordingly, the present invention provides for anti-IL6 sdAbs (that are particularly useful in in vitro or in vivo diagnostic applications or for continuous monitoring of IL6 levels) that comprise an off-rate constant (k_{off}) of about 4×10\hat{ }−4 s⁻¹-1×10\hat{ }−3 s⁻¹, preferably of about 4×10\hat{ }−4 s⁻¹-9×10\hat{ }−4 s⁻¹, more preferably of about 4×10\hat{ }−4 s⁻¹-8×10\hat{ }−4 s⁻¹, even more preferably of about 5×10\hat{ }−4 s⁻¹-7×10\hat{ }−4 s⁻¹, even more preferably of about 5.1×10\hat{ }−4 s⁻¹-6.9×10\hat{ }−4 s⁻¹, even more preferably of about 5.2×10\hat{ }−4 s⁻¹-6.8×10\hat{ }−4 s⁻¹, even more preferably of about 5.3×10\hat{ }−4 s⁻¹-6.7×10\hat{ }−4 s⁻¹, most preferably of about 5.35×10\hat{ }−4 s⁻¹-6.71×10\hat{ }−4 s⁻¹.

In this context, the off-rate constant (k_{off}) values are preferably determined using an SPR Biacore measurement (preferably on samples comprising purified sdAbs).

As is further detailed herein below and above, the present invention further provides for anti-IL6 sdAbs that are, inter alia, particularly useful in therapeutic applications, such as for use in the treatment of a disease associated with an inflammation as provided herein or the corresponding method of treatment as provided herein. Such anti-IL6 sdAbs may be any of the herein provided sdAbs. Preferably, the anti-IL6 sdAbs (that are particularly useful in therapeutic applications) comprise an off-rate constant (k_{off}) of about 6×10\hat{ }−4 s⁻¹-7×10\hat{ }−4 s⁻¹, more preferably of about 6.1×10\hat{ }−4 s⁻¹-6.9×10\hat{ }−4 s⁻¹, even more preferably of about 6.2×10\hat{ }−4 s⁻¹-6.8×10\hat{ }−4 s⁻¹, even more preferably of about 6.3×10\hat{ }−4 s⁻¹-6.8×10\hat{ }−4 s⁻¹, even more preferably of about 6.4×10\hat{ }−4 s⁻¹-6.8×10\hat{ }−4 s⁻¹, even more preferably of about 6.5×10\hat{ }−4 s⁻¹-6.8×10\hat{ }−4 s⁻¹, most preferably of about 6.53×10\hat{ }−4 s⁻¹-6.71×10\hat{ }−4 s⁻¹. In this context, the off-rate constant (k_{off}) values are preferably determined using an SPR Biacore measurement (preferably on samples comprising purified sdAbs).

Anti-IL6 sdAbs SIL-2D8 and SIL-2F5 are particularly preferred for therapeutic applications disclosed herein, such as the therapeutic uses and/or methods of treatments based on their respective k_{off} values.

In particular, the present invention provides for anti-IL6 sdAb SIL-3B7 (or variants thereof), comprising an on-rate constant (kₒₙ) of about 1.86×10\hat{ }5 s⁻¹M⁻¹ and/or an off-rate constant (k_{off}) of about 1.27×10\hat{ }−3 s⁻¹.

In particular, the present invention provides for anti-IL6 sdAb SIL-2E4 (or variants thereof), comprising an on-rate constant (kₒₙ) of about 7.33×10\hat{ }5 s⁻¹M⁻¹ and/or an off-rate constant (k_{off}) of about 5.52×10\hat{ }−4 s⁻¹.

In particular, the present invention provides for anti-IL6 sdAb SIL-2D1 (or variants thereof), comprising an on-rate constant (kₒₙ) of about 8.37×10\hat{ }5 s⁻¹M⁻¹ and/or an off-rate constant (k_{off}) of about 3.08×10\hat{ }−4 s⁻¹.

In particular, the present invention provides for anti-IL6 sdAb SIL-3B6 (or variants thereof), comprising an on-rate constant (kₒₙ) of about 8.45×10\hat{ }5 s⁻¹M⁻¹ and/or an off-rate constant (k_{off}) of about 5.35×10\hat{ }−4 s⁻¹.

In particular, the present invention provides for anti-IL6 sdAb SIL-3E7 (or variants thereof), comprising an on-rate constant (kₒₙ) of about 8.64×10\hat{ }5 s⁻¹M⁻¹ and/or an off-rate constant (k_{off}) of about 1.61×10\hat{ }−3 s⁻¹.

In particular, the present invention provides for anti-IL6 sdAb SIL-2D8 (or variants thereof), comprising an on-rate constant (kₒₙ) of about 1.82×10\hat{ }6 s⁻¹M⁻¹ and/or an off-rate constant (k_{off}) of about 6.71×10\hat{ }−4 s⁻¹.

In particular, the present invention provides for anti-IL6 sdAb SIL-2F5 (or variants thereof), comprising an on-rate constant (kₒₙ) of about 2.21×10\hat{ }6 s⁻¹M⁻¹ and/or an off-rate constant (k_{off}) of about 6.53×10\hat{ }−4 s⁻¹.

In this context, the on-rate constant (kₒₙ) values and/or the off-rate constant (k_{off}) values are preferably determined using an SPR Biacore measurement.

The herein provided anti-IL6 sdAbs SIL-2D1, SIL-2D8, SIL-2E4, SIL-2E7, SIL-2F5, SIL-3B6, SIL-3B7, SIL-3E7 may in the context of the present invention be defined as detailed herein above or below, by any function characterization detailed in, inter alia, Table 13, and/or by any of the sequences (e.g., CDRs, CDRs and FRs, full length sequences) summarized in Table 25 and provided in the enclosed sequence listing. As is also mentioned herein, such sdAbs (or the respective CDR, FR, or full length sequences) may comprise up to five amino acid substitutions. Accordingly, they may comprise about 1, about 2, about 3, about 4, or about 5 amino acid substitutions.

It is understood that the anti-interleukin 6 (IL6) single-domain antibody (sdAb) of the invention can comprise any of the herein disclosed on-rate constant (kₒₙ) and/or any of the herein disclosed off-rate constant (k_{off}). Any combinations of the disclosed on-rate constant (kₒₙ) and off-rate constant (k_{off}) are envisaged.

As explained further below, the anti-interleukin 6 (IL6) single-domain antibody (sdAb) of the invention can comprise an IC50 of about 1 nM or more than about 1 nM, and/or can detect and/or quantify IL6 concentrations of about or less than about 4.5 pM. The corresponding explanations and definitions apply mutatis mutandis here.

As is detailed in the herein provided non-limiting examples instead of determining or quantifying the binding affinities (such as kₒₙ, k_{off}, and K_{D} values) using SPR Biacore measurements (as can illustratively be seen in Table 10), such values can also be determined by other means, such as by Octet BLI kinetic measurements (as can, inter alia, illustratively be seen in Table 8).

Accordingly, in the context of the present invention an Interleukin-6 (IL6) single-domain antibody (sdAb) can comprise an on-rate constant (kₒₙ) of about 1.1×10\hat{ }5 s⁻¹M⁻¹-3×10\hat{ }6 s⁻¹M⁻¹ and/or an off-rate constant (k_{off}) of about or more than about 1.1×10\hat{ }−4 s⁻¹, preferably wherein said values are obtained by an Octet BLI kinetic measurement (preferably on samples comprising purified sdAbs).

Accordingly, the present invention relates to an anti-interleukin 6 (IL6) single-domain antibody (sdAb) preferably comprising an on-rate constant (kₒₙ) of about 1.1×10\hat{ }5 s⁻¹M⁻¹-3×10\hat{ }6 s⁻¹M⁻¹.

Preferably the lower limit is about 1.2×10\hat{ }5 s⁻¹M⁻¹, 1.3×10\hat{ }5 s⁻¹M⁻¹, 1.4×10\hat{ }5 s⁻¹M⁻¹, more preferably about 2×10\hat{ }5 s⁻¹M⁻¹, 3×10\hat{ }5 s⁻¹M⁻¹, 4×10\hat{ }5 s⁻¹M⁻¹, 5×10\hat{ }5 s⁻¹M⁻¹, 6×10\hat{ }5 s⁻¹M⁻¹, particularly preferred about 6.1×10\hat{ }5 s⁻¹M⁻¹, and most preferred about 6.11×10\hat{ }5 s⁻¹M⁻¹.

More preferably, the upper limit is about 3×10\hat{ }6 s⁻¹M⁻¹, 2.9×10\hat{ }6 s⁻¹M⁻¹, 2.8×10\hat{ }6 s⁻¹M⁻¹, 2.7×10\hat{ }6 s⁻¹M⁻¹, 2.6×10\hat{ }6 s⁻¹M⁻¹, 2.5×10\hat{ }6 s⁻¹M⁻¹, even more preferably about 2.4×10\hat{ }6 s⁻¹M⁻¹, most preferred about 2.33×10\hat{ }6 s⁻¹M⁻¹.

In this context, the on-rate constant (kₒₙ) values are preferably determined using an Octet BLI kinetic measurement (preferably on samples comprising purified sdAbs).

Accordingly, the present invention relates to an anti-interleukin 6 (IL6) single-domain antibody (sdAb) preferably comprising an off-rate constant (k_{off}) of about or more than about 1.1×10\hat{ }−4 s⁻¹.

Preferably the lower limit is about 1.1×10\hat{ }−4 s⁻¹, 1.11×10\hat{ }−4 s⁻¹, 1.12×10\hat{ }−4 s⁻¹, 1.13×10\hat{ }−4 s⁻¹, 1.14×10\hat{ }−4 s⁻¹, 1.15×10\hat{ }−4 s⁻¹, 1.16×10\hat{ }−4 s⁻¹, 1.17×10\hat{ }−4 s⁻¹, 1.18×10\hat{ }−4 s⁻¹, 1.19×10\hat{ }−4 s⁻¹, 1.2×10\hat{ }−4 s⁻¹, 1.21×10\hat{ }−4 s⁻¹, 1.22×10\hat{ }−4 s⁻¹, 1.23×10\hat{ }−4 s⁻¹, more preferably about 1.24×10\hat{ }−4 s⁻¹, most preferred about 1.25×10\hat{ }−4 s⁻¹.

In this context, the upper limit of the off-rate constant (k_{off}) of the herein provided anti-IL6 sdAbs is not particularly limited.

Nonetheless, the the off-rate constant (k_{off}) of the herein provided anti-IL6 sdAbs may preferably comprise an upper limit of about 1×10\hat{ }−1 s⁻¹, 9×10\hat{ }−2 s⁻¹, 8×10\hat{ }−2 s⁻¹, 7×10\hat{ }−2 s⁻¹, 6×10\hat{ }−2 s⁻¹, 5×10\hat{ }−2 s⁻¹, 4×10\hat{ }−2 s⁻¹, more preferably of about 3.9×10\hat{ }−2 s⁻¹, 3.8×10\hat{ }−2 s⁻¹, 3.7×10\hat{ }−2 s⁻¹, even more preferably of about 3.6×10\hat{ }−2 s⁻¹, 3.59×10\hat{ }−2 s⁻¹, 3.58×10\hat{ }−2 s⁻¹, and most preferably of about 3.57×10\hat{ }−2 s⁻¹.

In this context, the off-rate constant (k_{off}) values are preferably determined using an Octet BLI kinetic measurement (preferably on samples comprising purified sdAbs).

Accordingly, the present invention relates to an anti-interleukin 6 (IL6) single-domain antibody (sdAb) preferably comprising an on-rate constant (kₒₙ) of about 1.1×10\hat{ }5 s⁻¹M⁻¹-3×10\hat{ }6 s⁻¹M⁻¹ and/or an off-rate constant (k_{off}) of about or more than about 1.1×10\hat{ }−4 s⁻¹-1×10\hat{ }−1 s⁻¹, preferably wherein the lower limit of the on-rate constant (kₒₙ) is about 1.2×10\hat{ }5 s⁻¹M⁻¹, 1.3×10\hat{ }5 s⁻¹M⁻¹, 1.4×10\hat{ }5 s⁻¹M⁻¹, more preferably about 2×10\hat{ }5 s⁻¹M⁻¹, 3×10\hat{ }5 s⁻¹M⁻¹, 4×10\hat{ }5 s⁻¹M⁻¹, 5×10\hat{ }5 s⁻¹M⁻¹, 6×10\hat{ }5 s⁻¹M⁻¹, particularly preferred about 6.1×10\hat{ }5 s⁻¹M⁻¹, and most preferred about 6.11×10\hat{ }5 s⁻¹M⁻¹, and wherein the upper limit of the on-rate constant (kₒₙ) is about 3×10\hat{ }6 s⁻¹M⁻¹, 2.9×10\hat{ }6 s⁻¹M⁻¹, 2.8×10\hat{ }6 s⁻¹M⁻¹, 2.7×10\hat{ }6 s⁻¹M⁻¹, 2.6×10\hat{ }6 s⁻¹M⁻¹, 2.5×10\hat{ }6 s⁻¹M⁻¹, preferably about 2.4×10\hat{ }6 s⁻¹M⁻¹, most preferred about 2.33×10\hat{ }6 s⁻¹M⁻¹, and/or wherein the lower limit of the off-rate constant (k_{off}) is about 1.1×10\hat{ }−4 s⁻¹, 1.11×10\hat{ }−4 s⁻¹, 1.12×10\hat{ }−4 s⁻¹, 1.13×10\hat{ }−4 s⁻¹, 1.14×10\hat{ }−4 s⁻¹, 1.15×10\hat{ }−4 s⁻¹, 1.16×10\hat{ }−4 s⁻¹, 1.17×10\hat{ }−4 s⁻¹, 1.18×10\hat{ }−4 s⁻¹, 1.19×10\hat{ }−4 s⁻¹, 1.2×10\hat{ }−4 s⁻¹, 1.21×10\hat{ }−4 s⁻¹, 1.22×10\hat{ }−4 s⁻¹, 1.23×10\hat{ }−4 s⁻¹, preferably about 1.24×10\hat{ }−4 s⁻¹, most preferred about 1.25×10\hat{ }−4 s⁻¹, and wherein the upper limit of the off-rate constant (k_{off}) is about 1×10\hat{ }−1 s⁻¹, 9×10\hat{ }−2 s⁻¹, 8×10\hat{ }−2 s⁻¹, 7×10\hat{ }−2 s⁻¹, 6×10\hat{ }−2 s⁻¹, 5×10\hat{ }−2 s⁻¹, 4×10\hat{ }−2 s⁻¹, preferably of about 3.9×10\hat{ }−2 s⁻¹, 3.8×10\hat{ }−2 s⁻¹, 3.7×10\hat{ }−2 s⁻¹, even more preferably of about 3.6×10\hat{ }−2 s⁻¹, 3.59×10\hat{ }−2 s⁻¹, 3.58×10\hat{ }−2 s⁻¹, and most preferably of about 3.57×10\hat{ }−2 s⁻¹, preferably wherein the values are determined using an Octet BLI kinetic measurement (preferably on samples comprising purified sdAbs).

In the sense of the present invention, an anti-IL6 sdAb can comprise a kₒₙ of about 6.3×10∝-1.3×10\hat{ }7 and a k_{off} of about 1.2×10\hat{ }−6-0.31. An anti-IL6 sdAb can comprise a kₒₙ of about 0.15×10\hat{ }6-2.25×10\hat{ }6 and k_{off} of about 0.0002-0.002.

The invention provides in one aspect an anti-interleukin 6 (IL6) single-domain antibody (sdAb) comprising an on-rate constant (kₒₙ) of about 1.86×10\hat{ }5-2.33×10\hat{ }6 and an off-rate constant (k_{off}) of about 1.25×10\hat{ }−4-3.57×10\hat{ }−2.

The invention provides in one aspect an anti-interleukin 6 (IL6) single-domain antibody (sdAb) comprising an on-rate constant (kₒₙ) of about 1.86×10\hat{ }5-2.21×10\hat{ }6 and an off-rate constant (k_{off}) of about 3.08×10\hat{ }−4-1.16×10\hat{ }−3.

The invention provides in one aspect an anti-interleukin 6 (IL6) single-domain antibody (sdAb) comprising an on-rate constant (kₒₙ) of about 6.11×10\hat{ }5-2.33×10\hat{ }6 and an off-rate constant (k_{off}) of about 1.25×10\hat{ }−4-3.57×10\hat{ }−2.

An anti-IL6 sdAb can comprise a kₒₙ of about 1.86×10\hat{ }5-1.82×10\hat{ }6 and k_{off} of about 0.000308-0.00161 and optionally a K_{D} of about 3×10\hat{ }−10 M-6.8×10\hat{ }−9 M. An anti-IL6 sdAb can have a kₒₙ of about 1.86×10\hat{ }5-1.82×10\hat{ }6 and k_{off} of about 0.000308-0.00161, wherein the values are obtained by a SPR Biacore binding kinetic measurement. An anti-IL6 sdAb can have a kₒₙ of about 1.86×10\hat{ }5-1.82×10\hat{ }6 and k_{off} of about 0.000308-0.00161 and a K_{D} of about 3×10\hat{ }−10-6.8×10\hat{ }−9 M, wherein the values are obtained by a SPR Biacore binding kinetic measurement.

An anti-IL6 sdAb can comprise a kₒₙ of about 1.20×10\hat{ }6-1.30×10\hat{ }7 and k_{off} of about 1.20×10\hat{ }−6-7.40×10\hat{ }−2, and optionally a K_{D} of about 2.80×10\hat{ }−13 M-1.40×10\hat{ }−8 M. An anti-IL6 sdAb can comprise a kₒₙ of about 1.20×10\hat{ }6-1.30×10\hat{ }7 and k_{off} of about 1.20×10\hat{ }−6-7.40×10\hat{ }−2, wherein the values are obtained by an Octet BLI binding kinetic measurement. An anti-IL6 sdAb can comprise a kₒₙ of about 1.20×10\hat{ }6-1.30×10\hat{ }7 and k_{off} of about 1.20×10\hat{ }−6-7.40×10\hat{ }−2, and a K_{D} of about 2.80×10\hat{ }−13 M-1.40×10\hat{ }−8 M, wherein the values are obtained by an Octet BLI binding kinetic measurement.

The kₒₙ and k_{off} values of a sdAb of the present invention can vary depending on the sdAb, the method of measurement and the measurement setup used. Accordingly, the skilled person is aware that a sdAb of the present invention may not embody strict kₒₙ and k_{off} values, but these may be in a range defined by the above parameters. Accordingly, it is possible to define a certain range of a kₒₙ and k_{off} value by repeating measurements and calculating a standard deviation (SD). For example, a SD can be based on repeated measurements, deviations reported in the prior art, and/or minimum and maximum values obtained for SDs from repeated measurements and SDs reported in the prior art. Different means to arrive at standard deviations for the sdAbs of the present application are described in detail in the appended examples.

Accordingly, the invention provides in one aspect an anti-interleukin 6 (IL6) single-domain antibody (sdAb) comprising an on-rate constant (kₒₙ) of about 1.86×10\hat{ }5-2.21×10\hat{ }6±1.73×10\hat{ }5-2.05×10\hat{ }6 and an off-rate constant (k_{off}) of about 3.08×10\hat{ }−4-1.61×10\hat{ }−3±4.39×10\hat{ }−4-2.30×10\hat{ }−3 and optionally a K_{D} of about 0.30-6.80, optionally wherein the values are obtained by a SPR Biacore binding kinetic measurement, preferably wherein the sdAb is defined in any one of SEQ ID NOs: 1, 9, 17, 33, 41, 49, 57.

Terms like “on-rate constant (kₒₙ) of about 1.86×10\hat{ }5-2.21×10\hat{ }6±1.73×10\hat{ }5-2.05×10\hat{ }6 and an off-rate constant (k_{off}) of about 3.08×10\hat{ }−4-1.61×10\hat{ }−3±4.39×10\hat{ }−4-2.30×10\hat{ }−3” as disclosed herein are to be understood to relate to “on-rate constant (kₒₙ) of about between (from) 1.86×10\hat{ }5 to 2.21×10\hat{ }6 (wherein the “±1.73×10\hat{ }5-2.05×10\hat{ }6” is the range of the SD of “between (from) 1.73×10\hat{ }5 to 2.05×10\hat{ }6” associated with the indicated range of the on-rate constant (kₒₙ)) and an off-rate constant (k_{off}) of between (from) 3.08×10\hat{ }−4 to 1.61×10\hat{ }−3±4.39×10\hat{ }−4-2.30×10\hat{ }−3 (wherein the “±4.39×10\hat{ }−4-2.30×10\hat{ }−3” is the range of the SD of “between (from) 4.39×10\hat{ }−4 to 2.30×10\hat{ }−3” associated with the indicated range of the off-rate constant (k_{off})”. This explanation applies to all similar terms disclosed herein.

In some embodiments, the invention provides an anti-interleukin 6 (IL6) single-domain antibody (sdAb) comprising an on-rate constant (kₒₙ) of about 1.86×10\hat{ }5-2.21×10\hat{ }6±1.12×10\hat{ }3-1.69×10\hat{ }4 and an off-rate constant (k_{off}) of about 3.08×10\hat{ }−4-1.61×10\hat{ }−3±1.86×10\hat{ }−6-2.94×10\hat{ }−5 and optionally a K_{D} of about 0.30-6.80, optionally wherein the values are obtained by a SPR Biacore binding kinetic measurement, preferably wherein the sdAb is defined in any one of SEQ ID NOs: 1, 9, 17, 33, 41, 49, 57.

In some embodiments, the invention provides an anti-interleukin 6 (IL6) single-domain antibody (sdAb) comprising an on-rate constant (kₒₙ) of about 1.82×10\hat{ }6±1.69×10\hat{ }6 and an off-rate constant (k_{off}) of about 6.71×10\hat{ }−4±9.57×10\hat{ }−4 and optionally a K_{D} of about 0.37, optionally wherein the values are obtained by a SPR Biacore binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 1.82×10\hat{ }6±4.02×10\hat{ }3 and an off-rate constant (k_{off}) of about 6.71×10\hat{ }−4±2.08×10\hat{ }−6 and optionally a K_{D} of about 0.37, optionally wherein the values are obtained by a SPR Biacore binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 2.21×10\hat{ }6±2.05×10\hat{ }6 and an off-rate constant (k_{off}) of about 6.53×10\hat{ }−4±9.31×10\hat{ }−4 and optionally a K_{D} of about 0.30, optionally wherein the values are obtained by a SPR Biacore binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 2.21×10\hat{ }6±4.16×10\hat{ }3 and an off-rate constant (k_{off}) of about 6.53×10\hat{ }−4±1.86×10\hat{ }−6 and optionally a K_{D} of about 0.30, optionally wherein the values are obtained by a SPR Biacore binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 8.37×10\hat{ }5±7.77×10\hat{ }5 and an off-rate constant (k_{off}) of about 3.08×10\hat{ }−4±4.39×10\hat{ }−4 and optionally a K_{D} of about 0.37, optionally wherein the values are obtained by a SPR Biacore binding kinetic measurement, preferably wherein the sdAb is defined in SEQ ID NO: 1.

In some embodiments, the invention provides an anti-interleukin 6 (IL6) single-domain antibody (sdAb) comprising an on-rate constant (kₒₙ) of about 8.37×10\hat{ }5±2.91×10\hat{ }3 and an off-rate constant (k_{off}) of about 3.08×10\hat{ }−4±4.47×10\hat{ }−6 and optionally a K_{D} of about 0.37, optionally wherein the values are obtained by a SPR Biacore binding kinetic measurement, preferably wherein the sdAb is defined in SEQ ID NO: 1.

In some embodiments, the invention provides an anti-interleukin 6 (IL6) single-domain antibody (sdAb) comprising an on-rate constant (kₒₙ) of about 8.45×10\hat{ }5±7.85×10\hat{ }5 and an off-rate constant (k_{off}) of about 5.35×10\hat{ }−4±7.63×10\hat{ }−4 and optionally a K_{D} of about 0.63, optionally wherein the values are obtained by a SPR Biacore binding kinetic measurement, preferably wherein the sdAb is defined in SEQ ID NO: 41.

In some embodiments, the invention provides an anti-interleukin 6 (IL6) single-domain antibody (sdAb) comprising an on-rate constant (kₒₙ) of about 8.45×10\hat{ }5±2.46×10\hat{ }3 and an off-rate constant (k_{off}) of about 5.35×10\hat{ }−4±2.91×10\hat{ }−6 and optionally a K_{D} of about 0.63, optionally wherein the values are obtained by a SPR Biacore binding kinetic measurement, preferably wherein the sdAb is defined in SEQ ID NO: 41.

In some embodiments, the invention provides an anti-interleukin 6 (IL6) single-domain antibody (sdAb) comprising an on-rate constant (kₒₙ) of about 7.33×10\hat{ }5±6.81×10\hat{ }5 and an off-rate constant (k_{off}) of about 5.52×10\hat{ }−4±7.87×10\hat{ }−4 and optionally a K_{D} of about 0.75, optionally wherein the values are obtained by a SPR Biacore binding kinetic measurement, preferably wherein the sdAb is defined in SEQ ID NO: 17.

In some embodiments, the invention provides an anti-interleukin 6 (IL6) single-domain antibody (sdAb) comprising an on-rate constant (kₒₙ) of about 7.33×10\hat{ }5±2.46×10\hat{ }3 and an off-rate constant (k_{off}) of about 5.52×10\hat{ }−4±4.25×10\hat{ }−6 and optionally a K_{D} of about 0.75, optionally wherein the values are obtained by a SPR Biacore binding kinetic measurement, preferably wherein the sdAb is defined in SEQ ID NO: 17.

In some embodiments, the invention provides an anti-interleukin 6 (IL6) single-domain antibody (sdAb) comprising an on-rate constant (kₒₙ) of about 8.64×10\hat{ }5±8.02×10\hat{ }5 and an off-rate constant (k_{off}) of about 1.61×10\hat{ }−3±2.30×10\hat{ }−3 and optionally a K_{D} of about 1.9, optionally wherein the values are obtained by a SPR Biacore binding kinetic measurement, preferably wherein the sdAb is defined in SEQ ID NO: 57.

In some embodiments, the invention provides an anti-interleukin 6 (IL6) single-domain antibody (sdAb) comprising an on-rate constant (kₒₙ) of about 8.64×10\hat{ }5±1.69×10\hat{ }4 and an off-rate constant (k_{off}) of about 1.61×10\hat{ }−3±2.94×10\hat{ }−5 and optionally a K_{D} of about 1.9, optionally wherein the values are obtained by a SPR Biacore binding kinetic measurement, preferably wherein the sdAb is defined in SEQ ID NO: 57.

In some embodiments, the invention provides an anti-interleukin 6 (IL6) single-domain antibody (sdAb) comprising an on-rate constant (kₒₙ) of about 1.86×10\hat{ }5±1.73×10\hat{ }5 and an off-rate constant (k_{off}) of about 1.27×10\hat{ }−3±1.81×10\hat{ }−3 and optionally a K_{D} of about 6.8, optionally wherein the values are obtained by a SPR Biacore binding kinetic measurement, preferably wherein the sdAb is defined in SEQ ID NO: 49.

In some embodiments, the invention provides an anti-interleukin 6 (IL6) single-domain antibody (sdAb) comprising an on-rate constant (kₒₙ) of about 1.86×10\hat{ }5±1.12×10\hat{ }3 and an off-rate constant (k_{off}) of about 1.27×10\hat{ }−3±7.4×10\hat{ }−6 and optionally a K_{D} of about 6.8, optionally wherein the values are obtained by a SPR Biacore binding kinetic measurement, preferably wherein the sdAb is defined in SEQ ID NO: 49.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 1.86×10\hat{ }5-2.33×10\hat{ }6 and an off-rate constant (k_{off}) of about 1.25×10\hat{ }−4-3.57×10\hat{ }−2, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by a SPR Biacore binding kinetic measurement.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 1.86×10\hat{ }5-2.21×10\hat{ }6 and an off-rate constant (k_{off}) of about 3.08×10\hat{ }−4-1.16×10\hat{ }−3, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by a SPR Biacore binding kinetic measurement.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 6.11×10\hat{ }5-2.33×10\hat{ }6 and an off-rate constant (k_{off}) of about 1.25×10\hat{ }−4-3.57×10\hat{ }−2, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by a SPR Biacore binding kinetic measurement.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 1.86×10\hat{ }5-2.21×10\hat{ }6±1.73×10\hat{ }5-2.05×10\hat{ }6 and an off-rate constant (k_{off}) of about 3.08×10\hat{ }−4-1.61×10\hat{ }−3 4.39×10\hat{ }−4-2.30×10\hat{ }−3 and optionally a K_{D} of about 0.30-6.80, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by a SPR Biacore binding kinetic measurement.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 1.86×10\hat{ }5-2.21×10\hat{ }6±1.12×10\hat{ }3-1.69×10\hat{ }4 and an off-rate constant (k_{off}) of about 3.08×10\hat{ }−4-1.61×10\hat{ }−3±1.86×10\hat{ }−6-2.94×10\hat{ }−5 and optionally a K_{D} of about 0.30-6.80, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 8.37×10\hat{ }5±7.77×10\hat{ }5 and an off-rate constant (k_{off}) of about 3.08×10\hat{ }−4±4.39×10\hat{ }−4 and optionally a K_{D} of about 0.37, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 8.37×10\hat{ }5±2.91×10\hat{ }3 and an off-rate constant (k_{off}) of about 3.08×10\hat{ }−4±4.47×10\hat{ }−6 and optionally a K_{D} of about 0.37, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 1.82×10\hat{ }6±1.69×10\hat{ }6 and an off-rate constant (k_{off}) of about 6.71×10\hat{ }−4±9.57×10\hat{ }−4 and optionally a K_{D} of about 0.37, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 1.82×10\hat{ }6±4.02×10\hat{ }3 and an off-rate constant (k_{off}) of about 6.71×10\hat{ }−4±2.08×10\hat{ }−6 and optionally a K_{D} of about 0.37, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 7.33×10\hat{ }5±6.81×10\hat{ }5 and an off-rate constant (k_{off}) of about 5.52×10\hat{ }−4±7.87×10\hat{ }−4 and optionally a K_{D} of about 0.75, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 7.33×10\hat{ }5±2.46×10\hat{ }3 and an off-rate constant (k_{off}) of about 5.52×10\hat{ }−4±4.25×10\hat{ }−6 and optionally a K_{D} of about 0.75, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 2.21×10\hat{ }6±2.05×10\hat{ }6 and an off-rate constant (k_{off}) of about 6.53×10\hat{ }−4±9.31×10\hat{ }−4 and optionally a K_{D} of about 0.30, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 2.21×10\hat{ }6±4.16×10\hat{ }3 and an off-rate constant (k_{off}) of about 6.53×10\hat{ }−4±1.86×10\hat{ }−6 and optionally a K_{D} of about 0.30, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 43 or a variant thereof having up to two amino acid substitutions, CDR2 comprising SEQ ID NO: 45 or a variant thereof having up to two amino acid substitutions, CDR3 comprising SEQ ID NO: 47 or a variant thereof having up to five amino acid substitutions, comprising an on-rate constant (kₒₙ) of about 8.45×10\hat{ }5±7.85×10\hat{ }5 and an off-rate constant (k_{off}) of about 5.35×10\hat{ }−4±7.63×10\hat{ }−4 and optionally a K_{D} of about 0.63, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 43 or a variant thereof having up to two amino acid substitutions, CDR2 comprising SEQ ID NO: 45 or a variant thereof having up to two amino acid substitutions, CDR3 comprising SEQ ID NO: 47 or a variant thereof having up to five amino acid substitutions, comprising an on-rate constant (kₒₙ) of about 8.45×10\hat{ }5±2.46×10\hat{ }3 and an off-rate constant (k_{off}) of about 5.35×10\hat{ }−4±2.91×10\hat{ }−6 and optionally a K_{D} of about 0.63, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 1.86×10\hat{ }5±1.73×10\hat{ }5 and an off-rate constant (k_{off}) of about 1.27×10\hat{ }−3±1.81×10\hat{ }−3 and optionally a K_{D} of about 6.8, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 1.86×10\hat{ }5±1.12×10\hat{ }3 and an off-rate constant (k_{off}) of about 1.27×10\hat{ }−3±7.4×10\hat{ }−6 and optionally a K_{D} of about 6.8, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 8.64×10\hat{ }5±8.02×10\hat{ }5 and an off-rate constant (k_{off}) of about 1.61×10\hat{ }−3±2.30×10\hat{ }−3 and optionally a K_{D} of about 1.9, optionally wherein the values are obtained by a SPR Biacore binding kinetic measurement.

In some embodiments, the invention provides an anti-interleukin 6 (IL6) single-domain antibody (sdAb) comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 8.64×10\hat{ }5±1.69×10\hat{ }4 and an off-rate constant (k_{off}) of about 1.61×10\hat{ }−3±2.94×10\hat{ }−5 and optionally a K_{D} of about 1.9, optionally wherein the values are obtained by a SPR Biacore binding kinetic measurement.

Accordingly, the invention provides in one aspect an anti-interleukin 6 (IL6) single-domain antibody (sdAb) comprising an on-rate constant (kₒₙ) of about 6.11×10\hat{ }5-2.33×10\hat{ }6±5.67×10\hat{ }5-3.10×10\hat{ }6 and an off-rate constant (k_{off}) of about 1.25×10\hat{ }−4-3.57×10\hat{ }−2±1.78×10\hat{ }−4-5.09×10\hat{ }−2 and optionally a K_{D} of about 2.81×10\hat{ }−1-2.66×10\hat{ }1, optionally wherein the values are obtained by an Octet BLI binding kinetic measurement, preferably wherein the sdAb is defined in any one of SEQ ID NOs: 1, 9, 17, 25, 33, 41, 49, 57.

In some embodiments, the invention provides an anti-interleukin 6 (IL6) single-domain antibody (sdAb) comprising an on-rate constant (kₒₙ) of about 6.11×10\hat{ }5-2.33×10\hat{ }6±5.67×10\hat{ }5-2.16×10\hat{ }6 and an off-rate constant (k_{off}) of about 1.25×10\hat{ }−4-3.57×10\hat{ }−2±1.78×10\hat{ }−4-5.09×10\hat{ }−2 and optionally a K_{D} of about 2.81×10\hat{ }−1-2.66×10\hat{ }1, optionally wherein the values are obtained by an Octet BLI binding kinetic measurement, preferably wherein the sdAb is defined in any one of SEQ ID NOs: 1, 9, 17, 25, 33, 41, 49, 57.

In some embodiments, the invention provides an anti-interleukin 6 (IL6) single-domain antibody (sdAb) comprising an on-rate constant (kₒₙ) of about 6.11×10\hat{ }5-2.33×10\hat{ }6±2.60×10\hat{ }4-3.10×10\hat{ }6 and an off-rate constant (k_{off}) of about 1.25×10\hat{ }−4-3.57×10\hat{ }−2±6.11×10\hat{ }−5-2.32×10\hat{ }−2 and optionally a K_{D} of about 2.81×10\hat{ }−1-2.66×10\hat{ }1, optionally wherein the values are obtained by an Octet BLI binding kinetic measurement, preferably wherein the sdAb is defined in any one of SEQ ID NOs: 1, 9, 17, 25, 33, 41, 49, 57.

In some embodiments, the invention provides an anti-interleukin 6 (IL6) single-domain antibody (sdAb) comprising an on-rate constant (kₒₙ) of about 9.43×10\hat{ }6±8.76×10\hat{ }5 and an off-rate constant (k_{off}) of about 3.57×10\hat{ }−3±5.09×10\hat{ }−3 and optionally a K_{D} of about 3.78, optionally wherein the values are obtained by an Octet BLI binding kinetic measurement, preferably wherein the sdAb is defined in SEQ ID NO: 1.

In some embodiments, the invention provides an anti-interleukin 6 (IL6) single-domain antibody (sdAb) comprising an on-rate constant (kₒₙ) of about 9.43×10\hat{ }6±5.74×10\hat{ }4 and an off-rate constant (k_{off}) of about 3.57×10\hat{ }−3±8.55×10\hat{ }−5 and optionally a K_{D} of about 3.78, optionally wherein the values are obtained by an Octet BLI binding kinetic measurement, preferably wherein the sdAb is defined in SEQ ID NO: 1.

In some embodiments, the invention provides an anti-interleukin 6 (IL6) single-domain antibody (sdAb) comprising an on-rate constant (kₒₙ) of about 8.86×10\hat{ }5±9.11×10\hat{ }5 and an off-rate constant (k_{off}) of about 1.25×10\hat{ }−4±1.78×10\hat{ }−4 and optionally a K_{D} of about 2.81×10\hat{ }−1, optionally wherein the values are obtained by an Octet BLI binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 8.86×10\hat{ }5±8.23×10\hat{ }5 and an off-rate constant (k_{off}) of about 1.25×10\hat{ }−4±1.78×10\hat{ }−4 and optionally a K_{D} of about 2.81×10\hat{ }−1, optionally wherein the values are obtained by an Octet BLI binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 8.86×10\hat{ }5±9.11×10\hat{ }5 and an off-rate constant (k_{off}) of about 1.25×10\hat{ }−4±6.11×10\hat{ }−5 and optionally a K_{D} of about 2.81×10\hat{ }−1, optionally wherein the values are obtained by an Octet BLI binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 7.35×10\hat{ }5±6.83×10\hat{ }5 and an off-rate constant (k_{off}) of about 4.85×10\hat{ }−3±6.92×10\hat{ }−3 and optionally a K_{D} of about 6.60, optionally wherein the values are obtained by an Octet BLI binding kinetic measurement, preferably wherein the sdAb is defined in SEQ ID NO: 17.

In some embodiments, the invention provides an anti-interleukin 6 (IL6) single-domain antibody (sdAb) comprising an on-rate constant (kₒₙ) of about 7.35×10\hat{ }5±4.02×10\hat{ }5 and an off-rate constant (k_{off}) of about 4.85×10\hat{ }−3±9.23×10\hat{ }−5 and optionally a K_{D} of about 6.60, optionally wherein the values are obtained by an Octet BLI binding kinetic measurement, preferably wherein the sdAb is defined in SEQ ID NO: 17.

In some embodiments, the invention provides an anti-interleukin 6 (IL6) single-domain antibody (sdAb) comprising an on-rate constant (kₒₙ) of about 6.11×10\hat{ }5±5.67×10\hat{ }5 and an off-rate constant (k_{off}) of about 2.94×10\hat{ }−4±6.28×10\hat{ }−4 and optionally a K_{D} of about 4.81×10\hat{ }−1, optionally wherein the values are obtained by an Octet BLI binding kinetic measurement, preferably wherein the sdAb is defined in SEQ ID NO: 25.

In some embodiments, the invention provides an anti-interleukin 6 (IL6) single-domain antibody (sdAb) comprising an on-rate constant (kₒₙ) of about 6.11×10\hat{ }5±5.67×10\hat{ }5 and an off-rate constant (k_{off}) of about 2.94×10\hat{ }−4±4.19×10\hat{ }−4 and optionally a K_{D} of about 4.81×10\hat{ }−1, optionally wherein the values are obtained by an Octet BLI binding kinetic measurement, preferably wherein the sdAb is defined in SEQ ID NO: 25.

In some embodiments, the invention provides an anti-interleukin 6 (IL6) single-domain antibody (sdAb) comprising an on-rate constant (kₒₙ) of about 6.11×10\hat{ }5±2.65×10\hat{ }5 and an off-rate constant (k_{off}) of about 2.94×10\hat{ }−4±6.28×10\hat{ }−4 and optionally a K_{D} of about 4.81×10\hat{ }−1, optionally wherein the values are obtained by an Octet BLI binding kinetic measurement, preferably wherein the sdAb is defined in SEQ ID NO: 25.

In some embodiments, the invention provides an anti-interleukin 6 (IL6) single-domain antibody (sdAb) comprising an on-rate constant (kₒₙ) of about 1.34×10\hat{ }6±2.09×10\hat{ }6 and an off-rate constant (k_{off}) of about 1.90×10\hat{ }−4±2.71×10\hat{ }−4 and optionally a K_{D} of about 4.99×10\hat{ }−1, optionally wherein the values are obtained by an Octet BLI binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 1.34×10\hat{ }6±1.24×10\hat{ }6 and an off-rate constant (k_{off}) of about 1.90×10\hat{ }−4±2.71×10\hat{ }−4 and optionally a K_{D} of about 4.99×10\hat{ }−1, optionally wherein the values are obtained by an Octet BLI binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 1.34×10\hat{ }6±2.09×10\hat{ }6 and an off-rate constant (k_{off}) of about 1.90×10\hat{ }−4±9.85×10\hat{ }−5 and optionally a K_{D} of about 4.99×10\hat{ }−1, optionally wherein the values are obtained by an Octet BLI binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 6.35×10\hat{ }5±5.90×10\hat{ }5 and an off-rate constant (k_{off}) of about 3.75×10\hat{ }−3±5.35×10\hat{ }−3 and optionally a K_{D} of about 5.91, optionally wherein the values are obtained by an Octet BLI binding kinetic measurement, preferably wherein the sdAb is defined in SEQ ID NO: 41.

In some embodiments, the invention provides an anti-interleukin 6 (IL6) single-domain antibody (sdAb) comprising an on-rate constant (kₒₙ) of about 6.35×10\hat{ }5±2.60×10\hat{ }4 and an off-rate constant (k_{off}) of about 3.75×10\hat{ }−3±6.18×10\hat{ }−5 and optionally a K_{D} of about 5.91, optionally wherein the values are obtained by an Octet BLI binding kinetic measurement, preferably wherein the sdAb is defined in SEQ ID NO: 41.

In some embodiments, the invention provides an anti-interleukin 6 (IL6) single-domain antibody (sdAb) comprising an on-rate constant (kₒₙ) of about 2.33×10\hat{ }6±3.10×10\hat{ }6 and an off-rate constant (k_{off}) of about 3.57×10\hat{ }−2±5.09×10\hat{ }−2 and optionally a K_{D} of about 2.66×10\hat{ }1, optionally wherein the values are obtained by an Octet BLI binding kinetic measurement, preferably wherein the sdAb is defined in SEQ ID NO: 49.

In some embodiments, the invention provides an anti-interleukin 6 (IL6) single-domain antibody (sdAb) comprising an on-rate constant (kₒₙ) of about 2.33×10\hat{ }6±2.16×10\hat{ }6 and an off-rate constant (k_{off}) of about 3.57×10\hat{ }−2±5.09×10\hat{ }−2 and optionally a K_{D} of about 2.66×10\hat{ }1, optionally wherein the values are obtained by an Octet BLI binding kinetic measurement, preferably wherein the sdAb is defined in SEQ ID NO: 49.

In some embodiments, the invention provides an anti-interleukin 6 (IL6) single-domain antibody (sdAb) comprising an on-rate constant (kₒₙ) of about 2.33×10\hat{ }6±3.10×10\hat{ }6 and an off-rate constant (k_{off}) of about 3.57×10\hat{ }−2±2.32×10\hat{ }−2 and optionally a K_{D} of about 2.66×10\hat{ }1, optionally wherein the values are obtained by an Octet BLI binding kinetic measurement, preferably wherein the sdAb is defined in SEQ ID NO: 49.

In some embodiments, the invention provides an anti-interleukin 6 (IL6) single-domain antibody (sdAb) comprising an on-rate constant (kₒₙ) of about 8.02×10\hat{ }5±7.45×10\hat{ }5 and an off-rate constant (k_{off}) of about 6.1×10\hat{ }−3±8.7×10\hat{ }−3 and optionally a K_{D} of about 7.60, optionally wherein the values are obtained by an Octet BLI binding kinetic measurement, preferably wherein the sdAb is defined in SEQ ID NO: 57.

In some embodiments, the invention provides an anti-interleukin 6 (IL6) single-domain antibody (sdAb) comprising an on-rate constant (kₒₙ) of about 8.02×10\hat{ }5±4.47×10\hat{ }4 and an off-rate constant (k_{off}) of about 6.1×10\hat{ }−3±1.09×10\hat{ }−4 and optionally a K_{D} of about 7.60, optionally wherein the values are obtained by an Octet BLI binding kinetic measurement, preferably wherein the sdAb is defined in SEQ ID NO: 57.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 6.11×10\hat{ }5-2.33×10\hat{ }6±5.67×10\hat{ }5-3.10×10\hat{ }6 and an off-rate constant (k_{off}) of about 1.25×10\hat{ }−4-3.57×10\hat{ }−2±1.78×10\hat{ }−4-5.09×10\hat{ }−2 and optionally a K_{D} of about 2.81×10\hat{ }−1-2.66×10\hat{ }1, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by an Octet BLI binding kinetic measurement.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 6.11×10\hat{ }5-2.33×10\hat{ }6±5.67×10\hat{ }5-2.16×10\hat{ }6 and an off-rate constant (k_{off}) of about 1.25×10\hat{ }−4-3.57×10\hat{ }−2±1.78×10\hat{ }−4-5.09×10\hat{ }−2 and optionally a K_{D} of about 2.81×10\hat{ }−1-2.66×10\hat{ }1, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by an Octet BLI binding kinetic measurement.

In some embodiments, the invention provides an anti-interleukin 6 (IL6) single-domain antibody (sdAb) comprising a first complementary determining region (CDR1) or a variant thereof having up to two amino acid substitutions, a second CDR (CDR2) or a variant thereof having up to two amino acid substitutions and third CDR (CDR3) or a variant thereof having up to five amino acid substitutions, comprising an on-rate constant (kₒₙ) of about 6.11×10\hat{ }5-2.33×10\hat{ }6±2.60×10\hat{ }4-3.10×10\hat{ }6 and an off-rate constant (k_{off}) of about 1.25×10\hat{ }−4-3.57×10\hat{ }−2±6.11×10\hat{ }−5-2.32×10\hat{ }−2 and optionally a K_{D} of about 2.81×10\hat{ }−1-2.66×10\hat{ }1, wherein CDR1 comprises any one of SEQ ID NOs: 3, 11, 19, 27, 35, 43 or 51, wherein CDR2 comprises any one of SEQ ID NOs: 5, 13, 21, 29, 37, 45 or 53 and wherein CDR3 comprises any one of SEQ ID NOs: 7, 15, 23, 31, 39, 47 or 55, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 9.43×10\hat{ }6±8.76×10\hat{ }5 and an off-rate constant (k_{off}) of about 3.57×10\hat{ }−3±5.09×10\hat{ }−3 and optionally a K_{D} of about 3.78, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 9.43×10\hat{ }6±5.74×10\hat{ }4 and an off-rate constant (k_{off}) of about 3.57×10\hat{ }−3±8.55×10\hat{ }−5 and optionally a K_{D} of about 3.78, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 8.86×10\hat{ }5±9.11×10\hat{ }5 and an off-rate constant (k_{off}) of about 1.25×10\hat{ }−4±1.78×10\hat{ }−4 and optionally a K_{D} of about 2.81×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 8.86×10\hat{ }5±8.23×10\hat{ }5 and an off-rate constant (k_{off}) of about 1.25×10\hat{ }−4±1.78×10\hat{ }−4 and optionally a K_{D} of about 2.81×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 8.86×10\hat{ }5±9.11×10\hat{ }5 and an off-rate constant (k_{off}) of about 1.25×10\hat{ }−4±6.11×10\hat{ }−5 and optionally a K_{D} of about 2.81×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 7.35×10\hat{ }5±6.83×10\hat{ }5 and an off-rate constant (k_{off}) of about 4.85×10\hat{ }−3±6.92×10\hat{ }−3 and optionally a K_{D} of about 6.60, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 7.35×10\hat{ }5±4.02×10\hat{ }5 and an off-rate constant (k_{off}) of about 4.85×10\hat{ }−3±9.23×10\hat{ }−5 and optionally a K_{D} of about 6.60, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 6.11×10\hat{ }5±5.67×10\hat{ }5 and an off-rate constant (k_{off}) of about 2.94×10\hat{ }−4±6.28×10\hat{ }−4 and optionally a K_{D} of about 4.81×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 6.11×10\hat{ }5±5.67×10\hat{ }5 and an off-rate constant (k_{off}) of about 2.94×10\hat{ }−4±4.19×10\hat{ }−4 and optionally a K_{D} of about 4.81×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 6.11×10\hat{ }5±2.65×10\hat{ }5 and an off-rate constant (k_{off}) of about 2.94×10\hat{ }−4±6.28×10\hat{ }−4 and optionally a K_{D} of about 4.81×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 1.34×10\hat{ }6±2.09×10\hat{ }6 and an off-rate constant (k_{off}) of about 1.90×10\hat{ }−4±2.71×10\hat{ }−4 and optionally a K_{D} of about 4.99×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 1.34×10\hat{ }6±1.24×10\hat{ }6 and an off-rate constant (k_{off}) of about 1.90×10\hat{ }−4±2.71×10\hat{ }−4 and optionally a K_{D} of about 4.99×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 1.34×10\hat{ }6±2.09×10\hat{ }6 and an off-rate constant (k_{off}) of about 1.90×10\hat{ }−4±9.85×10\hat{ }−5 and optionally a K_{D} of about 4.99×10\hat{ }−1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 43 or a variant thereof having up to two amino acid substitutions, CDR2 comprising SEQ ID NO: 45 or a variant thereof having up to two amino acid substitutions, CDR3 comprising SEQ ID NO: 47 or a variant thereof having up to five amino acid substitutions, comprising an on-rate constant (kₒₙ) of about 6.35×10\hat{ }5±5.90×10\hat{ }5 and an off-rate constant (k_{off}) of about 3.75×10\hat{ }−3±5.35×10\hat{ }−3 and optionally a K_{D} of about 5.91, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 43 or a variant thereof having up to two amino acid substitutions, CDR2 comprising SEQ ID NO: 45 or a variant thereof having up to two amino acid substitutions, CDR3 comprising SEQ ID NO: 47 or a variant thereof having up to five amino acid substitutions, comprising an on-rate constant (kₒₙ) of about 6.35×10\hat{ }5±2.60×10\hat{ }4 and an off-rate constant (k_{off}) of about 3.75×10\hat{ }−3±6.18×10\hat{ }−5 and optionally a K_{D} of about 5.91, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 2.33×10\hat{ }6±3.10×10\hat{ }6 and an off-rate constant (k_{off}) of about 3.57×10\hat{ }−2±5.09×10\hat{ }−2 and optionally a K_{D} of about 2.66×10\hat{ }1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 2.33×10\hat{ }6±2.16×10\hat{ }6 and an off-rate constant (k_{off}) of about 3.57×10\hat{ }−2±5.09×10\hat{ }−2 and optionally a K_{D} of about 2.66×10\hat{ }1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 2.33×10\hat{ }6±3.10×10\hat{ }6 and an off-rate constant (k_{off}) of about 3.57×10\hat{ }−2±2.32×10\hat{ }−2 and optionally a K_{D} of about 2.66×10\hat{ }1, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 8.02×10\hat{ }5±7.45×10\hat{ }5 and an off-rate constant (k_{off}) of about 6.1×10\hat{ }−3±8.7×10\hat{ }−3 and optionally a K_{D} of about 7.60, optionally wherein the values are obtained by an Octet BLI binding kinetic measurement.

In some embodiments, the invention provides an anti-interleukin 6 (IL6) single-domain antibody (sdAb) comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 8.02×10\hat{ }5±4.47×10\hat{ }4 and an off-rate constant (k_{off}) of about 6.1×10\hat{ }−3±1.09×10\hat{ }−4 and optionally a K_{D} of about 7.60, optionally wherein the values are obtained by an Octet BLI binding kinetic measurement.

In some embodiments, the invention provides an anti-interleukin 6 (IL6) single-domain antibody (sdAb) comprising an on-rate constant (kₒₙ) of about 1.82×10\hat{ }6 and an off-rate constant (k_{off}) of about 6.71×10\hat{ }−4 and optionally a K_{D} of about 0.37, optionally wherein the values are obtained by a SPR Biacore binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 2.21×10\hat{ }6 and an off-rate constant (k_{off}) of about 6.53×10\hat{ }−4 and optionally a K_{D} of about 0.30, optionally wherein the values are obtained by a SPR Biacore binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 8.37×10\hat{ }5 and an off-rate constant (k_{off}) of about 3.08×10\hat{ }−4 and optionally a K_{D} of about 0.37, optionally wherein the values are obtained by a SPR Biacore binding kinetic measurement, preferably wherein the sdAb is defined in SEQ ID NO: 1.

In some embodiments, the invention provides an anti-interleukin 6 (IL6) single-domain antibody (sdAb) comprising an on-rate constant (kₒₙ) of about 8.45×10\hat{ }5 and an off-rate constant (k_{off}) of about 5.35×10\hat{ }−4 and optionally a K_{D} of about 0.63, optionally wherein the values are obtained by a SPR Biacore binding kinetic measurement, preferably wherein the sdAb is defined in SEQ ID NO: 41.

In some embodiments, the invention provides an anti-interleukin 6 (IL6) single-domain antibody (sdAb) comprising an on-rate constant (kₒₙ) of about 7.33×10\hat{ }5 and an off-rate constant (k_{off}) of about 5.52×10\hat{ }−4 and optionally a K_{D} of about 0.75, optionally wherein the values are obtained by a SPR Biacore binding kinetic measurement, preferably wherein the sdAb is defined in SEQ ID NO: 17.

In some embodiments, the invention provides an anti-interleukin 6 (IL6) single-domain antibody (sdAb) comprising an on-rate constant (kₒₙ) of about 8.64×10\hat{ }5 and an off-rate constant (k_{off}) of about 1.61×10\hat{ }−3 and optionally a K_{D} of about 1.9, optionally wherein the values are obtained by a SPR Biacore binding kinetic measurement, preferably wherein the sdAb is defined in SEQ ID NO: 57.

In some embodiments, the invention provides an anti-interleukin 6 (IL6) single-domain antibody (sdAb) comprising an on-rate constant (kₒₙ) of about 1.86×10\hat{ }5 and an off-rate constant (k_{off}) of about 1.27×10\hat{ }−3 and optionally a K_{D} of about 6.8, optionally wherein the values are obtained by a SPR Biacore binding kinetic measurement, preferably wherein the sdAb is defined in SEQ ID NO: 49.

In some embodiments, the invention provides an anti-interleukin 6 (IL6) single-domain antibody (sdAb) comprising an on-rate constant (kₒₙ) of about 2.0×10\hat{ }6 and an off-rate constant (k_{off}) of about 1.6×10\hat{ }−2 and optionally a K_{D} of about 7.8×10\hat{ }−9 M, optionally wherein the values are obtained by an Octet BLI binding kinetic measurement, preferably wherein the sdAb is defined in SEQ ID NO: 1.

In some embodiments, the invention provides an anti-interleukin 6 (IL6) single-domain antibody (sdAb) comprising an on-rate constant (kₒₙ) of about 2.0×10\hat{ }6 and an off-rate constant (k_{off}) of about 7.0×10\hat{ }−3 and optionally a K_{D} of about 3.6×10\hat{ }−9 M, optionally wherein the values are obtained by an Octet BLI binding kinetic measurement, preferably wherein the sdAb is defined in SEQ ID NO: 9.

In some embodiments, the invention provides an anti-interleukin 6 (IL6) single-domain antibody (sdAb) comprising an on-rate constant (kₒₙ) of about 1.3×10\hat{ }7 and an off-rate constant (k_{off}) of about 4.0×10\hat{ }−2 and optionally a K_{D} of about 3.0×10\hat{ }−9 M, optionally wherein the values are obtained by an Octet BLI binding kinetic measurement, preferably wherein the sdAb is defined in SEQ ID NO: 17.

In some embodiments, the invention provides an anti-interleukin 6 (IL6) single-domain antibody (sdAb) comprising an on-rate constant (kₒₙ) of about 1.8×10\hat{ }6 and an off-rate constant (k_{off}) of about 5.0×10\hat{ }−3 and optionally a K_{D} of about 2.8×10\hat{ }−9 M, optionally wherein the values are obtained by an Octet BLI binding kinetic measurement, preferably wherein the sdAb is defined in SEQ ID NO: 25.

In some embodiments, the invention provides an anti-interleukin 6 (IL6) single-domain antibody (sdAb) comprising an on-rate constant (kₒₙ) of about 4.5×10\hat{ }6 and an off-rate constant (k_{off}) of about 7.1×10\hat{ }−6 and optionally a K_{D} of about 1.6×10\hat{ }−12 M, optionally wherein the values are obtained by an Octet BLI binding kinetic measurement, preferably wherein the sdAb is defined in SEQ ID NO: 33.

In some embodiments, the invention provides an anti-interleukin 6 (IL6) single-domain antibody (sdAb) comprising an on-rate constant (kₒₙ) of about 4.3×10\hat{ }6 and an off-rate constant (k_{off}) of about 1.2×10\hat{ }−6 and optionally a K_{D} of about 2.8×10\hat{ }−13 M, optionally wherein the values are obtained by an Octet BLI binding kinetic measurement, preferably wherein the sdAb is defined in SEQ ID NO: 41.

In some embodiments, the invention provides an anti-interleukin 6 (IL6) single-domain antibody (sdAb) comprising an on-rate constant (kₒₙ) of about 1.2×10\hat{ }6 and an off-rate constant (k_{off}) of about 5.5×10\hat{ }−3 and optionally a K_{D} of about 4.6×10\hat{ }−9 M, optionally wherein the values are obtained by an Octet BLI binding kinetic measurement, preferably wherein the sdAb is defined in SEQ ID NO: 49.

In some embodiments, the invention provides an anti-interleukin 6 (IL6) single-domain antibody (sdAb) comprising an on-rate constant (kₒₙ) of about 5.5×10\hat{ }6 and an off-rate constant (k_{off}) of about 7.4×10\hat{ }−2 and optionally a K_{D} of about 1.4×10\hat{ }−8 M, optionally wherein the values are obtained by an Octet BLI binding kinetic measurement, preferably wherein the sdAb is defined in SEQ ID NO: 57.

In one aspect, the present invention provides an anti-interleukin 6 (IL6) single-domain antibody (sdAb) comprising an on-rate constant (kₒₙ), an off-rate constant (k_{off}) and optionally a K_{D} as defined in any one of Tables 6, 7, 10 or 11, optionally wherein the values are obtained by a SPR Biacore binding kinetic measurement or an Octet BLI binding kinetic measurement.

Accordingly, the present invention provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

A sdAb comprises framework regions (FRs) and complementary determining regions (CDRs). The FRs are structural components, whereas the CDRs determine the specificity. In general, a sdAb of the present invention consists of four FRs and three CDRs. A sdAb of the present invention can be defined by its CDRs which determine the specificity and the epitope bound. The invention is not particularly limited to sdAb comprising specific CDRs. In particular, the invention provides herein below a detailed description how sdAb having desired kₒₙ and k_{off} values may be obtained. Accordingly, the skilled person is in the position to generate and select further anti-IL6 sdAb according to the invention. In particular, the skilled person is in the position to select antibodies having CDRs having desired kₒₙ and k_{off} values according to the present invention. The invention provides exemplary sdAb having IL6 specific CDRs and specific kₒₙ and k_{off} values allowing for specific, reversible and therefore continuous association and dissociation with IL6 over time. Exemplary sdAb of the present invention can comprise a first CDR, a second CDR and a third CDR, wherein the first CDR is selected from SEQ ID NOs: 3, 11, 19, 27, 35, 43, 51, or 59, and wherein the second CDR is selected from SEQ ID NOs: 5, 13, 21, 29, 37, 45, 53, or 61, and wherein the third CDR is selected from SEQ ID NOs: 7, 15, 23, 31, 39, 47, 55, or 63.

It is envisioned herein, that the CDRs of a sdAb also comprise variants thereof having one or more amino acid modifications. An “amino-acid modification” at a specified position, e.g. of the CDR, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution. Accordingly, the invention provides CDRs or variants thereof having amino acid substitutions. It may be preferred herein, that said one or more amino acid substitutions do not interfere with the ability of the sdAb to continuously associate and dissociate with IL6 over time. A CDR of the present invention may have one or more amino acid substitutions. A CDR of the present invention may have 1-10 amino acid substitutions, such as 1, 2, 3, 4, 5, 6, 7, 8, 9, or 10 amino acid substitutions. A first CDR of the invention may have 1-2 amino acid substitutions, such as 1 or 2 amino acid substitutions and/or up to 2 amino acid substitutions. A second CDR may have 1-2 amino acid substitutions, such as 1 or 2 amino acid substitutions and/or up to 2 amino acid substitutions. A third CDR may have 1-5 amino acid substitutions, such as 1, 2, 3, 4, or 5 amino acid substitutions and/or up to 5 amino acid substitutions.

It is preferred herein, that a sdAb of the present invention comprises a first CDR, a second CDR and a third CDR, wherein,
(i) the first CDR comprises SEQ ID NO: 3, the second CDR comprises SEQ ID NO: 5 and the third CDR comprises SEQ ID NO: 7, or
(ii) the first CDR comprises SEQ ID NO: 11, the second CDR comprises SEQ ID NO: 13 and the third CDR comprises SEQ ID NO: 15, or
(iii) the first CDR comprises SEQ ID NO: 19, the second CDR comprises SEQ ID NO: 21 and the third CDR comprises SEQ ID NO: 23, or
(iv) the first CDR comprises SEQ ID NO: 27, the second CDR comprises SEQ ID NO: 29 and the third CDR comprises SEQ ID NO: 31, or
(v) the first CDR comprises SEQ ID NO: 35, the second CDR comprises SEQ ID NO: 37 and the third CDR comprises SEQ ID NO: 39, or
(vi) the first CDR comprises SEQ ID NO: 43, the second CDR comprises SEQ ID NO: 45 and the third CDR comprises SEQ ID NO: 47, or
(vii) the first CDR comprises SEQ ID NO: 51, the second CDR comprises SEQ ID NO: 53 and the third CDR comprises SEQ ID NO: 55, or
(viii) the first CDR comprises SEQ ID NO: 59, the second CDR comprises SEQ ID NO: 61 and the third CDR comprises SEQ ID NO: 63.

In another aspect, it is preferred herein that a sdAb of the present invention comprises a first CDR, a second CDR and a third CDR, wherein,
(i) the first CDR comprises SEQ ID NO: 3 or a variant thereof having one or two amino acid substitutions, the second CDR comprises SEQ ID NO: 5 or a variant thereof having one or two amino acid substitutions, and the third CDR comprises SEQ ID NO: 7 or a variant thereof having one, two, three, four or five amino acid substitutions, or
(ii) the first CDR comprises SEQ ID NO: 11 or a variant thereof having one or two amino acid substitutions, the second CDR comprises SEQ ID NO: 13 or a variant thereof having one or two amino acid substitutions, and the third CDR comprises SEQ ID NO: 15 or a variant thereof having one, two, three, four or five amino acid substitutions, or
(iii) the first CDR comprises SEQ ID NO: 19 or a variant thereof having one or two amino acid substitutions, the second CDR comprises SEQ ID NO: 21 or a variant thereof having one or two amino acid substitutions, and the third CDR comprises SEQ ID NO: 23 or a variant thereof having one, two, three, four or five amino acid substitutions, or
(iv) the first CDR comprises SEQ ID NO: 27 or a variant thereof having one or two amino acid substitutions, the second CDR comprises SEQ ID NO: 29 or a variant thereof having one or two amino acid substitutions, and the third CDR comprises SEQ ID NO: 31 or a variant thereof having one, two, three, four or five amino acid substitutions, or
(v) the first CDR comprises SEQ ID NO: 35 or a variant thereof having one or two amino acid substitutions, the second CDR comprises SEQ ID NO: 37 or a variant thereof having one or two amino acid substitutions, and the third CDR comprises SEQ ID NO: 39 or a variant thereof having one, two, three, four or five amino acid substitutions, or
(vi) the first CDR comprises SEQ ID NO: 43 or a variant thereof having one or two amino acid substitutions, the second CDR comprises SEQ ID NO: 45 or a variant thereof having one or two amino acid substitutions, and the third CDR comprises SEQ ID NO: 47 or a variant thereof having one, two, three, four or five amino acid substitutions, or
(vii) the first CDR comprises SEQ ID NO: 51 or a variant thereof having one or two amino acid substitutions, the second CDR comprises SEQ ID NO: 53 or a variant thereof having one or two amino acid substitutions, and the third CDR comprises SEQ ID NO: 55 or a variant thereof having one, two, three, four or five amino acid substitutions, or
(viii) the first CDR comprises SEQ ID NO: 59 or a variant thereof having one or two amino acid substitutions, the second CDR comprises SEQ ID NO: 61 or a variant thereof having one or two amino acid substitutions, and the third CDR comprises SEQ ID NO: 63 or a variant thereof having one, two, three, four or five amino acid substitutions.

Accordingly, the invention provides in one aspect an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, comprising a first complementary determining region (CDR1), a second CDR (CDR2) and third CDR (CDR3), wherein:
(i) CDR1 comprises SEQ ID NO: 3 or a variant thereof having up to two amino acid substitutions, CDR2 comprises SEQ ID NO: 5 or a variant thereof having up to two amino acid substitutions, CDR3 comprises SEQ ID NO: 7 or a variant thereof having up to five amino acid substitutions; or
(ii) CDR1 comprises SEQ ID NO: 11 or a variant thereof having up to two amino acid substitutions, CDR2 comprises SEQ ID NO: 13 or a variant thereof having up to two amino acid substitutions, CDR3 comprises SEQ ID NO: 15 or a variant thereof having up to five amino acid substitutions; or
(iii) CDR1 comprises SEQ ID NO: 19 or a variant thereof having up to two amino acid substitutions, CDR2 comprises SEQ ID NO: 21 or a variant thereof having up to two amino acid substitutions, CDR3 comprises SEQ ID NO: 23 or a variant thereof having up to five amino acid substitutions; or
(iv) CDR1 comprises SEQ ID NO: 27 or a variant thereof having up to two amino acid substitutions, CDR2 comprises SEQ ID NO: 29 or a variant thereof having up to two amino acid substitutions, CDR3 comprises SEQ ID NO: 31 or a variant thereof having up to five amino acid substitutions; or
(v) CDR1 comprises SEQ ID NO: 35 or a variant thereof having up to two amino acid substitutions, CDR2 comprises SEQ ID NO: 37 or a variant thereof having up to two amino acid substitutions, CDR3 comprises SEQ ID NO: 39 or a variant thereof having up to five amino acid substitutions; or
(vi) CDR1 comprises SEQ ID NO: 43 or a variant thereof having up to two amino acid substitutions, CDR2 comprises SEQ ID NO: 45 or a variant thereof having up to two amino acid substitutions, CDR3 comprises SEQ ID NO: 47 or a variant thereof having up to five amino acid substitutions; or
(vii) CDR1 comprises SEQ ID NO: 51 or a variant thereof having up to two amino acid substitutions, CDR2 comprises SEQ ID NO: 53 or a variant thereof having up to two amino acid substitutions, CDR3 comprises SEQ ID NO: 55 or a variant thereof having up to five amino acid substitutions; or
(viii) CDR1 comprises SEQ ID NO: 59 or a variant thereof having up to two amino acid substitutions, CDR2 comprises SEQ ID NO: 61 or a variant thereof having up to two amino acid substitutions, CDR3 comprises SEQ ID NO: 63 or a variant thereof having up to five amino acid substitutions.

As mentioned herein above, a sdAb of the present invention can also comprise structural framework regions (FR). In general, the sdAb of the present invention are not limited to any FRs, rather any FR that results in a functional sdAb according to the present invention may be used. The invention provides herein below a detailed description how feasible FRs can be selected. Accordingly, the skilled person is enabled to select FRs that can be used in the sense of the invention. The invention provides exemplary FRs that can be used with the CDRs described herein to form a sdAb according to the invention. Exemplary FRs can comprise a first FR, a second FR, a third FR and a fourth FR. It may be preferred herein, that a first FR comprises any one of SEQ ID NOs: 2, 10, 18, 26, 34, 42, 50, or 58, a second FR comprises any one of SEQ ID NOs: 4, 12, 20, 28, 36, 44, 52, or 60, a third FR comprises any one of SEQ ID NOs: 6, 14, 22, 30, 38, 46, 54, or 62 and a fourth FR comprises any one of SEQ ID NOs: 8, 16, 24, 32, 40, 48, 56 or 64. The FRs in a sdAb have structural function.

Accordingly, it is envisioned herein, that the FRs of a sdAb also comprise variants thereof having one or more amino acid modifications. The invention provides FRs or variants thereof having amino acid substitutions. It may be preferred herein, that said one or more amino acid substitutions do not interfere with the ability of the sdAb to continuously associate and dissociate with IL6 over time. It may further be preferred herein, that said one or more amino acid substitutions do not interfere with the structural integrity of the sdAb. A FR of the present invention may have one or more amino acid substitutions. A FR of the present invention may have 1-10 amino acid substitutions, such as 1, 2, 3, 4, 5, 6, 7, 8, 9, or 10 amino acid substitutions. A first FR of the invention may have 1-6 amino acid substitutions, such as 1, 2, 3, 4, 5, or 6 amino acid substitutions and/or up to 6 amino acid substitutions. A second CDR may have 1-4 amino acid substitutions, such as 1, 2, 3, or 4 amino acid substitutions and/or up to 4 amino acid substitutions. A third CDR may have 1-10 amino acid substitutions, such as 1, 2, 3, 4, 5, 6, 7, 8, 9, or 10 amino acid substitutions and/or up to 10 amino acid substitutions. A third CDR may have 1-3 amino acid substitutions, such as 1, 2, or 3 amino acid substitutions and/or up to 3 amino acid substitutions.

It is preferred herein, that a sdAb of the present invention comprises a first FR, a second FR, a third FR and a fourth FR, wherein,
(i) the first FR comprises SEQ ID NO: 2, the second FR comprises SEQ ID NO: 4, the third FR comprises SEQ ID NO: 6, and the fourth FR comprises SEQ ID NO: 8, or
(ii) the first FR comprises SEQ ID NO: 10, the second FR comprises SEQ ID NO: 12, the third FR comprises SEQ ID NO: 14, and the fourth FR comprises SEQ ID NO: 16, or
(iii) the first FR comprises SEQ ID NO: 18, the second FR comprises SEQ ID NO: 20, the third FR comprises SEQ ID NO: 22, and the fourth FR comprises SEQ ID NO: 24, or
(iv) the first FR comprises SEQ ID NO: 26, the second FR comprises SEQ ID NO: 28, the third FR comprises SEQ ID NO: 30, and the fourth FR comprises SEQ ID NO: 32, or
(v) the first FR comprises SEQ ID NO: 34, the second FR comprises SEQ ID NO: 36, the third FR comprises SEQ ID NO: 38, and the fourth FR comprises SEQ ID NO: 40, or
(vi) the first FR comprises SEQ ID NO: 42, the second FR comprises SEQ ID NO: 44, the third FR comprises SEQ ID NO: 46, and the fourth FR comprises SEQ ID NO: 48, or
(vii) the first FR comprises SEQ ID NO: 50, the second FR comprises SEQ ID NO: 52, the third FR comprises SEQ ID NO: 54, and the fourth FR comprises SEQ ID NO: 56, or
(viii) the first FR comprises SEQ ID NO: 58, the second FR comprises SEQ ID NO: 60, the third FR comprises SEQ ID NO: 62, and the fourth FR comprises SEQ ID NO: 64.

In another aspect, it is preferred herein, that a sdAb of the present invention comprises a first FR, a second FR and a third FR, wherein,
(i) the first FR comprises SEQ ID NO: 2 or a variant thereof having one, two, three, four, five or six amino acid substitutions, the second FR comprises SEQ ID NO: 4 or a variant thereof having one, two, three, or four amino acid substitutions, the third FR comprises SEQ ID NO: 6 or a variant thereof having one, two, three, four, five, six, seven, eight, nine, or ten amino acid substitutions, and the fourth FR comprises SEQ ID NO: 8 or a variant thereof having one, two, or three amino acid substitutions, or
(ii) the first FR comprises SEQ ID NO: 10 or a variant thereof having one, two, three, four, five or six amino acid substitutions, the second FR comprises SEQ ID NO: 12 or a variant thereof having one, two, three, or four amino acid substitutions, the third FR comprises SEQ ID NO: 14 or a variant thereof having one, two, three, four, five, six, seven, eight, nine, or ten amino acid substitutions, and the fourth FR comprises SEQ ID NO: 16 or a variant thereof having one, two, or three amino acid substitutions, or
(iii) the first FR comprises SEQ ID NO: 18 or a variant thereof having one, two, three, four, five or six amino acid substitutions, the second FR comprises SEQ ID NO: 20 or a variant thereof having one, two, three, or four amino acid substitutions, the third FR comprises SEQ ID NO: 22 or a variant thereof having one, two, three, four, five, six, seven, eight, nine, or ten amino acid substitutions, and the fourth FR comprises SEQ ID NO: 24 or a variant thereof having one, two, or three amino acid substitutions, or
(iv) the first FR comprises SEQ ID NO: 26 or a variant thereof having one, two, three, four, five or six amino acid substitutions, the second FR comprises SEQ ID NO: 28 or a variant thereof having one, two, three, or four amino acid substitutions, the third FR comprises SEQ ID NO: 30 or a variant thereof having one, two, three, four, five, six, seven, eight, nine, or ten amino acid substitutions, and the fourth FR comprises SEQ ID NO: 32 or a variant thereof having one, two, or three amino acid substitutions, or
(v) the first FR comprises SEQ ID NO: 34 or a variant thereof having one, two, three, four, five or six amino acid substitutions, the second FR comprises SEQ ID NO: 36 or a variant thereof having one, two, three, or four amino acid substitutions, the third FR comprises SEQ ID NO: 38 or a variant thereof having one, two, three, four, five, six, seven, eight, nine, or ten amino acid substitutions, and the fourth FR comprises SEQ ID NO: 40 or a variant thereof having one, two, or three amino acid substitutions, or
(vi) the first FR comprises SEQ ID NO: 42 or a variant thereof having one, two, three, four, five or six amino acid substitutions, the second FR comprises SEQ ID NO: 44 or a variant thereof having one, two, three, or four amino acid substitutions, the third FR comprises SEQ ID NO: 46 or a variant thereof having one, two, three, four, five, six, seven, eight, nine, or ten amino acid substitutions, and the fourth FR comprises SEQ ID NO: 48 or a variant thereof having one, two, or three amino acid substitutions, or
(vii) the first FR comprises SEQ ID NO: 50 or a variant thereof having one, two, three, four, five or six amino acid substitutions, the second FR comprises SEQ ID NO: 52 or a variant thereof having one, two, three, or four amino acid substitutions, the third FR comprises SEQ ID NO: 54 or a variant thereof having one, two, three, four, five, six, seven, eight, nine, or ten amino acid substitutions, and the fourth FR comprises SEQ ID NO: 56 or a variant thereof having one, two, or three amino acid substitutions, or
(viii) the first FR comprises SEQ ID NO: 58 or a variant thereof having one, two, three, four, five or six amino acid substitutions, the second FR comprises SEQ ID NO: 60 or a variant thereof having one, two, three, or four amino acid substitutions, the third FR comprises SEQ ID NO: 62 or a variant thereof having one, two, three, four, five, six, seven, eight, nine, or ten amino acid substitutions, and the fourth FR comprises SEQ ID NO: 64 or a variant thereof having one, two, or three amino acid substitutions.

Accordingly, the invention provides in one aspect an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, comprising a first framework region (FR1), a second framework region (FR2), a third framework region (FR3), and a fourth framework region (FR3) wherein,
(i) FR1 comprises SEQ ID NO: 2 or a variant thereof having up to six amino acid substitutions, FR2 comprises SEQ ID NO: 4 or a variant thereof having up to four amino acid substitutions, FR3 comprises SEQ ID NO: 6 or a variant thereof having up to ten amino acid substitutions, and FR4 comprises SEQ ID NO: 8 or a variant thereof having up to three amino acid substitutions; or
(ii) FR1 comprises SEQ ID NO: 10 or a variant thereof having up to six amino acid substitutions, FR2 comprises SEQ ID NO: 12 or a variant thereof having up to four amino acid substitutions, FR3 comprises SEQ ID NO: 14 or a variant thereof having up to ten amino acid substitutions, and FR4 comprises SEQ ID NO: 16 or a variant thereof having up to three amino acid substitutions; or
(iii) FR1 comprises SEQ ID NO: 18 or a variant thereof having up to six amino acid substitutions, FR2 comprises SEQ ID NO: 20 or a variant thereof having up to four amino acid substitutions, FR3 comprises SEQ ID NO: 22 or a variant thereof having up to ten amino acid substitutions, and FR4 comprises SEQ ID NO: 24 or a variant thereof having up to three amino acid substitutions; or
(iv) FR1 comprises SEQ ID NO: 26 or a variant thereof having up to six amino acid substitutions, FR2 comprises SEQ ID NO: 28 or a variant thereof having up to four amino acid substitutions, FR3 comprises SEQ ID NO: 30 or a variant thereof having up to ten amino acid substitutions, and FR4 comprises SEQ ID NO: 32 or a variant thereof having up to three amino acid substitutions; or
(v) FR1 comprises SEQ ID NO: 34 or a variant thereof having up to six amino acid substitutions, FR2 comprises SEQ ID NO: 36 or a variant thereof having up to four amino acid substitutions, FR3 comprises SEQ ID NO: 38 or a variant thereof having up to ten amino acid substitutions, and FR4 comprises SEQ ID NO: 40 or a variant thereof having up to three amino acid substitutions; or
(vi) FR1 comprises SEQ ID NO: 42 or a variant thereof having up to six amino acid substitutions, FR2 comprises SEQ ID NO: 44 or a variant thereof having up to four amino acid substitutions, FR3 comprises SEQ ID NO: 46 or a variant thereof having up to ten amino acid substitutions, and FR4 comprises SEQ ID NO: 48 or a variant thereof having up to three amino acid substitutions; or
(vii) FR1 comprises SEQ ID NO: 50 or a variant thereof having up to six amino acid substitutions, FR2 comprises SEQ ID NO: 52 or a variant thereof having up to four amino acid substitutions, FR3 comprises SEQ ID NO: 54 or a variant thereof having up to ten amino acid substitutions, and FR4 comprises SEQ ID NO: 56 or a variant thereof having up to three amino acid substitutions; or
(viii) FR1 comprises SEQ ID NO: 58 or a variant thereof having up to six amino acid substitutions, FR2 comprises SEQ ID NO: 60 or a variant thereof having up to four amino acid substitutions, FR3 comprises SEQ ID NO: 62 or a variant thereof having up to ten amino acid substitutions, and FR4 comprises SEQ ID NO: 64 or a variant thereof having up to three amino acid substitutions.

As mentioned herein above, a sdAb of the present invention can preferably comprise three CDRs and four FRs. Accordingly, a sdAb preferably comprises a first FR, a first CDR, a second FR, a second CDR, a third FR, a third CDR and a fourth FR. A sdAb of the present invention can consist of a first FR, a first CDR, a second FR, a second CDR, a third FR, a third CDR and a fourth FR. A sdAb of the present invention can comprise any one of SEQ ID NOs: 1, 9, 17, 25, 33, 41, 49, or 57. A sdAb of the present invention can also consist of SEQ ID NOs: 1, 9, 17, 25, 33, 41, 49, or 57. As described herein above for the individual CDRs and FRs, the sdAb can also comprise amino acid substitutions that essentially do not interfere with its function or structural integrity. A sdAb of the present invention can comprise any number of amino acid substitutions as long as it can still be used according to the invention. Accordingly, the present invention also provides variants of a sdAb of the present invention having 80, 81, 82, 83, 84, 85, 86, 87, 88, 89, 90, 91, 92, 93, 94, 95, 96, 97, 98, or 99% sequence identity when compared to the sdAb. Preferably, the invention provides variants of a sdAb of the invention having more than 90%, such as 91, 92, 93, 94, 95, 96, 97, 98, or 99%, or at least 90%, such as 90, 91, 92, 93, 94, 95, 96, 97, 98, or 99%, or 90-99%, such as 90, 91, 92, 93, 94, 95, 96, 97, 98, or 99% sequence identity when compared to the sdAb.

Accordingly, the present invention provides in another aspect an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the sdAb comprises any one of
(i) SEQ ID NO: 1 or a variant thereof having at least about 90% sequence identity to SEQ ID NO: 1; or
(ii) SEQ ID NO: 9 or a variant thereof having at least about 90% sequence identity to SEQ ID NO: 9; or
(iii) SEQ ID NO: 17 or a variant thereof having at least about 90% sequence identity to SEQ ID NO: 17; or
(iv) SEQ ID NO: 25 or a variant thereof having at least about 90% sequence identity to SEQ ID NO: 25; or
(v) SEQ ID NO: 33 or a variant thereof having at least about 90% sequence identity to SEQ ID NO: 33; or
(vi) SEQ ID NO: 41 or a variant thereof having at least about 90% sequence identity to SEQ ID NO: 41; or
(vii) SEQ ID NO: 49 or a variant thereof having at least about 90% sequence identity to SEQ ID NO: 49; or
(viii) SEQ ID NO: 57 or a variant thereof having at least about 90% sequence identity to SEQ ID NO: 57.

As mentioned herein above, the sdAbs of the invention can embody a specific on-rate constant (kₒₙ) and off-rate constant (k_{off}). sdAbs within the ranges defined herein have the advantage that they can associate and dissociate with IL6 over time. This means that a continuous and repeated association followed by dissociation with IL6 is achieved. This is particularly desired when the sdAbs are used in a measurement over time where the dynamic level of IL6 is to be determined. As described herein, determining and monitoring the dynamic level of IL6 is particularly advantageous for determining inflammation spikes, such as during the course of a disease or to monitor inflammatory reactions, e.g. to a treatment or drug. Accordingly, it is preferred herein, that the anti-IL6 sdAbs of the present invention continuously and reversibly bind IL6. The reversible binding of IL6 is particularly desired when the sdAbs are used on a sensor, e.g. biosensor. A sdAb having exceptionally high affinity cannot dissociate from IL6 and thus is not feasible in a measurement of dynamic IL6 levels over time. The present invention provides anti-IL6 sdAbs within kₒₙ and k_{off} ranges allowing continuous and reversible binding of IL6. For example, a sdAb of the invention can bind or associate with an IL6, followed by dissociation from said IL6. After dissociation from the first IL6, the same sdAb can bind a second (other) IL6. After dissociation from the second IL6, the same sdAb can bind a third (other) IL6. Association and dissociation of any further IL6 can be repeated as described before. Thus, the sdAbs of the present invention can associate and dissociate a multitude of IL6 (reversible binding), enabling measurement of dynamic IL6 levels over time (continuous binding).

Accordingly, the invention provides in one aspect an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb continuously and reversibly binds IL6.

The invention also provides an anti-interleukin 6 (IL6) single-domain antibody (sdAb) comprising an on-rate constant (kₒₙ) of about 0.15×10\hat{ }6-2.25×10\hat{ }6 and an off-rate constant (k_{off}) of about 0.0002-0.002, wherein the anti-IL6 sdAb continuously and reversibly binds IL6.

The invention also provides in one aspect an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb binds a second or further IL6 after dissociating from a first IL6.

The invention also provides in one aspect an anti-interleukin 6 (IL6) single-domain antibody (sdAb) comprising an on-rate constant (kₒₙ) of about 0.15×10\hat{ }6-2.25×10\hat{ }6 and an off-rate constant (k_{off}) of about 0.0002-0.002, wherein the anti-IL6 sdAb binds a second or further IL6 after dissociating from a first IL6.

The invention also provides an anti-interleukin 6 (IL6) single-domain antibody (sdAb) comprising an on-rate constant (kₒₙ) of about 1.86×10\hat{ }5-1.82×10\hat{ }6 and an off-rate constant (k_{off}) of about 0.000308-0.00161 and optionally a K_{D} of about 3×10\hat{ }−10 M-6.8×10\hat{ }−9 M, wherein the anti-IL6 sdAb continuously and reversibly binds IL6.

The invention also provides an anti-interleukin 6 (IL6) single-domain antibody (sdAb) comprising an on-rate constant (kₒₙ) of about 1.20×10\hat{ }6-1.30×10\hat{ }7 and an off-rate constant (k_{off}) of about 1.20×10\hat{ }−6-7.40×10\hat{ }−2 and optionally a K_{D} of about 2.80×10\hat{ }−13 M-1.40×10\hat{ }−8 M, wherein the anti-IL6 sdAb continuously and reversibly binds IL6.

The invention also provides in one aspect an anti-interleukin 6 (IL6) single-domain antibody (sdAb) comprising an on-rate constant (kₒₙ) of about 1.86×10\hat{ }5-1.82×10\hat{ }6 and an off-rate constant (k_{off}) of about 0.000308-0.00161 and optionally a K_{D} of about 3×10\hat{ }−10 M-6.8×10\hat{ }−9 M, wherein the anti-IL6 sdAb binds a second or further IL6 after dissociating from a first IL6.

The invention also provides in one aspect an anti-interleukin 6 (IL6) single-domain antibody (sdAb) comprising an on-rate constant (kₒₙ) of about 1.20×10\hat{ }6-1.30×10\hat{ }7 and an off-rate constant (k_{off}) of about 1.20×10\hat{ }−6-7.40×10\hat{ }−2 and optionally a K_{D} of about 2.80×10\hat{ }−13 M-1.40×10\hat{ }−8 M, wherein the anti-IL6 sdAb binds a second or further IL6 after dissociating from a first IL6.

Antibodies in general can be used to inhibit the biological function of their targets. One advantage of sdAb is that they do not necessarily inhibit the biological function of their targets. In the sense of the present invention, both can be desired, i.e. inhibition of biological function in the sense of a treatment or a non-inhibiting sdAb, in the sense of monitoring IL6 levels together with a treatment or independent thereof. Accordingly, the present invention provides sdAbs that do not inhibit the biological function of IL6. In another aspect, the present invention provides sdAbs that do inhibit the biological function of IL6. As used herein “biological function of IL6” relates in general to any effect of IL6, i.e. relates to anything that IL6 “does”. This can include any function related to IL6 or IL6 signalling or the IL6 signalling pathway. The biological function of IL6 also includes upstream and downstream effects of and on IL6 that may be related to IL6 or the corresponding inflammation response.

Accordingly, the invention provides in one aspect an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb inhibits the biological function of IL6.

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb inhibits the biological function of IL6, wherein the biological function of IL6 is IL6 signalling or the IL6 signalling pathway.

In another aspect, the invention provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb does not inhibit the biological function of IL6.

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb does not inhibit the biological function of IL6, wherein the biological function of IL6 is IL6 signalling or the IL6 signalling pathway.

The skilled person is aware of means and methods for the assessment of the ability of a sdAb to inhibit IL6 signaling. Such means and methods include the assessment of the half-maximal inhibitory concentration (IC50), i.e., the concentration of an antibody leading to half-maximal inhibition (e.g., of IL6 signalling). This is further detailed in the enclosed illustrative Example 6 and in Neubig et al. 2003, which is hereby incorporated by reference in its entirety. The skilled person is aware, that a higher IC50 value indicates that a given sdAb has a lower potential for the inhibition of e.g., IL6 signaling. Whereas a lower IC50 value indicates that a given sdAb has a higher potential for the inhibition of e.g., IL6 signaling. Accordingly, as further detailed herein below, sdAbs having high IC50 values are less likely to interfere with e.g., IL6 signaling, and are, thus, particularly useful/suitable/desired in in vitro or in vivo diagnostic applications or continuous monitoring of IL6 levels such as, e.g. wherein the sdAb is incorporated in a biosensor which can be used either in vitro or in vivo, i.e. being partially or fully implanted into a subject. This is advantageous, when a continuous monitoring of IL6, is desired without inhibiting the biological function of IL6.

It is evidently shown in enclosed Example 6 that the herein provided sdAbs comprise drastically higher IC50 values (and, thus, drastically lower potential for the inhibition of IL6 signaling) as compared to the commercially available therapeutic anti-IL6 sdAb Siltuximab. Accordingly, the present invention provides for an anti-interleukin 6 (IL6) single-domain antibody (sdAb) comprising a higher IC50 as compared to therapeutic sdAbs (e.g., as compared to Siltuximab), preferably wherein said therapeutic sdAb (e.g., Siltuximab) comprises an IC50 of about 0.1 to 0.2 nM, more preferably of 0.114 nM (as detailed in Table 15).

Accordingly, the present invention provides for an anti-interleukin 6 (IL6) single-domain antibody (sdAb) comprising an IC50 of about 0.2 nM or more than about 0.2 nM, preferably of about 0.5 nM or more than about 0.5 nM, more preferably of about 1 nM or more than about 1 nM, even more preferably of about 2 nM or more than about 2 nM, even more preferably of about 3 nM or more than about 3 nM, even more preferably of about 3.04 nM or more than about 3.04 nM. In particular, the present invention provides for anti-IL6 sdAbs SIL-2D8 (or variants thereof) and SIL-2F5 (or variants thereof) comprising an IC50 of about 0.5, 1, 2, or 3 nM or more than about 0.5, 1, 2, or 3 nM. In some embodiments, the invention provides an sdAb SIL-2D8 comprising an IC50 of about 3.97 nM. In some embodiments, the invention provides an sdAb SIL-2F5 comprising an IC50 of about 3.04 nM.

Preferably, the present invention provides for an anti-interleukin 6 (IL6) single-domain antibody (sdAb) comprising an IC50 of about 120 nM or more than about 120 nM, preferably more than about 120 nM. In particular, the present invention provides for anti-IL6 sdAbs SIL-2D1 (or variants thereof), SIL-3B6 (or variants thereof), and SIL-2E4 (or variants thereof) comprising an IC50 of about 120 nM or more than about 120 nM.

Even more preferably, the present invention provides for an anti-interleukin 6 (IL6) single-domain antibody (sdAb) comprising an IC50 of about 600 nM or more than about 600 nm, preferably more than about 600 nM. In particular, the present invention provides for anti-IL6 sdAbs SIL-3B7 (or variants thereof), and SIL-3E7 (or variants thereof) comprising an IC50 of about 600 nM or more than about 600 nM.

Accordingly, the present invention provides for sdAbs with surprisingly low potential to inhibit IL6 signaling, which renders them particularly useful in in vitro or in vivo diagnostic applications or continuous monitoring of IL6 levels e.g. when being comprised in a biosensor which can be used either in vitro or in vivo, i.e. being partially or fully implanted into a subject, as is, inter alia, detailed in enclosed Example 6.

Preferably, in the context of the present invention IC50 values are determined by a IL6 signaling inhibition assay. More preferably, in the context of the present invention IC50 values are determined using a luminescence-based and/or fluorescence-based IL6 signaling inhibition assay. Even more preferably, in the context of the present invention IC50 values are determined using a luminescence-based and/or fluorescence-based IL6 signaling inhibition assay employing cells comprising/expressing at least one IL6 specific reporter construct (such as, for example iLite® IL6 Assay Ready Cells; Svar Life Science; Cat. No 11888) (and IL6 for the induction the reporter construct).

As is detailed herein above, the present invention provides for anti-IL6 sdAbs that are, inter alia, particularly useful in the detection and/or quantification of IL6 across the entire range of IL6 concentrations from baseline to sever inflammation. As is further detailed herein below, due to the low (i.e., fM to pM) IL6 concentrations e.g. in a human biological fluid even at high or severe inflammation (see, for example, Table 1), in the context of the present invention it is particularly advantageous that the herein provided anti-IL6 sdAbs, due to their advantageous binding affinities (and kₒₙ and k_{off} values), are able to detect and/or quantify the entire range of IL6 concentrations from baseline to severe inflammation (i.e., a less than 45 fM to more than 4.5 pM) e.g. in a human biological fluid (see, for example, Table 12.

Accordingly, the present invention provides in one aspect an anti-interleukin 6 (IL6) single-domain antibody (sdAb) capable of detecting and/or quantifying IL6 at a concentration of about 45 fM or less to about 4.5 pM or more.

The invention also provides an anti-interleukin 6 (IL6) single-domain antibody (sdAb) capable of detecting and/or quantifying IL6 at a concentration of about 45 fM or less and/or 45 fM to 225 fM, and/or 225 fM to 450 fM and/or 450 fM to 4.5 pM and/or 4.5 pM or more.

The invention also provides an anti-interleukin 6 (IL6) single-domain antibody (sdAb) capable of detecting and/or quantifying IL6 at a concentration of about 45 fM or less and/or 45 fM to 225 fM, and/or 225 fM to 450 fM and/or 450 fM to 4.5 pM and/or 4.5 pM or more, corresponding to baseline, low, moderate, high and/or severe inflammation levels e.g. in a human biological fluid (see Table 12).

The invention provides an anti-interleukin 6 (IL6) single-domain antibody (sdAb) SIL-2D8, wherein the sdAb is capable of detecting and/or quantifying IL6 at a concentration of about 45 fM or less to 4.5 pM or more, such as 45 fM or less (baseline inflammation) and/or 45 fM to 225 fM (low inflammation), and/or 225 fM to 450 fM (moderate inflammation) and/or 450 fM to 4.5 pM (high inflammation) and/or 4.5 pM or more (severe inflammation), optionally in a human biological fluid.

The invention provides an anti-interleukin 6 (IL6) single-domain antibody (sdAb) SIL-3B6, wherein the sdAb is capable of detecting and/or quantifying IL6 at a concentration of about 225 fM to 4.5 pM, such as 225 fM or more (low inflammation), and/or 225 fM to 450 fM (moderate inflammation) and/or 450 fM to 4.5 pM (high inflammation) and/or 4.5 pM or more (severe inflammation), optionally in a human biological fluid.

The invention provides an anti-interleukin 6 (IL6) single-domain antibody (sdAb) SIL-3E7, wherein the sdAb is capable of detecting and/or quantifying IL6 at a concentration of about 225 fM to 4.5 pM, such as 225 fM or more (low inflammation), and/or 225 fM to 450 fM (moderate inflammation) and/or 450 fM to 4.5 pM (high inflammation) and/or 4.5 pM or more (severe inflammation), optionally in a human biological fluid.

The invention provides an anti-interleukin 6 (IL6) single-domain antibody (sdAb) SIL-2F5, wherein the sdAb is capable of detecting and/or quantifying IL6 at a concentration of about 45 fM or less to 4.5 pM or more, such as 45 fM or less (baseline inflammation) and/or 45 fM to 225 fM (low inflammation), and/or 225 fM to 450 fM (moderate inflammation) and/or 450 fM to 4.5 pM (high inflammation) and/or 4.5 pM or more (severe inflammation), optionally in a human biological fluid.

The invention provides an anti-interleukin 6 (IL6) single-domain antibody (sdAb) SIL-2D1, wherein the sdAb is capable of detecting and/or quantifying IL6 at a concentration of about 45 fM or less to 4.5 pM or more, such as 45 fM or less (baseline inflammation) and/or 45 fM to 225 fM (low inflammation), and/or 225 fM to 450 fM (moderate inflammation) and/or 450 fM to 4.5 pM (high inflammation) and/or 4.5 pM or more (severe inflammation), optionally in a human biological fluid.

The invention provides an anti-interleukin 6 (IL6) single-domain antibody (sdAb) SIL-2E4, wherein the sdAb is capable of detecting and/or quantifying IL6 at a concentration of about 225 fM to 4.5 pM, such as 225 fM or more (low inflammation), and/or 225 fM to 450 fM (moderate inflammation) and/or 450 fM to 4.5 pM (high inflammation) and/or 4.5 pM or more (severe inflammation), optionally in a human biological fluid.

The invention provides an anti-interleukin 6 (IL6) single-domain antibody (sdAb) SIL-3B7, wherein the sdAb is capable of detecting and/or quantifying IL6 at a concentration of about 4.5 pM (high inflammation) and/or 4.5 pM or more (severe inflammation), optionally in a human biological fluid.

It is evident for the skilled person, that a sdAb capable of detecting and/or quantifying, e.g., baseline inflammation is also capable of detecting any other inflammation range characterized by higher IL6 concentrations (such as low, moderate, high, and severe inflammation).

Accordingly, the present invention provides for sdAbs that can surprisingly detect and/or quantify a broad range of IL6 concentrations, e.g. in a human biological fluid, which renders them particularly useful in in vitro or in vivo diagnostic applications or continuous monitoring of IL6 levels e.g. when being comprised in a biosensor which can be used either in vitro or in vivo, i.e. being partially or fully implanted into a subject.

As is detailed herein above, in the context of the present invention, the herein provided anti-IL6 sdAbs may be particularly useful in vitro or in vivo diagnostic applications or continuous monitoring of IL6 levels e.g. when being comprised in a biosensor which can be used either in vitro or in vivo, i.e. being partially or fully implanted into a subject. Preferred applications of the sdAbs provided herein may include continuous monitoring of IL6 concentrations, may rely on dilution, may include a forced release mechanism (e.g. via heat, light/optogenetics, vibration, redox potential or pH), and/or a multisensory chip. All of the herein provided sdAbs may be suitable for any of the above listed applications.

TABLE 13
Summary characteristics of exemplary sdAbs provided herein.

| | | | | | Covered |
|---|---|---|---|---|---|
| | | | | | inflammation |
| | | k_{d} | K_{D} | IC50 | range (based |
| Clone | kₐ[s⁻¹M⁻¹] | [s⁻¹] | [nM] | [nM] | on Table 12) |
| SIL-2D8 | 1.82E6 | 6.71E−4 | 0.37 | 3.97 | Baseline to severe |
| SIL-2E7 | — | — | — | — | — |
| SIL-2F5 | 2.21E6 | 6.53E−4 | 0.30 | 3.04 | Baseline to severe |
| SIL-2D1 | 8.37E5 | 3.08E−4 | 0.37 | >120 | Baseline to severe |
| SIL-3B6 | 8.45E5 | 5.35E−4 | 0.63 | >120 | Low to severe |
| SIL-2E4 | 7.33E5 | 5.52E−4 | 0.75 | >120 | Low to severe |
| SIL-3E7 | 8.64E5 | 1.61E−3 | 1.9 | >600 | Low to severe |
| SIL-3B7 | 1.86E5 | 1.27E−3 | 6.8 | >600 | High to severe |

The column “Covered inflammation range” in the table below indicates the respective inflammation range that a given sdAb can at least partly cover (i.e., any inflammation range that is indicated with “*” in Table 12) or fully cover (i.e., any inflammation range that is indicated with “**” in Table 12). All other columns summarize data also already detailed herein above.

As mentioned above, a sdAb of the present invention is particularly useful as a sensor, such as a biosensor. In general, a biosensor is an object used for the detection of a substance. A biosensor can combine a biological component, e.g. sdAb of the invention, with a physiochemical detector. When used in a biosensor, the biological component (sdAb) can associate with IL6, and the physiochemical detector then detects the association/binding. As mentioned above, the sdAb of the invention can continuously and reversibly associate with and dissociate from IL6 allowing the physiochemical detector to measure association/dissociation and therefore IL6 levels over time. In the sense of the present invention, the physiochemical detector can be an electrochemical detector. In general, the invention is not particularly limited to the use of any physiochemical detector, rather any physiochemical detector that allows a quantitative readout of the association/dissociation of anti-IL6 sdAb of the present invention can be used. Exemplary physiochemical detectors that can be used to quantify anti-IL6 sdAb association/dissociation with IL6 are described herein below. The biosensor of the present invention comprises at least, a sdAb of the present invention, at least one working electrode (also known as the sensing or redox electrode) and a substrate (structural element). Association of IL6 with the sdAb induces an electrical signal in the working electrode which allows for a quantitative measurement of IL6 association. The electric signal that is generated can be readout by any electrochemical detection techniques commonly used, such as amperometry, chronoamperometry, potentiometry, conductometry, voltammetry, cyclic voltammetry, galvanic cell, impedance spectroscopy, and the like. A working electrode used in the biosensor may comprise any conductive material, such as carbon, metal or alloy, or a metallic compound. An exemplary carbon-based material that can be used in the working electrode is graphite. An exemplary metal or alloy that can be used in the working electrode is platinum, gold or a gold alloy. Exemplary metallic compounds are silicon, ruthenium dioxide or titanium dioxide. In some embodiments, a biosensor electrode can be or comprise a glassy carbon electrode, a carbon nanotube-modified electrode, an indium tin oxide (ITO) electrode, a platinum electrode, a silver electrode, a gold electrode, or a palladium electrode. In some embodiments, a biosensor electrode can be or comprise a gold nanostructured microelectrode or a gold wire electrode. In the sense of the present invention a biosensor electrode can be or comprise a working electrode and/or a counter electrode and/or a reference electrode. In some embodiments a biosensor electrode can be or can comprise a working electrode. In some embodiments a biosensor electrode can be or can comprise a counter electrode. In some embodiments a biosensor electrode can be or can comprise a reference electrode.

In a non-limiting example, a biosensor can comprise a working electrode (known as transduction element, where the biochemical reaction happens), a counter electrode (to close the circuit and allow current to flow between working and counter electrode), a substrate (where the electrodes are deposited on) and a reference electrode (e.g. made from Ag/AgCl), kept at a distance from the reaction site to maintain a known and stable potential.

The sdAbs of the present invention can be immobilized on the surface of the working electrode directly, e.g. by physisorption, or indirectly, e.g. by a linker. In the sense of the present invention a linker can be any conventional protein-tag, such as a His-tag (linking a sdAb to metal ions immobilized on working electrode), Strep-tag (linking a sdAb to Strep-tactin immobilized on working electrode), SpyTag (linking a sdAb to SpyCatcher immobilized on working electrode) or FLAG-tag (linking a sdAb to anti-FLAG-tag monoclonal antibodies immobilized on working electrode). A linker can also be any conventional protein-tag suitable for chemo-enzymatic conjugation, such as for Sortase-mediated linkage (linking an LPXTG-tagged sdAb to amines/lysines modified working electrode interface), BirA-mediated linkage (introducing biotin on sdAb, which then links to a strepdavidin or avidin modified working electrode interface), Lipoic Acid Ligase (LpIA)-mediated linkage (covalently attaching an azide bearing lipoic acid derivative on sdAb, which can then can be immobilized on sensor through azide-alkyne click chemistry), Tubulin tyrosine ligase (TTL)-mediated linkage (catalyzes the addition of tyrosine derivatives to an engineered Tub-tag, which enables subsequent bioorthogonal reactions with a great variety of probes or effector molecules immobilized on working electrode), or Transglutaminase-mediated linkage (catalyses the formation of stable isopeptide bond between glutamine on c-myc-tag and various primary amine functionalized proteins/substrates). A linker can also be any conventional chemistry techniques including click chemistry and copper-free click chemistry, such as EDC/NHS chemistry (linking primary amines presents on sdAb to a carboxylic acid terminated working electrode), maleimide/cysteine chemistry (linking cysteines presents on sdAb to a maleimide terminated working electrode), or DBCO chemistry (forming covalent triazole linkage to azide modified molecules). A linker can further comprise an amino acid sequence (“peptide tether”), a single- or double-stranded nucleic acid sequence (“ssDNA or dsDNA tether”), Polyethylenglycol (PEG) chains, or combinations thereof, acting as a bridge between the linker on the sdAb to the working electrode. A linker can comprise double-stranded DNA (dsDNA), single-stranded DNA (ssDNA), charged polymers, uncharged polymers, or any combination thereof. A linker can be negatively charged and comprise a DNA/DNA duplex, a peptide nucleic acid (PNA)/DNA duplex, a PNA/PNA duplex where one or both of the PNA are modified with negative charged amino acids, a rigid anionic polyelectrolyte, a rigid negatively charged peptide, or any combination thereof. A linker can also be positively charged and comprise a PNA/PNA duplex with lysines, a rigid cationic polyelectrolyte, a rigid positively charged peptide, or any combination thereof. A linker can have a length ranging from about 5 nm to about 20 nm. In some embodiments, a linker comprises ssDNA having a length ranging from about 10mer to about 100mer. In some embodiments, a linker comprises a dsDNA having a length ranging from about 15mer to about 60mer. In some embodiments a linker is a rigid linker. Alternatively, it may be preferred that a sdAb is directly immobilized (e.g.: via thiol-gold chemistry) on a gold working electrode surface. A DNA tether may also be used to bind the sdAb to the working electrode. In this case sdAbs are coupled to one end of a DNA tether via maleimide-cysteine chemistry. While the other end of the DNA strand is attached to the gold working electrode via thiol-gold chemistry. The linker can further comprise a detection label or a tag as defined herein, e.g. a redox reporter or an enzyme. A redox reporter can be bound to the linker.

A linker can have a first end and a second end, the first end of the linker can be bound to a surface of a biosensor electrode, e.g. working electrode, the second end can be bound to an anti-IL6 sdAb of the present invention.

The sequence of the sdAb of the present invention, i.e. SEQ ID NOs: 1, 9, 17, 25, 33, 41, 49, or 57 may be modified to include a linker sequence. The sdAb and working electrode may be attached to a substrate as a structural element. Exemplary substrates that can be used in the biosensor are glass, or ceramic or plastic (e.g.: flexible/semirigid polyimide). The biosensor of the present invention can further comprise a counter electrode and/or a reference electrode. The reference electrode, e.g. made from Ag/AgCl, is kept at a distance from the reaction site in order to maintain a known and stable potential. The working electrode serves as the transduction element in the biochemical reaction, while the counter electrode establishes a connection to the electrolytic solution so that a current can be applied to the working electrode. These electrodes preferably are conductive and chemically stable (see e.g. Grieshaber, Dorothee, et al. “Electrochemical biosensors-sensor principles and architectures.” Sensors 8.3 (2008): 1400-1458.). The biosensor of the present invention can further comprise a detection label or a tag, e.g. a red-ox reporter or an enzyme. Exemplary red-ox reporters/indicators are ferrocene, [Co(GA)₂(phen)](GA=glycolic acid, phen=1,10-phenathroline), methylene blue, metal nanoparticles (e.g., Au, Pt, Pd, Ag, Cu), pyrroloquinoline quinone (PQQ), benzoquine, Osmium(III) complexes such as Os(bpy)Cl₂³⁺, diphenylamine, or any combination thereof. These can e.g. be incorporated into a DNA linker and can be oxidized at an electrode potential, generating a measurable faraday current (see e.g. Das, Jagotamoy, et al. “Reagentless biomolecular analysis using a molecular pendulum.” Nature chemistry 13.5 (2021): 428-434.) Such a tag can be comprised in or fused e.g. to the sdAb of the present invention. A tag can be fused for example to the C-terminus or the N-terminus of a sdAb of the invention. A tag can also be comprised in a linker as disclosed herein. A sdAb of the invention can for example comprise a Cysteine-tag, a fluorescence tag, a metal tag or a poly-histidine tag (His-tag). Use of such a tag is optional for the biosensor as described herein. As used herein “conjugated” preferably means that a sdAb of the present invention is linked to/bound to/layered on to/attached to a surface of a biosensor electrode, such as a working electrode. In the sense of the present invention, an anti-IL6 sdAb can be comprised in a biosensor or can be part of a biosensor. A sdAb of the present invention may be attached to a surface of a biosensor electrode via a linker as disclosed herein or can physically be deposited (e.g. primarily by van der Waals forces) onto the surface of a biosensor electrode via adsorption physiosorption. Accordingly, “conjugated” as used herein can also mean physical deposition onto the surface of a biosensor electrode via adsorption physiosorption. As used herein “conjugated to a working electrode” can also be “conjugated to a biosensor electrode”.

Accordingly, the invention provides in one aspect a biosensor comprising a sdAb of the present invention and a working electrode.

The invention also provides a biosensor comprising a sdAb of the present invention and a working electrode, wherein the sdAb is conjugated to the working electrode.

The invention also provides a biosensor comprising a sdAb of the present invention and a working electrode, wherein the sdAb is conjugated to the working electrode via a linker.

The invention also provides a biosensor comprising a sdAb of the present invention and a working electrode, wherein the sdAb is conjugated to the working electrode via a linker, wherein the linker is selected from the group of:
(i) protein-tag, such as His-tag, Strep-tag, SpyTag, or FLAG-tag,
(ii) protein-tag suitable for chemo-enzymatic conjugation, such as for Sortase-mediated linkage, BirA-mediated linkage, Lipoic Acid Ligase (LpIA)-mediated linkage, Tubulin tyrosine ligase (TTL)-mediated linkage, Transglutaminase-mediated linkage,
(iii) conventional chemistry techniques, such as click chemistry, copper-free click chemistry, EDC/NHS chemistry, maleimide/cysteine chemistry, or DBCO chemistry,
(iv) amino acid sequence, such as a peptide tether,
(v) nucleic acid sequence, such as a single stranded DNA or a double stranded DNA,
(vi) Polyethylenglycol (PEG).

The invention also provides a biosensor comprising a sdAb of the present invention and a working electrode, wherein the sdAb is conjugated to the working electrode, and a counter electrode and/or a reference electrode, and a substrate.

The invention also provides a biosensor comprising at least a sdAb of the present invention and a working electrode, and a substrate.

The invention also provides a biosensor comprising a sdAb of the present invention and a working electrode, wherein the working electrode comprises a conductive material, such as carbon, metal or metallic compound.

The invention also provides a biosensor comprising a sdAb of the present invention and a working electrode, wherein the sdAb comprises a detection label or tag.

In another aspect, the invention provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb is conjugated a working electrode.

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb is conjugated to a working electrode via a nucleic acid (linker).

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb is conjugated to a working electrode, further comprising a counter electrode and/or a reference electrode, and a substrate.

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31 in a biosensor, comprising at least an anti-IL6 sdAb of the present invention, a working electrode, and a substrate.

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb is conjugated a working electrode, wherein the working electrode comprises a conductive material, such as carbon, metal or metallic compound.

The invention also provides an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31, wherein the anti-IL6 sdAb is conjugated a working electrode, wherein the sdAb comprises a detection label or tag.

It is envisioned herein that a sdAb of the invention can be combined with other antibodies e.g. other antibodies/sdAbs binding IL6 or other antibodies/sdAbs binding any other/further targets.

It is also envisioned herein, that one or more anti-IL6 sdAb of the invention can be combined in the products, methods, uses and purpose limited products provided herein. Accordingly, one or more sdAbs of the present invention can be any one of the sdAbs defined in SEQ ID NOs: 1, 9, 17, 25, 33, 41, 49, or 57. Similarly, two or more sdAbs of the present invention can be any one of the sdAbs defined in SEQ ID NOs: 1, 9, 17, 25, 33, 41, 49, or 57. In the sense of the present invention “one or more sdAb of the present invention” preferably means one, two, three, four, five, six, seven, or eight anti-IL6 sdAbs selected from the group of SEQ ID NOs: 1, 9, 17, 25, 33, 41, 49, or 57. In the sense of the present invention “two or more sdAb of the present invention” preferably means two, three, four, five, six, seven, or eight anti-IL6 sdAbs selected from the group of SEQ ID NOs: 1, 9, 17, 25, 33, 41, 49, or 57.

The present invention also provides a nucleic acid encoding an anti-IL6 sdAb of the invention. A “nucleic acid” in the sense of the present invention is a polymer of nucleotides, such as a polynucleotide. A nucleic acid can be any nucleic acid, preferably a deoxyribonucleic acid (DNA) or a ribonucleic acid (RNA). A nucleic acid encoding an anti-IL6 sdAb of the invention is particularly useful for producing the sdAb of the invention. For example, a nucleic acid can comprise a DNA plasmid or DNA vector encoding an anti-IL6 sdAb of the invention. A DNA plasmid or DNA vector can comprise additional elements that aid the transcription and translation of the sdAb in a host cell, such as artificial or viral promoters. For example, a DNA plasmid or DNA vector can further comprise a strong viral promoter, such as the simian vacuolating virus 40 (SV40) promoter, the Rous Sarcoma Virus (RSV) promoter, the cytomegalovirus (CMV) immediate early promoter or a retroviral cis-acting transcriptional element. A DNA plasmid or DNA vector can further comprise strong polyadenylation/transcriptional termination signals, such as bovine growth hormone or rabbit beta-globulin polyadenylation sequences. A DNA plasmid or DNA vector can further comprise modifications, such as insertion of enhancer sequences, synthetic introns, adenovirus tripartite leader (TPL) sequences and modifications to the polyadenylation and transcriptional termination sequences. The DNA plasmid or DNA vector of the present invention can be codon optimized for expression in a host, host cell or organism. Useful hosts, host cells or organisms for sdAb production are for example viruses or bacteria, such as bacteriophages, Escherichia coli, E. coli strain BL21, E. coli strain TG1, yeast, insect cells, or mammalian cells. For example the E. coli strain BL21 or E. coli strain TG1 can be used to produce sdAbs of the present invention. The DNA of the present invention can be single-stranded DNA or double-stranded DNA. The DNA of the present invention is preferably double stranded. The nucleic acid, such as a DNA, of the present invention can be present in a host cell, e.g. for the purpose of expressing or producing a sdAb of the invention. Accordingly, the invention provides a host cell comprising a nucleic acid encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

The invention also provides a nucleic acid encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

In another aspect, the invention provides a DNA encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

In another aspect, the invention provides a DNA vector encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

In another aspect, the invention provides a DNA expression vector encoding an anti-interleukin 6 (IL6) single-domain antibody (sdAb) comprising an on-rate constant (kₒₙ) of about 6.3×10∝-1.3×10\hat{ }7 and an off-rate constant (k_{off}) of about 1.2×10\hat{ }−6-0.31.

In another aspect the invention provides a RNA encoding an anti-IL6 sdAb of the invention. A sdAb of the present invention can be encoded by e.g. a messenger ribonucleic acid (mRNA) which is typically a single-stranded molecule of RNA that can be used by a ribosome for peptide synthesis. In the sense of the present invention a RNA encoding an anti-IL6 sdAb of the invention is preferably a mRNA. The mRNA encoding an anti-IL6 sdAb of the invention is delivered to a host cell where it is translated into an anti-IL6 sdAb or can be used in in vitro transcription. In general, any mRNA construct encoding an anti-IL6 sdAb that is capable of expressing an anti-IL6 sdAb can be used in the sense of the present invention.

Accordingly, the invention provides a RNA encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

In another aspect, the invention provides a mRNA encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

The skilled person is aware that, except where otherwise noted, polynucleotide sequences set forth in the present application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U”.

A mRNA can comprise at least an open reading frame encoding an anti-IL6 sdAb of the present invention. The basic components of an mRNA molecule typically include at least one coding regions, a 5′ untranslated region (UTR), a 3′ UTR, a 5′ cap and a poly-A tail. Polynucleotides of the present invention may function as mRNA but can be distinguished from wild-type mRNA in their functional and/or structural design features which serve to overcome existing problems of effective polypeptide expression e.g. when using nucleic-acid based therapeutics or when used for optimized expression/production of sdAbs. The mRNAs of the present invention may be codon optimized for expression in a host, host cell or organism. The nucleic acid, such as a RNA, of the present invention can be present in a host cell, e.g. for the purpose of expressing or producing a sdAb of the invention. Accordingly, the invention provides a host cell comprising a RNA encoding an anti-interleukin 6 (IL6) single-domain antibody (sdAb) comprising an on-rate constant (kₒₙ) of about 6.3×10\hat{ }4-1.3×10\hat{ }7 and an off-rate constant (k_{off}) of about 1.2×10\hat{ }−6-0.31.

The sample and/or biological fluid to be used and/or analyzed herein preferably have a physiological pH, e.g. about 7.0 to about 7.5, preferably about 7.2 to 7.4. For example, the pH of (arterial) blood normally is within a range of about 7.35-7.45. Intracellular pH (e.g. pH of interstitial fluid) can range between about 7.03 to 7.46.

It is preferred herein that the sample and/or biological fluid is blood or interstitial fluid.

The invention also provides a method for detecting and/or quantifying IL6, e.g. in a sample. Such a method makes use of the sdAbs provided herein which specifically bind IL6 within the entire dynamic range of the IL6 level. As described herein above, it is especially useful to detect and/or quantify IL6 levels in a sample to evaluate the inflammation status of a subject or monitor efficacy and tolerability of a treatment. Such a method can comprise contacting a sample with an anti-IL6 sdAb of the invention and detecting and/or quantifying binding of the anti-IL6 sdAb. A sample used in the sense of the present invention can be any sample that allows for detection and/or quantification of IL6 levels. A sample can be for example wherein a biological fluid, such as a bodily fluid. Exemplary samples are interstitial fluid (ISF), blood, urine, stool, serum, tear fluid, sweat, saliva and the like.

Accordingly, the invention provides in one aspect a method for detecting and/or quantifying IL6 in a sample, the method comprising:
(i) contacting a sample with the anti-IL6 sdAb of the invention, and
(ii) detecting binding of the anti-IL6 sdAb,
   wherein the sample comprises interstitial fluid (ISF), blood, urine, stool, serum, tear fluid, sweat, or saliva.

Similarly, the invention is not limited to means of contacting a sample, any means that are feasible for contacting an IL6 with an anti-IL6 sdAb of the invention can be used. Exemplary means of contacting a sample with an anti-IL6 sdAb of the invention are collection through a finger stick/prick or syringe and deposition on the anti-IL6 sdAb and/or biosensor, collection through microfluidic or capillary-based system to the anti-IL6 sdAb and/or biosensor surface, collection through needle or microneedle-based system, direct implantation of the biosensor comprising the anti-IL6 sdAb in contact with the bodily fluid of interest. Other means of contacting a sample with an anti-IL6 sdAb and/or biosensor of the invention comprise determining a body site where to collect a sample (e.g. bodily fluid) from (ex vivo) and/or contacting a sample (e.g. body fluid) with the anti-IL6 sdAb and/or the biosensor (in vivo). In the sense of the present invention, a sample may be contacted with an anti-IL6 sdAb conjugated to a working electrode in a biosensor. Similarly, a sdAb conjugated to a working electrode in a biosensor may be contacted with an IL6 or a sample comprising IL6.

Accordingly, the invention provides in one aspect a method for detecting and/or quantifying IL6 in a sample, the method comprising:
(i) contacting a sample with the anti-IL6 sdAb of the invention, and
(ii) detecting binding of the anti-IL6 sdAb,
   wherein the contacting step comprises contacting a sample via a syringe, a needle, a microfluidic or capillary-based system with the anti-IL6 sdAb of the invention.

In another aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   wherein the contacting step comprises contacting the sample with a biosensor of the invention, optionally wherein the biosensor comprises a sdAb of the invention conjugated to a working electrode.

In general, the invention is not limited to any means of detecting a sdAb, an IL6 and/or an IL6 bound by a sdAb of the present invention. Any means allowing for detection/quantification can be used in the sense of the invention. Exemplary antibody-based detection methods are well known in the art, and include, for example, ELISA, electrochemical ELISA, immunoblots, radioimmunoassays, immunofluorescence, immunoprecipitation, and other related techniques. Association of a sdAb of the invention and IL6 can also be physiochemically such as electrochemically detected/quantified using physiochemical or electrochemical techniques as described herein. The electric signal that is generated can be readout by any electrochemical techniques commonly used, such as amperometry, chronoamperometry, potentiometry, conductometry, voltammetry, cyclic voltammetry, galvanic cell, impedance spectroscopy, and the like resulting in detection/quantification of sdAb and IL6 association. It is preferred herein, that IL6 levels are detected/quantified making use of the biosensor as described herein above.

Accordingly, the method for detecting and/or quantifying IL6 in a sample can be performed using a biosensor as described herein, such as a biosensor comprising a sdAb of the invention conjugated to a working electrode.

Accordingly, the invention provides in one aspect a method for detecting and/or quantifying IL6 in a sample, the method comprising:
(i) contacting a sample with the anti-IL6 sdAb of the invention, and
(ii) detecting binding of the anti-IL6 sdAb,
   wherein the detection step comprises physiochemical or electrochemical detection of the binding of the anti-IL6 sdAb and IL6.

In another aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   wherein the detection step comprises detection of the binding of the anti-IL6 sdAb and IL6 via an electrical signal produced by the binding.

In another aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   wherein the detection step comprises detection of the binding of the anti-IL6 sdAb and IL6 with a biosensor of the invention, optionally, wherein the biosensor comprises a sdAb of the invention conjugated to a working electrode.

In a preferred aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   wherein the contacting step comprises contacting the sample with a biosensor of the invention, optionally wherein the biosensor comprises a sdAb of the invention conjugated to a working electrode and,
   wherein the detection step comprises detecting the binding of the binding of the anti-IL6 sdAb and IL6 with a biosensor of the invention, optionally, wherein the biosensor comprises a sdAb of the invention conjugated to a working electrode.

Electrochemical ELISA can be used as an exemplary means for detecting and/or quantifying IL6 or in a detecting unit for detecting and/or quantifying IL6. In brief, the anti-IL6 sdAb can be directly or indirectly immobilized/conjugated to a working electrode. The working electrode can be incubated with samples (e.g. interstitial fluid (ISF), blood, urine, stool, serum, tear fluid, sweat, or saliva) to be tested for IL6 levels. During incubation, IL6 can bind to the anti-IL6 sdAb. After incubation, the working electrode can be washed, e.g. with phosphate buffered saline (PBS). A solution with a secondary antibody (i.e. antibody binding the anti-IL6 sdAb) modified with biotin can be added and incubated, followed by another washing step of the working electrode. A solution containing streptavidin modified enzyme, such as alkaline phosphatase (ALP), polymeric alkaline phosphatease (PALP), or Horseradish Peroxidase (HRP), can be added to the working electrode, followed by another washing step of the working electrode. A solution containing enzyme substrate such as 4-APP, ABTS, AEC, DAB, ECL, TMB, Amplex Red, BCIP/NBT (5-Bromo-4-chloro-3-indolyl phosphate along with nitro blue tetrazolium), pNPP (para-Nitrophenylphosphate), or Fast Red, can then be added, and the electrochemical oxidation signal can be recorded, e.g. by using differential pulse voltammetry. An alternative approach of applying electrochemical ELISA is to directly or indirectly immobilize/conjugate the anti-IL6 sdAb to a working electrode. The working electrode can be incubated with samples (e.g. interstitial fluid (ISF), blood, urine, stool, serum, tear fluid, sweat, or saliva) to be tested for IL6 levels. During incubation, IL6 can bind to the anti-IL6 sdAb. After incubation, the working electrode can be washed, e.g. with phosphate buffered saline (PBS). A solution with a secondary antibody (i.e. antibody binding the anti-IL6 sdAb) modified with an enzyme, such as alkaline phosphatase (ALP), polymeric alkaline phosphatease (PALP), or Horseradish Peroxidase (HRP), can be added and incubated, followed by another washing step of the working electrode. A solution containing enzyme substrate such as 4-APP, ABTS, AEC, DAB, ECL, TMB, Amplex Red, BCIP/NBT (5-Bromo-4-chloro-3-indolyl phosphate along with nitro blue tetrazolium), pNPP (para-Nitrophenylphosphate), or Fast Red, can then be added, and the electrochemical oxidation signal can be recorded, e.g. by using differential pulse voltammetry.

Biosensing via electrochemical ELISA is for example disclosed in Arya, Sunil K., and Pedro Estrela. “Electrochemical ELISA protein biosensing in undiluted serum using a polypyrrole-based platform.” Sensors 20.10 (2020): 2857.

Accordingly, the invention provides in one aspect, a method for detecting and/or quantifying IL6 in a sample, the method comprising:
(i) contacting a sample with the anti-IL6 sdAb of the invention, and
(ii) detecting binding of the anti-IL6 sdAb,
   wherein the detection step comprises detection of the binding of the anti-IL6 sdAb and IL6 via electrochemical ELISA.

The method for detecting and/or quantifying IL6 in a sample as described above can be ex vivo or in vitro. Accordingly, the invention provides in one aspect an ex vivo or in vitro method for detecting and/or quantifying IL6 in a sample, the method comprising:
(i) contacting a sample with an anti-IL6 sdAb of the present invention, and
(ii) detecting binding of the anti-IL6 sdAb.

In some aspects of the invention, the methods described herein can further comprise quantifying the IL6 level in a sample or a subject and comparing the IL6 level with a reference sample or value. A reference sample may be a sample comprising an IL6 level corresponding to any specific inflammation conditions, such as inactive and active IBD, COVID, cancer, sepsis, and septic shock. A reference sample may be a standardized sample, i.e. a sample comprising IL6 levels that have been standardized to certain IL6 levels. Such IL6 levels may correspond to values or ranges of IL6 levels indicating e.g. baseline inflammation, low inflammation, moderate inflammation, high inflammation or severe inflammation in a sample or subject. Accordingly, the IL6 level in a sample or a subject may also be compared to a IL6 level value. An IL6 level value can be obtained from a reference sample as described herein or e.g. from the literature. IL6 level reference sample or values and thereby the inflammation status they indicate may vary dependent on the subject, the disease a subject may be suffering from and/or the treatment a subject is receiving. The skilled practitioner is aware and knows what reference samples or values can be used to properly assess a subject's inflammation status. Exemplary reference values may be obtained from Mavropoulou et al. 2020, Zhou et al. 2020, Lippitz et al. 2016 and/or Song et al. 2019.

For example, the following reference values may be used:

TABLE 1
IL6 concentrations in humans during homeostasis and disease.

| | | |
|---|---|---|
| | | Example |
| | Concentration | indications/ |
| Range | in blood | disease |

| Baseline | <45 | fM | |
|---|---|---|---|
| Low inflammation | 45 fM- | | Inactive IBD |
| | 225 fM | | |
| Moderate | 225 fM- | | COVID survivors |
| inflammation | 450 fM | | Cancer patients |
| | | | Active IBD |
| High inflammation | 450 fM- | | COVID non-survivors |
| | 4.5 pM | | Sepsis |
| Severe inflammation | >4.5 | pM | Septic shock |

Detection and/or quantification of IL6 in a sample as described herein above can for example be realized by a spot measurement of the IL6 levels making use of the sdAb of the present invention. For example, the method for detecting and/or quantifying IL6 can comprise obtaining a sample from a subject, contacting the sample with an anti-IL6 sdAb of the invention, and detecting binding of the anti-IL6 sdAb. The method can further comprise quantifying the IL6 level in the sample, which correlates with the binding kinetics between sdAb and IL6. In an exemplary embodiment, a spot measurement can be realized in the method for detecting and/or quantifying IL6 comprising obtaining a sample from a subject e.g. obtaining a drop of blood by fingerpick, contacting the sample with an anti-IL6 sdAb of the invention e.g. collecting the sample through capillary mechanisms or deposition onto an anti-IL6 sdAb or a biosensor comprising the same as disclosed herein, detecting binding of the anti-IL6 sdAb, thereby quantifying the IL6 level in the sample and the subject. In some embodiments of the method, the anti-IL6 sdAb may be conjugated to a working electrode in a biosensor as described herein. Accordingly, the invention provides a method for detecting and/or quantifying IL6 in a sample, the method comprising:
(i) contacting a sample with the anti-IL6 sdAb of the invention, and
(ii) detecting binding of the anti-IL6 sdAb.

The method can be repeated for samples from individual subjects. This is particularly desired when the development or dynamics of the IL6 levels in a subject are relevant for a therapy of the subject. Rising levels of IL6 over time correlate with increased inflammation, while declining levels of IL6 over time correlate with reduced inflammation. For example, a rising level of IL6 can indicate an infectious disease. Particularly, the IL6 levels can be useful to indicate whether a subject suffers from a bacterial or viral infection. IL6 levels are elevated in subjects suffering from infection, when treated with antibiotics, IL6 levels will decline if the subject suffers from a bacterial infection. In contrast, when treated with antibiotics, IL6 levels will not decline if the subject suffers from viral infection. Accordingly, it is especially useful to monitor the IL6 levels in a subject over time, e.g. to arrive at a clinical conclusion of the best treatment of a subject. By repeating the spot measurement as described above, the dynamic IL6 level in a subject can be detected over time allowing the skilled practitioner to make reasoned treatment decisions, e.g. in case of bacterial vs. viral infection. In addition, IL6 levels can correlate with inflammation spikes in inflammatory diseases and/or autoimmune diseases. Exemplary inflammatory and/or autoimmune diseases in the sense of the invention are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. Inflammatory and/or autoimmune diseases may require close medical attention and medication. During the course of an inflammatory and/or autoimmune disease it is especially useful to monitor the IL6 levels in a subject. By doing so, inflammation spikes can be detected enabling targeted therapy and medication which reduces the total amount of administered medication needed and thereby potential side effects compared to a continuous treatment. Accordingly, the detection and/or quantification of the IL6 levels in a subject enables the skilled practitioner to modulate and personalize the treatment of a subject while reducing the total amount of drugs administered, particularly, if the IL6 levels are detected/quantified over time. Thus, the detection and/or quantification of IL6 levels over time is desired for improved disease management. In some embodiments, the method for detecting and/or quantifying IL6 in a sample does not comprise a step of obtaining a sample from a subject.

Accordingly, in one aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   further comprising detecting the binding of the anti-IL6 sdAb over time.

In another aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   further comprising detecting the binding of the anti-IL6 sdAb over time, wherein steps (i) and (ii) are performed on a further sample, optionally wherein the sample is from the same subject.

In yet another aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   further comprising contacting and/or detecting the binding of the anti-IL6 sdAb over time, wherein steps (i) and (ii) are performed on a further sample, optionally wherein the sample is from the same subject, and
   wherein the contacting step comprises contacting the sample with a contacting a sample with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another preferred aspect, the invention provides a method for detecting and/or quantifying IL6 in a sample, the method comprising:
(i) contacting a sample with the anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are performed on a further sample, optionally wherein the sample is from the same subject, and
   wherein the contacting step comprises contacting the sample with a contacting a sample with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another preferred aspect, the invention provides a method for detecting and/or quantifying IL6 in a sample, the method comprising:
(i) contacting a sample comprising interstitial fluid (ISF), blood, urine, stool, serum, tear fluid, sweat, or saliva with the anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are performed on a further sample, optionally wherein the sample is from the same subject, and
   wherein the contacting step comprises contacting the sample with a contacting a sample with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another preferred aspect, the invention provides a method for detecting and/or quantifying IL6 in a sample, the method comprising:
(i) contacting a sample comprising interstitial fluid (ISF), blood, urine, stool, serum, tear fluid, sweat, or saliva with the anti-IL6 sdAb of the invention, and
(ii) detecting binding of the anti-IL6 sdAb,
   wherein the detection step comprises detection of the binding of the anti-IL6 sdAb and IL6 via electrochemical ELISA.

It is also envisioned herein to use more than one sdAb of the invention in a method for detecting and/or quantifying IL6 in a sample. Two or more anti-IL6 sdAb can be used to combine specific desired ranges of on-rate constant (kₒₙ) and off-rate constant (k_{off}) to arrive at the desired association and dissociation dynamics. Accordingly, the method for detecting and/or quantifying IL6 in a sample of the present invention can comprise contacting a sample with one or more, such as one, two, three, four, five, six, seven, or eight anti-IL6 sdAb of the present invention. In some embodiments, the method for detecting and/or quantifying IL6 in a sample of the present invention can comprise contacting a sample with two or more, such as two, three, four, five, six, seven, or eight anti-IL6 sdAb of the present invention.

Accordingly, the invention provides in one aspect a method for detecting and/or quantifying IL6 in a sample, the method comprising:
(i) contacting a sample with one or more anti-IL6 sdAb of the invention, and
(ii) detecting binding of the one or more anti-IL6 sdAb.

In another aspect, the invention provides a method for detecting and/or quantifying IL6 in a sample, the method comprising:
(i) contacting a sample with one or more, such as one, two, three, four, five, six, seven, or eight anti-IL6 sdAbs of the invention, and
(ii) detecting binding of the one or more, such as one, two, three, four, five, six, seven, or eight anti-IL6 sdAbs.

In another aspect, the invention provides a method for detecting and/or quantifying IL6 in a sample, the method comprising:
(i) contacting a sample with two or more anti-IL6 sdAb of the invention, and
(ii) detecting binding of the two or more anti-IL6 sdAb.

In another aspect, the invention provides a method for detecting and/or quantifying IL6 in a sample, the method comprising:
(i) contacting a sample with two or more, such as two, three, four, five, six, seven, or eight anti-IL6 sdAb of the invention, and
(ii) detecting binding of the two or more, such as two, three, four, five, six, seven, or eight anti-IL6 sdAb.

As described herein, a particular advantage of the sdAb of the invention is the reversible and continuous binding of IL6. Spot measurements as described herein are particularly useful to determine levels of IL6. In another aspect, it is also desired to continuously monitor the levels of IL6 in a subject, e.g. avoiding obtaining samples from a subject at multiple time points. Reducing the sample collection steps, e.g. blood draws on a subject, is especially advantageous, since it reduces treatment burden on the subject. For this, the measurement of IL6 levels can be in or on a subject, i.e. in vivo.

Accordingly, in one aspect the invention provides a method for detecting and/or quantifying IL6 in a subject, the method comprising:
(i) contacting a subject with the anti-IL6 sdAb of the invention, and
(ii) detecting binding of the anti-IL6 sdAb.

In general, the features provided for the method for detecting and/or quantifying IL6 in a sample herein above can be applied to the method for detecting and/or quantifying IL6 in a subject. In some aspects, it is not necessary to obtain a sample from a subject when a subject's interstitial fluid (ISF), blood, urine, stool, serum, tear fluid, sweat, saliva and the like is contacted with an anti-IL6 sdAb of the invention. When the IL6 level is detected on or in the subject, a subject's interstitial fluid (ISF), blood, urine, stool, serum, tear fluid, sweat, saliva and the like may be contacted directly with an anti-IL6 sdAb of the invention, or e.g. a biosensor comprising a sdAb of the invention conjugated to a working electrode. The invention is not particularly limited to any means of contacting, rather any means of contacting that allow a sdAb of the invention to come in close proximity with a IL6 of the subject (i.e. that binding/association of an anti-IL6 sdAb of the invention and an IL6 of the subject is realized) can be used. Exemplary forms of contacting a subject's interstitial fluid (ISF), blood, urine, stool, serum, tear fluid, sweat, saliva and the like can be via a syringe, a needle, a microfluidic or capillary-based system or the like. In one aspect, the invention provides a method for detecting and/or quantifying IL6 in a subject, the method comprising:
(i) contacting a subject's interstitial fluid (ISF), blood, urine, stool, serum, tear fluid, sweat, or saliva with an anti-IL6 sdAb of the invention, and
(ii) detecting binding of the anti-IL6 sdAb.

In another aspect, the invention provides a method for detecting and/or quantifying IL6 in a subject, the method comprising:
(i) contacting a subject's interstitial fluid (ISF) with an anti-IL6 sdAb of the invention, and
(ii) detecting binding of the anti-IL6 sdAb.

In the sense of the present invention, a subject may be contacted with an anti-IL6 sdAb conjugated to a working electrode in a biosensor. Similarly, a sdAb conjugated to a working electrode in a biosensor may be contacted with a subject's interstitial fluid (ISF), blood, urine, stool, serum, tear fluid, sweat, saliva and the like comprising IL6.

Accordingly, the invention provides in one aspect a method for detecting and/or quantifying IL6 in a subject, the method comprising:
(i) contacting a subject with the anti-IL6 sdAb of the invention, and
(ii) detecting binding of the anti-IL6 sdAb,
   wherein the contacting step comprises contacting the subject with a biosensor of the invention, optionally wherein the biosensor comprises a sdAb of the invention conjugated to a working electrode.

As described herein above, the method for detecting and/or quantifying IL6 can be performed using a biosensor as described herein, e.g. a biosensor comprising a sdAb of the invention conjugated to a working electrode. This can also be applied to the method for detecting and/or quantifying IL6 in a subject of the invention.

Accordingly, in another aspect the invention provides a method for detecting and/or quantifying IL6 in a subject, the method comprising:
(i) contacting a subject with the anti-IL6 sdAb of the invention, and
(ii) detecting binding of the anti-IL6 sdAb,
   wherein the contacting step comprises contacting the subject with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another aspect the invention provides a method for detecting and/or quantifying IL6 in a subject, the method comprising:
(i) contacting a subject with the anti-IL6 sdAb of the invention, and
(ii) detecting binding of the anti-IL6 sdAb,
   wherein the detection step comprises detection of the binding of the anti-IL6 sdAb and IL6 via electrochemical ELISA.

The method of the invention for detecting and/or quantifying IL6 in a subject, can further comprise quantifying the IL6 level in a subject and comparing the IL6 level with a reference sample or value. Thus, the statements described herein above regarding further comprising quantifying the IL6 level and comparing the IL6 level with a reference sample or value also apply to the method of the invention for detecting and/or quantifying IL6 in a subject.

Similarly, to the spot type measurements in a sample as described above, the invention also provides a means to measure the IL6 levels in or on a subject over time, i.e. a method for detecting and/or quantifying IL6 in a subject over time. Measuring IL6 levels over time is especially advantageous when done in or on a subject since no additional samples have to be obtained from the subject reducing the burden on the subject. This allows to reduce the time steps in which the IL6 levels are measured in the subject resulting in a superior resolution of the dynamic IL6 level in the subject over time. The thereby increased resolution of the dynamic IL6 levels in the subject allow the skilled practitioner to precisely modulate and personalize the treatment of a subject while reducing the total amount of drugs administered, particularly. Thus, the detection and/or quantification of IL6 levels over time in a subject result in improved disease management. The method for detecting and/or quantifying IL6 in or on a subject as described herein can be realizes within a device or apparatus in form of an applicator like appliance, such as a handheld or stationary device comprising the biosensor of the present invention, or in form of a permanently or reversibly implanted or attached (to the subject's body) like appliance, such as an implanted, wearable device comprising the biosensor of the present invention. As used herein “detecting binding of the anti-IL6 sdAb to IL6” in the context of a sdAb comprised in a biosensor or apparatus of the present invention can include or be “detecting an electrochemical signal (optionally where detection of said signal indicates the presence of IL-6 and/or wherein the signal strength indicates the amount of IL6)”. Devices and apparatuses useful for performing the methods provided herein are described below in more detail.

Accordingly, in one aspect the invention provides a method for detecting and/or quantifying IL6 in a subject over time, the method comprising:
(i) contacting a subject with the anti-IL6 sdAb of the invention, and
(ii) detecting binding of the anti-IL6 sdAb.

In another aspect, the invention provides a method for detecting and/or quantifying IL6 in a subject, the method comprising:
(i) contacting a subject with the anti-IL6 sdAb of the invention, and
(ii) detecting binding of the anti-IL6 sdAb,
   further comprising contacting and/or detecting the binding of the anti-IL6 sdAb over time, wherein steps (i) and (ii) are repeatedly performed in or on the subject.

In a preferred aspect, the invention provides a method for detecting and/or quantifying IL6 in a subject, the method comprising:
(i) contacting a subject with the anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are repeatedly performed in or on the subject, and
   wherein the contacting step comprises contacting the subject with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another preferred aspect, the invention provides a method for detecting and/or quantifying IL6 in a subject, the method comprising:
(i) contacting a subject's interstitial fluid (ISF), blood, urine, stool, serum, tear fluid, sweat, or saliva with the anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are repeatedly performed in or on the subject, and
   wherein the contacting step comprises contacting the subject with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

It is also envisioned herein to use more than one sdAb of the invention in a method for detecting and/or quantifying IL6 in a subject. Two or more anti-IL6 sdAb can be used to combine specific desired ranges of on-rate constant (kₒₙ) and off-rate constant (k_{off}) to arrive at the desired association and dissociation dynamics. Accordingly, the method for detecting and/or quantifying IL6 in a subject of the present invention can comprise contacting a subject with one or more, such as one, two, three, four, five, six, seven, or eight anti-IL6 sdAb of the present invention. In some embodiments, the method for detecting and/or quantifying IL6 in a subject of the present invention can comprise contacting a subject with two or more, such as two, three, four, five, six, seven, or eight anti-IL6 sdAb of the present invention.

Accordingly, the invention provides in one aspect a method for detecting and/or quantifying IL6 in a subject, the method comprising:
(i) contacting a subject with one or more anti-IL6 sdAb of the invention, and
(ii) detecting binding of one or more anti-IL6 sdAb.

In another aspect, the invention provides a method for detecting and/or quantifying IL6 in a subject, the method comprising:
(i) contacting a subject with one or more, such as one, two, three, four, five, six, seven, or eight anti-IL6 sdAb of the invention, and
(ii) detecting binding of one or more, such as one, two, three, four, five, six, seven, or eight anti-IL6 sdAb.

In another aspect, the invention provides a method for detecting and/or quantifying IL6 in a subject, the method comprising:
(i) contacting a subject with two or more anti-IL6 sdAb of the invention, and
(ii) detecting binding of two or more anti-IL6 sdAb.

In another aspect, the invention provides a method for detecting and/or quantifying IL6 in a subject, the method comprising:
(i) contacting a subject with two or more, such as two, three, four, five, six, seven, or eight anti-IL6 sdAb of the invention, and
(ii) detecting binding of two or more, such as two, three, four, five, six, seven, or eight anti-IL6 sdAb.

It is also encompassed herein that a level of IL6 can be detected and/or quantified in sample from a subject or in a subject in its original place, i.e. at the original location of the sample or at the location in a subject (in situ). Thus, the anti-IL6 sdAb, the biosensor comprising the same and the apparatuses comprising the same of the present invention are particularly useful for in situ detection and/or quantification of IL6. An in situ method as disclosed herein can comprise or be an in vivo method. Accordingly, the invention provides in one aspect an in situ method for detecting and/or quantifying IL6 in a subject, the method comprising:
(i) contacting a subject's biological fluid or sample e.g. a bodily fluid with an anti-IL6 sdAb of the invention, and
(ii) detecting binding of the anti-IL6 sdAb.

In another aspect, the invention provides an in situ method for detecting and/or quantifying IL6 in a subject, the method comprising:
(i) contacting a subject's bodily fluid e.g. interstitial fluid (ISF), blood, urine, stool, serum, tear fluid, sweat or saliva with an anti-IL6 sdAb of the invention, and
(ii) detecting binding of the anti-IL6 sdAb.

In general, the features provided for the method for detecting and/or quantifying IL6 in a subject herein above can also be applied mutatis mutandis to the in situ method for detecting and/or quantifying IL6 in a subject.

The features described herein in context of the method for detecting and/or quantifying IL6 in a sample apply mutatis mutandis to the method for detecting and/or quantifying IL6 in a subject. In some aspect, the method for detecting and/or quantifying IL6 in a subject may not comprise a step of obtaining a sample from the subject.

The methods for detecting and/or quantifying IL6 in a sample or in/on a subject can be performed for example in an apparatus or performed by an apparatus. An apparatus comprising components for performing the methods described herein is especially useful for efficient and quick measurement of IL6 levels in a sample or in/on a subject. Thus, an apparatus according to the present invention can be used in any spot measurement or any continuous measurement of IL6 as described herein. An apparatus of the present invention is not particularly limited, in general, an apparatus comprising an anti-IL6 sdAb of the invention and any means of measuring/detecting/quantifying association/binding of the anti-IL6 sdAb of the invention to IL6 can be used.

The apparatus of the present invention can comprise one or more anti-IL6 sdAb of the present invention. Using more than one sdAb can be preferred herein to take advantage of the individual properties of anti-IL6 sdAb provided herein. For example, a biosensor or apparatus provided herein comprising two or more anti-IL6 sdAb can be used to combine specific desired ranges of on-rate constant (kₒₙ) and off-rate constant (k_{off}) to arrive at the desired association and dissociation dynamics. In some embodiments, the apparatus of the present invention comprises one or more, such as one, two, three, four, five, six, seven, or eight anti-IL6 sdAb of the present invention. In some embodiments, the biosensor of the present invention comprises one or more, such as one, two, three, four, five, six, seven, or eight anti-IL6 sdAb of the present invention. In some embodiments, one or more, such as one, two, three, four, five, six, seven, or eight anti-IL6 sdAb of the present invention can be used in any of the products, methods or uses of the present invention.

Accordingly, the invention provides in one aspect an apparatus comprising:
(i) an anti-IL6 sdAb of the invention, and
(ii) a contacting unit for contacting a sample with the anti-IL6 sdAb, and
(iii) a detecting unit for detecting and/or quantifying binding of the anti-IL6 sdAb.

In another aspect, the invention provides an apparatus comprising:
(i) an anti-IL6 sdAb of the invention, and
(ii) a contacting unit for contacting a subject with the anti-IL6 sdAb, and
(iii) a detecting unit for detecting and/or quantifying binding of the anti-IL6 sdAb.

In another aspect, the invention provides an apparatus comprising:
(i) one or more, such as one, two, three, four, five, six, seven, or eight anti-IL6 sdAb of the invention, and
(ii) a contacting unit for contacting a sample with the one or more anti-IL6 sdAb, and
(iii) a detecting unit for detecting and/or quantifying binding of the one or more anti-IL6 sdAb.

In another aspect, the invention provides an apparatus comprising:
(i) one or more, such as one, two, three, four, five, six, seven, or eight anti-IL6 sdAb of the invention, and
(ii) a contacting unit for contacting a subject with the one or more anti-IL6 sdAb, and
(iii) a detecting unit for detecting and/or quantifying binding of the one or more anti-IL6 sdAb.

The detecting unit for detecting and/or quantifying binding of the anti-IL6 sdAb can comprise for example a contacting unit for contacting a sample or a subject with the anti-IL6 sdAb of the invention. Such means can be comprised in a first unit which allows a sample or a subject to be contacted. As described above, exemplary forms of contacting a sample or a subject can be via a syringe, a needle, a microfluidic or capillary-based system or the like. A sample to be contacted with the first unit can comprise interstitial fluid (ISF), blood, urine, stool, serum, tear fluid, sweat, saliva and the like. The contacting step is in a subject, a subject's interstitial fluid (ISF), blood, urine, stool, serum, tear fluid, sweat, saliva and the like can be contacted by the first unit. In some embodiments it is preferred that a sample comprises interstitial fluid or blood. In some embodiments it is preferred that a subject's interstitial fluid or blood is contacted. The detecting unit for detecting and/or quantifying binding of the anti-IL6 sdAb can comprise any means that allow a quantitative readout of the association of anti-IL6 sdAb and IL6 in the sample or subject. The detecting unit for detecting and/or quantifying binding of the anti-IL6 sdAb can be comprised in a second unit which allows to detect and/or quantify binding of the anti-IL6 sdAb. Accordingly, the detecting unit for detecting and/or quantifying binding of the anti-IL6 sdAb of the present invention described in the context of the methods herein also apply to the detecting unit for detecting and/or quantifying binding of the anti-IL6 sdAb in the apparatus. For example, an anti-IL6 sdAb conjugated to a working electrode, as described herein, can be used as a detecting unit for detecting and/or quantifying binding of the anti-IL6 sdAb in the apparatus. Similarly, a biosensor as described herein can be used as a detecting unit for detecting and/or quantifying binding of the anti-IL6 sdAb in the apparatus. Other exemplary detecting units for detecting and/or quantifying binding of the anti-IL6 sdAb in the apparatus can include electrochemical, optical, thermal, electronic, gravimetric, or acoustic means of detection. For example, a detecting unit for detecting and/or quantifying binding of the anti-IL6 sdAb to IL6 in the apparatus can comprise electrochemical ELISA. In some embodiments, the first and the second units can be combined in a single unit. In this case, the single unit may be the first unit.

Accordingly, the invention provides in one aspect an apparatus comprising:
(i) an anti-IL6 sdAb of the invention, and
(ii) a first unit comprising a contacting unit for contacting a sample with the anti-IL6 sdAb, and
(iii) a second unit comprising a detecting unit for detecting and/or quantifying binding of the anti-IL6 sdAb.

In another aspect, the invention provides an apparatus comprising:
(i) an anti-IL6 sdAb of the invention, and
(ii) a first unit comprising a contacting unit for contacting a subject with the anti-IL6 sdAb, and
(iii) a second unit comprising a detecting unit for detecting and/or quantifying binding of the anti-IL6 sdAb.

In another aspect, the invention provides an apparatus comprising:
(i) an anti-IL6 sdAb of the invention, and
(ii) a first unit comprising a contacting unit for contacting a sample with the anti-IL6 sdAb and comprising means for detecting and/or quantifying binding of the anti-IL6 sdAb.

In another aspect, the invention provides an apparatus comprising:
(i) an anti-IL6 sdAb of the invention, and
(ii) a first unit comprising a contacting unit for contacting a subject with the anti-IL6 sdAb and comprising a detecting unit for detecting and/or quantifying binding of the anti-IL6 sdAb.

In another aspect, the invention provides an apparatus comprising:
(i) an anti-IL6 sdAb of the invention, and
(ii) a first unit comprising a contacting unit for contacting a sample comprising interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb.

In another aspect, the invention provides an apparatus comprising:
(i) an anti-IL6 sdAb of the invention, and
(ii) a first unit comprising a contacting unit for contacting a subject's interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb.

In another aspect, the invention provides an apparatus comprising:
(i) an anti-IL6 sdAb of the invention, and
(ii) a first unit comprising a contacting unit for contacting a sample with the anti-IL6 sdAb, and
(iii) a second unit comprising a detecting unit for detecting and/or quantifying binding of the anti-IL6 sdAb, wherein the second unit comprises the anti-IL6 sdAb conjugated to a working electrode.

In another aspect, the invention provides an apparatus comprising:
(i) an anti-IL6 sdAb of the invention, and
(ii) a first unit comprising a contacting unit for contacting a subject with the anti-IL6 sdAb, and
(iii) a second unit comprising a detecting unit for detecting and/or quantifying binding of the anti-IL6 sdAb, wherein the second unit comprises the anti-IL6 sdAb conjugated to a working electrode.

In another aspect, the invention provides an apparatus comprising:
(i) an anti-IL6 sdAb of the invention, and
(ii) a first unit comprising a contacting unit for contacting a sample with the anti-IL6 sdAb, and
(iii) a second unit comprising a detecting unit for detecting and/or quantifying binding of the anti-IL6 sdAb, wherein the second unit comprises a biosensor of the present invention.

In another aspect, the invention provides an apparatus comprising:
(i) an anti-IL6 sdAb of the invention, and
(ii) a first unit comprising a contacting unit for contacting a subject with the anti-IL6 sdAb, and
(iii) a second unit comprising a detecting unit for detecting and/or quantifying binding of the anti-IL6 sdAb, wherein the second unit comprises a biosensor of the present invention.

In another aspect, the invention provides an apparatus comprising:
(i) an anti-IL6 sdAb of the invention, and
(ii) a first unit comprising a contacting unit for contacting a sample comprising interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor of the present invention.

In another aspect, the invention provides an apparatus comprising:
(i) an anti-IL6 sdAb of the invention, and
(ii) a first unit comprising a contacting unit for contacting a subject's interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor of the present invention.

In another aspect, the invention provides an apparatus comprising:
(i) an anti-IL6 sdAb of the invention, and
(ii) a first unit comprising a contacting unit for contacting a sample comprising interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode.

In another aspect, the invention provides an apparatus comprising:
(i) an anti-IL6 sdAb of the invention, and
(ii) a first unit comprising a contacting unit for contacting a subject's interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode.

In another aspect, the invention provides an apparatus comprising:
(i) an anti-IL6 sdAb of the invention, and
(ii) a first unit comprising a contacting unit for contacting a sample comprising interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode, wherein the anti-IL6 sdAb is conjugated to the working electrode, optionally via a nucleic acid (linker).

In another aspect, the invention provides an apparatus comprising:
(i) an anti-IL6 sdAb of the invention, and
(ii) a first unit comprising a contacting unit for contacting a subject's interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode, wherein the anti-IL6 sdAb is conjugated to the working electrode, optionally via a nucleic acid (linker).

In another aspect, the invention provides an apparatus comprising:
(i) an anti-IL6 sdAb of the invention, and
(ii) a first unit comprising a contacting unit for contacting a sample comprising interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode, a counter electrode and/or a reference electrode, and a substrate, wherein the anti-IL6 sdAb is conjugated to the working electrode, optionally via a nucleic acid (linker).

In another aspect, the invention provides an apparatus comprising:
(i) an anti-IL6 sdAb of the invention, and
(ii) a first unit comprising a contacting unit for contacting a subject's interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode, a counter electrode and/or a reference electrode, and a substrate, wherein the anti-IL6 sdAb is conjugated to the working electrode, optionally via a nucleic acid (linker).

In another aspect, the invention provides an apparatus comprising:
(i) an anti-IL6 sdAb of the invention, and
(ii) a first unit comprising a contacting unit for contacting a sample comprising interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode, a counter electrode and/or a reference electrode, and a substrate, wherein the anti-IL6 sdAb is conjugated to the working electrode, optionally via a nucleic acid (linker), and wherein the anti-IL6 sdAb comprises a detection label or tag.

In another aspect, the invention provides an apparatus comprising:
(i) an anti-IL6 sdAb of the invention, and
(ii) a first unit comprising a contacting unit for contacting a subject's interstitial fluid (ISF), blood, urine, stool, serum, tear fluid, sweat, or saliva with the anti-IL6 sdAb, and
(iii) a second unit comprising a detecting unit for detecting and/or quantifying binding of the anti-IL6 sdAb, wherein the second unit comprises a biosensor comprising the anti-IL6 sdAb and a working electrode, a counter electrode and/or a reference electrode, and a substrate, wherein the anti-IL6 sdAb is conjugated to the working electrode, optionally via a nucleic acid (linker), and wherein the anti-IL6 sdAb comprises a detection label or tag.

It is also envisioned herein that the apparatus of the present invention can comprise additional components, such as means for dispensing a drug, e.g. a dispensing unit for dispensing a drug. The means for dispensing a drug/dispensing unit for dispensing a drug can be comprised in a third unit which is further comprised the apparatus of the invention. For example, the apparatus as described herein can comprise a third unit comprising means for dispensing a drug/a dispensing unit for dispensing a drug. This is advantageous when it is desired to combine the measurement of IL6 levels in a subject with a therapy. For example, the measured level of IL6 in a subject can be compared with a reference value as described herein above. Depending on the difference of the IL6 level in the subject and the reference value, the third unit can dispense a drug, e.g. to counter inflammation or to decrease or increase levels of IL6. In the sense of the present invention, a drug can be any drug used in the treatment of an IL6 related disease, such as an inflammatory disease and/or autoimmune disease. Exemplary inflammatory diseases and/or autoimmune diseases are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. In general, the invention is not limited to a third unit dispensing a specific drug, the skilled practitioner is aware that any drug suitable for the means of dispensing the drug and for the therapy of the disease to be treated in the subject can be used. Exemplary drugs that can be used in the sense of the invention are remdevisir, nirmatrelvir, molnupiravir, baricitinib, bebtelovimab, etesevimab, casirivimab, imdevimab, aspirin, celecoxib, prednisone, methotrexate, hydroxychloroquine, leflunomide, sulfasalazine, abatacept, adalimumab, certolizumab, etanecept, golimumab, infliximab, rituximab, sarilumab, tocilizumab, baricitinib, tofacitinib, upadacitinib, anakinra, minocycline, azathioprine, cyclosporine, penicillamine, Statins, angiotensin-converting enzyme inhibitors, angiotensin receptor blockers, dapagliflozin, ibuprofen, naproxen, voclosporin, anifrolumab, mycophenolate, belimumab, glatiramer acetate, ocrelizumab, fingolimod, dimethyl fumarate, teriflunomide, methylprednisone, natalizumab, alemtuzumab, diroximel fumarate, siponimod, cladribine, Broad-spectrum antibiotics, amlodipine, diltiazem, Metformin, Insulin, SGLT2 inhibitors, leuprolide, anastrozole, letrozole, tamoxifen, fulvestrant, bevacizumab, alectinib, ibrutinib, imatinib, palbociclib, fluoxymesterone, methyltestosterone, tamoxifen citrate, estramustine, interferon alfa-2b recombinant, goserelin, flutamide, aldesleukin, bicalutamide, porfimer, nilutamide, imiquimod, toremifene, thalidomide, trastuzumab, alitretinoin, bexarotene, denileukin dfititox, exemestane, gemtuzumab, triptorelin, imatinib mesylate, peginterferon alfa-2b, ibritumomab tiuxetan, leuprolide acteate, abarelix, bortezomib, gefitinib, tositumomab, cetuximab, erlotinib hydrochloride, azacitidine, lenalidomide, sorafenib tosylate, dastinib, decitabine, panitumumab, sunitinib malate, vorinostat, lapatinib ditosylate, nilotinib, temsirolimus, degarelix, everolimus, ofatumumab, pazopanib hydrochloride, romodepsin, denosumab, hydroxiurea, sipuleucel-T, abiraterone acetate, brentuximab vedotin, crizotinib, ipilumab, ruxolinitib phosphate, vandetanib, vemurafenig, pertuzumab, axitinib, bosutinib, cabozantinib, cafilzomib, enzalutamide, ponatinib hydrochloride, regorafenib, vismodegib, Ziv-aflibercept, dabrafenib, trametinib, obinutuzumam, ado-trastuzumab emtansine, afatinib, pomalidomide, idelalisib, belinostat, ceritinib, avelumab, durvalumab, cemiplimab, dostarlimab, pembrolizumab, ramuricumab, lanreotide, blinatumomab, nivolumab, olaparib, atezolizumab, ipilimumab, Mechlorethamine, Mercaptopurine, Busulfan, Chlorambucil, Cyclophosphamide, Vincristine sulfate, dactinomycin, vinblastine sulfate, thioguanine, procarbazine hydrochloride, floxuridine, fluorouracil, mitotane, bleomycin, doxorubicin hydrochloride, dacarbazine, lomustine, carmustine, cisplatin, asparaginase, streptozocin, etoposide, ifosfamide, carboplatin, altreatime, fludarabine, pentostatin, paclitaxel, melphalan, teniposide, caldribine, vinorelbine tartrate, pegaspargase, thiotepa, docetaxel, gemcitabine, irinotecan, topotecan hydrochloride, idarubicin, capecitabine, daunorubucin hydrochloride, valrubicin, temozolomide, cytarabine, epirubicin, arsenic trioxide, mitomycin, oxaliplatin, pemetrexed disodium, clorabine, nelarabine, ixabepilone, bendamustine hydrochloride, pralatrexate, cabazitaxel, eribuline mesylate, asparaginase Erwinia chrysanthemi, oamcetaxine mepesuccinate, radium 223 dichloride, Amikacin, Gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cephradine, cephrapirin, cephalothin, cefalexin, cefaclor, cefoxitin, cefotetan, cefamandole, cefmetazole, cefonicid, cefprozil, cefuroxime, cefixime, cefdinir, cefditoren, ceforeprazone, cefotaxime, cefpodoxime, ceftazidime, ceftibuten, ceftizoxime, moxalactam, ceftriaxone, cefepime, ceftaroline fosamil, ceftobirprole, teicoplanin, vancomycin, telavancin, dalbavancin, oritavancin, clindamycin, lincomycin, daptomycin, azithormycin, clarithromycin, erythromycin, roxithromycin, telithromycin, spriamycin, fidaxomicin, aztreonam, furazolidone, nitrodurantoin, linezolid, posizolid, radezolid, torezolid, amoxicillin, ampicillin, azlocillini, dicloxacillin, flucloxacillin, mezlocillin, methicillin, nafcillin, oxacillin, penicillin, piperacillin, temocillin, ticarcillin, bacitracin, colistin, polymyxin, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temfloxacin, mafenied, sulfacetamibed, sulfadiazine, silver sulfadiazine, sulfadimethozine, sulfamethizole, sulfamethoxazole, sulfanilimide, sulfisoxazole, trimehoprim-sulfamethoxazole, sulfonamidochrysoidine, demeclocycline, doxycycline, metacycline, minocyclinie, oxytetracycline, tetracycline, clofazimine, dapsone, capreomycin, cycloserine, ethambutol, ethionamide, isoniazid, pyrazinmide, rifampicin, rifabutin, rifapentine, arsphenamine, chloramphenicol, fosfomycin, fusidic acid, metronidazole, mupirocin, platensimycin, quinupristin, thiampehnicol, tigecycline, tinidazole, tremethoprim, teixobactin, malacidins, halicin, fluticasone propionate, budesonide, ciclesonide, beclomethasone, mometasone, fluticasone furorate, montelukast, zafirlukast, zileuton, fluticasone-salmeterol, budesonide-formoterole, formoterol-mometasone, fluticasone furoate-vilanterol, Theophylline, methylprednisolone, ipratropium, tiotropium, Colchicine, canakinumab, rilonacept, siltuximab, olokizumab, elsilimomab, clazakizumab, sirukumab, levilimab, or any combination thereof. In some embodiments, a drug that can be used in the sense of the invention is one or more of corticosteroids, methotrexate, IL-6 inhibitors, TNF-alpha inhibitors, PD-1/PD-L1 inhibitors, or CTLA-4 inhibitors.

In some embodiments, the drug is an anti-IL6 antibody. In some embodiments the drug is an anti-IL6 sdAb of the present invention. In some embodiments the drug is one or more of the anti-IL6 sdAb as defined in SEQ ID NOs: 1, 9, 17, 25, 33, 41, 49, or 57. In some embodiments the drug is two or more, such as two, three, four, five, six, seven, or eight of the anti-IL6 sdAb as defined in SEQ ID NOs: 1, 9, 17, 25, 33, 41, 49, or 57. In some embodiments, the drug is the anti-IL6 sdAb as defined in SEQ ID NO: 1. In some embodiments, the drug is the anti-IL6 sdAb as defined in SEQ ID NO: 9. In some embodiments, the drug is the anti-IL6 sdAb as defined in SEQ ID NO: 17. In some embodiments, the drug is the anti-IL6 sdAb as defined in SEQ ID NO: 25. In some embodiments, the drug is the anti-IL6 sdAb as defined in SEQ ID NO: 33. In some embodiments, the drug is the anti-IL6 sdAb as defined in SEQ ID NO: 41. In some embodiments, the drug is the anti-IL6 sdAb as defined in SEQ ID NO: 49. In some embodiments, the drug is the anti-IL6 sdAb as defined in SEQ ID NO: 57. The features described herein above regarding the apparatus including the first and second unit apply mutatis mutandis to the apparatus comprising a third unit.

Accordingly, the invention provides in one aspect an apparatus comprising:
(i) an anti-IL6 sdAb of the invention, and
(ii) a contacting unit for contacting a sample with the anti-IL6 sdAb, and
(iii) a detecting unit for detecting and/or quantifying binding of the anti-IL6 sdAb, and
(iv) a dispensing unit for dispensing a drug.

In another aspect, the invention provides an apparatus comprising:
(i) an anti-IL6 sdAb of the invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug.

In another aspect, the invention provides an apparatus comprising:
(i) an anti-IL6 sdAb of the invention, and
(ii) a first unit comprising a contacting unit for contacting a sample with the anti-IL6 sdAb, and
(iii) a second unit comprising a detecting unit for detecting and/or quantifying binding of the anti-IL6 sdAb, and
(iv) a third unit comprising a dispensing unit for dispensing a drug.

In another aspect, the invention provides an apparatus comprising:
(i) an anti-IL6 sdAb of the invention, and
(ii) a first unit comprising a contacting unit for contacting a subject with the anti-IL6 sdAb, and
(iii) a second unit comprising a detecting unit for detecting and/or quantifying binding of the anti-IL6 sdAb, and
(iv) a third unit comprising a dispensing unit for dispensing a drug.

In another aspect, the invention provides an apparatus comprising:
(i) an anti-IL6 sdAb of the invention, and
(ii) a first unit comprising a contacting unit for contacting a sample with the anti-IL6 sdAb, and
(iii) a second unit comprising a detecting unit for detecting and/or quantifying binding of the anti-IL6 sdAb, and
(iv) a third unit comprising a dispensing unit for dispensing a drug, wherein the drug is one or more of an anti-IL6 sdAb of the present invention.

In another aspect, the invention provides an apparatus comprising:
(i) an anti-IL6 sdAb of the invention, and
(ii) a first unit comprising a contacting unit for contacting a subject with the anti-IL6 sdAb, and
(iii) a second unit comprising a detecting unit for detecting and/or quantifying binding of the anti-IL6 sdAb, and
(iv) a third unit comprising a dispensing unit for dispensing a drug, wherein the drug is one or more of an anti-IL6 sdAb of the present invention.

The apparatus of the present invention can be in form of an applicator like appliance, such as a handheld or stationary device comprising the biosensor of the present invention, or can be permanently or reversibly implanted or attached to the subject's body, such as an implanted, wearable device comprising the biosensor of the present invention. The implanted or attached apparatus may have a shape of lancet and may be implanted in the dermis layer of the skin e.g. of a subject for detecting binding of the anti-IL6 sdAb of the present invention to IL6, e.g. by contacting a sample, e.g. interstitial fluid (ISF) with the anti-IL6 sdAb. The thereby detected level of IL6 may be correlated and/or converted to overall IL6 levels in e.g. blood or other bodily fluids. The site and depth of implantation may affect the shape of the implanted or attached apparatus, components, and configuration of the apparatus. The apparatus may be attached or implanted using an insertion mechanism. The signal readout, i.e. detection of IL6 level, can be performed by the apparatus. The apparatus can send the results, i.e. detected IL6 levels, to any external device, e.g. a server or smartphone, optionally via an application. The advantage of having a reversibly or permanently implanted apparatus is that continuous measurement of IL6 is enabled which can increase the reliability of the obtained IL6 dynamics over time. This can be especially useful if the apparatus is to be used for disease management and treatment decisions in a subject, e.g. in combination with the in vivo diagnosis or methods of monitoring the level of IL6 described herein below. A reversibly or permanently implanted apparatus can for example detect inflammation spikes in a subject and indicate when medication is necessary and what amount of medication is necessary, thereby greatly improving management of an inflammatory disease in a subject. In a further embodiment, an apparatus as described above can detect when medication is necessary and what amount of medication is necessary and dispense the necessary amount at the relevant time into the subject's circulatory system. The apparatus can establish the temporal variable (time when medication is necessary) by detecting that a certain threshold of IL6 level is overcome, such as a reference value as described herein. The apparatus can establish the quantitative variable (necessary amount of medication) by detecting the difference of the IL6 level measured in the subject and the reference value as described herein.

The above similarly applies to an apparatus of the present invention in form of an applicator like appliance. Applicator like appliance means that the apparatus is not reversibly or permanently attached to or implanted into a subject. It is rather used as a device on the subject's body to obtain a single measurement at a time (spot measurement). The apparatus used in this way can indicate when and if the measurement is indicative that medication is necessary and can indicate what amount of medication is necessary like explained above, i.e. by comparing the measurements with a reference value.

As mentioned herein, the apparatus of the present invention can comprise the biosensor of the present invention. In such a case, the biosensor could be seen as a subsystem within the apparatus. The apparatus of the present invention can also be partially implanted. For example the biosensor subsystem of the apparatus may be the implanted part, i.e. being within the body of a subject (under the skin layer). The entire apparatus does not necessarily have to be entirely within the body (under the skin layer) but can also be superficially attached e.g. on the skin. In such a setup the signal readout electronics is wearable (e.g. being superficially attached on the skin of the subject) and connected with the implanted part, e.g. the biosensor.

When the apparatus is an applicator like appliance, e.g. for the “spot measurement system” it can comprise a biosensor strip (e.g. on a bench), and a portable electronics reader that can be connected to the biosensor strip or vice versa to provide a readout.

Accordingly, the invention provides in one aspect an apparatus comprising:
(i) an anti-IL6 sdAb of the present invention, and
(ii) a contacting unit for contacting a sample or a subject with the anti-IL6 sdAb, and
(iii) a detecting unit for detecting and/or quantifying binding of the anti-IL6 sdAb, and optionally
(iv) a dispensing unit for dispensing a drug, optionally wherein the drug is the anti-IL6 sdAb of the present invention,
   wherein the apparatus is reversibly attached to or implanted into the subject.

In another aspect, the invention provides an apparatus comprising:
(i) an anti-IL6 sdAb of the present invention, and
(ii) a contacting unit for contacting a sample or a subject with the anti-IL6 sdAb, and
(iii) a detecting unit for detecting and/or quantifying binding of the anti-IL6 sdAb, and optionally
(iv) a dispensing unit for dispensing a drug, optionally wherein the drug is the anti-IL6 sdAb of the present invention,
   wherein the apparatus is permanently attached to or implanted into the subject.

In another aspect, the invention provides an apparatus comprising:
(i) an anti-IL6 sdAb of the present invention, and
(ii) a contacting unit for contacting a sample or a subject with the anti-IL6 sdAb, and
(iii) a detecting unit for detecting and/or quantifying binding of the anti-IL6 sdAb, and optionally
(iv) a dispensing unit for dispensing a drug, optionally wherein the drug is the anti-IL6 sdAb of the present invention,
   wherein the apparatus is fully or partially implantable.

In another aspect, the invention provides an apparatus comprising:
(i) an anti-IL6 sdAb of the present invention, and
(ii) a contacting unit for contacting a sample or a subject with the anti-IL6 sdAb, and
(iii) a detecting unit for detecting and/or quantifying binding of the anti-IL6 sdAb, and optionally
(iv) a dispensing unit for dispensing a drug, optionally wherein the drug is the anti-IL6 sdAb of the present invention,
   wherein the apparatus is fully or partially implanted into a subject.

Any of apparatuses as described herein can be permanent, reusable, single use, disposable or for temporary use (e.g. implant). For example, a biosensor of the present invention can be implanted for a limited time e.g. 14 days for a continuous measurement of IL6 level.

In yet another aspect, the invention provides the following items:
1. An apparatus (for detecting a target analyte) comprising:
   (i) a collector for collecting a sample,
   (ii) a sensing component comprising a biosensor comprising an anti-IL6 sdAb of the present invention,
   (iii) a connector for connecting the sensing component to an electrochemical measurement device.
2. The apparatus of item 1, wherein the collector is designed to at least partially encase the sensing component.
3. The apparatus of item 1 or 2, wherein the collector comprises a first portion and a second portion, the first portion being able to contain the collected sample and the second portion encasing the sensing component.
4. The apparatus of item 3, wherein the first portion comprises a nozzle and a sampling reservoir.
5. The apparatus of item 4, wherein the nozzle comprises microfluidic channels optionally treated with a hydrophilic coating.
6. The apparatus of item 4, wherein the nozzle comprises silica capillary tubes.
7. The apparatus of any one of items 1-6, wherein the collector and the connector are provided with complementary locking means to secure the sensing component within the device.
8. The apparatus of any one of items 1-7, wherein the sensing component further comprises two working electrodes, a counter electrode and a reference electrode.
9. The apparatus of item 8, wherein the electrode of the biosensor and the two working electrodes comprise gold.
10. The apparatus of items 8 or 9, wherein the counter electrode comprises platinum and the reference electrode comprises silver.
11. The apparatus of items 8-10, wherein each electrode is in the form of a wire.
12. The apparatus of item 11, wherein the sensing component has a cylindrical form and the wires are positioned spaced apart within the sensing component along a length thereof.
13. The apparatus of any one of items 8-12, wherein the electrodes are held in a matrix of a non-conductive material.
14. The apparatus of item 13, wherein the non-conductive material comprises a silicon resin.
15. The apparatus of item any one of items 11-14, wherein the anti-IL6 sdAb is bound to a first end of the biosensor electrode wire and the anti-IL6 sdAb is exposed to the sample when the first end is in contact with the collected sample.
16. The apparatus of any one of items 11-15, wherein a first end of each electrode wire is in contact with the sample when collected in the collector.
17. The apparatus of any one of items 11-15, wherein a second end of the wires are in contact with the connector.
18. The apparatus of any one of items 1-17, wherein the sample comprises a biological fluid, such as a bodily fluid e.g. interstitial fluid (ISF), blood, urine, stool, serum, tear fluid, sweat or saliva.
19. The apparatus of any one of items 1-18, wherein the target analyte is IL6.

The sdAbs of the present invention are particularly useful for therapy since they can inhibit the biological function of IL6 thereby reducing inflammation. The sdAbs of the present invention do not necessarily inhibit the biological function of IL6. In some embodiments, it is preferred that a sdAbs of the invention do not inhibit the biological function of IL6, e.g. when detection but not treatment is desired. Accordingly, in some embodiments it is preferred that a sdAb of the invention does inhibit the biological function of IL6, e.g. when detection and therapy or only therapy is desired. As used herein “treatment” preferably relates to the acute treatment, prophylactic treatment, or the disease management of an inflammatory disease and/or autoimmune disease related to an increased IL6 production, IL6 secretion, IL6 release or IL6 level or increased inflammation values that are treated with the anti-IL6 sdAbs of the invention. Accordingly, the present invention provides in one aspect an anti-IL6 sdAb of the invention for use as a medicament.

In another aspect, the invention provides the anti-IL6 sdAb as defined in SEQ ID NO: 1 for use as a medicament.

In another aspect, the invention provides the anti-IL6 sdAb as defined in SEQ ID NO: 9 for use as a medicament.

In another aspect, the invention provides the anti-IL6 sdAb as defined in SEQ ID NO: 17 for use as a medicament.

In another aspect, the invention provides the anti-IL6 sdAb as defined in SEQ ID NO: 25 for use as a medicament.

In another aspect, the invention provides the anti-IL6 sdAb as defined in SEQ ID NO: 33 for use as a medicament.

In another aspect, the invention provides the anti-IL6 sdAb as defined in SEQ ID NO: 41 for use as a medicament.

In another aspect, the invention provides the anti-IL6 sdAb as defined in SEQ ID NO: 49 for use as a medicament.

In another aspect, the invention provides the anti-IL6 sdAb as defined in SEQ ID NO: 57 for use as a medicament.

In another aspect, the invention provides the anti-IL6 sdAb as defined in any one of SEQ ID NOs: 1, 9, 17, 25, 33, 41, 49, or 57 for use as a medicament.

In another aspect, the invention provides one or more, such as one, two, three, four, five, six, seven, or eight anti-IL6 sdAbs as defined in SEQ ID NOs: 1, 9, 17, 25, 33, 41, 49, or 57 for use as a medicament.

Secretion of IL6 can induce inflammation. Thus, the sdAbs of the invention are preferably used in the treatment of IL6 associated diseases, such as diseases associated with inflammation or systemic inflammation. In particular, the disease may be associated with an increased production or release of IL6. Inhibiting the biological function of IL6 can thus ameliorate this effect and reduce the resulting inflammation. The invention is not particularly limited to a disease to be treated, rather any disease that results in increased IL6 production, IL6 secretion, IL6 release or IL6 level or increased inflammation values can be treated with the sdAbs of the invention. Such a disease can be associated with an increased production, secretion, release, or level of inflammatory markers. Inflammatory markers can indicate an inflammation event which may then be treated with the anti-IL6 sdAbs of the present invention. Exemplary inflammatory markers are Adenosine deaminase (ADA), Artemin (ARTN), Axin-1 (AXIN1), Beta-nerve growth factor (NGF), C-reactive protein (CRP), C-C motif chemokine 13 (CCL13), C-C motif chemokine 19 (CCL19), C-C motif chemokine 2 (CCL2), C-C motif chemokine 20 (CCL20), C-C motif chemokine 23 (CCL23), C-C motif chemokine 25 (CCL25), C-C motif chemokine 28 (CCL28), C-C motif chemokine 3 (CCL3), C-C motif chemokine 4 (CCL4), C-C motif chemokine 7 (CCL7), C-C motif chemokine 8 (CCL8), C-X-C motif chemokine 10 (CXCL10), C-X-C motif chemokine 11 (CXCL11), C-X-C motif chemokine 5 (CXCL5), C-X-C motif chemokine 6 (CXCL6), C-X-C motif chemokine 9 (CXCL9), CUB domain-containing protein 1 (CDCP1), Caspase-8 (CASP8), Cystatin-D (CST5), Delta and Notch-like epidermal growth factor-related receptor (DNER), Eotaxin (CCL11), Eukaryotic translation initiation factor 4E-binding protein 1 (EIF4EBP1), Fibroblast growth factor 19 (FGF19), Fibroblast growth factor 21 (FGF21), Fibroblast growth factor 23 (FGF23), Fibroblast growth factor 5 (FGF5), Fms-related tyrosine kinase 3 ligand (FLT3LG), Fractalkine (CX3CL1), Glial cell line-derived neurotrophic factor (GDNF), Growth-regulated alpha protein (CXCL1), Hepatocyte growth factor (HGF), Interferon gamma (IFNG), Interleukin-1 alpha (IL1A), Interleukin-10 (IL10), Interleukin-10 receptor subunit alpha (IL10RA), Interleukin-10 receptor subunit beta (IL10RB), Interleukin-12 subunit beta (IL12B), Interleukin-13 (IL13), Interleukin-15 receptor subunit alpha (IL15RA), Interleukin-17A (IL17A), Interleukin-17C (IL17C), Interleukin-18 (IL18), Interleukin-18 receptor 1 (IL18R1), Interleukin-2 (IL2), Interleukin-2 receptor subunit beta (IL2RB), Interleukin-20 (IL20), Interleukin-20 receptor subunit alpha (IL20RA), Interleukin-22 receptor subunit alpha-1 (IL22RA1), Interleukin-24 (IL24), Interleukin-33 (IL33), Interleukin-4 (IL4), Interleukin-5 (IL5), Interleukin-6 (IL6), Interleukin-7 (IL7), Interleukin-8 (CXCL8), Interstitial collagenase (MMP1), Kit ligand (KITLG), Leukemia inhibitory factor (LIF), Leukemia inhibitory factor receptor (LIFR), Lymphotoxin-alpha (LTA), Macrophage colony-stimulating factor 1 (CSF1), NAD-dependent protein deacetylase sirtuin-2 (SIRT2), Natural killer cell receptor 2B4 (CD244), Neurotrophin-3 (NTF3), Neurturin (NRTN), Oncostatin-M (OSM), Programmed cell death 1 ligand 1 (CD274), Protein S100-A12 (S100A12), Protransforming growth factor alpha (TGFA), STAM-binding protein (STAMBP), Signaling lymphocytic activation molecule (SLAMF1), Stromelysin-2 (MMP10), Sulfotransferase 1A1 (SULTIA1), T-cell differentiation antigen CD6 (CD6), T-cell surface glycoprotein CD5 (CD5), T-cell surface glycoprotein CD8 alpha chain (CD8A), Thymic stromal lymphopoietin (TSLP), Transforming growth factor beta-1 proprotein (TGFB1), Tumor necrosis factor alpha (TNF-a), Tumor necrosis factor ligand superfamily member 10 (TNFSF10), Tumor necrosis factor ligand superfamily member 11 (TNFSF11), Tumor necrosis factor ligand superfamily member 12 (TNFSF12), Tumor necrosis factor ligand superfamily member 14 (TNFSF14), Tumor necrosis factor receptor superfamily member 11B (TNFRSF11B), Tumor necrosis factor receptor superfamily member 5 (CD40), Tumor necrosis factor receptor superfamily member 9 (TNFRSF9), Urokinase-type plasminogen activator (PLAU), Vascular endothelial growth factor A (VEGFA) and the like or any combination thereof.

Inflammatory markers can also include the following markers. Exemplary additional biomarkers are: Adiponectin, CD44var (v6), Elafin, Lactoferrin, MMP-3, NRP-1, RANTES (CCL5), Resistin (ADSF), VCAM-1, CD14, CD62L (L-Selectin), Endoglin, Leptin, MMP-9, Osteopontin (OPN), RBP4, SCGF beta, ZAG, CD44, Cystatin C, ICAM-1, MBL, NGAL, PAI-1 (Serpin), REG-3a, TIMP-1.

Additionally, markers can indicate vascular injuries. Exemplary vascular injury markers are Serum Amyloid A, C-Reactive Protein CRP, Vascular Cell Adhesion Molecule-1 (VCAM-/CD106), Intercellular Adhesion Molecule-1 (ICAM-1/CD54).

In a preferred embodiment an inflammatory marker is IL6. Exemplary diseases are inflammatory diseases and/or autoimmune diseases. Exemplary inflammatory diseases and/or autoimmune diseases are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. Accordingly, the invention provides in one aspect an anti-IL6 sdAb of the invention for use in the treatment of a disease associated with an inflammation.

In another aspect, the invention provides an anti-IL6 sdAb of the invention for use in the treatment of a disease associated with an inflammation, optionally a systemic inflammation.

It is also envisioned herein that the anti-IL6 sdAb of the invention can be used in co-therapy, wherein more than one, such as two, three, four, five, six, seven, or eight anti-IL6 sdAb are selected from SEQ ID NOs: 1, 9, 17, 25, 33, 41, 49, or 57. Accordingly, the invention provides two or more, such as two, three, four, five, six, seven, or eight anti-IL6 sdAb selected form SEQ ID NOs: 1, 9, 17, 25, 33, 41, 49, or 57 for use in the treatment of a disease associated with an inflammation, optionally a systemic inflammation.

Accordingly, the invention provides in one aspect one or more anti-IL6 sdAb selected from the group of SEQ ID NOs: 1, 9, 17, 25, 33, 41, 49, or 57 for use in the treatment of a disease associated with an inflammation, optionally a systemic inflammation. In general, it is envisioned that sdAbs of the present invention can also be used in combination, e.g. sdAbs having specific kₒₙ/k_{off} values can be combined to achieve coverage of continuous and reversible binding over a desired dynamic range of e.g. IL6.

In another aspect, the invention provides an anti-IL6 sdAb of the invention for use in the treatment of a disease associated with an inflammation, optionally a systemic inflammation, wherein the disease is associated with an increased production or release of inflammatory markers.

In another aspect, the invention provides an anti-IL6 sdAb of the invention for use in the treatment of a disease associated with an inflammation, optionally a systemic inflammation, wherein the disease is associated with an increased production or release of IL6.

In another aspect, the invention provides an anti-IL6 sdAb of the invention for use in the treatment of an inflammatory and/or autoimmune disease.

In another aspect, the invention provides an anti-IL6 sdAb of the invention for use in the treatment of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

Methods of treatment are also envisioned herein. In general, the features described in context of the anti-IL6 sdAb of the present invention for use in the treatment of a disease also apply to the methods of treatment provided herein. The method of treatment can comprise a step of administering one or more anti-IL6 sdAb of the invention to a subject. Similarly, co-therapy can be practiced in a method of treatment, wherein more than one, such as two, three, four, five, six, seven, or eight anti-IL6 sdAb selected from SEQ ID NOs: 1, 9, 17, 25, 33, 41, 49, or 57 are administered to a subject.

Accordingly, the invention provides in one aspect a method of treatment comprising a step of administering an anti-IL6 sdAb of the invention to a subject in need thereof.

In another aspect, the invention provides a method of treatment comprising a step of administering one or more of an anti-IL6 sdAb selected from the group of SEQ ID NOs: 1, 9, 17, 25, 33, 41, 49, or 57 to a subject in need thereof.

In another aspect, the invention provides a method of treatment comprising a step of administering two or more of an anti-IL6 sdAb selected from the group of SEQ ID NOs: 1, 9, 17, 25, 33, 41, 49, or 57 to a subject in need thereof.

In another aspect, the invention provides a method of treatment comprising a step of administering an anti-IL6 sdAb of the invention to a subject in need thereof, wherein the subject suffers from a disease associated with an increased production or release of inflammatory markers.

In another aspect, the invention provides a method of treatment comprising a step of administering an anti-IL6 sdAb of the invention to a subject in need thereof, wherein the subject suffers from a disease associated with an increased production or release of IL6.

In another aspect, the invention provides a method of treatment comprising a step of administering an anti-IL6 sdAb of the invention to a subject in need thereof, wherein the subject suffers from an inflammatory and/or autoimmune disease.

In another aspect, the invention provides a method of treatment comprising a step of administering an anti-IL6 sdAb of the invention to a subject in need thereof, wherein the subject suffers from rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

Any of the treatments described herein can be performed by or in or by an apparatus as described herein.

Accordingly, the invention provides in one aspect a method of treatment comprising contacting or administering an apparatus of the present invention to a subject.

In another aspect, the invention provides a method of treatment comprising contacting or administering an apparatus of the present invention to a subject, wherein the apparatus administers a drug to the subject, optionally wherein the drug is a sdAb of the present invention.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from a disease associated with an increased production or release of inflammatory markers.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from a disease associated with an increased production or release of IL6.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from an inflammatory and/or autoimmune disease.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying IL6 in or on a subject of the present invention prior to administering the drug.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying IL6 in or on a subject of the present invention prior to administering the drug, and wherein the administration of said drug depends on the measurement obtained from the method for detecting and/or quantifying IL6 in or on a subject.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying IL6 in or on a subject of the present invention and compares the level of IL6 to a reference value prior to administering the drug, and wherein the administration of said drug depends on the difference between the measured IL6 level and the reference value.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying IL6 in or on a subject of the present invention and compares the level of IL6 to a reference value prior to administering the drug, and wherein the administration and amount of said drug depends on the difference between the measured IL6 level and the reference value.

The anti-IL6 sdAb of the invention can also be used in in vivo diagnosis. The anti-IL6 sdAb are particularly useful for in vivo diagnosis since the potential of detecting the dynamic range of IL6 in a subject, optionally over time, can allow the skilled practitioner to draw conclusions on the underlying disease or condition. As mentioned above, the level of IL6 can be for example indicative to distinguish a bacterial infection from a viral infection. Similarly, the level of IL6 can indicate that a subject suffers from rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. In the sense of the present invention, a disease diagnosed in the in vivo diagnosis is preferably a disease associated with an inflammation. A disease associated with an inflammation may be an inflammatory disease and/or an autoimmune disease. Exemplary inflammatory diseases and/or autoimmune diseases that can be diagnosed in the in vivo diagnosis described herein are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. A contacting step in the sense of any in vivo methods or in situ methods disclosed herein can comprise contacting a subject's interstitial fluid (ISF), blood, urine, stool, serum, tear fluid, sweat, or saliva with an anti-IL6 sdAb of the invention. In some embodiments a contacting step of a method of in vivo diagnosis of a disease associated with an inflammation of the present invention comprises contacting a subject's interstitial fluid (ISF), blood, urine, stool, serum, tear fluid, sweat, or saliva with an anti-IL6 sdAb of the invention.

Accordingly, the invention provides in one aspect an anti-IL6 sdAb of the present invention for use in in vivo diagnosis.

In another aspect, the invention provides an anti-IL6 sdAb of the present invention for use in a method of in vivo diagnosis of a disease associated with an inflammation, the method comprising:
(i) contacting a subject with an anti-IL6 sdAb of the present invention, and
(ii) detecting the binding of the anti-IL6 sdAb.

In another aspect, the invention provides an anti-IL6 sdAb of the present invention for use in a method of in vivo diagnosis of a disease associated with an inflammation, the method comprising:
(i) contacting a subject's biological fluid or sample, such as bodily fluid, e.g. interstitial fluid (ISF), blood, urine, stool, serum, tear fluid, sweat, or saliva with an anti-IL6 sdAb of the invention, and
(ii) detecting the binding of the anti-IL6 sdAb.

In another aspect, the invention provides an anti-IL6 sdAb of the present invention for use in a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb.

In another aspect, the invention provides an anti-IL6 sdAb of the present invention for use in a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein the disease associated with an inflammation or systemic inflammation is an inflammatory disease and/or an autoimmune disease.

In another aspect, the invention provides an anti-IL6 sdAb of the present invention for use in a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein the disease associated with an inflammation or systemic inflammation is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In particular, the detection of the IL6 level over time achieved by repeated spot measurements of IL6 over time or by the continuous measurement of IL6, e.g. in vivo as described herein above, can indicate inflammation spikes caused by diseases requiring constant disease management, close medical attention and/or regular medication. This can be advantageous in disease management e.g. of inflammatory bowel disease, rheumatoid arthritis, or to monitor efficacy and tolerability e.g. of corticosteroid therapy or immunotherapy in cancer patients. The spikes can be detected by the in vivo diagnosis provided herein. This is advantageous for the underlying treatment insofar it allows the subject suffering from a disease to precisely target inflammation spikes with drugs e.g. an IL6 drug such as an anti-IL6 sdAb of the invention, thereby reducing the total amount of drugs taken, the effectiveness of the treatment and reducing potential side effects from an otherwise regular medication. The in vivo diagnosis can for example be performed by an apparatus of the present invention.

Accordingly, the invention provides in one aspect an anti-IL6 sdAb of the present invention for use in a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein steps (i) and (ii) are repeatedly performed.

In another aspect, the invention provides an anti-IL6 sdAb of the present invention for use in a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein steps (i) and (ii) are repeatedly performed in a subject over time.

In another aspect, the invention provides an anti-IL6 sdAb of the present invention for use in a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein steps (i) and (ii) are repeatedly performed by an apparatus of the present invention permanently or reversibly attached to or implanted into a subject.

Diagnostic methods are also envisioned herein. In general, the features described in context of the anti-IL6 sdAb of the present invention for use in in vivo diagnosis also apply to the methods of treatment provided herein.

Accordingly, the invention provides in one aspect a method of in vivo diagnosis of a disease associated with an inflammation, the method comprising:
(i) contacting a subject with an anti-IL6 sdAb of the present invention, and
(ii) detecting the binding of the anti-IL6 sdAb.

In another aspect, the invention provides a method of in vivo diagnosis of a disease associated with an inflammation, the method comprising:
(i) contacting a subject's biological fluid or sample, such as bodily fluid, e.g. interstitial fluid (ISF), blood, urine, stool, serum, tear fluid, sweat, or saliva with an anti-IL6 sdAb of the invention, and
(ii) detecting the binding of the anti-IL6 sdAb.

In another aspect, the invention provides a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb.

In another aspect, the invention provides a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein the disease associated with an inflammation or systemic inflammation is an inflammatory disease and/or an autoimmune disease.

In another aspect, the invention provides a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein the disease associated with an inflammation or systemic inflammation is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein steps (i) and (ii) are repeatedly performed.

In another aspect, the invention provides a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein steps (i) and (ii) are repeatedly performed in a subject over time.

In another aspect, the invention provides a method of in vivo diagnosis of a disease associated with an inflammation, optionally a systemic inflammation, the method comprising:
(i) contacting a subject with an anti-IL6 sdAb of the invention, and
(ii) detecting the binding of the anti-IL6 sdAb,
   wherein steps (i) and (ii) are repeatedly performed by an apparatus of the present invention permanently or reversibly attached to or implanted into a subject.

Any of the in vivo diagnosis described herein can further comprise a step of comparing the level of anti-IL6 with a reference value or reference sample as described herein.

Any of the in vivo diagnosis described herein can be performed by or in an apparatus as described herein. The in vivo diagnosis as described herein can be further indicative for a necessary treatment which can be performed by the same apparatus as described herein.

The invention also provides methods for monitoring the level of IL6 in a sample or subject. As described above, the sdAb are especially useful for the detection of the IL6 level over time achieved by repeated in vitro or in vivo spot measurements of IL6 over time or by the continuous measurement of IL6, e.g. in vitro or preferably in vivo, which can indicate inflammation spikes in a subject (patient). It is desired to monitor these inflammation spikes, i.e. increased IL6 levels in a sample of a subject or in a subject over time. This is advantageous since it allows the skilled practitioner or the subject itself to perform targeted treatment of an underlying inflammatory or autoimmune disease. Such a monitoring of inflammation spikes allows for target treatment and reduces the amount of medication/drugs a subject has to take for properly managing their inflammatory or autoimmune disease and consequently reduces the disease burden and potential burden of side effects of the subject. A method of monitoring the level of IL6 can be performed similar to the method for detecting and/or quantifying IL6 in a sample or a subject as described herein above and to the in vivo diagnosis as described herein above. For monitoring IL6 level, a sample of a subject can be contacted with an anti-IL6 sdAb of the invention and the binding of the anti-IL6 sdAb to IL6 can be detected. When monitoring IL6 levels in a sample of a subject over time, the method might be repeatedly applied to two or more samples of a subject that have been obtained in time steps. Thus, the method can repeatedly detect the binding of an anti-IL6 sdAb of the present invention to IL6 over time, i.e. at certain times which can be distinguished by time steps between detection events. Accordingly, the ex vivo or in vitro method of monitoring the level of IL6 can be performed similar to the spot measurements in a sample of a subject or the measurements in a sample of a subject over time as described herein above.

Accordingly, the invention provides in one aspect an ex vivo or in vitro method of monitoring the level of IL6 comprising:
(i) contacting a sample with an anti-IL6 sdAb of the invention, and
(ii) detecting binding of the anti-IL6 sdAb.

The ex vivo or in vitro method of monitoring the level of IL6 can be performed by or in an apparatus as described herein.

The features described herein in context of the method for detecting and/or quantifying IL6 in a sample apply mutatis mutandis to the ex vivo or in vitro method of monitoring the level of IL6. In some aspect, the ex vivo or in vitro method of monitoring the level of IL6 may not comprise a step of obtaining a sample from the subject.

Accordingly, the invention provides in another aspect an ex vivo or in vitro method of monitoring the level of IL6 comprising:
(i) contacting a sample with an anti-IL6 sdAb of the invention, and
(ii) detecting binding of the anti-IL6 sdAb,
   wherein the sample comprises interstitial fluid (ISF), blood, urine, stool, serum, tear fluid, sweat, or saliva.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the contacting step comprises contacting a sample via a syringe, a needle, a microfluidic or capillary-based system with the anti-IL6 sdAb of the invention.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the contacting step comprises contacting the sample with a biosensor of the invention, optionally wherein the biosensor comprises a sdAb of the invention conjugated to a working electrode.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the detection step comprises physiochemical or electrochemical detection of the binding of the anti-IL6 sdAb and IL6.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the detection step comprises detection of the binding of the anti-IL6 sdAb and IL6 via an electrical signal produced by the binding.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the detection step comprises detection of the binding of the anti-IL6 sdAb and IL6 with a biosensor of the invention, optionally, wherein the biosensor comprises a sdAb of the invention conjugated to a working electrode.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the contacting step comprises contacting the sample with a biosensor of the invention, optionally wherein the biosensor comprises a sdAb of the invention conjugated to a working electrode and,
   wherein the detection step comprises detecting the binding of the binding of the anti-IL6 sdAb and IL6 with a biosensor of the invention, optionally, wherein the biosensor comprises a sdAb of the invention conjugated to a working electrode.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   further comprising detecting the binding of the anti-IL6 sdAb over time.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   further comprising detecting the binding of the anti-IL6 sdAb over time, wherein steps (i) and (ii) are performed on a further sample, optionally wherein the sample is from the same subject.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are performed on a further sample, optionally wherein the sample is from the same subject, and
   wherein the contacting step comprises contacting the sample with a contacting a sample with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In a preferred aspect, the invention provides an ex vivo or in vitro method of monitoring the level of IL6 comprising:
(i) contacting a sample comprising interstitial fluid (ISF), blood, urine, stool, serum, tear fluid, sweat, or saliva with an anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are performed on a further sample, optionally wherein the sample is from the same subject, and
   wherein the contacting step comprises contacting the sample with a contacting a sample with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another aspect, the invention provides an ex vivo or in vitro method of monitoring the level of IL6 comprising:
(i) contacting a sample with an anti-IL6 sdAb of the invention, and
(ii) detecting binding of the anti-IL6 sdAb,
   wherein the detection step comprises detection of the binding of the anti-IL6 sdAb and IL6 via electrochemical ELISA.

The ex vivo or in vitro method of monitoring the level of IL6 described herein can be performed by or in an apparatus as described herein.

In another aspect, it may be preferred to monitor the level of IL6 in a subject, i.e. in vivo. A method of monitoring the level of IL6 in a subject in vivo is especially advantageous since no samples have to be obtained from the subject, e.g. in time steps, which reduces the workload of the practitioner and the burden on a subject, e.g. from multiple consecutive blood draws. Accordingly, the invention provides in one aspect a method of monitoring the level of IL6 in a subject comprising:
(i) contacting the subject with an anti-IL6 sdAb of the invention, and
(ii) detecting binding of the anti-IL6 sdAb.

The features described herein in context of the method for detecting and/or quantifying IL6 in a subject apply mutatis mutandis to the method of monitoring the level of IL6 in a subject.

In another aspect, the invention provides a method of monitoring the level of IL6 in a subject comprising:
(i) contacting a subject's biological fluid or sample, such as bodily fluid, e.g. interstitial fluid (ISF), blood, urine, stool, serum, tear fluid, sweat, or saliva with an anti-IL6 sdAb of the invention, and
(ii) detecting binding of the anti-IL6 sdAb.

In another aspect, the invention provides a method of monitoring the level of IL6 in a subject comprising:
(i) contacting a subject's interstitial fluid (ISF), blood, urine, stool, serum, tear fluid, sweat, or saliva with an anti-IL6 sdAb of the invention, and
(ii) detecting binding of the anti-IL6 sdAb.

In another aspect, the invention provides a method of monitoring the level of IL6 in a subject comprising:
(i) contacting a subject's interstitial fluid (ISF) with an anti-IL6 sdAb of the invention, and
(ii) detecting binding of the anti-IL6 sdAb.

In another aspect, the invention provides a method of monitoring the level of IL6 in a subject comprising:
(i) contacting the subject with an anti-IL6 sdAb of the invention, and
(ii) detecting binding of the anti-IL6 sdAb,
   wherein the contacting step comprises contacting the subject with a biosensor of the invention, optionally wherein the biosensor comprises a sdAb of the invention conjugated to a working electrode.

In another aspect, the invention provides a method of monitoring the level of IL6 in a subject comprising:
(i) contacting the subject with an anti-IL6 sdAb of the invention, and
(ii) detecting binding of the anti-IL6 sdAb,
   wherein the contacting step comprises contacting the subject with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another aspect, the invention provides a method of monitoring the level of IL6 in a subject over time comprising:
(i) contacting the subject with an anti-IL6 sdAb of the invention, and
(ii) detecting binding of the anti-IL6 sdAb.

In another aspect, the invention provides a method of monitoring the level of IL6 in a subject comprising:
(i) contacting the subject with an anti-IL6 sdAb of the invention, and
(ii) detecting binding of the anti-IL6 sdAb,
   further comprising contacting and/or detecting the binding of the anti-IL6 sdAb over time, wherein steps (i) and (ii) are repeatedly performed in or on the subject.

In another aspect, the invention provides a method of monitoring the level of IL6 in a subject comprising:
(i) contacting the subject with an anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are repeatedly performed in or on the subject, and
   wherein the contacting step comprises contacting the subject with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In a preferred aspect, the invention provides a method of monitoring the level of IL6 in a subject comprising:
(i) contacting the subject's interstitial fluid (ISF), blood, urine, stool, serum, tear fluid, sweat, or saliva with an anti-IL6 sdAb of the invention, and
(ii) detecting binding of the anti-IL6 sdAb,
   further comprising contacting and/or detecting the binding of the anti-IL6 sdAb over time,
   wherein steps (i) and (ii) are repeatedly performed in or on the subject, and
   wherein the contacting step comprises contacting the subject with a biosensor of the invention, optionally wherein the biosensor comprises a sdAb of the invention conjugated to a working electrode, and
   wherein the detection step comprises detecting the binding of the binding of the anti-IL6 sdAb and IL6 with a biosensor of the invention, optionally, wherein the biosensor comprises a sdAb of the invention conjugated to a working electrode.

In another aspect, the invention provides a method of monitoring the level of IL6 in a subject comprising:
(i) contacting the subject with an anti-IL6 sdAb of the invention, and
(ii) detecting binding of the anti-IL6 sdAb,
   wherein the detection step comprises detection of the binding of the anti-IL6 sdAb and IL6 via electrochemical ELISA.

The method of monitoring the level of IL6 in a subject described herein can be performed by or in an apparatus as described herein.

As described herein above, the apparatus of the present invention can comprise additional components, such as means for dispensing a drug/a dispensing unit for dispensing a drug. Accordingly, it is encompassed that the apparatus, either as an applicator like appliance or as a permanently or reversibly implanted appliance, can be used in the treatment of a disease or in a method of treatment. As mentioned, the apparatus of the present invention can comprise an anti-IL6 sdAb of the present invention. Accordingly, the apparatus is especially useful in the treatment of inflammatory and autoimmune diseases provided herein. As mentioned herein above, it is envisioned that the apparatus of the present invention can measure and detect inflammation spikes, e.g. increased IL6 levels and dispense a drug, e.g. anti-inflammation drug in response to such inflammation spikes. In general, all features described in the context of the apparatus and treatments of the present invention apply mutatis mutandis to the apparatus for use in the treatment of a disease provided herein. When used in the context of a treatment or therapy, the treatment may further comprise a set of dispensing a drug as described herein, e.g. a sdAb of the invention or any drug described in the context of treatments herein.

Accordingly, the invention provides in one aspect an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in therapy.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, optionally a systemic inflammation.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, optionally a systemic inflammation, wherein the disease is associated with an increased production or release of inflammatory markers.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, optionally a systemic inflammation, wherein the disease is associated with an increased production or release of IL6.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, optionally a systemic inflammation, wherein the disease is an inflammatory and/or autoimmune disease.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus performs the method for detecting and/or quantifying IL6 in or on a subject of the present invention prior to dispensing a drug.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus performs the method for detecting and/or quantifying IL6 in or on a subject of the present invention prior to dispensing a drug, and wherein the dispensing of said drug depends on the measurement obtained from the method for detecting and/or quantifying IL6 in or on a subject.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus performs the method for detecting and/or quantifying IL6 in or on a subject of the present invention and compares the level of IL6 to a reference value prior to dispensing a drug, and wherein the dispensing of said drug depends on the difference between the measured IL6 level and the reference value.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus performs the method for detecting and/or quantifying IL6 in or on a subject of the present invention and compares the level of IL6 to a reference value prior to dispensing a drug, and wherein the dispensing and amount of said drug depends on the difference between the measured IL6 level and the reference value.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus is permanently or reversibly attached to or implanted into a subject.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the treatment further comprises comparing the level of anti-IL6 with a reference value or reference sample as described herein.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus repeatedly contacts the subject with the anti-IL6 sdAb and repeatedly detects and/or quantifies binding of the anti-IL6 sdAb over time.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus repeatedly contacts the subject with the anti-IL6 sdAb and repeatedly detects and/or quantifies binding of the anti-IL6 sdAb over time.

In a preferred aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, and
(iv) a dispensing unit for dispensing a drug,
   for use in the treatment of a disease associated with an inflammation, wherein the apparatus repeatedly contacts the subject with the anti-IL6 sdAb and repeatedly detects and/or quantifies binding of the anti-IL6 sdAb over time, wherein the treatment further comprises comparing the level of anti-IL6 with a reference value or reference sample as described herein after every detection and/or quantification step, and wherein the apparatus is permanently or reversibly attached to or implanted into a subject.

In addition, the invention provides an apparatus for use in in vivo diagnosis of a disease associated with an inflammation. In general, all features described herein in the context of the apparatus and (in vivo) diagnosis of the present invention apply mutatis mutandis to the apparatus for use in in vivo diagnosis of a disease provided herein.

Accordingly, the invention provides in one aspect an apparatus comprising:
(i) an anti-IL6 sdAb of the present invention, and
(ii) a contacting unit for contacting a subject with the anti-IL6 sdAb, and
   (iii) a detecting unit for detecting and/or quantifying binding of the anti-IL6 sdAb,
   for use in in vivo diagnosis.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb, for use in in vivo diagnosis of a disease associated with inflammation, optionally a systemic inflammation.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the disease associated with an inflammation or systemic inflammation is an inflammatory disease and/or an autoimmune disease.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the disease associated with an inflammation or systemic inflammation is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the apparatus repeatedly contacts the subject with the anti-IL6 sdAb and repeatedly detects and/or quantifies binding of the anti-IL6 sdAb.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the apparatus repeatedly contacts the subject with the anti-IL6 sdAb and repeatedly detects and/or quantifies binding of the anti-IL6 sdAb over time.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the apparatus is permanently or reversibly attached to or implanted into a subject.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the diagnosis further comprises comparing the level of anti-IL6 with a reference value or reference sample as described herein.

In another aspect, the invention provides an apparatus comprising:
(i) an anti-IL6 sdAb of the present invention, and
(ii) a contacting unit for contacting a subject with the anti-IL6 sdAb, and
(iii) a detecting unit for detecting and/or quantifying binding of the anti-IL6 sdAb,
   for use in in vivo diagnosis of a disease associated with inflammation, optionally a systemic inflammation, wherein the apparatus repeatedly contacts the subject with the anti-IL6 sdAb and repeatedly detects and/or quantifies binding of the anti-IL6 sdAb over time and wherein the diagnosis further comprises comparing the level of anti-IL6 with a reference value or reference sample as described herein after every detection and/or quantification step, and wherein the apparatus is permanently or reversibly attached to or implanted into a subject.

The invention also provides a method of producing an anti-IL6 sdAb. As mentioned herein above, the anti-IL6 sdAb of the present invention are particularly useful since they embody specific on-rate constant and off-rate constant ranges. Accordingly, an anti-IL6 sdAb in the sense of the present invention can be produced by specifically selecting for a desired on-rate constant and off-rate constant. An exemplary range of an on-rate constant in the sense of the invention is about 6.3×10\hat{ }4-1.3×10\hat{ }7. An exemplary range of an off-rate constant is about 1.2×10\hat{ }−6-0.31. Accordingly, an anti-IL6 sdAb can be specifically selected for an on-rate constant (kₒₙ) of about 6.3×10\hat{ }4-1.3×10\hat{ }7 and an off-rate constant (k_{off}) of about 1.2×10\hat{ }−6-0.31. Another exemplary range of range of an on-rate constant in the sense of the invention is about 0.15×10\hat{ }6-2.25×10\hat{ }6. Another exemplary range of an off-rate constant is about 0.0002-0.002. Accordingly, an anti-IL6 sdAb can be specifically selected for an on-rate constant (kₒₙ) of about 0.15×10\hat{ }6-2.25×10\hat{ }6 and an off-rate constant (k_{off}) of about 0.0002-0.002. The skilled person is aware how to select for useful on-rate and off-rate constant ranges depending on the desired application.

Accordingly, the invention provides in one aspect a method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 6.3×10\hat{ }4-1.3×10\hat{ }7 and an off-rate constant (k_{off}) of about 1.2×10\hat{ }−6-0.31.

In another aspect, the invention provides a method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 0.15×10\hat{ }6-2.25×10\hat{ }6 and an off-rate constant (k_{off}) of about 0.0002-0.002.

A particular desired property of an anti-IL6 sdAb of the present invention is the ability to continuously and reversibly bind IL6. In addition, it is desired that a sdAb of the present invention can bind to multiple IL6 molecules, i.e. binding to a second or a further IL6 molecule after dissociating from a first IL6 molecule.

Accordingly, the present invention provides in one aspect a method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 6.3×10\hat{ }4-1.3×10\hat{ }7 and an off-rate constant (k_{off}) of about 1.2×10\hat{ }−6-0.31, and
(iv) selecting for continuous and reversible binding of IL6.

In another aspect, the present invention provides a method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 6.3×10\hat{ }4-1.3×10\hat{ }7 and an off-rate constant (k_{off}) of about 1.2×10\hat{ }−6-0.31, and
(iv) selecting for continuous and reversible binding of IL6, and
(v) selecting for binding to a second or further IL6 after dissociation from a first IL6.

As mentioned herein above, it can in some embodiments be desired for a sdAb to be able to inhibit the biological function, while it is in other embodiments desired that a sdAb does not inhibit the biological function of IL6. Thus, the method of producing an anti-IL6 sdAb can further comprise a step of selecting if a sdAb inhibits or does not inhibit the biological function of IL6. The biological function of IL6 may be IL6 signalling or the IL6 signalling pathway, i.e. an inflammation response.

Accordingly, the invention provides in one aspect a method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 6.3×10\hat{ }4-1.3×10\hat{ }7 and an off-rate constant (k_{off}) of about 1.2×10\hat{ }−6-0.31, and
(iv) selecting for continuous and reversible binding of IL6, and
(v) selecting for inhibiting the biological function of IL6 or not inhibiting the biological function of IL6.

In another aspect, the invention provides a method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 6.3×10\hat{ }4-1.3×10\hat{ }7 and an off-rate constant (k_{off}) of about 1.2×10\hat{ }−6-0.31, and
(iv) selecting for continuous and reversible binding of IL6, and
(v) selecting for inhibiting the biological function of IL6 or not inhibiting the biological function of IL6, wherein the biological function of IL6 is IL6 signalling or the IL6 signalling pathway, such as an inflammation response.

A subject in the sense of the method of producing an anti-IL6 sdAb can be a camelid. A camelid is an animal of the biological family of Camelidae. Exemplary camelids are dromedary camels, Bactrian camels, wild Bactrian camels, llamas, alpacas, vicunas, guanacos, and huarizos. In a preferred embodiment a subject in the context of the method of producing an anti-IL6 sdAb as described herein is a camelid. In another preferred embodiment a subject in the context of the method of producing an anti-IL6 sdAb as described herein is a huarizo.

The invention also provides anti-IL6 sdAbs produced by the method of producing an anti-IL6 sdAb as described herein. The skilled person is aware how to select and produce desired sdAbs in the sense of the present invention. One way of producing sdAbs in the sense of the present invention is provided in detail in the appended examples. Exemplary anti-IL6 sdAbs produced by the method of producing an anti-IL6 sdAb described herein and in the appended examples are provided in SEQ ID NOs: 1, 9, 17, 25, 33, 41, 49, or 57.

In any of the embodiments provided herein, an anti-IL6 sdAb can also be used in combination with an anti-C-reactive protein (anti-CRP) sdAb.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer-defined protocols and conditions unless otherwise noted.

As used herein, the singular forms “a,” “an”, and “the” include the plural referents unless the context clearly indicates otherwise. The terms “include”, “such as”, and the like are intended to convey inclusion without limitation, unless otherwise specifically indicated.

As used herein, the term “or” is generally employed in its usual sense including “and/or” unless the content clearly dictates otherwise. The term “and/or” means one or all of the listed elements or a combination of any two or more of the listed elements.

As used herein, the term “comprising” also specifically includes embodiments “consisting of” and “consisting essentially of” the recited elements, unless specifically indicated otherwise. The terms “comprising” and “having” may be used interchangeably herein.

As used herein, the term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

As used herein “association” preferably means that an anti-IL6 sdAb of the invention “associates” i.e. binds with an IL6. As used herein “bind” or “binds” preferably means binding of an anti-IL6 sdAb of the invention with IL6, e.g. in a sample from a subject, or in/on a subject. Binding or association of an anti-IL6 sdAb with IL6 may be detected in vitro, ex vivo or in vivo.

As used herein “subject” means an individual. In general, a subject in the sense of the invention can be a mammal, preferably a human. In the sense of the treatments and diagnoses of the invention, a subject is preferably a human. In the sense of the treatments and diagnoses of the invention, a subject may be a patient, such as a human or animal patient. In the sense of the method of producing an anti-IL6 sdAb, a subject is preferably a camelid, more preferably a huarizo.

As used herein “measurement” in general means the detection and/or quantification of IL6. Thus, the term measurement may relate to a detection of IL6 and/or the quantification of an IL6 level. In all aspects of the invention, a measurement of an IL6 level can be compared to a reference sample or reference value as described herein.

As used herein “contacting” in general means that two compounds are being brought into close proximity so that a reaction can happen. In the context of contacting a sample or a subject with an anti-IL6 sdAb of the invention, the term “contacting” means that the anti-IL6 sdAb is brought into close proximity with a sample comprising interstitial fluid (ISF), blood, urine, stool, serum, tear fluid, sweat, saliva in vitro or a subject's interstitial fluid (ISF), blood, urine, stool, serum, tear fluid, sweat, or saliva in vivo, which comprise IL6, thereby enabling binding/association of the anti-IL6 sdAb with IL6.

The term “contacting” can also relate to administering a compound, e.g. a drug to a subject. In the sense of the apparatus per se of the present invention, the term “contacting” can mean that the apparatus is applied to a subject, e.g. a subject's skin. In addition, it can encompass that the apparatus is permanently or reversibly attached to or implanted into a subject. In the sense of the contacting steps performed by the apparatus of the invention, the term “contacting” means that an anti-IL6 sdAb of the invention is brought into close proximity with an IL6, thereby enabling binding/association of the anti-IL6 sdAb with IL6.

As used herein, terms like “variant thereof having up to [n] amino acid substitutions, means that the variant can have any of the concrete number of n amino acid substitutions. For example, if the “variant has up to 5 amino acid substitutions”, the variant can have 1, 2, 3, 4 or 5 amino acid substitutions.

As used herein “apparatus” may also be referred to as device.

The methods provided herein can be performed on a sample, such as a sample obtained from a subject. In some embodiments a step of obtaining a sample is not part of any one of the methods described herein. In some embodiments any of the methods described herein do not comprise a step of obtaining a sample from a subject, such as a human or animal.

In some embodiments any one of the methods or uses described herein can be in vitro and/or ex vivo or in vivo. In some embodiments any of the method steps described herein can be in vitro and/or ex vivo or in vivo.

The disclosures in context of the methods described herein are disclosed as corresponding use mutatis mutandis. The disclosures in context of the use described herein are disclosed as corresponding methods mutatis mutandis.

In one aspect, the methods of the present invention are not methods for treatment of the human or animal body by therapy. In a further aspect, the methods of the present invention are not processes for modifying the germ line genetic identity of human beings. In one aspect, the methods of the present invention are in vitro or ex vivo methods. In a further aspect, the methods of the present invention are not processes for modifying the genetic identity of animals which are likely to cause them suffering without any substantial medical benefit to man or animal.

The methods and uses of the present invention can be in vivo, ex vivo or in vitro.

Biacore systems allow monitoring the interaction between molecules in real time using the phenomenon of surface plasmon resonance (SPR). This involves attaching one interacting partner to the surface of a sensor chip, and then passing a sample containing the second interaction partner over the surface. Binding of molecules to the sensor surface generates a response which is proportional to the bound mass. Accordingly, as used herein the term “SPR Biacore” means a SPR binding kinetic measurement. Both terms can be used interchangeably herein. Accordingly, as used herein “wherein the values are obtained by a SPR Biacore binding kinetic measurement” or “wherein the values are determined/assessed using a SPR Biacore binding kinetic measurement” and “SPR Biacore measurement” are equivalent to and can be interchangeably used with “wherein the values are obtained by/determined/assessed using a SPR binding kinetic measurement” and “SPR binding kinetic measurement”, respectively.

When referring herein to any measurement obtained, determined, performed “by a SPR Biacore” and similar formulations, the SPR Biacore is preferably a SPR Biacore T200. Furthermore, when referring herein to any measurements “obtained/determined/assessed by a SPR Biacore” and similar formulations, the measurements were done preferably according to the standard protocol provided by the manufacturer, such as in the “Biacore™ sensor surface handbook” published in 2022 and available on https://cdn.cytivalifesciences.com/api/public/content/digi-16475-pdf (availability as of Oct. 5, 2024) which is incorporated herein in its entirety. An exemplary method to obtain measurements by SPR binding kinetic measurements is provided in Stenberg et al. 1991, which is incorporated herein in its entirety. An exemplary method to obtain measurements by a SPR Biacore or SPR binding kinetic measurements in general is provided in the appended examples. When referring herein to any measurements “obtained/determined by a SPR Biacore” and similar formulations, the measurement method preferably comprises:
a. immobilizing sdAbs of the invention on a SPR COOH-chip (e.g. CM5 sensor chip, Cytiva, Cat. 29149603) through EDC/NHS coupling, wherein coupling is done individually optimized for each sdAb based on its isoelectric point (pI) by altering pH with sodium acetate buffer,
b. setting the coupling to reach the same coating density for all sdAbs to be measured (e.g. 200 response units),
c. using different IL6 concentrations (e.g. as listed in the Table 1) wherein the concentration range depends on the K_{D} of each sdAb (e.g. an estimate of which can be obtained from Octet BLI screening),
d. obtaining kinetic curves and applying them for fitting the 1:1 Langmuir binding model (e.g. using the Biacore Evaluation software 3.2.),
   wherein the measurement length is 240-300 sec for association and 600-900 sec for dissociation,
   wherein a running buffer is used comprising the following components: 1×PBS+0.01% Tween20, pH 7.4,
   wherein the sdAbs are provided in 10 mM glycine pH 2.0,
   preferably wherein the method is performed on a Biacore T200.

Unless otherwise stated herein the “SPR Biacore binding kinetic measurement” as used herein is performed according to standard manufacturer's conditions (as e.g. described in the “Biacore™ sensor surface handbook”) and/or according to Stenberg et al. In one preferred aspect, a running buffer can be used comprising the following components: 1×PBS+0.01% Tween20, pH 7.4.

Bio-Layer Interferometry (BLI) is an optical analytical technique used to study biomolecular interactions in real-time. It works by measuring the interference pattern of white light reflected from two surfaces: a biosensor surface and an internal reference surface.

When biomolecules bind to the biosensor surface, the optical thickness at the surface changes, resulting in a change in the interference pattern. This change in the interference pattern is measured and used to determine the kinetics and affinity of the molecular interactions occurring on the biosensor surface.

BLI is commonly used for applications such as:
1. Characterizing biomolecular interactions: BLI can measure the binding kinetics, affinity constants, and thermodynamics of interactions between proteins, nucleic acids, small molecules, and other biomolecules.
2. Analyzing protein-protein interactions: BLI is particularly useful for studying protein-protein interactions, which are crucial for understanding biological processes and developing therapeutics.
3. Measuring protein-small molecule interactions: BLI can detect and quantify the binding of small molecules to proteins, which is important in drug discovery and development.
4. Epitope mapping: BLI can map the binding sites (epitopes) of antibodies on target proteins, aiding in the design of therapeutic antibodies and vaccine development.
5. Concentration analysis: BLI can be used to determine the concentration of biomolecules in solution by measuring the binding response to a known target.

BLI offers advantages such as label-free detection, low sample consumption, and the ability to analyze interactions in complex matrices like cell lysates or serum.

BLI is an optical biosensing technique that utilizes an integrated waveguide and interferometer to monitor biomolecular interactions in real-time. The core component is a biosensor, which consists of a linear optical waveguide and two measurement surfaces: a biological surface and an internal reference surface.

The biological surface is functionalized with specific capture molecules (e.g., proteins, nucleic acids, small molecules) that can bind to the analyte of interest. The reference surface is designed to account for any non-specific binding or bulk refractive index changes.

White light is coupled into the waveguide, where it propagates and reflects off the two surfaces, creating an interference pattern. The phase shift in the interference pattern is directly proportional to the thickness and refractive index of the biological layer on the sensor surface. As biomolecules bind to the capture molecules, the optical thickness of the biological layer changes, resulting in a shift in the interference pattern. This shift is detected and quantified by the interferometer, providing a real-time measurement of the binding kinetics and affinity between the analyte and the immobilized capture molecules.

The rate of binding (association rate constant, kₐ) and the rate of dissociation (dissociation rate constant, k_{d}) can be determined by fitting the binding curves to suitable mathematical models. The equilibrium dissociation constant (K_{D}), which is a measure of affinity, can be calculated from the ratio of k_{d}/kₐ.

BLI offers several advantages over other biosensing techniques, such as:
1. Label-free detection: No labeling of biomolecules is required, reducing the risk of altering their native structure and behavior.
2. High sensitivity: BLI can detect biomolecular interactions with picomolar to nanomolar sensitivity.
3. Wide molecular weight range: BLI can analyze interactions involving small molecules (<1 kDa) to large complexes (>1 MDa).
4. Real-time kinetics: BLI provides continuous monitoring of binding events, enabling the determination of kinetic rate constants.
5. Multiplexing capabilities: Multiple biosensors can be used simultaneously to study different interactions in parallel.

In Octet bio-layer interferometry (BLI) systems, one molecule is immobilized to a Dip and Read Biosensor and binding to a second molecule is measured. A change in the number of molecules bound to the end of the biosensor tip causes a shift in the interference pattern that is measured in real-time. Accordingly, as used herein the term “Octet BLI” means a BLI binding kinetic measurement. Both terms can be used interchangeably herein. Accordingly, as used herein “wherein the values are obtained by an Octet BLI binding kinetic measurement” or “wherein the values are determined/assessed using an Octet BLI kinetic measurement” and “Octet BLI measurement” are equivalent to and can be interchangeably used with “wherein the values are obtained by/determined/assessed using a BLI binding kinetic measurement” and “BLI binding kinetic measurement”, respectively.

When referring herein to any measurement obtained, determined, performed “by a Octet BLI” and similar formulations, the Octet BLI is preferably a ForteBio Octet RED96. Furthermore, when referring herein to any measurements “obtained/determined/assessed by a Octet BLI” and similar formulations, the measurements were done preferably according to the standard protocol provided by the manufacturer, such as in the “Octet® System Data Acquisition User Guide” Release 11.0 available on https://facilities.bioc.cam.ac.uk/files/media/data_acquisition_octet web.pdf (availability as of Oct. 5, 2024) which is incorporated herein in its entirety. An exemplary method to obtain measurements by an Octet BLI or BLI binding kinetic measurements in general is provided in the appended examples. When referring herein to any measurements “obtained/determined by a Octet BLI” and similar formulations, the measurement method preferably comprises:
a. immobilizing Biotinylated hIL6 (e.g. RnD Systems, Cat BT7270) on Streptavidin-coated sensors (e.g. Cat 18-5019) for 60 sec with the biotinylated hIL-6 was at 5 μg/mL in PBS (pH 7.4) with 0.01% Tween20,
b. providing a assay running buffer used for the kinetic measurements comprising PBS (pH 7.4) supplemented with 0.01% Tween20 and 0.01% BSA,
c. diluting purified sdAbs of the present invention in the running buffer to a range of concentrations, e.g. 400-0.16 nM, wherein the exact concentrations depend on the respective sdAb clone,
d. dipping the (Octet) BLI sensors into the wells of a 96-well plate containing the sdAb samples,
e. regenerating between readings using 100 mM NaOH+1 M NaCl,
   wherein the readings were done with the following parameters:
Association: 180 sec
Dissociation 300 sec
Assay temperature: 25 °C
Agitation at 1,000 rpm,
f. repeating all measurements with reference sensors (without biotinylated hIL6) to measure background signal, which was subsequently subtracted from the binding signal of the sdAbs,
   preferably wherein the method is performed on a ForteBio Octet RED96.

Unless otherwise stated herein the “BLI binding kinetic measurement” as used herein is performed according to standard manufacturer's conditions (as e.g. described in the “Octet® System Data Acquisition User Guide”). In one preferred aspect, a running buffer can be used comprising the following components: PBS (pH 7.4) supplemented with 0.01% Tween20 and 0.01% BSA.

### BRIEF DESCRIPTION OF THE DRAWINGS

FIG. 1: Steps for the selection of anti-IL6 sdAbs tuned towards the application of continuous IL6 sensing at physiologically relevant concentrations.

FIG. 2: Modeling of the proportion of bound sdAbs for different association rate values kₐ. The solid black line, highlighted by an arrow, represents the modelled concentration change of IL6 molecule in vivo (left y axis). The markers plots represent the proportion of occupied sdAb as predicted by the TEM for different values of association rate kₐ (right y axis). The values used to generate these plots are: low kₐ=0.3×10³ M⁻¹ s⁻¹, medium kₐ=1×103 M⁻¹ s⁻¹, high kₐ=3×10³ M⁻¹ s⁻¹. The dissociation rate is kept constant, k_{d}=1×10⁻³ s⁻¹.

FIG. 3: Modeling of the proportion of bound sdAbs for different dissociation rate values k_{d}. The solid black line, highlighted by an arrow, represents the modelled concentration change of IL6 molecules in vivo (left y axis). The markers plots represent the proportion of occupied sdAbs as predicted by the TEM for different values of dissociate rate k_{d} (right y axis). The values used to generate these plots are: low k_{d}=0.3×10⁻³ s⁻¹, medium k_{d}=10⁻³ s⁻¹, high k_{d}=3×10⁻³ s⁻¹. The association rate is kept constant, kₐ=1×10⁷ M⁻¹×s⁻¹.

FIG. 4: Signal reconstruction algorithm results—A) mean absolute error between a set of IL6 concentration dynamics C(t) and the corresponding reconstructed signal R(t). Reconstruction is achieved using the signal reconstruction algorithm described in “Signal reconstruction algorithm implementation” section below. The output signal R(t) is first corrected for attenuation and time shift and then compared to the input signal C(t). The error is computed for kₐ ∈[10⁴-10⁷] M⁻¹ s⁻¹ and k_{d}∈[10⁻⁵-10⁻²] s⁻¹ and averaged across signals. B) Example of signal reconstruction outputs R(t) for (1) k_{d}=10⁻² s⁻¹, (2) k_{d}=3.10⁻⁴ s⁻¹, and (3) k_{d}=10⁻⁵ s⁻¹. The initial IL6 concentration dynamic C(t) is shown as dashed line (left y axis). The output of the TEM, i.e., percentage of bound sdAbs with additive noise is shown as dark line (right y axis). The R(t) signal obtained after applying the reconstruction algorithm is shown as marker plot (left y axis).

FIG. 5: Signal processing pipeline. Sequence of signal processing steps applied from the initial concentration dynamic of IL6 C(t) to the reconstructed signal R(t), including the computation of differences between the two signals.

FIG. 6: Schematic of the overall experimental procedure to raise and isolate anti-IL6 sdAbs from an adult Huarizo.

FIG. 7: Assessment of the immune response on day 0, 47, 75 and 124 against human IL6 by ELISA. Bound antibodies were detected using a goat-anti-llama-HRP IgG GaLPO.

FIG. 8: Generation of the sdAb-phagemid libraries of day 43, day 75 and day 124. A) Gel electrophoresis of 5pI of the precipitated RNA samples on a 1% TBE agarose gel. The 28S and 18S ribosomal RNA bands are indicated. B) Amplification of the sdAb/VHH (~700 bp) and the VH (~900 bp) cDNA, analyzed on a 1% TBE agarose gel. C) 10-fold dilutions of TG1 E. coli cells transformed with the sdAb libraries, spotted onto LB-agar plates supplemented with 100 μg/ml ampicillin and 2% glucose. D) Colony PCR of different clones from each transformation/library.

FIG. 9: Results of the first round of selections on IL6. A) Input phages were diluted up to 1×10\hat{ }10 and used to infect E. coli TG1 before spotting on selective LB agar plates. B) Results of the selection output on wells coated with indicated amount of directly coated IL6.

FIG. 10: Results of the second round of selections on IL6. A) Input phages were diluted up to 1×10\hat{ }10 and used to infect E. coli TG1 before spotting on selective LB agar plates. B) Results of the selection output on wells coated with indicated amount of directly coated IL6 and with the indicated incubation times and washing conditions.

FIG. 11: Analysis of sequences from master plates SIL-1, SIL-2 and SIL-3 using the PipeBio software. A) sdAb annotation score indicated the percentages of successfully annotated sdAb genes in the dataset. B) Treemap indicating the relative size of the sdAb clusters found amongst master plates SIL-1, SIL-2 and SIL-3. Cluster number and CDR-H3 sequence are depicted. C) Cluster locations indicated in the master plates layouts, empty well: no correct/annotated sequence.

FIG. 12: Concentration standard curve established by Octet BLI used to derive anti-IL6 sdAb concentration in periplasmic extracts.

FIG. 13: PageBlue-stained SDS-PAGE gel containing 1 μg of tagless reference sdAb in lane 2, and PageRuler™ Prestained Protein Ladder in lanes 1 and 11 and 1 μg of the indicated sdAb in the other lanes.

FIG. 14: Assessing binding of purified sdAb from masterplate SIL-2 and SIL-3 to immobilized IL6 by ELISA. Bound sdAb were detected using rabbit-anti-sdAb, followed by donkey-anti-rabbit HRP and OPD as substrate.

FIG. 15: SPR Biacore binding kinetic measurements done for anti-IL6 sdAb SIL-2F5. Curves selected for fitting. Gray scale curves indicate IL6 binding signal at different concentrations, black curves indicate fitted model curves.

FIG. 16: SPR Biacore binding kinetic measurements done for anti-IL6 sdAb SIL-3B6. Curves selected for fitting. Gray scale curves indicate IL6 binding signal at different concentrations, black curves indicate fitted model curves.

FIG. 17: SPR Biacore binding kinetic measurements done for anti-IL6 sdAb SIL-3E7. Curves selected for fitting. Gray scale curves indicate IL6 binding signal at different concentrations, black curves indicate fitted model curves.

FIG. 18: SPR Biacore binding kinetic measurements done for anti-IL6 sdAb SIL-2D1. Curves selected for fitting. Gray scale curves indicate IL6 binding signal at different concentrations, black curves indicate fitted model curves.

FIG. 19: SPR Biacore binding kinetic measurements done for anti-IL6 sdAb SIL-2E4. Curves selected for fitting. Gray scale curves indicate IL6 binding signal at different concentrations, black curves indicate fitted model curves.

FIG. 20: SPR Biacore binding kinetic measurements done for anti-IL6 sdAb SIL-3B7. Curves selected for fitting. Gray scale curves indicate IL6 binding signal at different concentrations, black curves indicate fitted model curves.

FIG. 21: SPR Biacore binding kinetic measurements done for anti-IL6 sdAb SIL-2D8. Curves selected for fitting. Gray scale curves indicate IL6 binding signal at different concentrations, black curves indicate fitted model curves.

FIG. 22: SPR Biacore binding kinetic measurements done for anti-IL6 sdAb SIL-2E7. Curves selected for fitting. Gray scale curves indicate IL6 binding signal at different concentrations, black curves indicate fitted model curves. There was minimal signal over background (curves that were negative were excluded from the graph with fitted curves), such that kₒₙ and k_{off} could not be assessed. The amine group of the lysine in the CDR1 region could possibly react with the EDC/NHS coupling chemistry used to attach sdAbs to the SPR chips, such that the CDR1 region would be blocked from being able to bind free antigen.

FIG. 23: Steady state analysis of analyte binding. A: BLI signal increase with analyte concentration. B: BLI signal increase with the log of analyte concentration. The prediction from the TEM using the average kₐ and k_{d} computed across sensor repeats and analyte concentration steps is shown as a line for each concentration value.

FIG. 24: Linear binding regime. Binding signal as a function of K_{D} for each sensor. A linear fit computed for concentrations comprised in the range [0.1-1.3] K_{D} is shown with the 95% prediction interval.

FIG. 25: Anti-IL6 sdAbs (SIL-2D8, SIL-3B6, SIL-3E7) target concentration range for various inflammation levels in vivo. The range of IL6 concentration that can be detected using the sdAbs of the present invention is highlighted. The assumed level of detection (LOD) is highlighted as a dashed line. The data shown in FIG. 25 is based on Table 12.

FIG. 26: Anti-IL6 sdAbs (SIL-2F5, SIL-2D1, STL-2E4, SIL-3B7) target concentration range for various inflammation levels in vivo. The range of IL6 concentration that can be detected using the sdAbs of the present invention is highlighted. The assumed level of detection (LOD) is highlighted as a dashed line. The data shown in FIG. 26 is based on Table 12.

FIG. 27: Effect of sdAbs and controls alone, without IL6 antigen, on IL6 signaling tested on two independent days, A) day 1, B) day 2. RLU: Relative light units. FL: Firefly Luciferase.

FIG. 28: Inhibition curves of IL6 signaling by Siltuximab. A) showing RLU signal depending on concentration, B) showing calculated % inhibition depending on concentration. RLU: Relative light units. FL: Firefly Luciferase.

FIG. 29: Inhibition curves of IL6 signaling by control sdAbs. A) showing RLU signal depending on concentration, B) showing calculated % inhibition depending on concentration. RLU: Relative light units. FL: Firefly Luciferase.

FIG. 30: Inhibition curves of IL6 signaling by SIL-2D8. A) showing RLU signal depending on concentration, B) showing calculated % inhibition depending on concentration. RLU: Relative light units. FL: Firefly Luciferase.

FIG. 31: Inhibition curves of IL6 signaling by SIL-3B6. A) showing RLU signal depending on concentration, B) showing calculated % inhibition depending on concentration. RLU: Relative light units. FL: Firefly Luciferase.

FIG. 32: Inhibition curves of IL6 signaling by SIL-3E7. A) showing RLU signal depending on concentration, B) showing calculated % inhibition depending on concentration. RLU: Relative light units. FL: Firefly Luciferase.

FIG. 33: Inhibition curves of IL6 signaling by SIL-2D1. A) showing RLU signal depending on concentration, B) showing calculated % inhibition depending on concentration. RLU: Relative light units. FL: Firefly Luciferase.

FIG. 34: Inhibition curves of IL6 signaling by SIL-2E4. A) showing RLU signal depending on concentration, B) showing calculated % inhibition depending on concentration. RLU: Relative light units. FL: Firefly Luciferase.

FIG. 35: Inhibition curves of IL6 signaling by SIL-2E7. A) showing RLU signal depending on concentration, B) showing calculated % inhibition depending on concentration. RLU: Relative light units. FL: Firefly Luciferase.

FIG. 36: Inhibition curves of IL6 signaling by SIL-2F5. A) showing RLU signal depending on concentration, B) showing calculated % inhibition depending on concentration. RLU: Relative light units. FL: Firefly Luciferase.

FIG. 37: Inhibition curves of IL6 signaling by SIL-3B7. A) showing RLU signal depending on concentration, B) showing calculated % inhibition depending on concentration. RLU: Relative light units. FL: Firefly Luciferase.

FIG. 38: Summary graphs of the inhibition of IL6 signaling sdAbs and controls tested on two independent days, A) day 1, B) day 2.

The invention is illustrated in the following examples.

### EXAMPLE 1: GENERATION OF ANTI-IL6 sdAbs (VHHs)

### Key Abbreviations

BLI: Bio-Layer Interferometry
K_{D}: dissociation constant (affinity)=k_{d}/kₐ
k_{d}: kinetic dissociation rate constant (also k_{off}, or off-rate)
kₐ: kinetic association rate constant (also kₒₙ, or on-rate)
IL6: Interleukin 6
PBMCs: Peripheral Blood Mononuclear Cells
PBS: Phosphate-buffered saline
PBST: PBS with 0.05% Tween-20
MPBS: 4% milk powder in PBS
RT: Room temperature
TEM: Thermodynamic equilibrium model
VHH: heavy chain variable domain of a camelid single-chain antibody; also: single-domain antibody (sdAb)

### Material and Methods

### Analytical Modeling of IL6 Dynamics

Gaussian functions were used to model IL6 concentration dynamics that mimic levels measured in human serum during homeostasis and disease (Blackburn et al., 2006; Emerson et al., 2017; Esposito et al., 2002; Herieka & Erridge, 2014; Lippitz & Harris, 2016; Mavropoulou et al., 2020; Song et al., 2019; Zhou et al., 2020).

The IL6 concentration equation was defined as follows:

$$C ( t ) = {C}_{baseline} + {C}_{high} \cdot {e}^{- \frac{2 {( t - μ )}^{2}}{{w}^{2}}} \qquad Equation 1$$

With C_{baseline} and C_{high} the baseline and peak concentration values respectively, t the time step, μ the time step at which the concentration value is maximum, and w reflecting the duration of the concentration peak. Multi-gaussian distributions were obtained by summing multiple Gaussian signals with different parameters μ. A typical IL6 concentration dynamic was represented by the following input parameters: C_{baseline}=0.01 pM, C_{high}=0.1 pM, μ=2 h, w=2 h and a time step t=5 min.

### Analytical Implementation of the Thermodynamic Equilibrium Model (TEM)

Binding kinetics between IL6 and anti-IL6 sdAb were approximated by the Langmuir absorption model (Kusnezow et al., 2006). This simple analytical formula describes the kinetic interactions between an analyte, and a capture molecule, here respectively: the IL6 antigen and the anti-IL6 sdAbs.

According to this thermodynamic equilibrium model, the proportion of bound sdAbs S(t) can be expressed as:

$$S ( t ) = {S}_{eq} ( 1 - {e}^{- \frac{t}{τ}} ) \qquad Equation 2$$

$$With :$$

$${S}_{eq} = {S}_{max} \frac{C ( t )}{C ( t ) + {K}_{D}} \qquad Equation 3$$

$${K}_{D} = \frac{{k}_{d}}{{k}_{a}} , and \qquad Equation 4$$

$$τ = \frac{1}{{k}_{d} + {k}_{a} \cdot C ( t )} = \frac{1}{{k}_{a} ( {K}_{D} + C ( t ) )} \qquad Equation 5$$

Where C(t) is the analyte concentration at time t, Sₘₐₓ the maximum proportion of bound active sites (i.e., 100%), S_{eq} the signal at equilibrium, kₐ the association rate, k_{d} the dissociation rate, K_{D} the dissociation constant, and r the reaction time constant.

This simplified model assumes no limitation from analyte diffusion. It implies that, for a dissociation constant K_{D} largely superior to the analyte concentration C(t), the readout signal is proportional to C(t) at any time t. When K_{D} approaches or exceeds C(t), a delay is introduced between the readout signal and the concentration dynamics, and the linear relationship does not hold anymore.

To calculate the proportion of bound sdAbs S(t) for a given IL6 concentration target range, the limit of detection of the system was assumed to be equivalent to 0.01% of bound sdAbs, or 100 parts per million.

### Signal Reconstruction Algorithm Implementation

The use of a custom transfer function for reconstructing the initial IL6 concentration dynamics in case the proportion of bound sdAbs did not vary linearly with the input IL6 concentration, for example for slow k_{d} values, was evaluated. The approach was inspired by Ravelo et al. (Ravelo et al., 2020) and modified to be applied to the thermodynamic binding reaction between a capture molecule and an analyte. Essentially, the TEM was assimilated to a distorting low pass RC filter whose effects can be reversed applying an inverse low pass element. Thus, an inversed low pass filter was implemented to reconstruct the initial signal C(t) from the TEM output signal S(t). The transfer function of this circuit was defined as

$${H}_{high} ( s ) = \frac{{τ}_{algo} + \frac{{τ}_{algo}}{K} + s}{\frac{{τ}_{algo}}{K} + s}$$

with τ_{algo} the time constant of the system. τ_{algo} was simplified to

$$\frac{1}{{k}_{d}}$$

to not depend on the initial concentration. This is valid for concentration C(t) lower or close to K_{D} (equation (3)). K is a parameter mimicking a shunt resistor. It governs the reconstruction strength of the algorithm and ranges from 0.1 to 10⁴Ω. High K values lead to smoother reconstruction but more errors with respect to the initial IL6 concentration C(t). Conversely, low K values result in more faithful but noisier signal reconstruction. The reconstruction circuit takes as input the proportion of bound sdAb S(t) at instant t, and returns the reconstructed signal R(t). The initial value of the reconstructed signal was fixed to

$$C ( t = 0 ) \cdot \frac{τ}{K}$$

after heuristically testing dependencies. The robustness of the reconstruction was investigated by adding relative noise to the TEM output S(t). The noise level was set proportional to the highest percentage of bound sdAbs.

Next, a population of five IL6 concentration dynamics was defined. The input dynamics C(t) consisted in single and double gaussian signals. Signals contained either a low or high concentration peak, two low or two high concentration peaks, or a low and a high concentration peak. Concentrations were fixed to C_{baseline}=0.1 pM, C_{peak low}=0.2 pM, C_{peak high}=1 pM with μ_{first peak}=8 h, μ_{second peak}=14 h and W=1.6 h for both peaks. These concentration values were chosen to cover the large span of IL6 responses observed in humans in homeostasis and disease (Blackburn et al., 2006; Emerson et al., 2017; Esposito et al., 2002; Herieka & Erridge, 2014; Lippitz & Harris, 2016; Mavropoulou et al., 2020; Song et al., 2019; Zhou et al., 2020). The computation time step t was set to 100 s and the total signal length to 20 hours. K was set to 20Ω and the noise level to 10%. For each concentration dynamics C(t), the output of the TEM, S(t), and the reconstructed signal, R(t), was computed. The process was repeated for kinetic rates kₐ ∈[10⁴-10⁷] M⁻¹ s⁻¹ and k_{d}∈[10⁻⁵-10⁻²] s⁻¹. Upper bound of kₐ and lower bound for k_{d} were based on literature (Foote & Eisen, 1995). Additionally, kₐ values lower than 10⁴ M⁻¹ s⁻¹ and k_{d} values larger than 10⁻² s⁻¹ were not considered as they would lead to very low sdAb binding. The reconstruction error between C(t) and R(t) was evaluated by first correcting for the time shift between the two signals, applying cross-correlation, and for the attenuation, applying normalization. The mean absolute error between C(t) and R(t) for each time t was then computed. Errors were averaged across the population of IL6 concentration dynamics.

Immunization of a Huarizo with Recombinant Human IL6

One adult Huarizo was immunized with a glycosylated human IL6 (Proteintech, Cat. HZ-1019) and GERBU FAMA adjuvant. Six successive immunizations were performed every 2 weeks (starting at day 0), injecting 50 pg IL6 antigen plus adjuvant intramuscularly. Antigen and adjuvant were injected as a 1:1 (v/v) mixture except for the third and fifth injection where antigen and adjuvant were injected at the same time but at separate locations. Two rounds of immunizations with glycosylated human IL6 (Proteintech, HZ-1019) and Incomplete Freund's adjuvant (IFA) were performed on day 105 and day 119. For these two immunizations 50 pg IL6 antigen plus adjuvant were injected subcutaneously. On day 124, serum and PBMCs were collected. PBMCs were used for total RNA isolation. RNA was precipitated in EtOH and NaAc and dissolved in rNase-free milli-Q.

### Testing the Huarizo's Immune Response Against Human IL6 by ELISA

The immune response after immunization is determined by ELISA using sera from day 0 (before immunization), day 47, day 75 and day 124. The ELISA is done according to standard laboratory procedures. In short, MaxiSorp plates are coated overnight at 4 °C with 50 μl of antigen at 2 μg/ml in PBS. The next day, the antigen is removed, plates are washed with PBS and then blocked with 4% milk powder in PBS (MPBS). Next, the wells are incubated with serially diluted sera for 1 hour at room temperature. Bound antibodies in the sera are detected with rabbit-anti-sdAb, followed by donkey-anti-rabbit HRP. In between incubations, plates are washed with PBS with 0.05% Tween-20 (PBST) or PBS. Bound HRP-coupled antibodies are detected using o-phenylenediamine dihydrochloride (OPD) with H₂O₂ as substrate and converted substrate is measured at 490 nm wavelength.

Generating sdAb Phagemid Libraries

Integrity of the total RNA is determined by assessing the 28S and 18S rRNA on a 1% agarose gel. A total of 40 μg RNA is transcribed into cDNA using a reverse transcriptase reaction (Thermo Fisher Scientific), which is followed by purification using a PCR clean-up kit (Macherey Nagel). cDNAs encoding for heavy chain immunoglobulin domains (both conventional and heavy chain-only) are amplified by PCR using proprietary primer sets. This results in two types of DNA fragments (sizes of ~900 bp and ~700 bp) which represent the conventional and heavy chain-only antibody cDNAs respectively. The ~700 bp fragments (CH1-VHH of the heavy chain-only antibodies) are excised from the gel, purified and used as a template for a nested PCR that introduces an 5′ flanking SfiI restriction site. Amplified nested-PCR fragments are then purified as before and subsequently digested with SfiI and Eco91I. This results in ~400 bp gene fragments containing the sdAb genes, which are purified using a PCR clean-up kit (Machery Nagel). Digested sdAb genes are ligated in frame with geneIII into the pQ81 phagemid vector and transformed into TG1 by electroporation. To assess the transformation efficiency, the transformed TG1 are titrated using 10-fold dilutions and then spotted on LB-agar plates supplemented with 100 μg/ml ampicillin and 2% glucose. The total number of transformants was estimated by counting colonies in the highest dilution and using the formula: Library size=(amount of colonies)*(dilution)*3 ml/0.005 ml (spotted volume). The insert frequency was determined by randomly picking 24 clones from each library and performing a colony PCR using primers that anneal in the pQ81 vector. In this PCR, successfully inserted sdAb fragments will yield PCR-fragments of ~700 bp, compared to a ~300 bp for empty plasmids. All libraries were stored at −80 °C in 2×YT medium supplemented with 20% glycerol, 2% glucose and 100 μg/ml ampicillin.

### Phage Production and Panning

Phages expressing the sdAbs via fusion to the minor coat protein pIII were based on the VCSM13 bacteriophage and were produced in E. coli strain TG1. For the first round of selections, phages were produced from the libraries in TG1 bacteria. TG1 containing the sdAb-phagemid libraries were diluted from glycerol stocks in 2×YT medium containing 2% glucose and 100 μg/ml ampicillin. The cultures were inoculated up to an OD600 of 0.05, while the number of bacteria of the inoculum is >10× the library size (>1×10⁸ bacteria inoculum). Inoculated cultures were grown at 37 °C for 2 hours to reach an OD600 of ~0.5. Subsequently, 7 ml of the cultures were infected with helper phage VCSM13 using a MOI (multiplicity of infection) of ~100. After 30 minutes, infected bacteria were spun down and resuspended into 50 ml fresh 2×YT medium supplemented with both ampicillin (100 μg/ml) and kanamycin (25 pg/ml). Phages were produced overnight at 37 °C, shaking. For the second round of selections, phages were produced from rescued outputs. Overnight cultures from rescued outputs were first diluted a 100-fold in 5 ml fresh 2×YT medium supplemented with 2% glucose and 100 μg/ml ampicillin and grown for 2 hours until the logarithmic growth phase. Subsequently, these cultures were infected with helper phage and phages were produced overnight as described above. In general, produced phages were precipitated from the supernatant of the cultures using PEG-NaCl precipitation. Titers of the produced phages were calculated by serial dilution of the phage sample and infection of E. coli TG1.

### Phage-Display and Panning Selections

For the first round of selections, precipitated phages were pre-blocked and applied to wells coated overnight at 4 °C with 100pI IL6 at 5 g/mL. The next day, unbound antigen was removed, the wells were washed three times with PBS and then blocked for 1 hour at room temperature (RT) with 200 μl of 4% MPBS. The amount of phages used was at least 10-fold of the transformation efficiency (estimated diversity) of the library. Phages were pre-blocked with 100 μl of 2% MPBS for 30 minutes at RT (phage dilution of 1:5). For the panning, the coated and blocked wells were incubated with the pre-blocked phages for 2 hours at RT. Upon extensive washing with PBS-Tween and PBS, bound phages were eluted with 0.1M TEA-solution, which was immediately neutralized with 1M Tris/HCl pH 7.5. Eluted phages were allowed to infect TG1 bacteria (rescued output). In order to assess the size of the output, eluted phages were serially diluted, infected into TG1 and then spotted on LB-agar plates supplemented with 2% glucose and 100 μg/ml of ampicillin. For the second round of selections, phages produced from the outputs of the day 124 library were used to generate input titers. 1 μl of the precipitated and PBS-resuspended phages per selection condition were then pre-blocked in 100 μl of 2% MPBS (phage dilution of 1:100) and subsequently applied to IL6-coated and blocked wells at RT. To direct the panning towards selecting sdAbs with a high on-rate, IL6 was coated at increasingly high concentrations, namely 20, 2 and 0.2 g/mL. In addition to the standard 120 minutes of incubation, the phages were in some conditions incubated for a shorter time period, namely 30 minutes and 5 minutes. Lastly, to prevent loss of sdAb with higher off-rate, a shorter wash was applied with only 5 minutes of washing compared to >30 minutes of washing in some conditions. Eluted phages were rescued in TG1 and spotted on LB-agar plates as indicated above. All rescued outputs were stored at −80 °C as glycerol stocks. Single clones from the selection outputs were picked and combined in three 96-wells masterplates (192 sdAb clones in total), which were cultivated overnight at 37 °C in 2×YT medium supplemented with 2% glucose and 100 μg/ml ampicillin. Next, 20% glycerol was added, and the plates stored at −80 °C.

Production of Periplasmic Extracts from Masterplates

In order to test individual sdAb clones from masterplates for antigen-binding, periplasmic extracts were produced. Masterplates were duplicated into deep well plates containing 1 ml 2×YT medium supplemented with 0.1% glucose and 100 μg/ml ampicillin per well. These cultures were grown for 3 hours at 37 °C after which production of sdAb was induced with 1 mM of isopropyl-beta-D-thiogalactopyranoside (IPTG). The production of sdAb was conducted overnight at RT. Periplasmic extracts were prepared by collecting the bacteria by centrifugation, their resuspension into 120 μl of PBS, and a single overnight freeze-thaw cycle at −20 °C. The next day, bacteria debris was spun down by centrifugation to yield the soluble periplasmic fraction containing the sdAb in the supernatant. Periplasmic extracts were either used for ELISA immediately or stored at −20 °C.

### Periplasmic Extract ELISA

MaxiSorp plates were coated overnight at 4 °C with 50 μl of IL6 antigen at 2 μg/ml in PBS. The next day, the antigen was removed, plates were washed, and then blocked with 4% milk powder in PBS (MPBS). Next, the wells were incubated with 5× diluted periplasmic extracts for 1 hour at RT. Bound antibodies in the sera were detected with rabbit-anti-sdAb, followed by donkey-anti-rabbit-HRP. In between incubations, plates were washed with PBS with 0.05% Tween-20 (PBST) or PBS. Bound HRP-coupled antibodies were detected using o-phenylenediamine dihydrochloride (OPD) with H₂O₂ as substrate and converted substrate was measured at 490 nm wavelength.

Periplasmic Anti-IL6 sdAb Binding Kinetics Screening Using Octet BLI
Quantification of sdAbs in the Periplasmic Extracts

In order to determine the binding constants of the nanobodies, the concentration of the sdAbs in the periplasmic extracts had to be determined. The sdAbs contained a poly-histidine tag (His-tag) and their presence could be detected with Octet instrument by using anti-penta-HIS sensors (Sartorius, Cat 18-5120). The quantification standard curve was determined using a control sdAb in PBS (pH 7.4) with 0.01% Tween. Sensors were regenerated in 10 mM glycine (pH 1.7). Periplasmic extracts containing anti-IL6 sdAbs were diluted 1:5 in the running buffer (PBS pH 7.4, 0.01% Tween, 0.01% BSA) prior to the measurements. The readings were done with the following parameters:
Association: 120 sec
Assay temperature: 25 °C
Agitation at 400 rpm
   Binding Assay (i.e., Kinetic Screening of Non-Purified sdAbs in Periplasmic Extracts)

Biotinylated hIL6 (RnD Systems, Cat BT7270) was immobilized on Streptavidin-coated sensors (Cat 18-5019). The sensor loading was done for 60 sec with the biotinylated hIL-6 was at 5 μg/mL in the PBS (pH 7.4) with 0.01% Tween20. The assay running buffer used for the kinetic measurements was PBS (pH 7.4) supplemented with 0.01% Tween20 and 0.01% BSA. Periplasmic extracts were diluted 10-fold in the running buffer (20 μL periplasmic extract+180 μL buffer). The Octet BLI sensors were dipped into the wells of 96-well plate that contained the diluted periplasmic extracts. Regeneration between readings was done using 100 mM NaOH +1 M NaCl. The readings were done with the following parameters:
Association: 180 sec
Dissociation 300 sec
Assay temperature: 25 °C
Agitation at 1,000 rpm

All measurements were repeated with reference sensors (without biotinylated hIL6) to measure background signal, which was subsequently subtracted from the binding signal of the sdAbs.

E. coli Production and Purification of Anti-IL6 sdAbs

Selected clones were transformed into the E. coli strain BL21. Pre-cultures were prepared by growing bacteria containing the selected sdAb in pQ81 overnight at 37 °C in 8 ml 2×YT medium supplemented with ampicillin (100 μg/ml) and 2% glucose. The next day, pre-cultures were diluted into 800 ml pre-warmed 2-YT medium and supplemented with 100 μg/ml ampicillin and 0.1% glucose. The bacteria were grown until OD600 of 0.6-0.9 at 37 °C before induction of the sdAb expression with 1 mM of IPTG. sdAbs were expressed for 4 hours at 37 °C and bacteria were then harvested by centrifugation. Bacteria pellets were resuspended into 30 ml PBS and frozen overnight at −20 °C. Frozen bacteria were thawed at RT and spun down to separate the sdAb-containing soluble fraction from cell debris. sdAbs were purified from the soluble fraction using immobilized metal affinity chromatography (IMAC) using the C-terminal His-tag on the sdAb and sepharose resin charged with cobalt. Bound sdAbs were eluted with 150 mM imidazole, which was removed by repetitive dialysis against PBS. sdAb concentration was determined by UV-VIS spectrometry at 280 nm wavelength. In addition, the purity and integrity of 1 μg of purified sdAbs was assessed by Coomassie blue staining of a 15% SDS-PAGE gel after electrophoresis.

ELISA for apparent sdAb binding affinity to IL6 (i.e., Kinetic screening of purified sdAbs)

MaxiSorp plates were coated with 2 μg/ml in PBS 50 pI/well antigen and incubated overnight. The next day, the antigen was removed, plates were washed with PBS and then blocked with 4% skimmed milk powder in PBS (MPBS). Next, the wells were incubated with sdAb dilution starting at 1000 nM 1:10 for 1 hour at RT. Bound sdAbs were detected with rabbit-anti-sdAb, followed by donkey-anti-rabbit-HRP. In between incubations, plates were washed with PBS with 0.05% Tween-20 (PBST) or PBS. Bound HRP-coupled sdAbs were detected using o-phenylenediamine dihydrochloride (OPD) with H₂O₂ as substrate and converted substrate is measured at 490 nm wavelength.

Anti-IL6 Binding Kinetics Characterization of Purified sdAb Samples Using Octet BLI

Biotinylated hIL6 (RnD Systems, Cat BT7270) was immobilized on Streptavidin-coated sensors (Cat 18-5019). The sensor loading was done for 60 sec with the biotinylated hIL-6 was at 5 μg/mL in the PBS (pH 7.4) with 0.01% Tween20. The assay running buffer used for the kinetic measurements was PBS (pH 7.4) supplemented with 0.01% Tween20 and 0.01% BSA. Purified samples were diluted in the running buffer to the range of concentrations (400-0.16 nM, the exact concentrations depending on the sdAb clone).

The Octet BLI sensors were dipped into the wells of 96-well plate that contained the samples. Regeneration between readings was done using 100 mM NaOH+1 M NaCl. The readings were done with the following parameters:
Association: 180 sec
Dissociation 300 sec
Assay temperature: 25 °C
Agitation at 1,000 rpm

All measurements were repeated with reference sensors (without biotinylated hIL6) to measure background signal, which was subsequently subtracted from the binding signal of the sdAbs.

Anti-IL6 sdAb Binding Kinetics Characterization Using Surface Plasmon Resonance (SPR) Biacore (of Purified Samples)

sdAbs were immobilized on SPR COOH-chip (CM5 sensor chip, Cytiva, Cat. 29149603) through EDC/NHS coupling. Coupling was individually optimized for each sdAb based on its isoelectric point (pI) by altering pH with sodium acetate buffer. Coupling was set to reach the same coating density for all sdAbs (200 response units). 7 different IL6 concentrations (as listed in the Table 1) were measured on a Biacore T200, with the concentration range depending on the K_{D} of each sdAb (the estimate of which was obtained from Octet BLI screening). The 4-6 best kinetic curves were used for fitting the 1:1 Langmuir binding model using the Biacore Evaluation software 3.2. The measurement length was 240-300 sec for association, 600-900 sec for dissociation. The running buffer was 1×PBS+0.01% Tween20, pH 7.4 and sdAbs were regenerated in 10 mM glycine pH 2.0.

Experimental validation of Thermodynamic Equilibrium Model (TEM) with Anti-IL6 sdAb clone

Anti-IL-6 sdAb SIL-3E7 was used as analyte. Biotinylated monomer IL6 (R&D systems Cat. #BT7270-025/CF) was used as immobilized ligand to ensure 1:1 binding interaction. A BLI benchmark setup was chosen to measure binding kinetics. We prepared analyte solutions at different concentrations C(t)∈[0.001K_{d}-10K_{d}], which corresponded to 0.75 nM (0.1×K_{D}), 1 nM, 2 nM, 3 nM, 6 nM, 7.5 nM (=K_{D}), 18.75 nM, 37.5 nM, 75 nM (10×K_{D}). PBS (pH 7.4), 0.01% Tween20 was used as assay buffer. BLI sensors were functionalized with biotinylated IL6 diluted in PBS+0.01% Tween20 to 2.5 pg/mL and incubated for 180 sec, then regenerated once to remove weakly bound ligand. Measurements were performed at 25 °C, with agitation at 1,000 rpm. Concentration scouting was performed prior to the validation experiment to identify optimal analyte concentration for achieving low ligand density on the sensor surface and prevent a titration regime (Jarmoskaite et al., 2020). For each concentration step, the input concentration was recorded, and IL6-bound sensors were immersed into sdAb samples. Six sensors were measured simultaneously. The assay steps were performed as follows: baseline (buffer only), association (sdAbs at specific concentration), dissociation (buffer only). The association and dissociation phases lasted 600 s and 900 s respectively to allow sufficient time for reaching thermodynamic equilibrium and (near) complete dissociation. Data were recorded as csv file and analyzed using Matlab software (Mathworks vR2021b).

Calculation of Standard Deviations for kₒₙ and k_{off} Values

The standard deviation values provided for in Tables 7 and 11 have been calculated with three different methods. The standard deviation (SD) has been obtained by repeating BLI Octet and SPR Biacore binding kinetics measurements, respectively, as described herein and in section “Anti-IL6 sdAb binding kinetics characterization using Octet BLI” below.

The standard deviation (Rich et al) (SD (Rich et al)) has been obtained considering the average standard deviation of different machines in the prior art. In particular, an average kₒₙ value of 1.40×10\hat{ }5 and a corresponding average standard deviation of 1.30×10\hat{ }5 has been extracted from Table 1 of Rich et al. Similarly, an average k_{off} value of 6.10×10\hat{ }−5 and a corresponding average standard deviation of 8.70×10\hat{ }−5 has been extracted from Table 1 of Rich et al. A standard deviation factor has been obtained by dividing the standard deviation by the corresponding average kₒₙ or k_{off} values. Mean values of the kₒₙ k_{off} measurement values of the sdAb obtained by BLI Octet and/or SPR Biacore binding kinetics then have been multiplied by the standard deviation factor to obtain the SD (Rich et al). The standard deviation factor obtained for the kₒₙ value was 9.29×10\hat{ }−1, the standard deviation factor obtained for the k_{off} value was 1.43.

The standard deviation (Conservative SD) corresponds to the maximum standard deviation value obtained from SD or SD (Rich et al) as described above.

### Results

Establishing a Predictive Thermodynamic Equilibrium Model to Tune the Selection of Anti-IL6 sdAbs

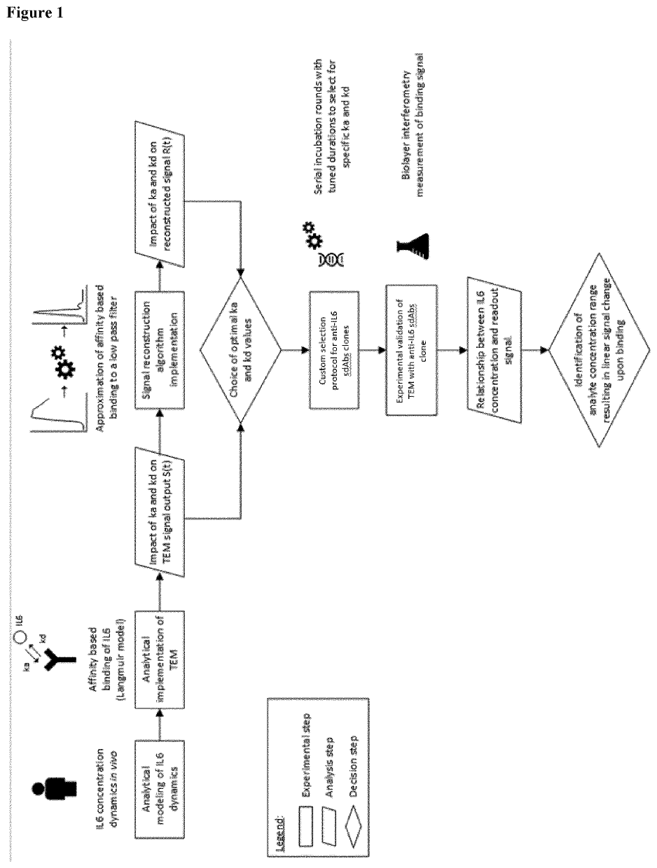

FIG. 1 describes the various steps underlying the selection of anti-IL6 sdAbs tuned towards the detection of IL6 at physiologically relevant concentrations. The target application ranges for IL6-based inflammation sensing in humans in vivo were derived from literature (Blackburn et al., 2006; Emerson et al., 2017; Esposito et al., 2002; Herieka & Erridge, 2014; Lippitz & Harris, 2016; Mavropoulou et al., 2020; Song et al., 2019; Zhou et al., 2020) and summarized in Table 1 (reference value(s)).

TABLE 1
IL6 concentrations in humans during homeostasis and disease.

| | | |
|---|---|---|
| | | Example |
| | Concentration | indications/ |
| Range | in blood | disease |

| Baseline | <45 | fM | |
|---|---|---|---|
| Low inflammation | 45 fM- | | Inactive IBD |
| | 225 fM | | |
| Moderate | 225 fM- | | COVID survivors |
| inflammation | 450 fM | | Cancer patients |
| | | | Active IBD |
| High inflammation | 450 fM- | | COVID non-survivors |
| | 4.5 pM | | Sepsis |
| Severe inflammation | >4.5 | pM | Septic shock |

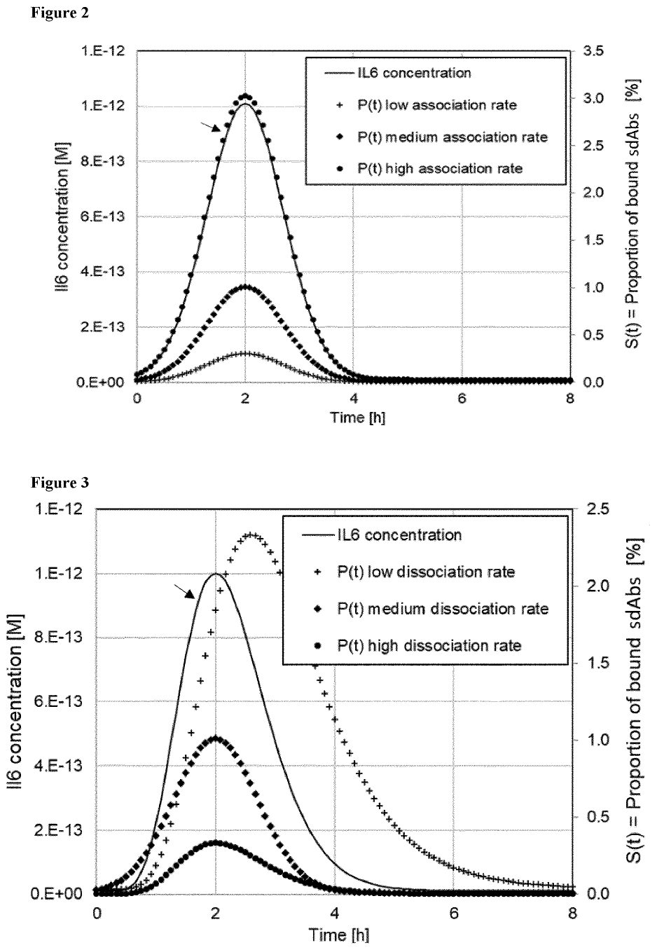

A typical IL6 concentration dynamic is shown in FIG. 2 (arrow). The sdAb selection was based on a predictive analytical model, labelled hereafter as thermodynamic equilibrium model (TEM). The TEM was used to investigate the impact that varying kinetic binding rates, kₐ and k_{d}, had on the signal readout.

The implementation of the TEM demonstrated that for a fixed dissociation kinetic rate k_{d}, increasing the value of the association rate kₐ increased the proportion of occupied sdAbs S(t), and thus the signal readout (FIG. 2). Instead, for a fixed value of association rate kₐ, increasing the dissociation rate resulted in a decreased signal readout amplitude but no significant time shift in the signal (FIG. 3). Conversely, decreasing k_{d} resulted in a large signal amplitude but a greater time delay in the readout signal (FIG. 3). These results indicated that kₐ must be high to maximize signal readout amplitude. In contrast, a tradeoff must be found for k_{d} as low values lead to large measurement errors and high values result in reduced output signal amplitude.

Combining TEM with a Signal Processing Pipeline Identifies the Range of kₐ and k_{d} Values That Maximize Signal to Noise Ratio and Lead to Low Errors in Signal Readout

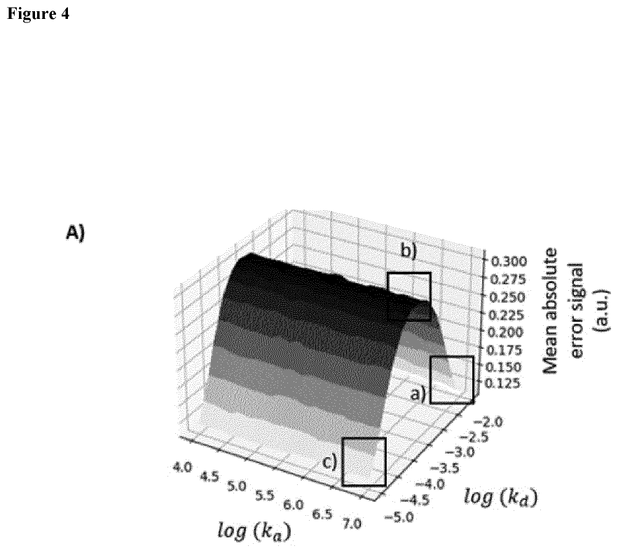

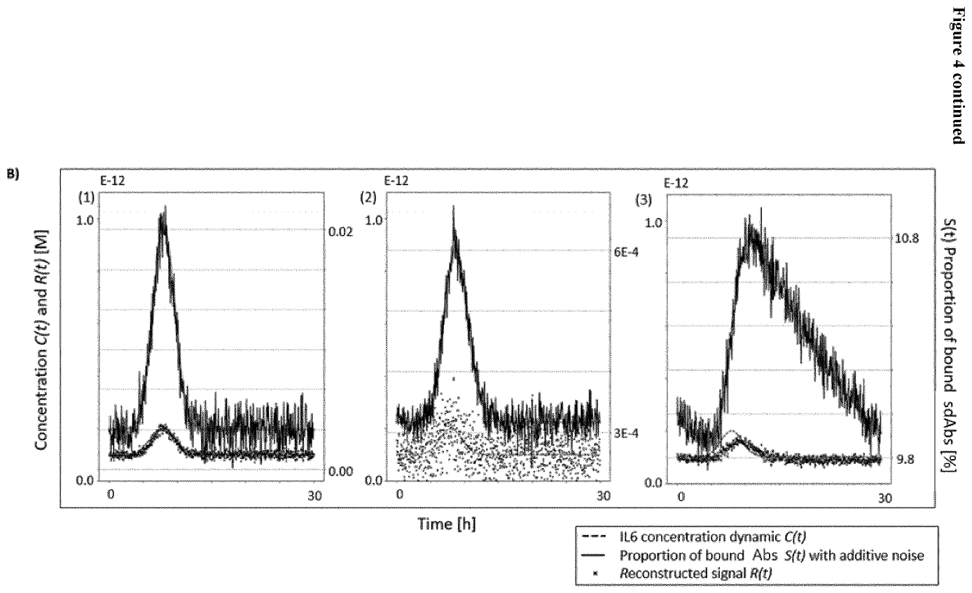

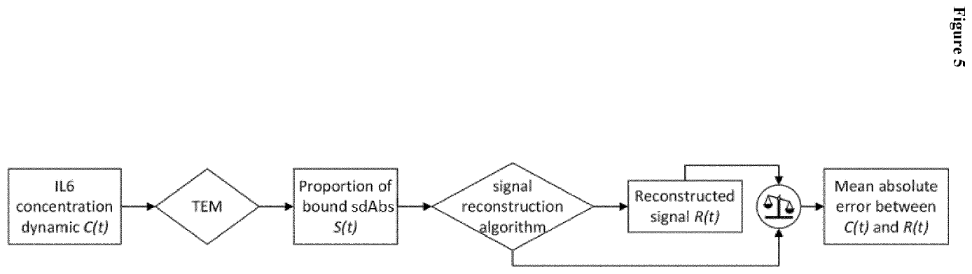

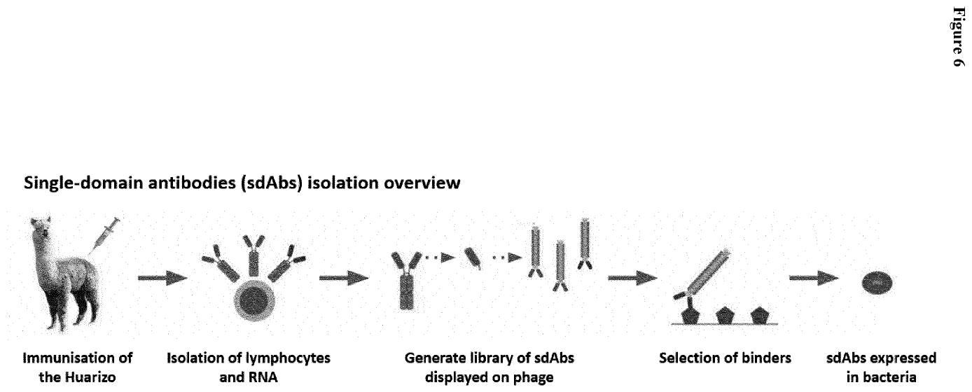

Next, the impact of different kₐ and k_{d} values on the proportion of occupied sdAbs, S(t), as well as the possibility to reconstruct the initial IL6 concentration C(t) for ranges of kinetic rates that lead to strong time shifts, thus distorting the signal, was investigated. The readout error between our reconstructed signal R(t) and C(t) was then quantified. FIG. 4A shows the mean absolute error between R(t) and C(t), averaged over a defined population of concentration dynamics. FIG. 4B displays examples of IL6 concentration dynamic C(t) with the corresponding TEM output S(t) and reconstruction output R(t). The three graphs display the signals obtained for different combinations of kₐ and k_{d} values. One could observe that the kₐ value does not impact the error between the initial concentration and the reconstructed signal FIG. 4A. This was to be expected as the time constant of the reconstruction algorithm was defined as depending only on k_{d}. We can see that k_{d} values larger than 10⁻³ s⁻¹ led to very low output signal amplitude but no signal reconstruction is needed, and the initial shape of IL6 concentration dynamics can be easily extracted from S(t) (FIG. 4B (1)). For k_{d} values ranging from 3.10⁻⁵ to from 3.10⁻⁴ s⁻¹, the amplitude of S(t) increases. In this case, the reconstruction algorithm did not improve the final signal to noise ratio and introduced noise in R(t) (FIG. 4B (2)). For k_{d} values lower than 3.10⁻⁵ s⁻¹, the TEM indicates a strong distortion of the signal S(t) with respect to the initial concentration C(t). However, the amplitude of this distorted signal is very high, and the reconstruction algorithm allows to precisely recover the shape of C(t) and to reach large signal to noise ratio (FIG. 4B (3)). The complete signal processing pipeline is summarized in (FIG. 5).

Immunization of a Huarizo with Recombinant Human IL6 to Raise Diverse Anti-IL6 Antibody Responses

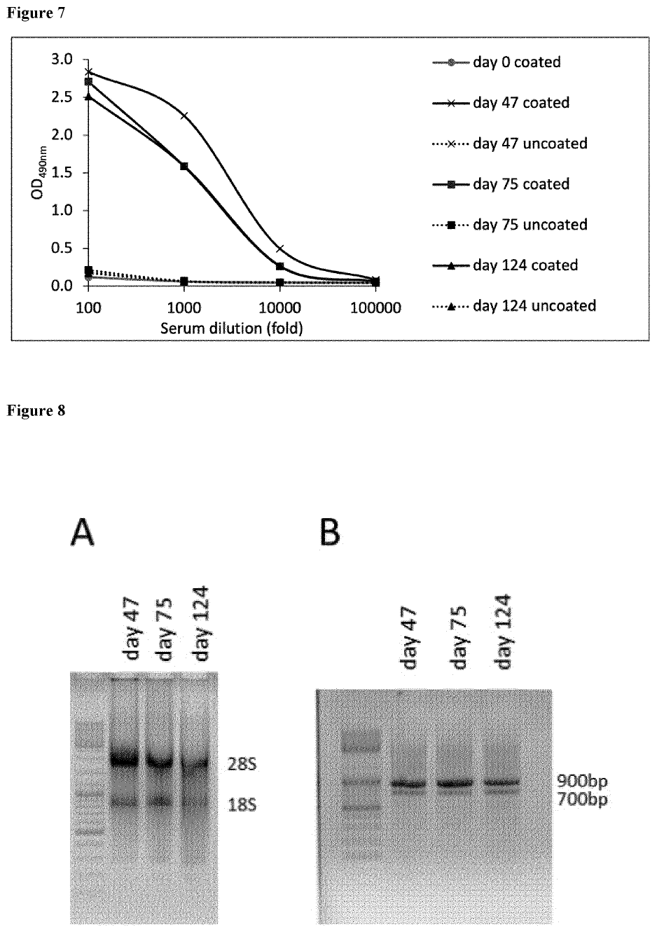

In order to raise a diverse set of anti-IL6 sdAbs, from which to rationally select the ones with optimal kₐ/k_{d} values, natural immunization followed by phage-display and guided panning was performed (FIG. 6). One adult Huarizo was immunized six times with human IL6 and GERBU FAMA adjuvant and twice with human IL6 and Incomplete Freund's adjuvant (IFA). To assess the immune response against IL6, a serial dilution of serum collected on day 0, 47, 75 and 124 was tested by ELISA (FIG. 7). The data shows a moderate immune response against IL6 on either day 47, 75 and 124.

Phage-Display and Guided Panning to Drive sdAb Selection Towards Relevant kₐ and k_{d}

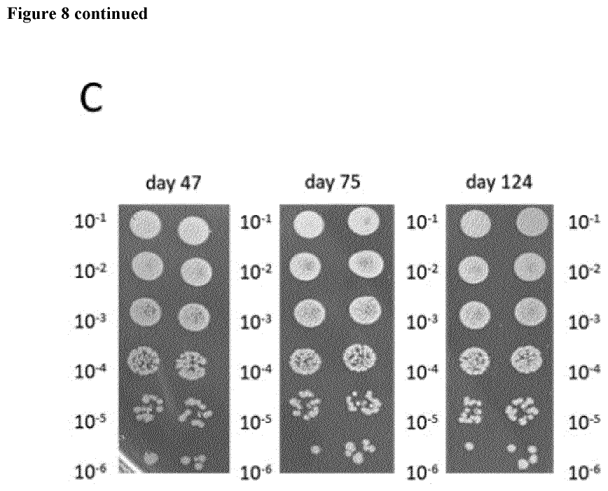

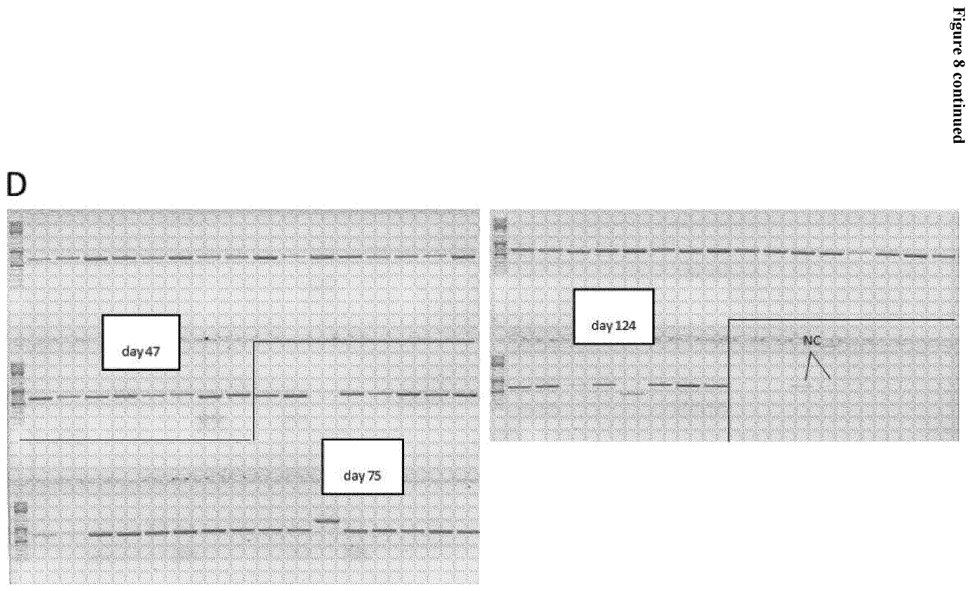

For the sdAb-phagemid library construction, RNA integrity was confirmed by the clear visibility of intact 28S and 18S rRNA on an 1% agarose gel (FIG. 8A). Transcribed cDNA fragments of immunoglobulin domains (both conventional and heavy chain) were amplified using primers annealing at the IGH leader sequence region and the CH2 region (FIG. 8B). The 700 bp DNA fragment was excised and purified before being ligated into the pQ81 phagemid vector. The ligated library was transformed into TG1 E. coli by electroporation. This transformation was serially diluted to determine the number of transformants, which is indicative of the library size (FIG. 8C). The number of transformants was 1.2×10\hat{ }9 for the day 47 library, 1.8×10\hat{ }9 for the day 75 library and 1.8×10\hat{ }9 for the day 124 library (FIG. 8C). The insertion frequency was 100% for the day 43 library, 95% for the day 75 library and 95% the day 124 library (FIG. 8D). The results of the library construction are summarized in the table in Table 2.

TABLE 2
Summary table of library construction

| | # of | Correct insertion | |
|---|---|---|---|
| Sample | transformants | frequency (%) | OD600 |
| Day 47 | 1.2 × 10\hat{ }9 | 100 | 99 |
| Day 75 | 1.8 × 10\hat{ }9 | 95 | 114 |
| Day 124 | 1.8 × 10\hat{ }9 | 95 | 102 |

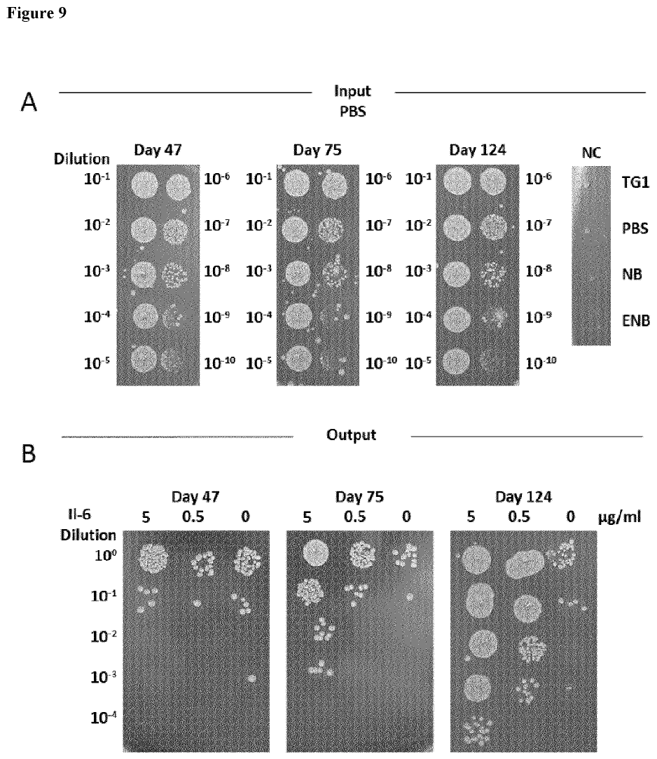

For the first round of phage-display panning selections, the amount of phages used corresponded to at least 10-fold of the transformation efficiency (estimated diversity) of the library (Table 3 and FIG. 9A).

TABLE 3
Input titers round 1 phage selection

| | Phage | Input titer |
|---|---|---|
| Library | titer (cfu/ml) | (cfu/condition) |
| Day 47 | 6 × 10\hat{ }12 | 1.2 × 10\hat{ }11 |
| Day 75 | 3 × 10\hat{ }12 | 86.0 × 10\hat{ }10 |
| Day 124 | 6 × 10\hat{ }12 | 1.2 × 10\hat{ }11 |

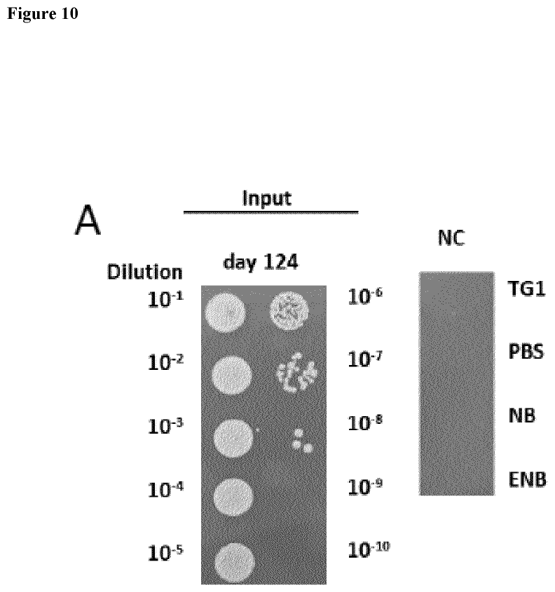

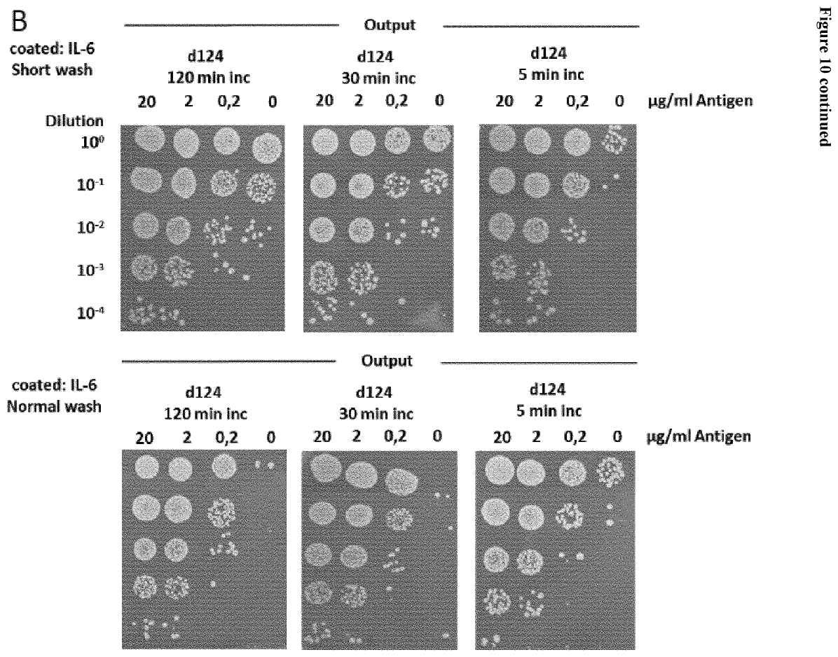

For the panning, the coated and blocked wells were incubated with the pre-blocked phages for 2 hours at RT. Upon extensive washing, bound phages were eluted and allowed to infect TG1 bacteria (rescued output). In order to assess the size of the output, eluted phages were serially diluted, infected into TG1 and then spotted on LB-agar. The outputs of the selections with the day 47 library showed no significant enrichment of phages. In contrast, phages from the day 75 library showed clear enrichment compared to uncoated wells, and phages from the day 124 library showed most enrichment (FIG. 9B). Therefore, for the second round of selections, phages produced from the outputs of the day 124 library were used to generate input titers (Table 4 and FIG. 10A).

TABLE 4
Input titers round 2 phage selection

| Output | Phage titer (cfu/mL) | Input titer (cfu/condition) |
|---|---|---|
| Day | 6.0 × 10\hat{ }12 | 1.2 × 10\hat{ }11 |
| 124 | | |

Based on the predictions retrieved from in silico modeling (TEM and signal processing), the panning was directed towards the selection of sdAb clones with high on-rates allowing fast binding of antigen and high off-rates allowing fast release of antigen at physiological concentrations of IL6. To direct the panning towards selecting sdAb with a high on-rate, IL6 was coated at increasing concentrations. In addition to the standard incubation time of 120 minutes, the phages were then incubated for shorter time periods in some conditions, namely 30 minutes and 5 minutes. Lastly, to prevent loss of sdAb with higher off-rate, a shorter wash was applied in some conditions with only 5 minutes of washing compared to >30 minutes of usually performed. All conditions showed good output titration and enrichment of phages on the IL6 coated wells (FIG. 10B). Eluted phages were rescued in TG1 and spotted on LB-agar plates from which single clones were picked and combined in three 96-wells masterplates (192 sdAb clones in total).

Sequence Analysis of sdAbs Identifies Clusters as Well as Unique sdAb Sequences

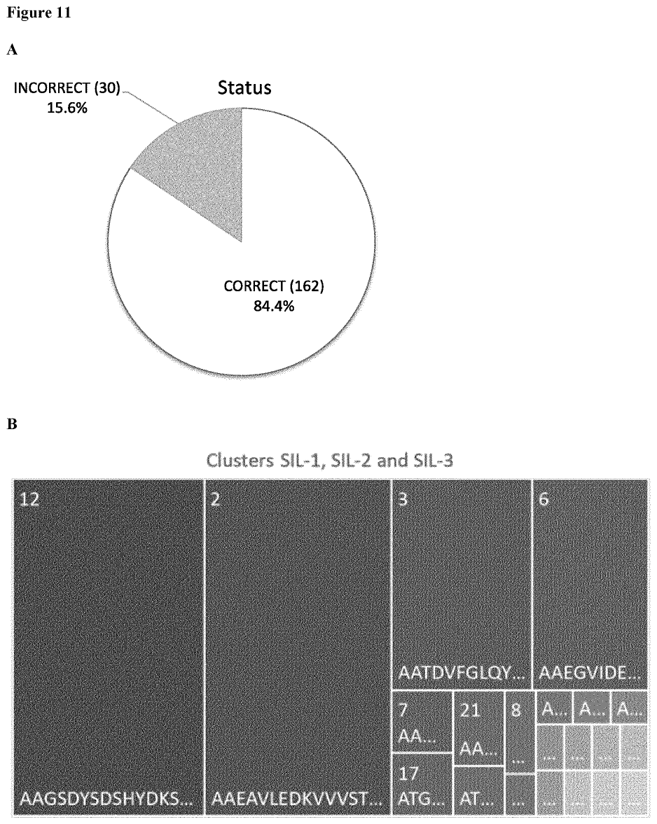

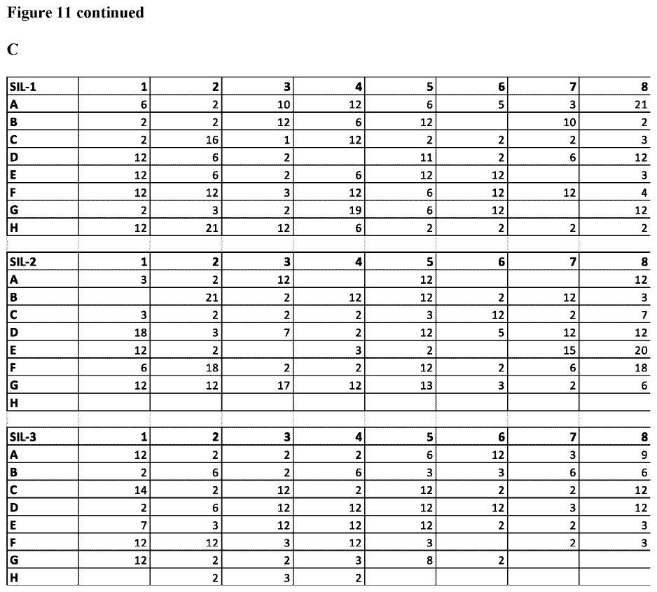

To determine the diversity of the sdAbs, all 3 master plates were sequenced by Sanger sequencing. The nucleic acid sequences were automatically analyzed and processed into sdAb amino acid sequences using the PipeBio Antibody Sequence Analysis platform. Then, the output sdAb sequences were annotated for sdAb sequence (FIG. 11A) and subsequently aligned and clustered using 80% CDR-H3 homology (FIGS. 11B and 11C).

Periplasmic Anti-IL6 sdAb ELISA and Kinetics Screening Using Octet BLI to Characterize Binding Properties to Target Antigen (i.e., Non-Purified Periplasmic Samples Comprising Anti-IL6 sdAbs were Measured Using Octet BLI for a Preliminary Assessment of the Binding Properties of the Respective sdAbs)

To evaluate binding to IL6, periplasmic extracts of all 192 sdAb clones were diluted and tested for antigen binding in an anti-IL6 ELISA (Table 5). Performing a kinetic screening with non-purified periplasmic sdAb samples in a high-throughput manner this early in the development process is unusual, but offers the opportunity to guide selection and further processing of sdAb clones at an early point towards desired kinetic properties.

TABLE 5
OD490 of periplasmic extract ELISA

| Name | OD490 |
|---|---|
| SIL-1A1 | 0.256 |
| SIL-1A2 | 0.097 |
| SIL-1A3 | 0.083 |
| SIL-1A4 | 0.998 |
| SIL-1A5 | 0.155 |
| SIL-1A6 | 0.202 |
| SIL-1A7 | 0.81 |
| SIL-1A8 | 0.224 |
| SIL-1B1 | 1.153 |
| SIL-1B2 | 0.135 |
| SIL-1B3 | 1.032 |
| SIL-1B4 | 0.547 |
| SIL-1B5 | 0.861 |
| SIL-1B6 | 0.309 |
| SIL-1B7 | 0.126 |
| SIL-1B8 | 0.116 |
| SIL-1C1 | 0.177 |
| SIL-1C2 | 0.047 |
| SIL-1C3 | 0.194 |
| SIL-1C4 | 0.952 |
| SIL-1C5 | 0.09 |
| SIL-1C6 | 0.085 |
| SIL-1C7 | 0.213 |
| SIL-1C8 | 1.187 |
| SIL-1D1 | 1.123 |
| SIL-1D2 | 0.357 |
| SIL-1D3 | 0.886 |
| SIL-1D4 | 0.195 |
| SIL-1D5 | 0.161 |
| SIL-1D6 | 0.08 |
| SIL-1D7 | 0.268 |
| SIL-1D8 | 0.984 |
| SIL-1E1 | 1.277 |
| SIL-1E2 | 0.2637 |
| SIL-1E3 | 0.268 |
| SIL-1E4 | 1.044 |
| SIL-1E5 | 0.916 |
| SIL-1E6 | 0.819 |
| SIL-1E7 | 0.049 |
| SIL-1E8 | 0.499 |
| SIL-1F1 | 1.183 |
| SIL-1F2 | 1.029 |
| SIL-1F3 | 1.416 |
| SIL-1F4 | 1.041 |
| SIL-1F5 | 0.393 |
| SIL-1F6 | 0.886 |
| SIL-1F7 | 0.99 |
| SIL-1F8 | 0.076 |
| SIL-1G1 | 0.122 |
| SIL-1G2 | 1.452 |
| SIL-1G3 | 0.132 |
| SIL-1G4 | 0.049 |
| SIL-1G5 | 0.18 |
| SIL-1G6 | 0.854 |
| SIL-1G7 | 1.033 |
| SIL-1G8 | 0.958 |
| SIL-1H1 | 1.236 |
| SIL-1H2 | 0.619 |
| SIL-1H3 | 1.108 |
| SIL-1H4 | 0.219 |
| SIL-1H5 | 0.149 |
| SIL-1H6 | 0.087 |
| SIL-1H7 | 0.121 |
| SIL-1H8 | 0.161 |
| SIL-2A1 | 1.024 |
| SIL-2A2 | 0.364 |
| SIL-2A3 | 0.967 |
| SIL-2A4 | 1.228 |
| SIL-2A5 | 1.198 |
| SIL-2A6 | 0.441 |
| SIL-2A7 | 0.332 |
| SIL-2A8 | 1.016 |
| SIL-2B1 | 1.007 |
| SIL-2B2 | 0.457 |
| SIL-2B3 | 0.219 |
| SIL-2B4 | 0.908 |
| SIL-2B5 | 1.255 |
| SIL-2B6 | 0.483 |
| SIL-2B7 | 1.008 |
| SIL-2B8 | 0.769 |
| SIL-2C1 | 0.254 |
| SIL-2C2 | 0.108 |
| SIL-2C3 | 0.281 |
| SIL-2C4 | 0.36 |
| SIL-2C5 | 0.937 |
| SIL-2C6 | 1.065 |
| SIL-2C7 | 0.31 |
| SIL-2C8 | 0.207 |
| SIL-2D1 | 0.245 |
| SIL-2D2 | 0.098 |
| SIL-2D3 | 0.33 |
| SIL-2D4 | 0.316 |
| SIL-2D5 | 1.14 |
| SIL-2D6 | 0.428 |
| SIL-2D7 | 1.197 |
| SIL-2D8 | 1.242 |
| SIL-2E1 | 0.862 |
| SIL-2E2 | 0.188 |
| SIL-2E3 | 0.805 |
| SIL-2E4 | 0.597 |
| SIL-2E5 | 0.265 |
| SIL-2E6 | 0.575 |
| SIL-2E7 | 1.463 |
| SIL-2E8 | 0.11 |
| SIL-2F1 | 0.416 |
| SIL-2F2 | 0.251 |
| SIL-2F3 | 0.153 |
| SIL-2F4 | 0.302 |
| SIL-2F5 | 1.344 |
| SIL-2F6 | 1.121 |
| SIL-2F7 | 0.255 |
| SIL-2F8 | 0.29 |
| SIL-2G1 | 0.851 |
| SIL-2G2 | 1.1 |
| SIL-2G3 | 0.829 |
| SIL-2G4 | 1.328 |
| SIL-2G5 | 1.231 |
| SIL-2G6 | 1.513 |
| SIL-2G7 | 0.132 |
| SIL-2G8 | 0.201 |
| SIL-2H1 | 1.222 |
| SIL-2H2 | 1.869 |
| SIL-2H3 | 1.176 |
| SIL-2H4 | 1.256 |
| SIL-2H5 | 0.57 |
| SIL-2H6 | 1.209 |
| SIL-2H7 | 0.212 |
| SIL-2H8 | 2.521 |
| SIL-3A1 | 1.119 |
| SIL-3A2 | 0.076 |
| SIL-3A3 | 0.057 |
| SIL-3A4 | 0.07 |
| SIL-3A5 | 0.088 |
| SIL-3A6 | 0.964 |
| SIL-3A7 | 0.161 |
| SIL-3A8 | 0.151 |
| SIL-3B1 | 0.078 |
| SIL-3B2 | 0.204 |
| SIL-3B3 | 0.053 |
| SIL-3B4 | 0.133 |
| SIL-3B5 | 0.171 |
| SIL-3B6 | 0.669 |
| SIL-3B7 | 0.888 |
| SIL-3B8 | 0.658 |
| SIL-3C1 | 0.39 |
| SIL-3C2 | 0.085 |
| SIL-3C3 | 0.889 |
| SIL-3C4 | 0.073 |
| SIL-3C5 | 0.711 |
| SIL-3C6 | 0.066 |
| SIL-3C7 | 0.071 |
| SIL-3C8 | 0.914 |
| SIL-3D1 | 0.085 |
| SIL-3D2 | 0.222 |
| SIL-3D3 | 0.73 |
| SIL-3D4 | 0.505 |
| SIL-3D5 | 1.126 |
| SIL-3D6 | 0.801 |
| SIL-3D7 | 0.138 |
| SIL-3D8 | 0.956 |
| SIL-3E1 | 0.096 |
| SIL-3E2 | 0.1581 |
| SIL-3E3 | 0.747 |
| SIL-3E4 | 0.966 |
| SIL-3E5 | 0.736 |
| SIL-3E6 | 0.059 |
| SIL-3E7 | 0.102 |
| SIL-3E8 | 0.082 |
| SIL-3F1 | 0.98 |
| SIL-3F2 | 0.805 |
| SIL-3F3 | 0.146 |
| SIL-3F4 | 0.78 |
| SIL-3F5 | 0.076 |
| SIL-3F6 | 0.912 |
| SIL-3F7 | 0.06 |
| SIL-3F8 | 0.792 |
| SIL-3G1 | 0.829 |
| SIL-3G2 | 0.105 |
| SIL-3G3 | 0.079 |
| SIL-3G4 | 0.059 |
| SIL-3G5 | 0.047 |
| SIL-3G6 | 0.085 |
| SIL-3G7 | 0.092 |
| SIL-3G8 | 0.091 |
| SIL-3H1 | 0.363 |
| SIL-3H2 | 0.073 |
| SIL-3H3 | 0.118 |
| SIL-3H4 | 0.062 |
| SIL-3H5 | 0.786 |
| SIL-3H6 | 0.704 |
| SIL-3H7 | 0.076 |
| SIL-3H8 | 0.091 |

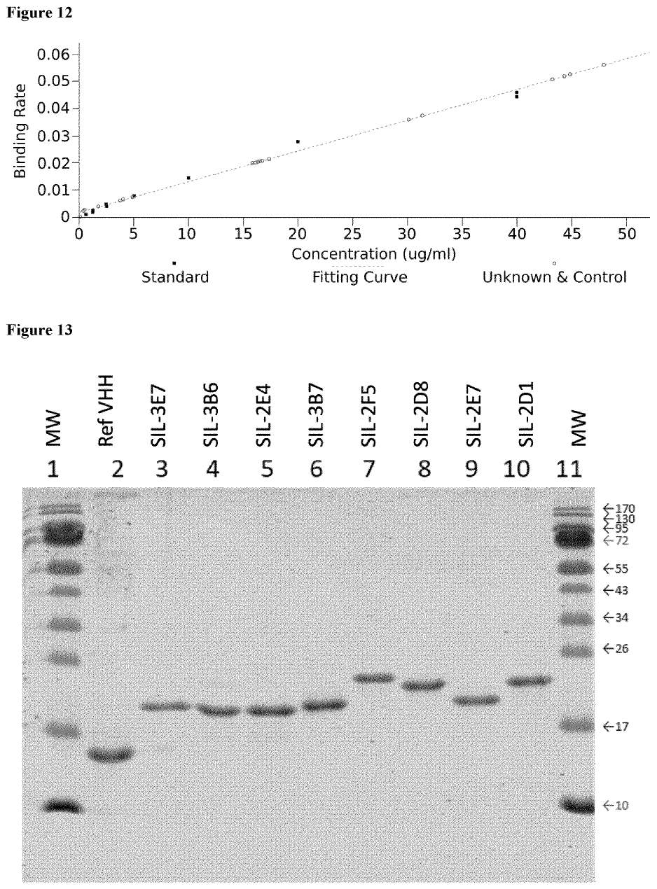

To evaluate the binding kinetics of each sdAb to IL6, periplasmic extracts of all 192 sdAb clones underwent kinetic screening using BLI Octet. First, a standard curve was generated used to derive sdAb concentration in the periplasmic extracts (FIG. 12). Secondly, binding assays were performed by immobilizing biotinylated IL6 on streptavidin-coated sensors and performing kinetic measurements on each sdAb. Of all 192 sdAb clones, the k_{d} of 137 sdAb clones could be evaluated (Table 6).

TABLE 6
BLI Octet screening results of the periplasmic extracts

| Clone | Concentration | | | |
|---|---|---|---|---|
| name | [nM] | kₐ[s⁻¹M⁻¹] | k_{d} [s⁻¹] | K_{D} [M] |
| SIL-1A1 | 1220 | 2.0E+06 | 7.2E−02 | 3.6E−08 |
| SIL-1A2 | 1233 | 1.9E+06 | 1.5E−01 | 7.6E−08 |
| SIL-1A3 | | | 2.4E−02 | |
| SIL-1A4 | 1867 | 1.0E+06 | 9.2E−03 | 8.9E−09 |
| SIL-1A6 | 147 | 6.4E+06 | 2.9E−02 | 4.5E−09 |
| SIL-1A7 | 1067 | 3.7E+06 | 7.7E−02 | 2.1E−08 |
| SIL-1A8 | | | 8.1E−03 | |
| SIL-1B1 | 1440 | 6.9E+05 | 7.1E−03 | 1.0E−08 |
| SIL-1B2 | 357 | 4.0E+06 | 5.0E−02 | 1.3E−08 |
| SIL-1B3 | 2520 | 9.6E+05 | 7.7E−03 | 8.1E−09 |
| SIL-1B4 | 2767 | 4.1E+05 | 9.3E−03 | 2.3E−08 |
| SIL-1B5 | 188 | 3.8E+06 | 7.7E−03 | 2.0E−09 |
| SIL-1B6 | 257 | 3.8E+06 | 4.8E−02 | 1.2E−08 |
| SIL-1B7 | 238 | 4.1E+06 | 4.6E−02 | 1.1E−08 |
| SIL-1C1 | 485 | 3.3E+06 | 3.6E−02 | 1.1E−08 |
| SIL-1C4 | 1153 | 1.1E+06 | 6.7E−03 | 6.4E−09 |
| SIL-1C8 | 1487 | 2.9E+06 | 5.9E−02 | 2.0E−08 |
| SIL-1D1 | 2720 | 8.3E+05 | 6.6E−03 | 7.9E−09 |
| SIL-1D2 | 1487 | 1.5E+06 | 1.1E−01 | 7.5E−08 |
| SIL-1D4 | 549 | 3.8E+06 | 6.5E−02 | 1.7E−08 |
| SIL-1D5 | | | 4.2E−02 | |
| SIL-1D8 | 1700 | 1.4E+06 | 6.5E−03 | 4.8E−09 |
| SIL-1E1 | 521 | 2.3E+06 | 5.5E−03 | 2.4E−09 |
| SIL-1E2 | 1747 | 1.3E+06 | 1.1E−01 | 8.6E−08 |
| SIL-1E3 | 2140 | 1.4E+06 | 1.7E−01 | 1.2E−07 |
| SIL-1E4 | 1913 | 2.6E+05 | 8.6E−03 | 3.3E−08 |
| SIL-1E5 | 1413 | 1.6E+06 | 8.8E−03 | 5.6E−09 |
| SIL-1E6 | 1980 | 9.9E+05 | 8.2E−03 | 8.2E−09 |
| SIL-1E8 | 401 | 3.6E+06 | 8.5E−02 | 2.3E−08 |
| SIL-1F1 | 2193 | 8.4E+05 | 6.8E−03 | 8.1E−09 |
| SIL-1F2 | 2680 | 8.3E+05 | 8.7E−03 | 1.0E−08 |
| SIL-1F3 | 1500 | 2.1E+06 | 4.9E−02 | 2.4E−08 |
| SIL-1F4 | 2240 | 9.8E+05 | 9.1E−03 | 9.3E−09 |
| SIL-1F6 | 2827 | 7.4E+05 | 8.6E−03 | 1.2E−08 |
| SIL-1F7 | 1187 | 1.3E+06 | 1.4E−02 | 1.1E−08 |
| SIL-1G1 | 2180 | 1.5E+06 | 1.2E−01 | 7.7E−08 |
| SIL-1G2 | 1300 | 2.0E+06 | 3.2E−02 | 1.6E−08 |
| SIL-1G6 | 2967 | 7.7E+05 | 8.1E−03 | 1.0E−08 |
| SIL-1G7 | 2200 | 9.9E+05 | 1.3E−02 | 1.3E−08 |
| SIL-1G8 | 1960 | 1.1E+06 | 8.1E−03 | 7.6E−09 |
| SIL-1H1 | 1713 | 1.2E+06 | 7.0E−03 | 5.7E−09 |
| SIL-1H2 | 1693 | 1.1E+06 | 1.3E−02 | 1.2E−08 |
| SIL-1H3 | 1360 | 1.6E+06 | 8.4E−03 | 5.3E−09 |
| SIL-2A1 | 813 | 3.1E+06 | 3.2E−02 | 1.0E−08 |
| SIL-2A2 | 3120 | 1.4E+06 | 1.4E−01 | 1.0E−07 |
| SIL-2A3 | 1607 | 1.7E+06 | 9.0E−03 | 5.2E−09 |
| SIL-2A4 | 336 | 9.0E+06 | 4.5E−02 | 5.0E−09 |
| SIL-2A5 | 2053 | 1.1E+06 | 1.1E−05 | 9.7E−12 |
| SIL-2A6 | 1973 | 9.8E+05 | 2.8E−01 | 2.8E−07 |
| SIL-2A7 | 247 | 1.1E+07 | 1.2E−01 | 1.1E−08 |
| SIL-2A8 | 1600 | 1.6E+06 | 7.9E−03 | 5.1E−09 |
| SIL-2B1 | 2707 | 8.2E+05 | 8.5E−03 | 1.0E−08 |
| SIL-2B2 | 4213 | 7.3E+05 | 1.8E−02 | 2.5E−08 |
| SIL-2B3 | 608 | 4.8E+06 | 2.4E−01 | 5.0E−08 |
| SIL-2B4 | 573 | 3.1E+06 | 7.4E−03 | 2.4E−09 |
| SIL-2B5 | 2260 | 1.2E+06 | 3.9E−04 | 3.4E−10 |
| SIL-2B7 | 2320 | 1.2E+06 | 8.9E−03 | 7.2E−09 |
| SIL-2B8 | | | 9.0E−02 | |
| SIL-2C1 | 1840 | 1.5E+06 | 3.0E−02 | 2.1E−08 |
| SIL-2C3 | 1760 | 2.1E+06 | 7.4E−02 | 3.5E−08 |
| SIL-2C5 | 347 | 8.1E+06 | 5.0E−02 | 6.2E−09 |
| SIL-2C6 | 1627 | 9.8E+05 | 5.9E−03 | 6.0E−09 |
| SIL-2C8 | | | 5.7E−02 | |
| SIL-2D1 | 297 | 2.0E+06 | 1.6E−02 | 7.8E−09 |
| SIL-2D2 | | | 3.1E−02 | |
| SIL-2D3 | 621 | 1.6E+06 | 4.9E−02 | 3.1E−08 |
| SIL-2D5 | 1820 | 1.3E+06 | 9.2E−04 | 6.9E−10 |
| SIL-2D6 | | | 4.1E−02 | |
| SIL-2D7 | 2107 | 1.5E+06 | 7.1E−03 | 4.8E−09 |
| SIL-2D8 | 1240 | 2.0E+06 | 7.0E−03 | 3.6E−09 |
| SIL-2E1 | 2193 | 1.0E+06 | 8.7E−03 | 8.7E−09 |
| SIL-2E2 | 1593 | 2.0E+06 | 6.7E−02 | 3.4E−08 |
| SIL-2E3 | 2340 | 1.4E+06 | 5.1E−02 | 3.8E−08 |
| SIL-2E4 | 189 | 1.3E+07 | 4.0E−02 | 3.0E−09 |
| SIL-2E5 | 1633 | 1.5E+06 | 3.1E−01 | 2.1E−07 |
| SIL-2E6 | | | 1.7E−02 | |
| SIL-2E7 | 569 | 1.8E+06 | 5.0E−03 | 2.8E−09 |
| SIL-2F2 | 1140 | 5.0E+05 | 1.2E−02 | 2.4E−08 |
| SIL-2F5 | 474 | 4.5E+06 | 7.1E−06 | 1.6E−12 |
| SIL-2F6 | 2053 | 3.0E+05 | 5.3E−03 | 1.8E−08 |
| SIL-2F8 | | | 1.3E−02 | |
| SIL-2G1 | 2553 | 1.0E+06 | 8.1E−03 | 8.1E−09 |
| SIL-2G2 | 1900 | 1.3E+06 | 1.2E−02 | 9.0E−09 |
| SIL-2G3 | | | 9.1E−02 | |
| SIL-2G4 | 2093 | 1.2E+06 | 9.4E−03 | 7.6E−09 |
| SIL-2G5 | 2520 | 6.3E+05 | 1.2E−03 | 1.9E−09 |
| SIL-2G6 | 437 | 7.4E+06 | 6.1E−02 | 8.1E−09 |
| SIL-2H1 | 3760 | 8.6E+05 | 7.7E−03 | 8.9E−09 |
| SIL-2H2 | 2413 | 3.0E+05 | 3.8E−02 | 1.3E−07 |
| SIL-2H3 | 640 | 1.3E+06 | 7.6E−03 | 5.8E−09 |
| SIL-2H4 | 2973 | 1.0E+06 | 8.8E−03 | 8.6E−09 |
| SIL-2H5 | 169 | 8.7E+06 | 8.0E−02 | 9.2E−09 |
| SIL-2H6 | 566 | 3.2E+06 | 7.3E−03 | 2.3E−09 |
| SIL-2H7 | | | 8.2E−02 | |
| SIL-2H8 | 1113 | 3.4E+06 | 3.1E−02 | 9.2E−09 |
| SIL-3A1 | 3820 | 5.9E+05 | 5.4E−03 | 9.2E−09 |
| SIL-3A6 | 3247 | 8.8E+05 | 1.5E−06 | 1.7E−12 |
| SIL-3A7 | 444 | 6.8E+06 | 3.7E−02 | 5.5E−09 |
| SIL-3A8 | 421 | 6.3E+04 | 1.1E−02 | 1.7E−07 |
| SIL-3B5 | 361 | 7.3E+06 | 3.0E−02 | 4.1E−09 |
| SIL-3B6 | 311 | 4.3E+06 | 1.2E−06 | 2.8E−13 |
| SIL-3B7 | 251 | 1.2E+06 | 5.5E−03 | 4.6E−09 |
| SIL-3B8 | 933 | 7.0E+05 | 5.1E−02 | 7.3E−08 |
| SIL-3C1 | 6380 | 1.3E+05 | 2.9E−02 | 2.2E−07 |
| SIL-3C3 | 1547 | 1.3E+06 | 6.3E−03 | 4.8E−09 |
| SIL-3C5 | 656 | 1.0E+06 | 5.6E−03 | 5.6E−09 |
| SIL-3C8 | 459 | 2.3E+06 | 8.2E−03 | 3.6E−09 |
| SIL-3D1 | 2067 | 1.7E+06 | 1.2E−01 | 7.4E−08 |
| SIL-3D2 | 1927 | 1.5E+06 | 9.0E−02 | 5.8E−08 |
| SIL-3D3 | 1420 | 1.2E+06 | 6.5E−03 | 5.5E−09 |
| SIL-3D4 | 1213 | 1.1E+06 | 7.4E−03 | 6.5E−09 |
| SIL-3D5 | 2820 | 8.2E+05 | 7.4E−03 | 9.1E−09 |
| SIL-3D6 | 2880 | 1.0E+06 | 2.4E−06 | 2.4E−12 |
| SIL-3D7 | 245 | 9.5E+06 | 5.4E−02 | 5.7E−09 |
| SIL-3D8 | 2960 | 8.9E+05 | 1.2E−02 | 1.3E−08 |
| SIL-3E1 | | | 3.4E−02 | |
| SIL-3E2 | 1420 | 2.0E+06 | 2.7E−02 | 1.3E−08 |
| SIL-3E3 | 296 | 3.4E+06 | 5.3E−03 | 1.6E−09 |
| SIL-3E4 | 2387 | 9.5E+05 | 5.9E−03 | 6.1E−09 |
| SIL-3E5 | 4227 | 8.5E+05 | 9.2E−03 | 1.1E−08 |
| SIL-3E7 | 517 | 5.5E+06 | 7.4E−02 | 1.4E−08 |
| SIL-3E8 | | | 5.7E−02 | |
| SIL-3F1 | 3433 | 8.5E+05 | 6.4E−03 | 7.5E−09 |
| SIL-3F2 | 1807 | 9.7E+05 | 4.7E−03 | 4.8E−09 |
| SIL-3F3 | 351 | 6.1E+06 | 3.5E−02 | 5.8E−09 |
| SIL-3F4 | 2207 | 8.1E+05 | 6.6E−03 | 8.2E−09 |
| SIL-3F5 | | | 4.3E−02 | |
| SIL-3F6 | 2280 | 8.3E+05 | 1.7E−06 | 2.0E−12 |
| SIL-3F8 | 687 | 2.5E+06 | 1.2E−02 | 4.9E−09 |
| SIL-3G1 | 3167 | 9.4E+05 | 5.7E−03 | 6.1E−09 |
| SIL-3G4 | | | 2.6E−02 | |
| SIL-3H1 | 2433 | 1.4E+06 | 2.9E−02 | 2.1E−08 |
| SIL-3H2 | 773 | 2.3E+06 | 5.0E−02 | 2.1E−08 |
| SIL-3H3 | 319 | 5.9E+06 | 2.9E−02 | 4.9E−09 |
| SIL-3H5 | 2260 | 9.3E+05 | 4.7E−03 | 5.0E−09 |
| SIL-3H6 | 2467 | 3.2E+05 | 1.6E−06 | 5.1E−12 |
| SIL-3H8 | | | 7.8E−02 | |

Rational Selection of Anti-IL6 sdAbs Based on Binding Characteristics and Sequence

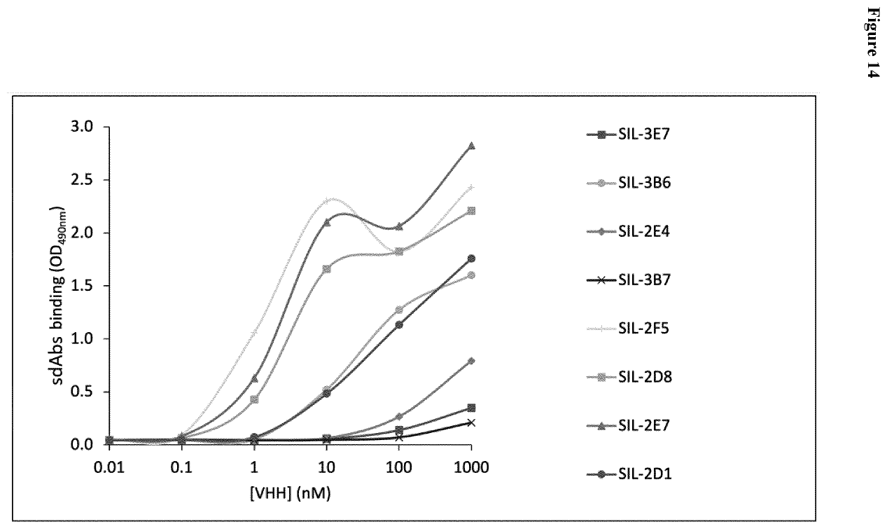

8 anti-IL6 sdAb clones were selected based on ELISA results, Octet BLI screening results (k_{d}) (Table 6) and sequence identity. The clones were selected such as to represent a variety of CDR sequences and to either represent high affinity binders and/or to fit the kₐ/k_{d} criteria defined by in silico modeling (TEM and signal processing). Selected clones were transformed into the E. coli strain BL21, expressed and purified. The purity of those sdAbs was assessed by SDS-PAGE (FIG. 13) and their apparent binding affinity determined by ELISA (FIG. 14 and Table 7). The rational selection was performed after the Octet screening of non-purified samples (see Table 6 above) and before running the Octet BLI and SPR Biacore with purified samples, see below Table 8.

TABLE 7
Estimated apparent affinities for IL6

| Clone name | Relative Bmax (OD) | Apparent affinity for IL6 (nM) |
|---|---|---|
| SIL-3E7 | NA | >1000 |
| SIL-3B6 | 1.5 | 20 |
| SIL-2E4 | NA | >1000 |
| SIL-3B7 | NA | >1000 |
| SIL-2F5 | 2.5 | 1 |
| SIL-2D8 | 2 | 5 |
| SIL-2E7 | 3 | 2 |
| SIL-2D1 | 2 | 80 |

Anti-IL6 sdAb Binding Kinetics Characterization Using Octet BLI

Biotinylated hIL6 (RnD Systems, Cat BT7270) was immobilized on Streptavidin-coated sensors (Cat 18-5019). The sensor loading was done for 60 sec with the biotinylated hIL-6 was at 5 μg/mL in the PBS (pH 7.4) with 0.01% Tween20. The assay running buffer used for the kinetic measurements was PBS (pH 7.4) supplemented with 0.01% Tween20 and 0.01% BSA. Purified samples were diluted in the running buffer to the range of concentrations (400-0.16 nM, the exact concentrations depend on the nanobody). The measurement that resulted in a good fit were used for calculating the kinetic constants (as listed in the Table 8).

The Octet BLI sensors were dipped into the wells of 96-well plate that contained the diluted periplasmic extracts. Regeneration between readings was done using 100 mM NaOH+1 M NaCl. The readings were done with the following parameters:
Association: 180 sec
Dissociation 300 sec
Assay temperature: 25 °C
Agitation at 1,000 rpm

All measurements were repeated with reference sensors (without biotinylated hIL6) to measure background signal, which was subsequently subtracted from the binding signal of the sdAbs. The results are shown in Table 6 (screening of periplasmic samples) and Table 8 (purified samples).

TABLE 8
BLI Octet screening results of purified samples with standard deviations

| | Con- | | | SD | Conser- | | | SD | Conser- | | |
|---|---|---|---|---|---|---|---|---|---|---|---|
| | centration | kₐ[s⁻¹ | | (Rich | vative | k_{d} | | (Rich | vative | K_{D} | |
| Clone | [nM] | M⁻¹] | SD | et al) | SD | [s⁻¹] | SD | et al) | SD | [M] | K_{D} nM |
| SIL- | 50, 25, | 8.86E+05 | 9.11E+05 | 8.23E+05 | 9.11E+05 | 1.25E−04 | 6.11E−05 | 1.78E−04 | 1.78E−04 | 2.81E−10 | 2.81E−01 |
| 2D8 | 12.5, 6.3 | | | | | | | | | | |
| SIL- | 100, 50, 25, | 6.11E+05 | 2.65E+05 | 5.67E+05 | 5.67E+05 | 2.94E−04 | 6.28E−04 | 4.19E−04 | 6.28E−04 | 4.81E−10 | 4.81E−01 |
| 2E7 | 12.5, 6.3, | | | | | | | | | | |
| | 3.1, 1.6 | | | | | | | | | | |
| SIL- | 50, 25, 12.5, | 1.34E+06 | 2.09E+06 | 1.24E+06 | 2.09E+06 | 1.90E−04 | 9.85E−05 | 2.71E−04 | 2.71E−04 | 4.99E−10 | 4.99E−01 |
| 2F5 | 6.3, 3.1, 1.6, | | | | | | | | | | |
| | 0.8, 0.4 | | | | | | | | | | |
| SIL- | 100, 50, | 9.43E+05 | 5.74E+04 | 8.76E+05 | 8.76E+05 | 3.57E−03 | 8.55E−05 | 5.09E−03 | 5.09E−03 | 3.78E−09 | 3.78E+00 |
| 2D1 | 25, 12.5, | | | | | | | | | | |
| | 6.3, 3.1, 1.6 | | | | | | | | | | |
| SIL- | 100, 50, | 6.35E+05 | 2.60E+04 | 5.90E+05 | 5.90E+05 | 3.75E−03 | 6.18E−05 | 5.35E−03 | 5.35E−03 | 5.91E−09 | 5.91E+00 |
| 3B6 | 25, 12.5, | | | | | | | | | | |
| | 6.3, 3.1, 1.6 | | | | | | | | | | |
| SIL- | 100, 50, | 7.35E+05 | 4.02E+04 | 6.83E+05 | 6.83E+05 | 4.85E−03 | 9.23E−05 | 6.92E−03 | 6.92E−03 | 6.60E−09 | 6.60E+00 |
| 2E4 | 25, 12.5, | | | | | | | | | | |
| | 6.3, 3.1, 1.6 | | | | | | | | | | |
| SIL- | 100, 50, | 8.02E+05 | 4.47E+04 | 7.45E+05 | 7.45E+05 | 6.10E−03 | 1.09E−04 | 8.70E−03 | 8.70E−03 | 7.60E−09 | 7.60E+00 |
| 3E7 | 25, 12.5, | | | | | | | | | | |
| | 6.3, 3.1, 1.6 | | | | | | | | | | |
| SIL- | 400, 200, | 2.33E+06 | 3.10E+06 | 2.16E+06 | 3.10E+06 | 3.57E−02 | 2.32E−02 | 5.09E−02 | 5.09E−02 | 2.66E−08 | 2.66E+01 |
| 3B7 | 100, 50, 25, | | | | | | | | | | |
| | 12.5, 6.3 | | | | | | | | | | |
| MIN | | 6.11E+05 | 2.60E+04 | 5.67E+05 | 5.67E+05 | 1.25E−04 | 6.11E−05 | 1.78E−04 | 1.78E−04 | 2.81E−10 | 2.81E−01 |
| MAX | | 2.33E+06 | 3.10E+06 | 2.16E+06 | 3.10E+06 | 3.57E−02 | 2.32E−02 | 5.09E−02 | 5.09E−02 | 2.66E−08 | 2.66E+01 |

Anti-IL6 sdAb Binding Kinetics Characterization Using SPR

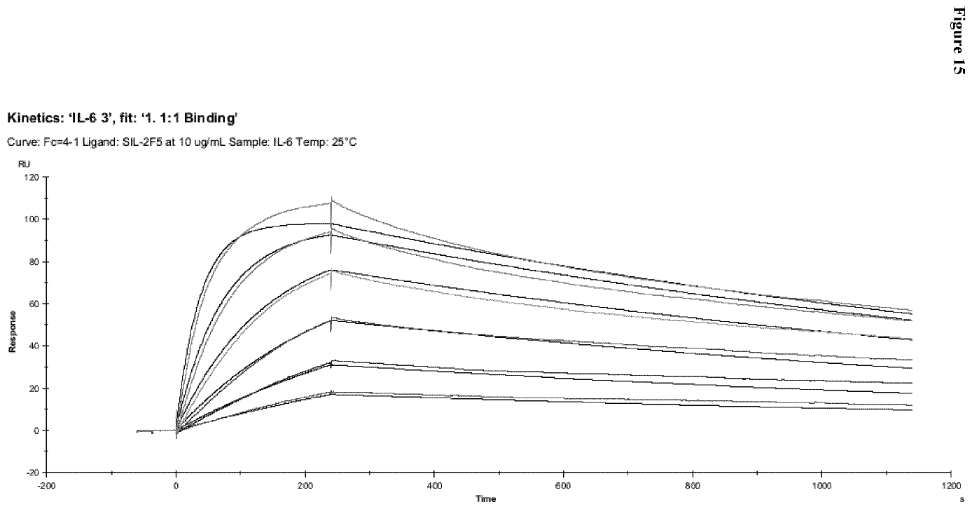

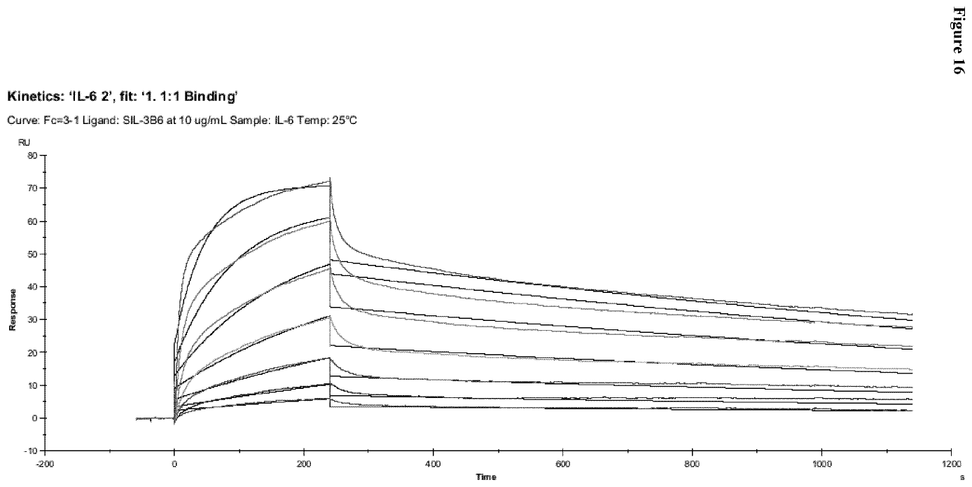

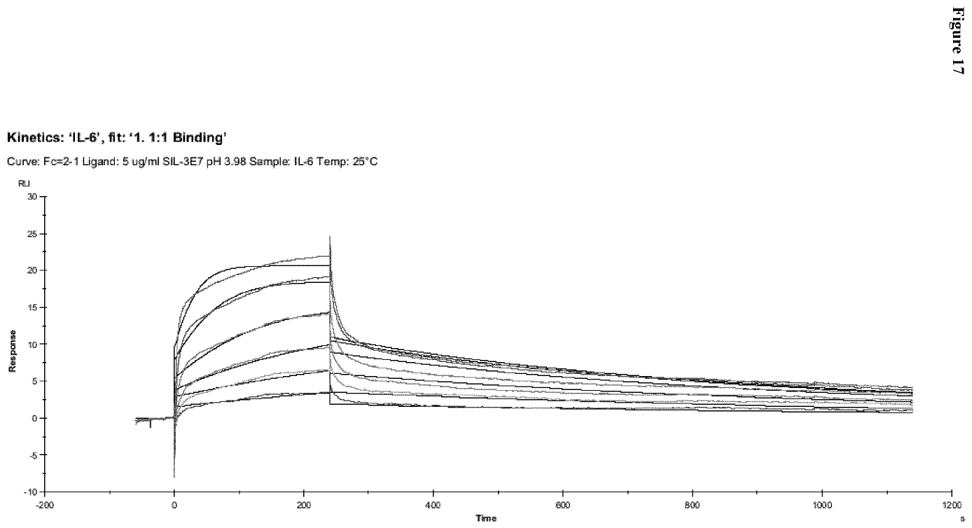

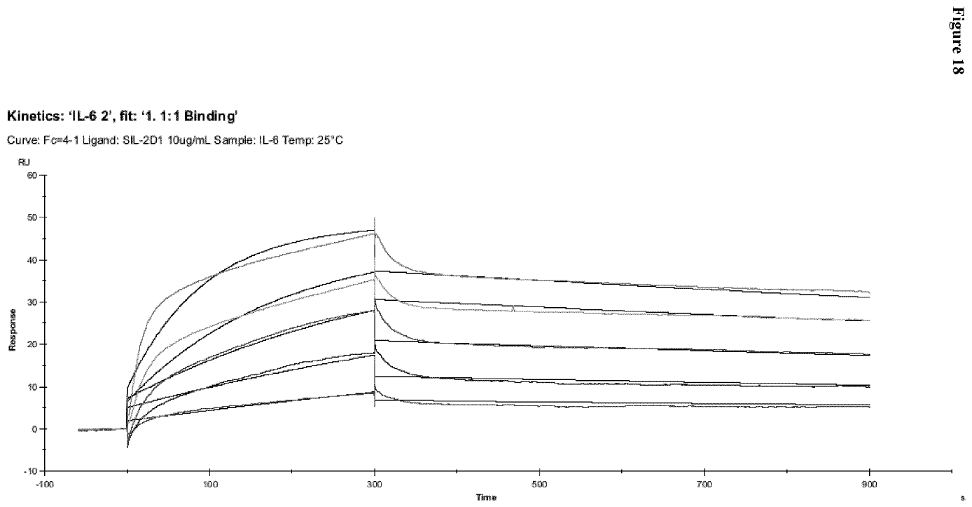

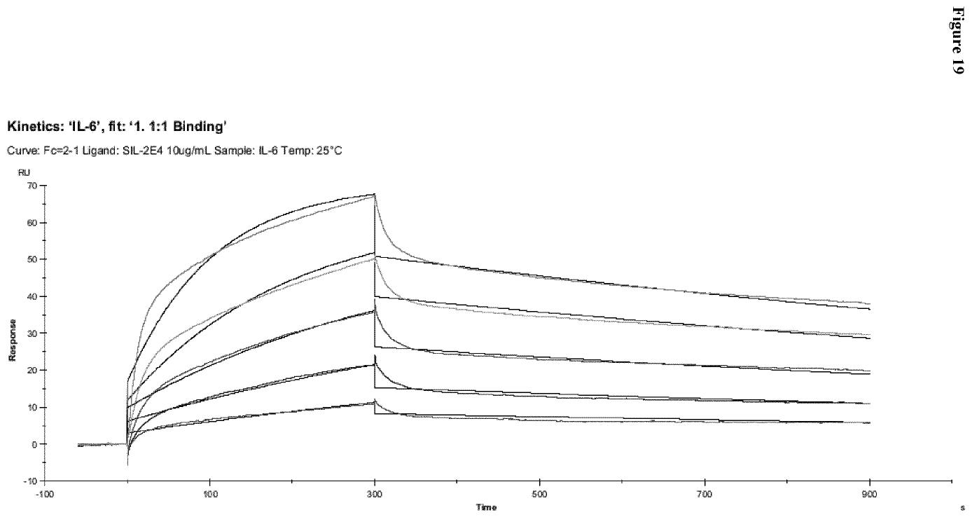

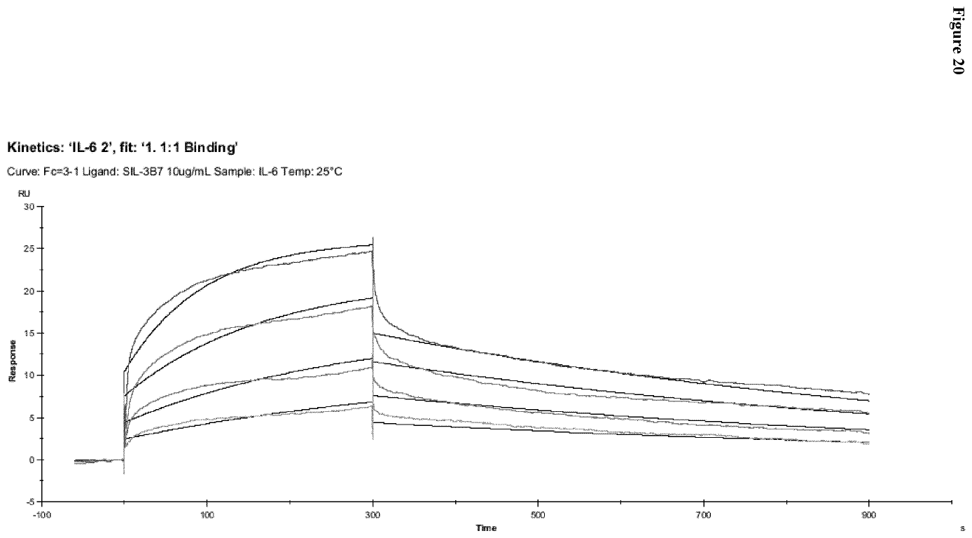

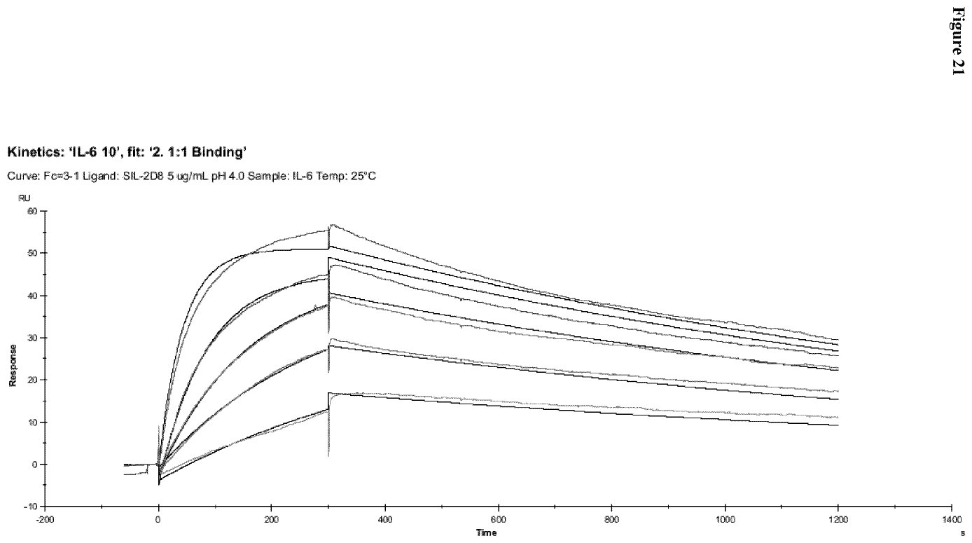

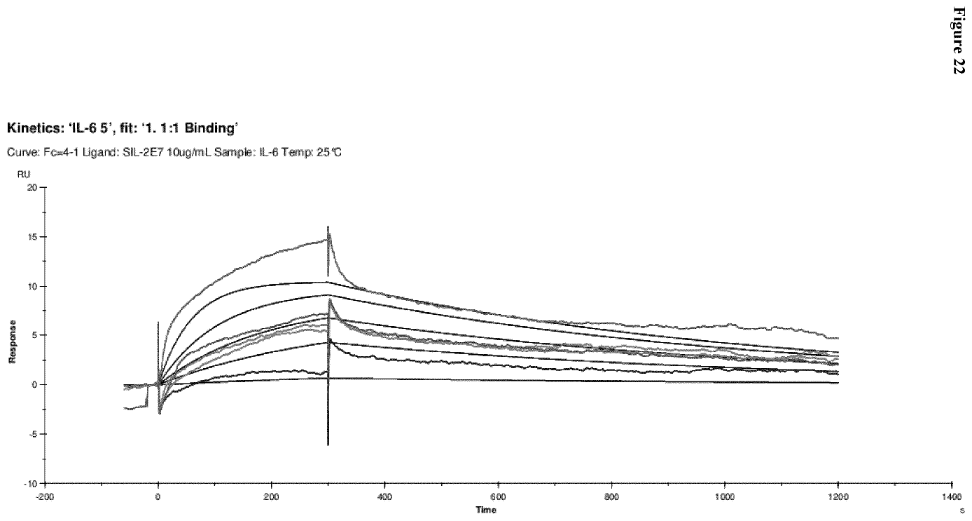

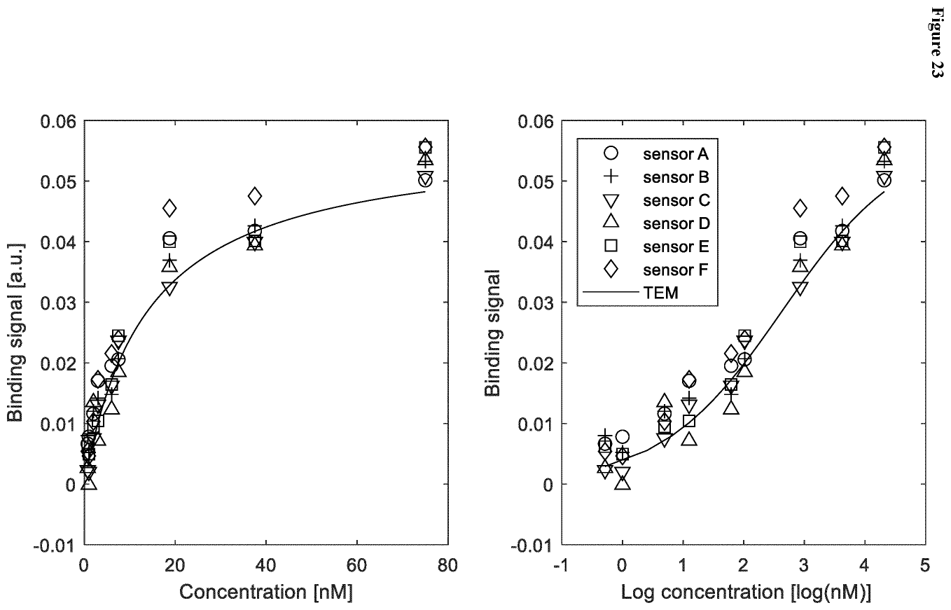

In order to characterize the binding kinetics of the 8 lead anti-IL6 sdAbs, SPR Biacore measurements were performed. Experimental parameters of SPR Biacore characterization are plotted in Table 9, and results are plotted in Table 10. Individual kinetic binding curves for each sdAb are shown in FIGS. 15-22. Table 11 shows standard deviations obtained by repeated measurements using SPR (=SD), standard deviation values obtained from the prior art (=SD (Rich et al)) and the maximum standard deviation value obtained from either SD or SD (Rich et al) (=Conservative SD).

TABLE 9
Experimental conditions to run SPR Biacore characterization for each sdAb
immobilized on the CM5 chip.

| | | | | | | Analyte |
|---|---|---|---|---|---|---|
| | | | | | | concentrations |
| | Concent ration | | Immobilization | | | range [nM] |
| | used for | pH used | target level/ | Time for | Time for | 7-point 2- |
| sdAb | immobilization | for | actual level | association | dissociation | fold |
| name | [μg/mL] | immobilization | [r · u] | (sec) | (sec) | dilution: |
| SIL-3E7 | 5 | 4 | 200/183 | 240 | 900 | 200-3.125 |
| SIL-3B6 | 10 | 4 | 200/228 | 240 | 900 | 200-3.125 |
| SIL-2F5 | 10 | 4 | 200/264 | 240 | 900 | 50-0.78 |
| SIL-2E4 | 10 | 4 | 200/235 | 300 | 600 | 50-0.78 |
| SIL-3B7 | 10 | 4 | 200/223 | 300 | 600 | 50-0.78 |
| SIL-2D1 | 10 | 4 | 200/190 | 300 | 600 | 50-0.78 |
| SIL-2D8 | 5 | 4 | 200/200 | 300 | 600 | 50-0.78 |
| SIL-2E7 | 10 | 4 | 200/207 | 300 | 600 | 50-0.78 |

TABLE 10
SPR Biacore binding kinetics of lead anti-IL6 sdAb clones

| Clone | kₐ[s⁻¹M⁻¹] | k_{d} [s⁻¹] | K_{D} [M] |
|---|---|---|---|
| SIL-2D8 | 1.82E6 | 6.71E−4 | 0.37 |
| SIL-2E7 | — | — | — |
| SIL-2F5 | 2.21E6 | 6.53E−4 | 0.30 |
| SIL-2D1 | 8.37E5 | 3.08E−4 | 0.37 |
| SIL-3B6 | 8.45E5 | 5.35E−4 | 0.63 |
| SIL-2E4 | 7.33E5 | 5.52E−4 | 0.75 |
| SIL-3E7 | 8.64E5 | 1.61E−3 | 1.9 |
| SIL-3B7 | 1.86E5 | 1.27E−3 | 6.8 |

During SPR Biacore measurements, the sdAbs are immobilized on the sensor surface by EDC/NHS chemistry, with IL6 antigens flowing over the surface at different concentrations. Whereas during the Octet BLI measurements described above, the IL6 antigens were immobilized and the anti-IL6 sdAbs were present in solution. Accordingly, SPR Biacore more closely reflects a biosensor setup (comprising immobilized anti-IL6 sdAbs and IL6 antigens in solution e.g. for in vitro or in vivo application) as compared to Octet BLL. SPR Biacore measurements (Table 10) may thus be preferred herein compared to the BLI Octet measurements using purified sdAbs (Table 8), which in turn may be preferred herein compared to the BLI Octet measurements using periplasmic extract sdAbs (Table 6).

Accordingly, in the context of the present invention, when assessing binding affinities of the herein provided sdAbs, measurements performed on purified sdAbs (i.e., the values disclosed in Table 8 and Table 10) are preferred, whereas SPR Biacore measurements (i.e., the values disclosed in Table 10) are particularly preferred.

TABLE 11
SPR Biacore binding kinetics and standard deviations of anti-IL6 sdAb clones

| | kₐ[s⁻¹ | | SD (Rich | Conservative | | | SD (Rich | Conservative | K_{D} |
|---|---|---|---|---|---|---|---|---|---|
| Clone | M⁻¹] | SD | et al) | SD | k_{d} [s⁻¹] | SD | et al) | SD | [nM] |
| SIL-2D8 | 1.82E+06 | 4.02E+03 | 1.69E+06 | 1.69E+06 | 6.71E−04 | 2.08E−06 | 9.57E−04 | 9.57E−04 | 0.37 |
| SIL-2E7 | — | — | — | — | — | — | — | — | — |
| SIL-2F5 | 2.21E+06 | 4.16E+03 | 2.05E+06 | 2.05E+06 | 6.53E−04 | 1.86E−06 | 9.31E−04 | 9.31E−04 | 0.3 |
| SIL-2D1 | 8.37E+05 | 2.91E+03 | 7.77E+05 | 7.77E+05 | 3.08E−04 | 4.47E−06 | 4.39E−04 | 4.39E−04 | 0.37 |
| SIL-3B6 | 8.45E+05 | 2.46E+03 | 7.85E+05 | 7.85E+05 | 5.35E−04 | 2.91E−06 | 7.63E−04 | 7.63E−04 | 0.63 |
| SIL-2E4 | 7.33E+05 | 2.46E+03 | 6.81E+05 | 6.81E+05 | 5.52E−04 | 4.25E−06 | 7.87E−04 | 7.87E−04 | 0.75 |
| SIL-3E7 | 8.64E+05 | 1.69E+04 | 8.02E+05 | 8.02E+05 | 1.61E−03 | 2.94E−05 | 2.30E−03 | 2.30E−03 | 1.9 |
| SIL-3B7 | 1.86E+05 | 1.12E+03 | 1.73E+05 | 1.73E+05 | 1.27E−03 | 7.40E−06 | 1.81E−03 | 1.81E−03 | 6.8 |
| MIN | 1.86E+05 | 1.12E+03 | 1.73E+05 | 1.73E+05 | 3.08E−04 | 1.86E−06 | 4.39E−04 | 4.39E−04 | 3.00E−01 |
| MAX | 2.21E+06 | 1.69E+04 | 2.05E+06 | 2.05E+06 | 1.61E−03 | 2.94E−05 | 2.30E−03 | 2.30E−03 | 6.80E+00 |

Validating TEM and Readout Signal Dependency on Analyte Concentration Using Anti-IL6 sdAb SIL-3E7

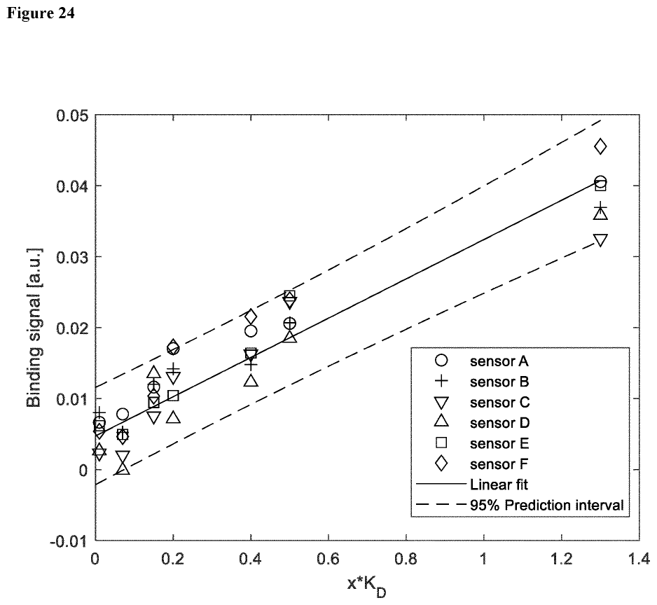

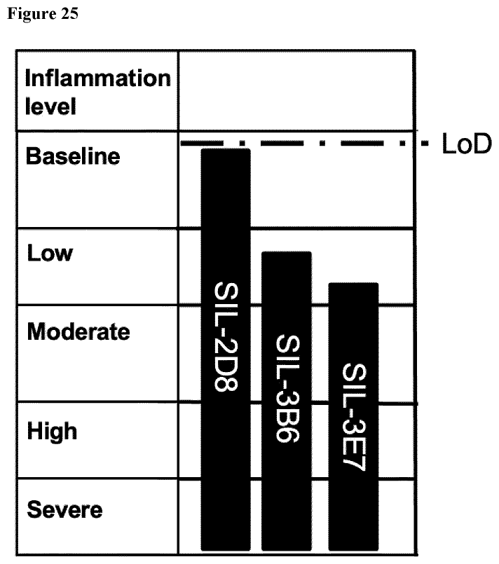

To validate the TEM with the anti-IL6 sdAb and to identify the concentration range in which the binding signal varies linearly with the molar concentration of analyte, BLI measurements of 9 sensors coated with biotinylated IL6 that were exposed to various concentration of anti-IL6 sdAb were performed. The kₐ and k_{d} of the sdAb-antigen interaction were extracted by fitting the TEM on each sensor, for each concentration step and averaged over sensor repeats. Kinetic rates found were k_{d}=1.25×10⁻² s⁻¹±2.8×10⁻³ and kₐ=8.99×10⁵ M⁻¹ s⁻¹±4.14×10⁵. The association rate obtained through subsequent characterization of the sdAb by SPR was in the same range as from the TEM validation, while the dissociation rate was found to be slower with SPR. This discrepancy may be explained by the fact that fast k_{d} dynamics are difficult to capture using exponential fitting on a small number of experimental repeats. Results obtained through SPR screening with a limited number of conditions are thus likely to slightly differ from the TEM validation results. The obtained kₐ and k_{d} were then used as an input into the TEM to calculate binding signal at equilibrium S_{eq} for each analyte concentration. It was then evaluated how closely the model fits the experimental data (FIG. 23). FIG. 23 shows a good fit between experimental data and theoretical TEM values over the entire analyte concentration range. We then evaluated which concentration range resulted in a linear sensor response. FIG. 24 shows that the readout signal S(t) varies linearly with the analyte concentration up to 1.3 K_{D}. Within this range, a linear model fits the binding data with a determination coefficient R² value of 0.95, thus explaining about 95% of the variability in the binding signal.

Assessing the Impact of Custom Anti-IL6 sdAbs Kinetic Rates on Different Biosensing Applications

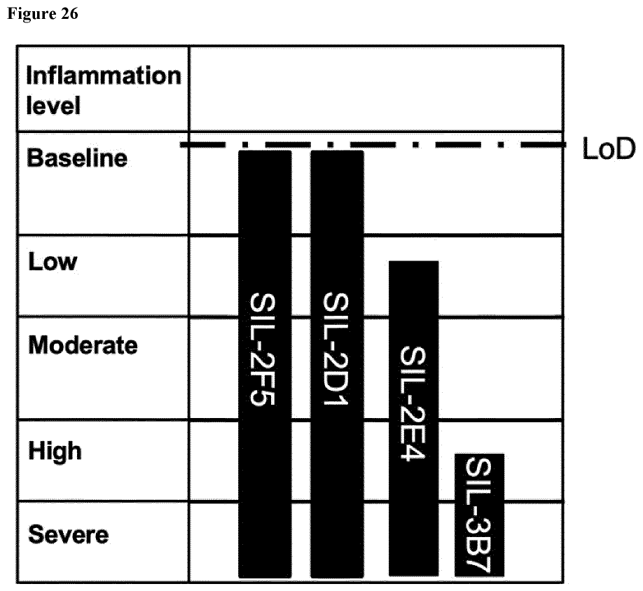

For each anti-IL6 sdAb, the inflammation range that can be detected in vivo was derived from the proportion of bound sdAbs at equilibrium. For the inflammation levels considered, the K_{D} of the anti-IL6 sdAbs (Table 10) is higher than/above the IL6 concentration during severe inflammation, that is 4.5 pM (Table 1). Thus, the proportion of bound sdAbs at equilibrium is always proportional to the input IL6 concentration (C(t)<<K_{D} in Equation 3). From this property derives the possibility to measure IL6 concentration in both single shot measurement and continuous biosensing applications. In single shot measurements, the medium of interest can be sampled at time t₀ and the sensor signal measured at different time steps t. For known kₐ and k_{d}, the TEM association equation (Equation 2), can be fitted to the measurements to extract the input concentration C(t=0). In continuous measurements, t₀ is unknown. Knowing the kₐ and k_{d} of the sensor, the scaling and time delay between the input concentration C(t) and the sensor signal S(t) must be determined through calibration prior to sensor use. The need to correct for time delay will depend on kinetic rates of the sdAb and can be predicted calculating the error between the TEM output and the initial concentration (see methods). Assuming a limit of detection for our system of 0.01% of bound sdAbs, the applicable inflammation ranges are summarized in FIGS. 25 and 26. SIL-2D8, SIL-2F5 a SIL-2D1 cover the largest IL6 concentration range, from baseline to severe inflammation. SIL-3B6, SIL-3E7 and SIL-2E4 cover an inflammation range from low to severe. SIL-3B7 still covers an inflammation range from high to severe. Accordingly, Table 12 demonstrates that the herein provided anti-IL6 sdAbs are surprisingly able to detect and/or quantify IL6 concentrations across the entire range of IL6 concentration during inflammation, i.e., from baseline to severe inflammation, e.g. in a human biological fluid, such as blood. Thus, the herein provided sdAbs are particularly useful in the (continuous) measurement or (continuous) monitoring of IL6 concentrations (such as in biosensors) over the entire inflammatory range of IL6 concentration e.g. in a human biological fluid such as blood.

Alternatively or additionally to using the sdAbs kinetic parameters to achieve continuous monitoring via sdAbs, repeated measurements could be achieved by recovering the sdAb-antigen complex e.g. by forced release through an optogenetic release mechanism, increasing temperature, inducing vibration, pH modifications, or changing the redox potential. Accordingly, forced release approaches may be employed in order to further enhance the ability of the herein provided sdAbs to continuously detect and/or quantify IL6 across the entire range of inflammation (e.g., across the full range of IL6 concentrations detailed in Table 1). This may be used to finetune binding dynamics for continuous monitoring of IL6 concentrations using the sdAbs provided herein. Further details on forced release approaches may be found in Govaert et al. 2012, Kunz et al. 2017, Kunz et al. 2019, Kunz et al. 2018, Gil et al. 2019, Renicke et al. 2013, Turton et al. 2014, Reverberi et al. 2007, Klaus and Deshmukh 2012, and Thompson et al. 2020, all of which are herewith incorporated by reference in their entirety. Alternatively, microfluidic approaches could be used in a multi-sensor chip, where for each measurement, only a single dedicated sensor chip is flooded with sample and used for measurement. Such approaches are detailed e.g. in Glatz et al. 2022, Chen et al. 2023 and Pour et al. 2024, all of which are incorporated herein in their entirety.

TABLE 12
Proportion of the occupied sites at the equilibrium of the sdAbs clones against the human inflammation ranges.

| | Concentration | | | | | | | |
|---|---|---|---|---|---|---|---|---|
| | in a human | | | | | | | |
| Inflam- | biological | Proportion of occupied sites at equilibrium [%] | | | | | | |
| mation | fluid, e.g. | SIL- | SIL- | SIL- | SIL- | SIL- | SIL- | SIL- |
| range | blood | 2D8 | 3B6 | 3E7 | 2F5 | 2D1 | 2E4 | 3B7 |
| Baseline | 45 fM (or less) | 0.012 ** | 0.007 | 0.002 | 0.015 ** | 0.012 ** | 0.006 | 0.001 |
| Low | 45 fM-225 fM | 0.012-0.06 ** | 0.007-0.03 * | 0.002-0.012 * | 0.015-0.076 ** | 0.012-0.06 ** | 0.006-0.030 * | 0.001- 0.003 |
| | 0.045 pM- | | | | | | | |
| | 0.225 pM | | | | | | | |
| Moderate | 225 fM-450 fM | 0.06-0.12 ** | 0.03-0.07 ** | 0.012-0.025 ** | 0.076-0.15 ** | 0.06-0.12 ** | 0.03-0.06 ** | 0.003-0.007 |
| | 0.225 pM- | | | | | | | |
| | 0.45 pM | | | | | | | |
| High | 450 fM-4.5 pM | 0.12-1.2 ** | 0.07-0.7 ** | 0.025-0.25 ** | 0.15-1.50 ** | 0.12-1.21 ** | 0.06-0.6 ** | 0.007-0.07 * |
| Severe | 4.5 pM (or more) | 1.2 ** | 0.7 ** | 0.25 ** | 1.50 ** | 1.21 ** | 0.6 ** | 0.07 ** |

The limit of detection of the system was assumed to be equivalent to 0.01% of bound sdAbs, or 100 parts per million (Kongsuphol (2014) Biosensors and Bioelectronics 61: 274-279). In other words, if a sdAb is indicated in the table below to comprise about 0.01% or more than about 0.01% of occupied sites at equilibrium at a given inflammation range (e.g. IL6 concentration in a human biological fluid), the sdAb is particularly preferred for the detection of IL6 when IL6 is present in the respective concentrations in a human biological fluid. The upper limit of detection corresponds to the saturation of the binding sites (i.e., 100% of occupied sites at equilibrium) of the sdAb. Whenever a certain sdAb is capable of detecting IL6 across a complete inflammation range and the sdAb is, thus, particularly preferred for diagnostic assessment of inflammation or monitoring of inflammation levels based on IL6 concentrations in a human biological fluid, this is indicated by “**” in the table below. If a sdAb does not cover a full inflammation range, however, may still be particularly preferred for detecting IL6 concentrations within a given inflammation range, the respective sdAb may still be particularly preferred for diagnostic assessment or monitoring of inflammation based on IL6 concentrations in the respective range, this is indicated by “*” in the table below. The IL6 concentrations are based on Table 1. Further details on the methods and models underlying this table can be found in Example 1.

TABLE 13
Summary characteristics of exemplary sdAbs provided herein.
The column “Covered inflammation range” in the table
below indicates the respective inflammation range that a
given sdAb can at least partly cover (i.e., any inflammation
range that is indicated with “*” in Table 12) or
fully cover (i.e., any inflammation range that is indicated
with “**” in Table 12). All other columns summarize
data also already detailed herein above or below.

| | | | | | Covered |
|---|---|---|---|---|---|
| | | | | | inflammation |
| | | | | | range |
| | | | | IC50 | (based on |
| Clone | kₐ[s⁻¹M⁻¹] | k_{d} [s⁻¹] | K_{D} [M] | [nM] | Table 12) |
| SIL-2D8 | 1.82E6 | 6.71E−4 | 0.37 | 3.97 | Baseline to severe |
| SIL-2E7 | — | — | — | — | — |
| SIL-2F5 | 2.21E6 | 6.53E−4 | 0.30 | 3.04 | Baseline to severe |
| SIL-2D1 | 8.37E5 | 3.08E−4 | 0.37 | >120 | Baseline to severe |
| SIL-3B6 | 8.45E5 | 5.35E−4 | 0.63 | >120 | Low to severe |
| SIL-2E4 | 7.33E5 | 5.52E−4 | 0.75 | >120 | Low to severe |
| SIL-3E7 | 8.64E5 | 1.61E−3 | 1.9 | >600 | Low to severe |
| SIL-3B7 | 1.86E5 | 1.27E−3 | 6.8 | >600 | High to severe |

### EXAMPLE 2: YEAST PRODUCTION AND PURIFICATION OF ANTI-IL6 sdAbs

Production is started from a single colony which is inoculated in 5 ml YP with 2% glucose and is grown for 24 hours at 30 °C at 200 rpm. The overnight culture is diluted 1:20 in 100 mL YNB with 2% glucose in a 2 L flask and grown for 24 hours to make a pre-culture. Addition of 750 mL YP, 100 mL 20% glucose, 50 mL 20% galactose to the 100 mL pre-culture starts the production which is continued for 72 hours. After the production ends OD600 is measured (dilute 1:50 in water before measuring). The culture should reach OD600 >20. Cultures are spun down for 20 min at 4800 rpm in centrifuge and supernatant is directly after centrifugation poured gently into a clean bottle. The supernatant is filter sterilized using 0.45 μm bottle top filters on an autoclaved bottle. Take 30 μl culture supernatant (taken at the flame with a sterile pipette tip), add 10 d 4×SDS-sample buffer and boil the samples at >95 °C for 5 min and load 10 μl of the samples on a 15% SDS-polyacrylamide gel next to a 1 μg reference sdAb to estimate the production level. An appropriate amount of supernatant containing sdAb is used for AKTA purification using a JSR protA column according to the manufacturers protocol.

### EXAMPLE 3: ANALYSIS OF THE INHIBITORY POTENTIAL OF sdAb for IL6 SIGNALING BY ALPHASCREEN (FROM PATENT US 2011/0059101 A9)

To differentiate inhibitory from non-inhibitory anti-IL6 sdAbs, sdAbs capable of inhibiting the interaction between IL6 and IL6R are identified by Alphascreen. In this assay, periplasmic extracts prepared from anti-IL6 sdAb expressing E. coli cells (25-fold diluted) are incubated with 3 nM biotinylated human IL6 in a 384-wells plate for 15 min. Subsequently a mixture of IL6R (1 nM) and acceptor beads (20 ug/ml) coated with anti-IL6R MAb BN-12 (Diaclone) are added and incubated for 30 min. Finally, streptavidin coated donor beads (20 ug/ml) are added.

After 1 hour of incubation the plates were read on the Envision Alphascreen reader (PerkinElmer). To further differentiate inhibitory from non-inhibitory anti-IL6 sdAbs, sdAbs against the gp130 binding sites on IL6 are identified by an indirect Alphascreen assay in which MAbs BE-4 (Diaclone) and CLB16 (Sanquin, Amsterdam) are employed. These two anti-IL6 antibodies recognize gp130 binding site II and III, respectively. In this assay, periplasmic extracts are incubated for 15 min with 1 nM biotinylated IL6. Acceptor beads (20 ug/ml) coated with either BE-4 or CLB16 are added and after 30 min streptavidin coated donor beads (20 ug/ml) are added. Reaction mixtures are incubated for 1 hour and then read on the Envison Alphascreen reader (PerkinElmer).

### EXAMPLE 4: ANALYSIS OF THE INHIBITORY POTENTIAL OF sdAb FOR IL6 SIGNALING IN B9 ASSAY (FROM PATENT US 2011/0059101 A9)

To differentiate inhibitory from non-inhibitory anti-IL6 sdAbs, purified samples sdAbs are tested in a sensitive and commonly used method for measuring biological IL6 activity based on IL6 dependent proliferation of a murine cell line. This proliferation assay employs the murine hybridoma cell line B9 and is performed as described by Aarden et al. (Eur J Immunol. 17 (1987):1411-1416). Inhibition curves and IC50 values are established by sdAb titration and measuring B9 proliferation.

### EXAMPLE 5: sdAb BINDING TO IL6-PEPSCAN PEPTIDE EPITOPE MAPPING

The linear and CLIPS peptides are synthesized based on the amino acid sequence of human IL6 using standard Fmoc-chemistry and deprotected using trifluoric acid with scavengers. For conformational mapping, the constrained peptides are synthesized on chemical scaffolds in order to reconstruct conformational epitopes, using Chemically Linked Peptides on Scaffolds (CLIPS) technology (Timmerman et al. 2007 (J. Mol. Recognit. 20: 283-299)). For example, the single looped peptides are synthesized containing a dicysteine, which was cyclized by treating with alpha, alpha′-dibromoxylene and the size of the loop is varied by introducing cysteine residues at variable spacing. If other cysteines besides the newly introduced cysteines are present, they are replaced by cysteine-acetamydomethyl. The side-chains of the multiple cysteines in the peptides are coupled to CLIPS templates by reacting onto credit-card format polypropylene PEPSCAN cards (455 peptide formats/card) with a 0.5 mM solution of CLIPS template such as 1,3-bis(bromomethyl) benzene in ammonium bicarbonate (20 mM, pH 7.9)/acetonitrile (1:1 (v/v)). The cards are gently shaken in the solution for 30 to 60 minutes while completely covered in solution. Finally, the cards are washed extensively with excess of H₂O and sonicated in disrupt-buffer containing 1% SDS/0.1% beta-mercaptoethanol in PBS (pH 7.2) at 70 °C for 30 minutes, followed by sonication in H₂O for another 45 minutes. The binding of sdAb to each peptide is tested in a PEPSCAN-based ELISA. The 455-well credit card format polypropylene cards containing the covalently linked peptides are incubated with primary antibody solution, for example, consisting of 1 μg/ml diluted in blocking solution, for example 4% horse serum, 5% ovalbumin (w/v) in PBS/1% Tween. After washing, the peptides are incubated with a 1/1000 dilution of antibody peroxidase conjugate for one hour at 25 °C. After washing, the peroxidase substrate 2,2′-azino-di-3-ethylbenzthiazoline sulfonate (ABTS) and 2 μl of 3% H₂O₂ are added. After one hour, the color development is measured. The color development is quantified with a charge coupled device (CCD)-camera and an image processing system (as firstly described in Slootstra et al., 1996 (Mol. Divers. 1: 87-96)). The raw data are optical values obtained by a CCD-camera calibrated to export absorption values compatible with a standard 96-well plate ELISA-reader. First the CCD-camera takes a picture of the card before peroxidase coloring and then again after the peroxidase coloring. These two pictures are subtracted from each other yielding a binding value for each peptide. This data is entered in the Peplab™ database for secure storage and retrieval.

### EXAMPLE 6: sdAb INHIBITION OF IL6 RECEPTOR SIGNALING USING iLite® IL6 ASSAY READY CELLS

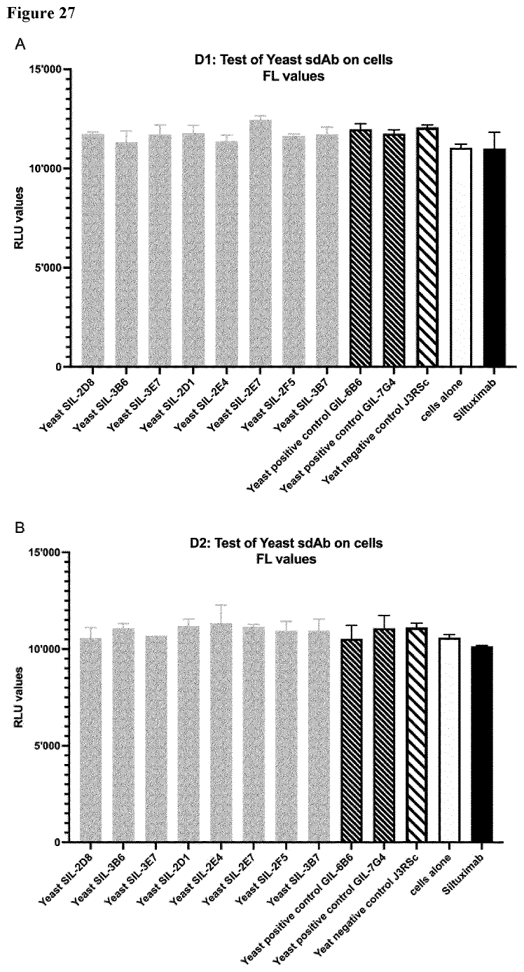

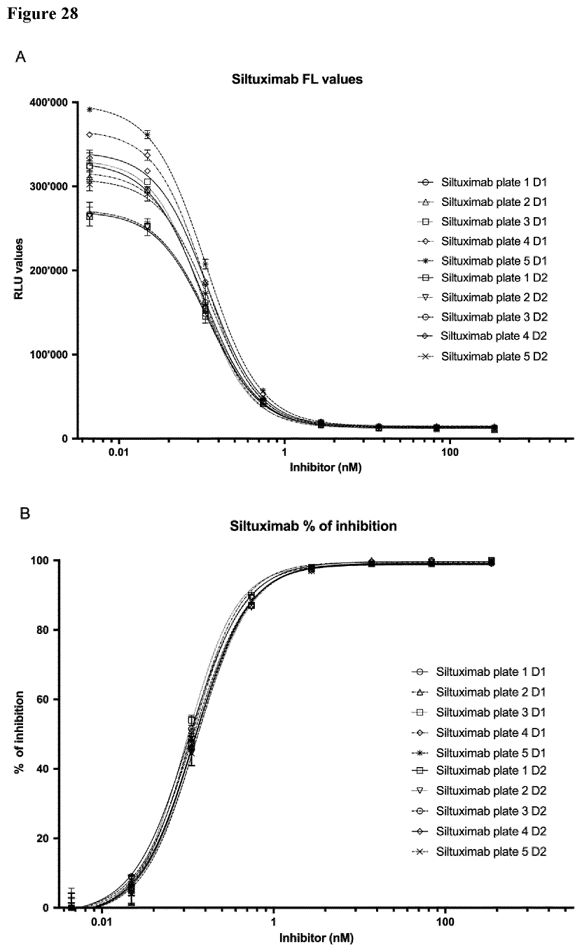

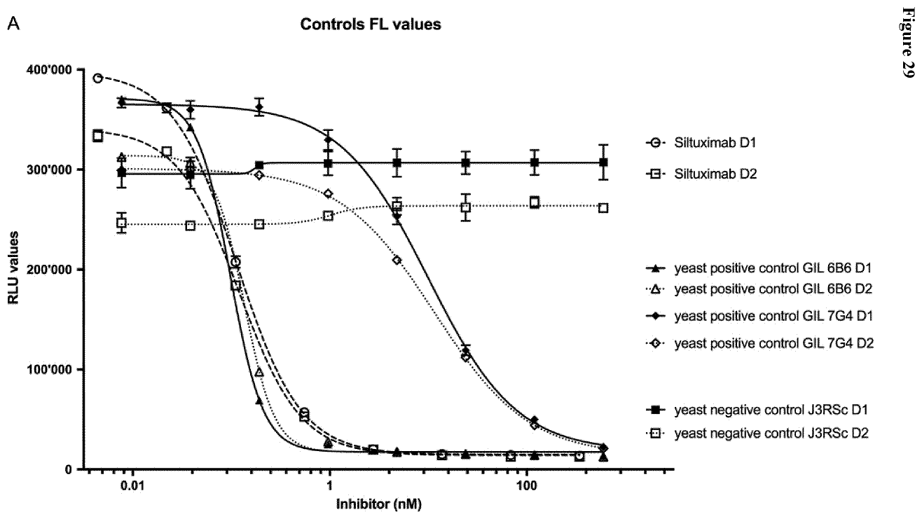

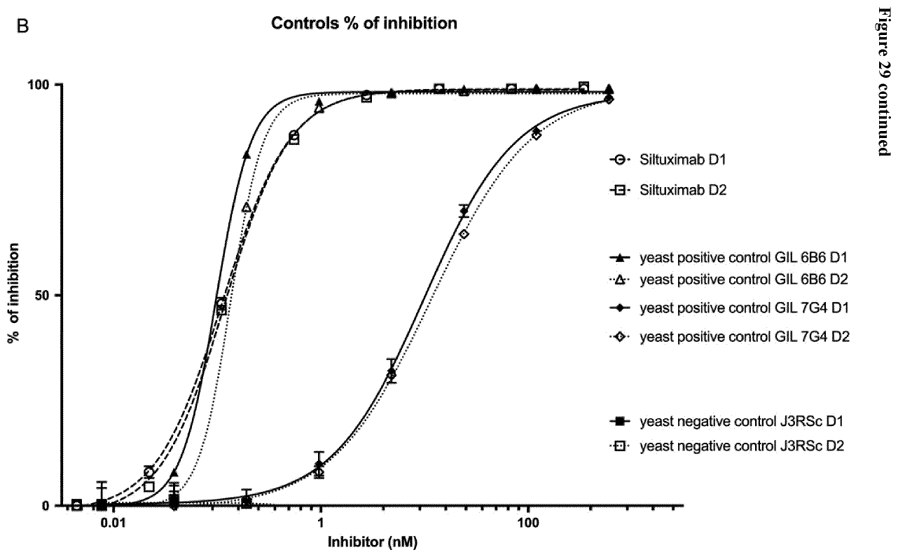

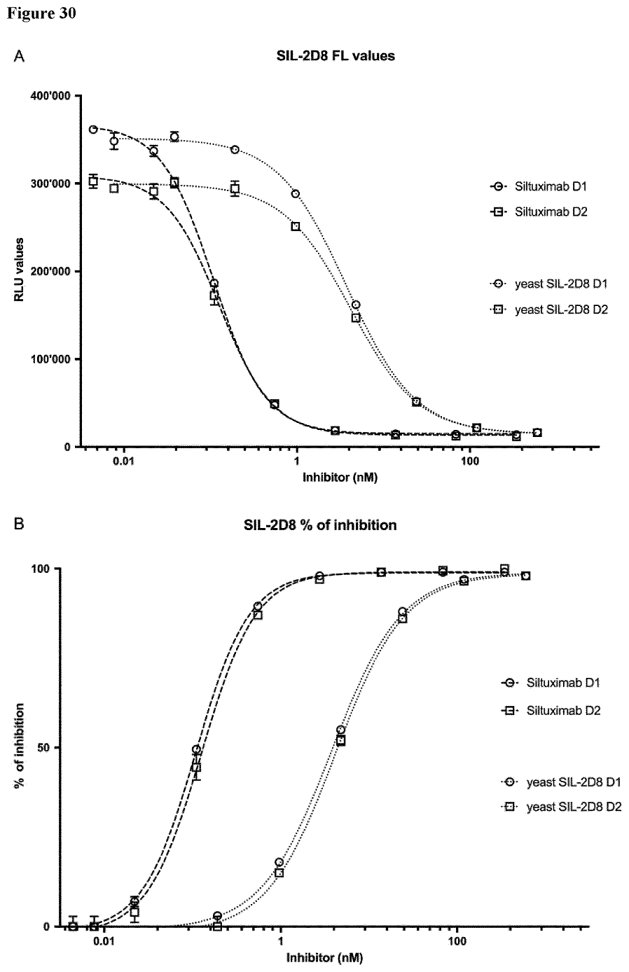

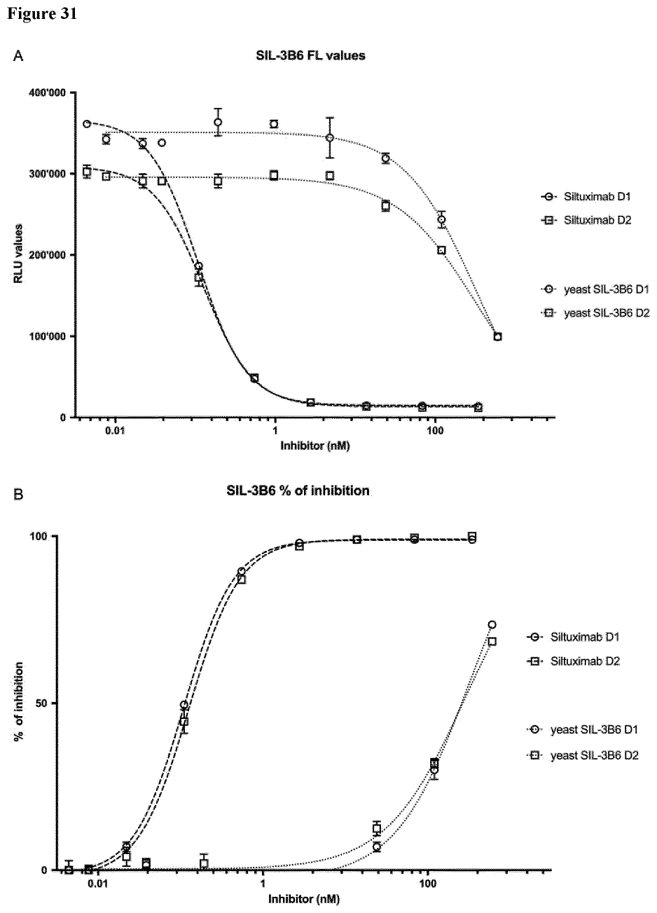

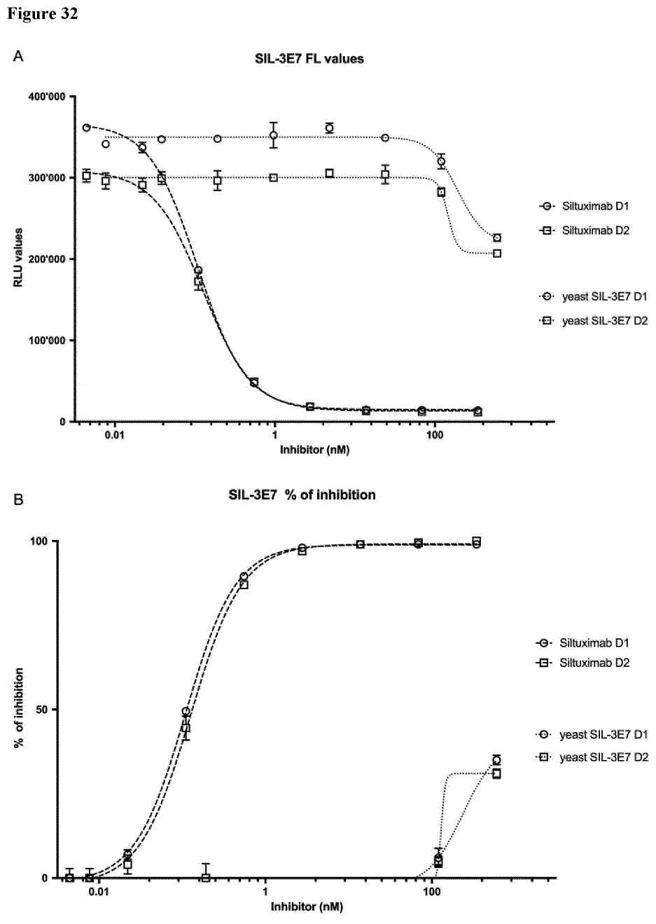

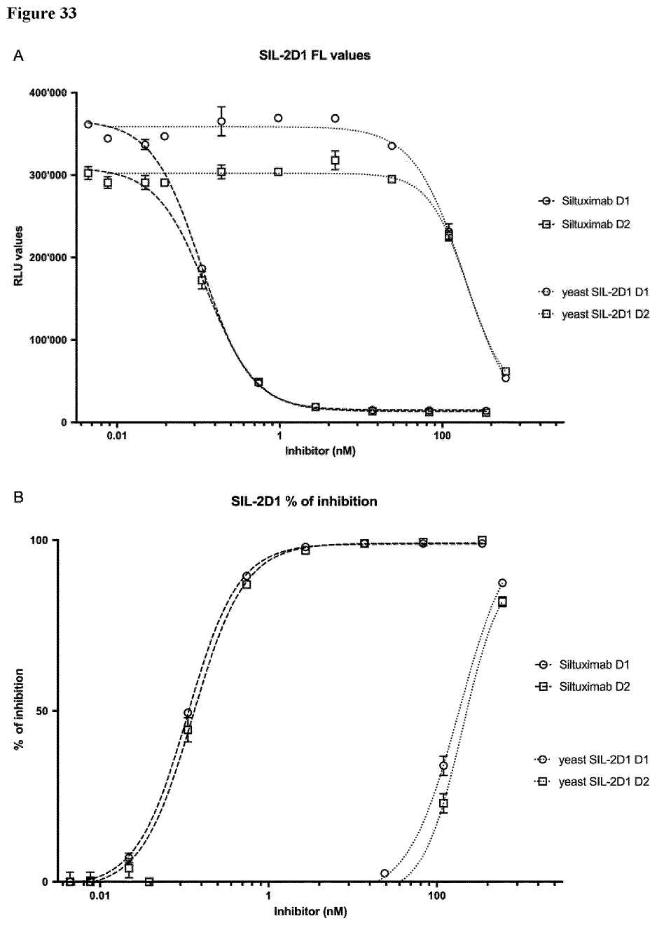

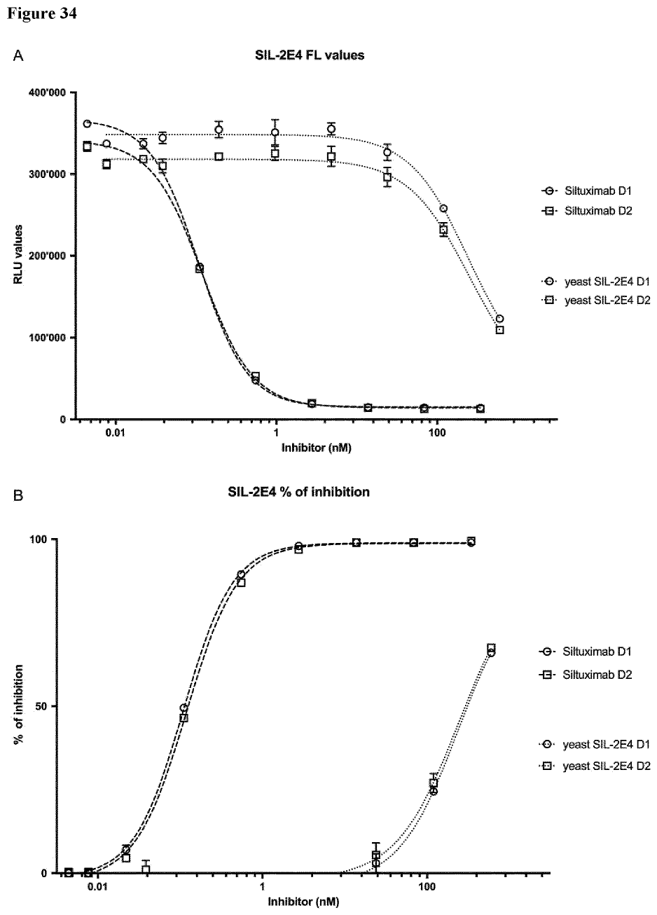

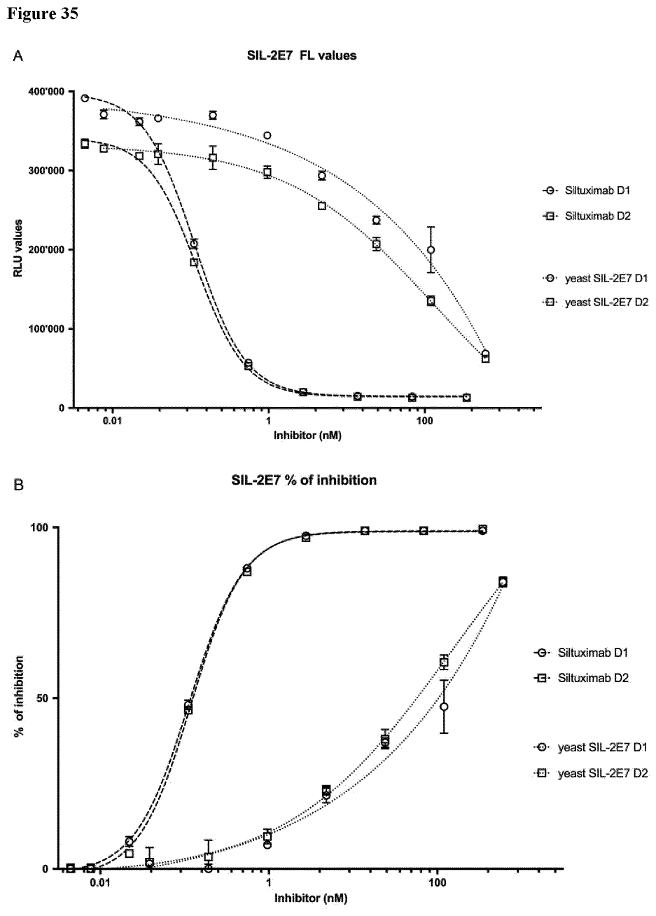

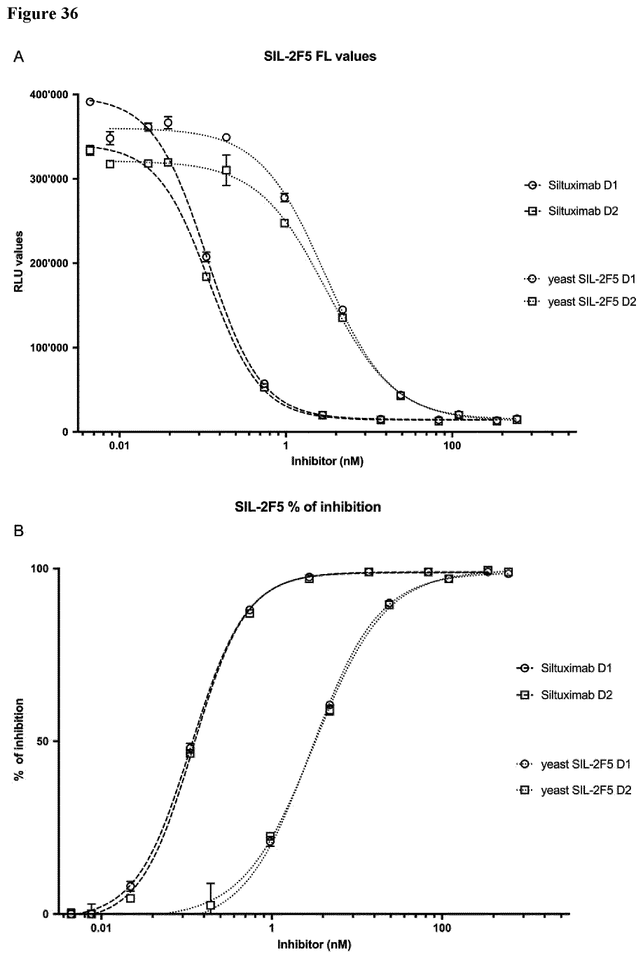

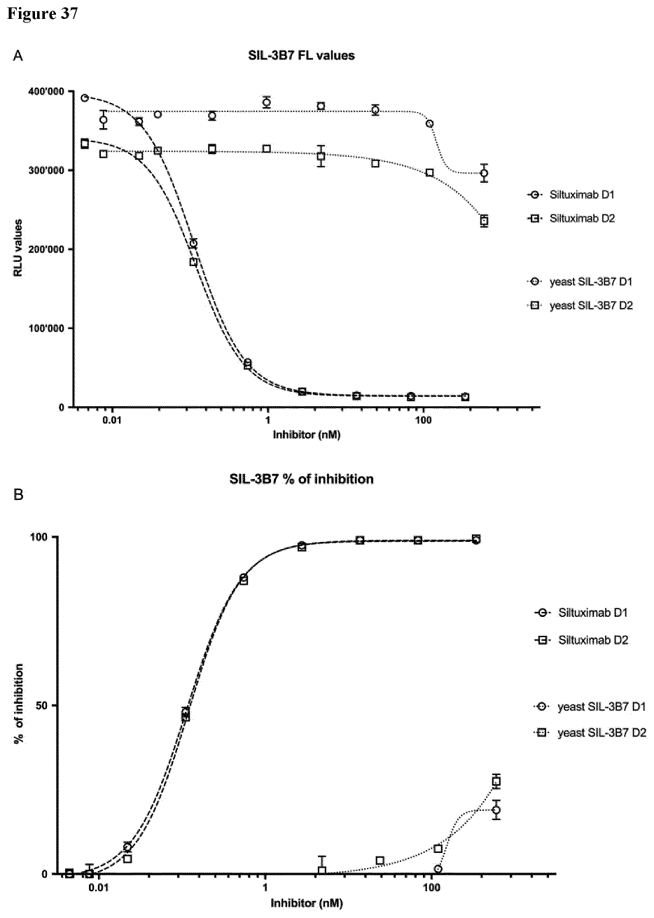

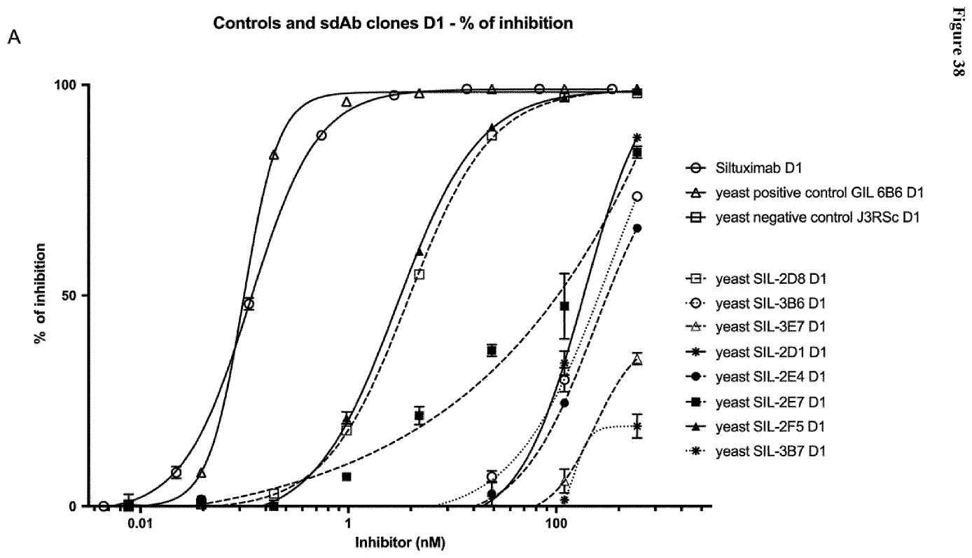

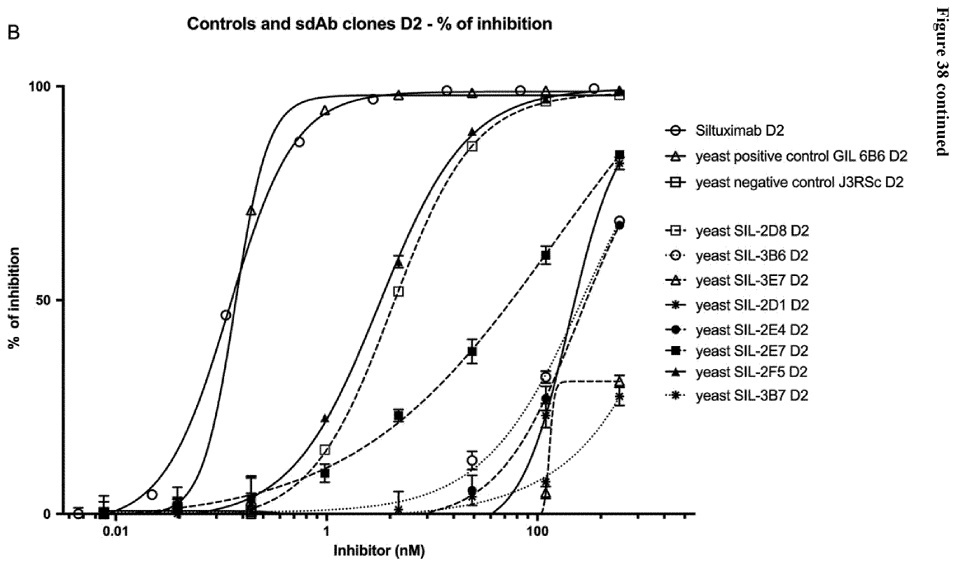

The iLite® IL6 Assay Ready Cells (Svar Life Science) are engineered cells optimized to express Firefly luciferase under the control of a IL6 responsive promoter. Binding of IL6 to the membrane bound IL6R (IL6RA and IL6ST) results in activation of the IL6 regulated Firefly luciferase reporter gene construct. iLite® IL6 Assay Ready Cells also contain the Renilla Luciferase (RL) reporter gene, under the control of a constitutive promoter. The constitutive expression of RL allows normalization of IL6 induced FL activity, and renders assay results independent of variations in cell number or serum matrix effects. The luciferase signal can be measured in a luminometer following addition and incubation of luciferase substrate. The Firefly luciferase signal is proportional to the functional activity of IL6 in the sample. In the presence of inhibitory activity against the IL6R, the functional activity of the present IL6 is reduced, resulting in a decreased stimulation of Firefly luciferase production. Thus, the Firefly luciferase signal is inversely proportional to the amount of inhibitory activity against IL6R in a sample. The iLite® IL6 Assay Ready Cells can therefore be utilized as a highly sensitive assay for quantification of IL6R inhibitor activity of anti-IL6 sdAbs. To assess the inhibitory potential of the herein above detailed anti-IL6 sdAbs, they were tested against GIL-6B6 and GIL-7G4 (both positive anti-IL6 sdAb controls derived from US 2011/0059101 A9), J3RSc-P55T (negative control, anti-HIV sbAb derived from McCoy (2012) The Journal of experimental medicine, 209(6), 1091-1103) and Siltuximab (SILVANT™ by Janssen Biotech) (therapeutic conventional monoclonal antibody against IL6, serving as positive control). IL6 reporter cells were incubated for 30 min at 37 °C with different concentrations of sdAbs or controls according to manufacturer's instructions. Siltuximab, a bivalent anti-IL6 antibody was used at concentrations from 50,000 ng/ml to 3.2 ng/ml (345 nM to 0.02 nM). In contrast, sdAbs are approx. 10-fold smaller and only monovalent binders (i.e., only comprise a single antigen binding site) as compared to conventional antibodies. Therefore, sdAbs were used at 5-times lower concentrations than Siltuximab in order to adjust for the molarity of available variable antibody domains, from around 10,000 ng/ml to 0.64 ng/ml (600 nM to 0.038 nM). Afterwards, 2 ng/ml of IL6 antigen was added to the cells for 6 h, after which FL and the RL measurements were performed using Dual-Glo® Luciferase Assay System (Promega). The percentage of inhibition was calculated as follows based on the relative light units (RLU) obtained from the Dual-Glo® measurements: [(mean RLU of cells without inhibitor)−(mean RLU of cells only)]−[(RLU of sample)−(mean RLU of cells only)](mean RLU of cells without inhibitor)−(mean RLU of cells only). In this context, “cells without inhibitor” refer to cells being exposed to IL6, however, not to any sdAb nor to any control antibody. Further, “cells only” indicates that cells were neither contacted with IL6 or any sdAb or control antibody. The half-maximal inhibitory concentration (IC50), i.e., the concentration of an antibody leading to half-maximal inhibition (Neubig et al. 2003), was calculated by fitting a sigmoidal dose-response curve to the data. In case of incomplete dose-response curves (i.e., dose response curves that at the concentrations measured do not reach a saturation of inhibition, in other words there is no sigmoid dose-response curve for a given sdAb, as exemplified in FIG. 32), the IC50 could not be determined. In those cases, the IC50 was set to the last measured sdAb concentration that led to less than 50% inhibition, which was either at 120 nM or at 600 nM (see Table 13). D1 and D2 represent two independent runs of the same assay. Importantly, no IL6 signaling could be observed when cells were incubated with either sdAbs or controls alone (i.e., in the absence of IL6; FIG. 27), meaning there were no contaminations (such as IL6 or endotoxins driving IL6 signaling) in the sdAb or control samples that would induce IL6 signaling. As compared to Siltuximab (IC50 in the picomolar range, FIG. 28 and Table 14-15), which is suitable for therapeutic purposes, the positive control sdAbs show similar (GIL-6B6) or higher (GIL-7G4) IC50 (FIG. 29 and Table 16), and no inhibition was observed for the negative control J3RSc-P55T (FIG. 29 and Table 16). The rationally selected sdAbs showed different degrees of IL6 inhibition, with many of the IC50 being orders of magnitude lower (FIGS. 30-37 and Table 17-24) than the one of Siltuximab. The inhibition curves are summarized in FIG. 38a-b and the respective IC50 values for the herein provided sdAb are summarized in Table 13. Indeed, sdAbs that are rationally selected for repeated IL6 sampling at physiological concentrations of IL6 representative of health and disease status (i.e. on the gradient from baseline inflammation levels to severe inflammation levels, inter alia, described in Table 12), but that do not strongly interfere with IL6 signaling, are advantageous for diagnostic or monitoring applications of IL6 concentrations, e.g. in biosensors. This can be especially valuable when sdAbs might be directly exposed to the human body, for example when such sdAbs are comprised in e.g. a permanently or reversibly implanted or attached (to the subject's body) like appliance, such as an implanted, partly implanted or wearable device comprising the biosensor of the present invention as described herein that (continuously) monitor IL6 concentrations in biological fluids, e.g. human ISF or other biofluids. Here, suitable and specific sdAbs need to be rationally selected depending on the desired application e.g., in vivo biosensor, or ex vivo biosensor, in vitro biosensor or a therapeutic application and the concentration range of IL6 that is desired to be covered for successful monitoring and/or diagnosis and/or therapy of IL6-associated diseases, especially when used as part of a biosensor or any apparatus provided herein. Exemplary characteristics that may serve as an indication for preferred sdAb with advantageous properties when used in a biosensor or any other apparatus provided herein are summarized in Table 13. As an example, SIL-2D1 and SIL-3E7 may be preferred for use as a capture molecule for an in vivo biosensor, with fast association (kₐ) and dissociation (k_{d}) allowing for continuous monitoring of IL6 covering the entire detection range for inflammatory conditions (FIGS. 25 and 26, Tables 12 and 13), while having a low risk for strong interference with host IL6 signaling (FIGS. 32, 33 and 38, Table 13). This can be advantageous, inter alia, from a safety and thus regulatory perspective as there can be a risk of sdAbs diffusing from a biosensor device into the systemic circulation, where inhibition of the IL6 pathway by such sdAbs would be seen as an unintended side effect. Accordingly, the low inhibition of the biological function of IL6 is advantageous in such an application. In contrast, based on these data SIL-2D8 and SIL-2F5 given their strong inhibitory potential may both rather be well suited for ex vivo or in vitro biosensors, where inhibition of the IL6 pathway would not be of relevance as well as for therapeutic applications given their low nanomolar IC50, 3.97 nM and 3.04 nM respectively (see FIGS. 30, 36 and 38, Table 13). SIL-3B6 and SIL-2E4, similar to SIL-2D1 and SIL-3E7, may be preferred for use as a capture molecule for an in vivo biosensor, tough covering a slightly more narrow inflammation range as compared to SIL-2D1 and while having a slightly increased inhibitory potential as compared to SIL-3E7 (see FIGS. 31 and 34, Tables 12 and 13). SIL-3B7 may be preferred for use as a capture molecule for an in vivo biosensor in settings where conditions that present high to severe IL6 concentrations need to be monitored (see FIGS. 37, Tables 12 and 13).

TABLE 14
FL Parameters of IL6 inhibition by Siltuximab

| Siltuximab | Plate 1 | Plate 2 | Plate 3 | Plate 4 | Plate 5 | Plate 1 | Plate 2 | Plate 3 | Plate 4 | Plate 5 | | |
|---|---|---|---|---|---|---|---|---|---|---|---|---|
| FL | D1 | D1 | D1 | D1 | D1 | D2 | D2 | D2 | D2 | D2 | Mean | CV |
| Upper | 329′505 | 317′172 | 331′384 | 366′760 | 398′016 | 270′513 | 269′948 | 272′420 | 341′199 | 308′948 | 320′587 | 13% |
| asymptote | | | | | | | | | | | | |
| Lower | 12′275 | 13′007 | 14′808 | 14′951 | 14′679 | 12′623 | 12′713 | 12′837 | 14′195 | 13′396 | 13′548 | 7% |
| asymptote | | | | | | | | | | | | |
| Background | 323′700 | 311′150 | 324′400 | 361′500 | 391′500 | 264′100 | 265′050 | 267′100 | 333′850 | 302′500 | 314′485 | 13% |
| Hill Slope | −1.343 | −1.49 | −1.495 | −1.442 | −1.341 | −1.371 | −1.368 | −1.379 | −1.385 | −1.409 | −1.402 | −4% |
| IC50 (nM) | 0.1029 | 0.1064 | 0.09658 | 0.108 | 0.1126 | 0.1191 | 0.1261 | 0.1207 | 0.1197 | 0.1263 | 0.114 | 9% |
| R² | 0.9993 | 0.9992 | 0.9977 | 0.9997 | 0.9998 | 0.9983 | 0.9991 | 0.9976 | 0.9993 | 0.9984 | 0.999 | 0% |
| Span | 317′230 | 304′165 | 316′577 | 351′809 | 383′337 | 257′890 | 257′235 | 259′583 | 327′004 | 295′552 | 307′038 | 14% |
| RL mean | 232′706 | 218′825 | 233′700 | 252′869 | 259′081 | 241′306 | 239′850 | 248′231 | 264′694 | 263′625 | 245′489 | 6% |

TABLE 15
Parameters of the % of IL6 inhibition by Siltuximab

| Siltuximab | | | | | | | | | | | | |
|---|---|---|---|---|---|---|---|---|---|---|---|---|
| % of | Plate 1 | Plate 2 | Plate 3 | Plate 4 | Plate 5 | Plate 1 | Plate 2 | Plate 3 | Plate 4 | Plate 5 | | |
| inhibition | D1 | D1 | D1 | D1 | D1 | D2 | D2 | D2 | D2 | D2 | Mean | CV |
| Upper | 99.67 | 99.51 | 98.72 | 98.91 | 98.96 | 99.2 | 99.27 | 99 | 98.79 | 99.11 | 99 | 0% |
| asymptote | | | | | | | | | | | | |
| Lower | −1.848 | −2.024 | −2.066 | −1.431 | −1.53 | −2.393 | −1.975 | −1.982 | −2.327 | −2.164 | −1.974 | 16% |
| asymptote | | | | | | | | | | | | |
| Hill Slope | 1.338 | 1.476 | 1.51 | 1.443 | 1.348 | 1.37 | 1.374 | 1.378 | 1.4 | 1.412 | 1.40 | 4% |
| IC50 (nM) | 0.103 | 0.106 | 0.0975 | 0.109 | 0.114 | 0.118 | 0.127 | 0.121 | 0.119 | 0.126 | 0.114 | 9% |
| R² | 0.9991 | 0.9991 | 0.998 | 0.9998 | 0.9998 | 0.9985 | 0.9989 | 0.9976 | 0.9992 | 0.9983 | 0.999 | 0% |

TABLE 16
Parameters of the % of IL6 inhibition by the control sdAbs

| | Yeast | Yeast | | Yeast | Yeast | | | | |
|---|---|---|---|---|---|---|---|---|---|
| Control | GIL | GIL | Mean | GIL | GIL | Mean | Yeast | Yeast | Mean |
| % of | 6B6 | 6B6 | GIL | 7G4 | 7G4 | GIL | J3RSc | J3RSc | J3RSc |
| inhibition | D1 | D2 | 6B6 | D1 | D2 | 7G4 | D1 | D2 | D2 |
| Max FL | 370′400 | 312′600 | 341′500 | 366′750 | 299′000 | 332′875 | 296′700 | 246′800 | 271′750 |
| Min FL | 13′730 | 12′565 | 13′147 | 22′400 | 20′950 | 21′675 | 307′300 | 261′700 | 284′500 |
| Hill | 2.557 | 2.770 | 2.663 | 0.9826 | 0.8986 | 0.9406 | / | / | / |
| Slope | | | | | | | | | |
| IC50 | 0.09769 | 0.1353 | 0.1165 | 9.857 | 11.69 | 10.77 | / | / | / |
| (nM) | | | | | | | | | |
| R² | 0.9995 | 0.9989 | 0.9992 | 0.9979 | 0.9997 | 0.9988 | / | / | / |
| RL | 256′550 | 268′250 | 262′400 | 270′650 | 279′406 | 275′028 | 259′506 | 261′969 | 260′737 |
| mean | | | | | | | | | |

TABLE 17
Parameters of the % of IL6 inhibition by SIL-2D8

| SIL-2D8 | | | | |
|---|---|---|---|---|
| % | Yeast 2D8 | Yeast 2D8 | | |
| inhibition | D1 | D2 | Mean | CV |
| Max FL | 348′250 | 294′500 | 321′375 | 12% |
| Min FL | 16′680 | 16′250 | 16′465 | 2% |
| Hill Slope | 1.091 | 1.117 | 1.1045 | 2% |
| IC50 (nM) | 3.762 | 4.181 | 3.9735 | 7% |
| R² | 0.9993 | 0.9990 | 0.9991 | 0% |
| RL mean | 261′425 | 267′150 | 264′288 | 2% |

TABLE 18
Parameters of the % of IL6 inhibition by SIL-3B6

| SIL-3B6 | | | | |
|---|---|---|---|---|
| % | Yeast 3B6 | Yeast 3B6 | | |
| inhibition | D1 | D2 | Mean | CV |
| Max FL | 342′400 | 296′500 | 319′450 | 10% |
| Min FL | 99′020 | 99′800 | 99′410 | 1% |
| Hill Slope | 0.9609 | 0.8973 | 0.9215 | 5% |
| IC50 (nM) | >120 | >120 | >120 | / |
| R² | 0.9801 | 0.9911 | 0.98755 | 1% |
| RL mean | 262′288 | 288′531 | 275′410 | 7% |

TABLE 19
Parameters of the % of IL6 inhibition by SIL-3E7

| SIL-3E7 | | | | |
|---|---|---|---|---|
| % | Yeast 3E7 | Yeast 3E7 | | |
| inhibition | D1 | D2 | Mean | CV |
| Max FL | 341′450 | 296′000 | 318′725 | 10% |
| Min FL | 226′100 | 207′000 | 216′550 | 6% |
| Hill Slope | 2.055 | / | / | / |
| IC50 (nM) | >600 | >600 | >600 | / |
| R² | 0.9771 | 0.9551 | 0.91725 | 2% |
| RL mean | 270′619 | 283′344 | 276′982 | 3% |

TABLE 20
Parameters of the % of IL6 inhibition by SIL-2D1

| SIL-2D1 | | | | |
|---|---|---|---|---|
| % | Yeast 2D1 | Yeast 2D1 | | |
| inhibition | D1 | D2 | Mean | CV |
| Max FL | 344′450 | 291′000 | 317′725 | 12% |
| Min FL | 53′705 | 61′800 | 57′753 | 10% |
| Hill Slope | 1.410 | 1.844 | 1.618 | 19% |
| IC50 (nM) | >120 | >120 | >120 | / |
| R² | 0.9893 | 0.9876 | 0.98335 | 0% |
| RL mean | 268′231 | 275′919 | 272′075 | 2% |

TABLE 21
Parameters of the % of IL6 inhibition by SIL-2E4

| SIL-2E4 | | | | |
|---|---|---|---|---|
| % | Yeast 2E4 | Yeast 2E4 | | |
| inhibition | D1 | D2 | Mean | CV |
| Max FL | 337′150 | 312′200 | 324′675 | 5% |
| Min FL | 123′000 | 109′450 | 116′225 | 8% |
| Hill Slope | 1.210 | 1.138 | 1.1565 | 4% |
| IC50 (nM) | >120 | >120 | >120 | / |
| R² | 0.9883 | 0.9876 | 0.9720 | 0% |
| RL mean | 267′163 | 279′481 | 273′322 | 3% |

TABLE 22
Parameters of the % of IL6 inhibition by SIL-2E7

| SIL-2E7 | | | | |
|---|---|---|---|---|
| % | Yeast 2E7 | Yeast 2E7 | | |
| inhibition | D1 | D2 | Mean | CV |
| Max FL | 370′800 | 327′700 | 349′250 | 9% |
| Min FL | 68′615 | 62′065 | 65′340 | 7% |
| Hill Slope | 0.2619 | 0.4481 | 0.355 | 37% |
| IC50 (nM) | * | * | * | / |
| R² | 0.9802 | 0.9946 | 0.9874 | 1% |
| RL mean | 283′300 | 280′913 | 282′107 | 1% |

*could not be assessed as no sigmoid curve was observed, in other words, no saturation of inhibition was observed at the measured concentrations.

TABLE 23
Parameters of the % of IL6 inhibition by SIL-2F5

| SIL-2F5 | | | | |
|---|---|---|---|---|
| % | Yeast 2F5 | Yeast 2F5 | | |
| inhibition | D1 | D2 | Mean | CV |
| Max FL | 348′150 | 317′500 | 332′825 | 7% |
| Min FL | 15′900 | 14′525 | 15′213 | 6% |
| Hill Slope | 1.099 | 1.039 | 1.069 | 4% |
| IC50 (nM) | 2.918 | 3.156 | 3.037 | 6% |
| R² | 0.9978 | 0.9979 | 0.9979 | 0% |
| RL mean | 268′813 | 264′919 | 266′866 | 1% |

TABLE 24
Parameters of the % of IL6 inhibition by SIL-3B7

| SIL-3B7 | | | | |
|---|---|---|---|---|
| % | Yeast 3B7 | Yeast 3B7 | | |
| inhibition | D1 | D2 | Mean | CV |
| Max FL | 363′850 | 320′600 | 342′225 | 9% |
| Min FL | 296′400 | 235′950 | 266′175 | 16% |
| Hill Slope | 5.910 | 0.6579 | 3.28395 | 113% |
| IC50 (nM) | >600 | >600 | >600 | / |
| R² | 0.9037 | 0.9684 | 0.9361 | 5% |
| RL mean | 288′188 | 286′850 | 287′519 | 0% |

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

### LIST OF REFERENCES

Blackburn, P., Després, J., Lamarche, B., Tremblay, A., Bergeron, J., Lemieux, I., & Couillard, C. (2006). Postprandial Variations of Plasma Inflammatory Markers in Abdominally Obese Men. Obesity, 14(10), 1747-1754. https://doi.org/10.1038/oby.2006.201
Chen, F.; Hu, Q.; Li, H.; Xie, Y.; Xiu, L.; Zhang, Y.; Guo, X.; Yin, K. Multiplex Detection of Infectious Diseases on Microfluidic Platforms. Biosensors 2023, 13, 410. https://doi.org/10.3390/bios13030410
Emerson, S. R., Kurti, S. P., Harms, C. A., Haub, M. D., Melgarejo, T., Logan, C., & Rosenkranz, S. K. (2017). Magnitude and Timing of the Postprandial Inflammatory Response to a High-Fat Meal in Healthy Adults: A Systematic Review. Advances in Nutrition: An International Review Journal, 8(2), 213-225. https://doi.org/10.3945/an.116.014431
Esposito, K., Nappo, F., Marfella, R., Giugliano, G., Giugliano, F., Ciotola, M., Quagliaro, L., Ceriello, A., & Giugliano, D. (2002). Inflammatory Cytokine Concentrations Are Acutely Increased by Hyperglycemia in Humans. Circulation, 106(16), 2067-2072. https://doi.org/10.1161/01.cir.0000034509.14906.ae
Foote, J., & Eisen, H. N. (1995). Kinetic and affinity limits on antibodies produced during immune responses. Proceedings of the National Academy of Sciences, 92(5), 1254-1256. https://doi.org/10.1073/pnas.92.5.1254
Gil, Agnieszka A., Evan M. Zhao, Maxwell Z. Wilson, Alexander G. Goglia, Cesar Carrasco-Lopez, Jose L. Avalos, and Jared E. Toettcher. 2019. “Optogenetic Control of Protein Binding Using Light-Switchable Nanobodies.” Preprint. Biochemistry. https://doi.org/10.1101/739201.
Glatz R T, Ates H C, Mohsenin H, Weber W, Dincer C. Designing electrochemical microfluidic multiplexed biosensors for on-site applications. Anal Bioanal Chem. 2022 September; 414(22):6531-6540. doi: 10.1007/s00216-022-04210-4. Epub 2022 Jul. 6. PMID: 35794347; PMCID: PMC9411084.
Govaert, Jochen, Mireille Pellis, Nick Deschacht, Cécile Vincke, Katja Conrath, Serge Muyldermans, and Dirk Saerens. 2012. “Dual Beneficial Effect of Interloop Disulfide Bond for Single Domain Antibody Fragments.” Journal of Biological Chemistry 287 (3): 1970-79.
Herieka, M., & Erridge, C. (2014). High-fat meal induced postprandial inflammation. Molecular Nutrition & Food Research, 58(1), 136-146. https://doi.org/10.1002/mnfr.201300104
Jarmoskaite, I., AlSadhan, I., Vaidyanathan, P. P., & Herschlag, D. (2020). How to measure and evaluate binding affinities. ELife, 9, e57264. https://doi.org/10.7554/elife.57264
Kongsuphol, P., Ng, H. H., Pursey, J. P., Arya, S. K., Wong, C. C., Stulz, E., & Park, M. K. (2014). EIS-based biosensor for ultra-sensitive detection of TNF-α from non-diluted human serum. Biosensors & bioelectronics, 61, 274-279. https://doi.org/10.1016/j.bios.2014.05.017
Kunz, Patrick, Tilman Flock, Nicolas Soler, Moritz Zaiss, Cécile Vincke, Yann Sterckx, Damjana Kastelic, Serge Muyldermans, and Jorg D. Hoheisel. 2017. “Exploiting Sequence and Stability Information for Directing Nanobody Stability Engineering.” Biochimica et Biophysica Acta (BBA) General Subjects 1861 (9): 2196-2205. https://doi.org/10.1016/j.bbagen.2017.06.014.
Kunz, Patrick, Aurelio Ortale, Norbert Mucke, Katinka Zinner, and Jorg D Hoheisel. 2019. “Nanobody Stability Engineering by Employing the ΔTm Shift; a Comparison with Apparent Rate Constants of Heat-Induced Aggregation.” Protein Engineering, Design and Selection, July, gzz017. https://doi.org/10.1093/protein/gzz017.
Kunz, Patrick, Katinka Zinner, Norbert Mücke, Tanja Bartoschik, Serge Muyldermans, and Jörg D. Hoheisel. 2018. “The Structural Basis of Nanobody Unfolding Reversibility and Thermoresistance.” Scientific Reports 8 (1): 7934. https://doi.org/10.1038/s41598-018-26338-z.
Kusnezow, W., Syagailo, Y. V., Rüffer, S., Klenin, K., Sebald, W., Hoheisel, J. D., Gauer, C., & Goychuk, I. (2006). Kinetics of antigen binding to antibody microspots: Strong limitation by mass transport to the surface. PROTEOMICS, 6(3), 794-803. https://doi.org/10.1002/pmic.200500149
Lippitz, B. E., & Harris, R. A. (2016). Cytokine patterns in cancer patients: A review of the correlation between interleukin 6 and prognosis. Oncoimmunology, 5(5), e1093722. https://doi.org/10.1080/2162402x.2015.1093722
Mavropoulou, E., Mechie, N.-C., Knoop, R., Petzold, G., Ellenrieder, V., Kunsch, S., Pilavakis, Y., & Amanzada, A. (2020). Association of serum interleukin-6 and soluble interleukin-2-receptor levels with disease activity status in patients with inflammatory bowel disease: A prospective observational study. PLoS ONE, 15(5), e0233811. https://doi.org/10.1371/journal.pone.0233811
McCoy L E, Quigley A F, Strokappe N M, et al. Potent and broad neutralization of HIV-1 by a llama antibody elicited by immunization. J Exp Med. 2012; 209(6):1091-1103. doi:10.1084/jem.20112655
Neubig R R, Spedding M, Kenakin T, Christopoulos A; International Union of Pharmacology Committee on Receptor Nomenclature and Drug Classification. International Union of Pharmacology Committee on Receptor Nomenclature and Drug Classification. XXXVIII. Update on terms and symbols in quantitative pharmacology. Pharmacol Rev. 2003 December; 55(4):597-606. doi: 10.1124/pr.55.4.4. PMID: 14657418.
Pour S R S, Calabria D, Emamiamin A, Lazzarini E, Pace A, Guardigli M, Zangheri M, Mirasoli M. Microfluidic-Based Non-Invasive Wearable Biosensors for Real-Time Monitoring of Sweat Biomarkers. Biosensors (Basel). 2024 Jan. 4; 14(1):29. doi: 10.3390/bios14010029. PMID: 38248406; PMCID: PMC10813635.
Ravelo, B., Rahajandraibe, W., Gan, Y., Wan, F., Murad, N. M., & Douyére, A. (2020). Reconstruction Technique of Distorted Sensor Signals With Low-Pass NGD Function. IEEE Access, 8, 92182-92195. https://doi.org/10.1109/access.2020.2994630
Renicke, Christian, Daniel Schuster, Svetlana Usherenko, Lars-Oliver Essen, and Christof Taxis. 2013. “A LOV2 Domain-Based Optogenetic Tool to Control Protein Degradation and Cellular Function.” Chemistry & Biology 20 (4): 619-26. https://doi.org/10.1016/j.chembiol.2013.03.005.
Reverberi, Roberto, and Lorenzo Reverberi. 2007. “Factors Affecting the Antigen-Antibody Reaction.” Blood Transfusion. https://doi.org/10.2450/2007.0047-07
Rich, Rebecca L., et al. “A global benchmark study using affinity-based biosensors.” Analytical biochemistry 386.2 (2009): 194-216.
Stenberg, Esa, et al. “Quantitative determination of surface concentration of protein with surface plasmon resonance using radiolabeled proteins.” Journal of colloid and interface science 143.2 (1991): 513-526.
Song, J., Park, D. W., Moon, S., Cho, H.-J., Park, J. H., Seok, H., & Choi, W. S. (2019). Diagnostic and prognostic value of interleukin-6, pentraxin 3, and procalcitonin levels among sepsis and septic shock patients: a prospective controlled study according to the Sepsis-3 definitions. BMC Infectious Diseases, 19(1), 968. https://doi.org/10.1186/s12879-019-4618-7
Thompson, I. A. P., Zheng, L., Eisenstein, M. et al. Rational design of aptamer switches with programmable pH response. Nat Commun 11, 2946 (2020). https://doi.org/10.1038/s41467-020-16808-2
Turton, David A., Hans Martin Senn, Thomas Harwood, Adrian J. Lapthorn, Elizabeth M. Ellis, and Klaas Wynne. 2014. “Terahertz Underdamped Vibrational Motion Governs Protein-Ligand Binding in Solution.” Nature Communications 5 (1): 3999. https://doi.org/10.1038/ncomms4999.
Zhou, F., Yu, T., Du, R., Fan, G., Liu, Y., Liu, Z., Xiang, J., Wang, Y., Song, B., Gu, X., Guan, L., Wei, Y., Li, H., Wu, X., Xu, J., Tu, S., Zhang, Y., Chen, H., & Cao, B. (2020). Clinical course and risk factors for mortality of adult inpatients with COVID-19 in Wuhan, China: a retrospective cohort study. Lancet (London, England), 395(10229), 1054-1062. https://doi.org/10.1016/s0140-6736(20)30566-3

Sequence listing:
full length SIL-2D1 sdAb
SEQ ID NO: 1
EVQLVESGGGSVQPGGSLRLSCAASGFTLADESSGWFRQAPGKER
EGVSCISITDGITYYADSVKGRFTISRDNAKNTLYLQMNSLKPED
TAVYYCATGNPYCSGHAGTEYDDWGQGTQVTVSS
SIL-2D1 FR1
SEQ ID NO: 2
EVQLVESGGGSVQPGGSLRLSCAAS
SIL-2D1 CDR1
SEQ ID NO: 3
GFTLADES
SIL-2D1 FR2
SEQ ID NO: 4
SGWFRQAPGKEREGVSC
SIL-2D1 CDR2
SEQ ID NO: 5
ISITDGIT
SIL-2D1 FR3
SEQ ID NO: 6
YYADSVKGRFTISRDNAKNTLYLQMNSLKPEDTAVYYC
SIL-2D1 CDR3
SEQ ID NO: 7
ATGNPYCSGHAGTEYDD
SIL-2D1 FR4
SEQ ID NO: 8
WGQGTQVTVSS
full length SIL-2D8 sdAb
SEQ ID NO: 9
EVQLVESGGGLVQAGGSLRLSCTASGHNADTYAMGWFRQAPGKER
EFVTGISWTGVGTYIADSVKGRFTISRDNAKNTVYLQMNSLKPED
TAVYRCAAGSDYSDSHYDKSYEYDYWGQGTQVTVSS
SIL-2D8 FR1
SEQ ID NO: 10
EVQLVESGGGLVQAGGSLRLSCTAS
SIL-2D8 CDR1
SEQ ID NO: 11
GHNADTYA
SIL-2D8 FR2
SEQ ID NO: 12
MGWFRQAPGKEREFVTG
SIL-2D8 CDR2
SEQ ID NO: 13
ISWTGVGT
SIL-2D8 FR3
SEQ ID NO: 14
YIADSVKGRFTISRDNAKNTVYLQMNSLKPEDTAVYRC
SIL-2D8 CDR3
SEQ ID NO: 15
AAGSDYSDSHYDKSYEYDY
SIL-2D8 FR4
SEQ ID NO: 16
WGQGTQVTVSS
full length SIL-2E4 sdAb
SEQ ID NO: 17
EVQLVESGGGLVQPGGSLRLSCAASGSIFDDHAIGWFRQAPGKER
EGVSSIMTSDDSTHYADSVKGRFTISRDKAKNTVYLQMNNLKPED
TAVYYCAATDVFGLQYSSFLKSYEYDYWGQGTQVTVSS
SIL-2E4 FR1
SEQ ID NO: 18
EVQLVESGGGLVQPGGSLRLSCAAS
SIL-2E4 CDR1
SEQ ID NO: 19
GSIFDDHA
SIL-2E4 FR2
SEQ ID NO: 20
IGWFRQAPGKEREGVSS
SIL-2E4 CDR2
SEQ ID NO: 21
IMTSDDST
SIL-2E4 FR3
SEQ ID NO: 22
HYADSVKGRFTISRDKAKNTVYLQMNNLKPEDTAVYYC
SIL-2E4 CDR3
SEQ ID NO: 23
AATDVFGLQYSSFLKSYEYDY
SIL-2E4 FR4
SEQ ID NO: 24
WGQGTQVTVSS
full length SIL-2E7 sdAb
SEQ ID NO: 25
EVQLVESGGGLVQAGGSLRLSCAASGRTFSKNTMGWFRQAPGKER
EFVAAISWSVGSTYYGDSVNGRFTISRDDAKNTVYLQMNSLKPED
TAIYYCAADSSHYMLHPELREYDYWGQGTQVTVSS
SIL-2E7 FR1
SEQ ID NO: 26
EVQLVESGGGLVQAGGSLRLSCAAS
SIL-2E7 CDR1
SEQ ID NO: 27
GRTFSKNT
SIL-2E7 FR2
SEQ ID NO: 28
MGWFRQAPGKEREFVAA
SIL-2E7 CDR2
SEQ ID NO: 29
ISWSVGST
SIL-2E7 FR3
SEQ ID NO: 30
YYGDSVNGRFTISRDDAKNTVYLQMNSLKPEDTAIYYC
SIL-2E7 CDR3
SEQ ID NO: 31
AADSSHYMLHPELREYDY
SIL-2E7 FR4
SEQ ID NO: 32
WGQGTQVTVSS
full length SIL-2F5 sdAb
SEQ ID NO: 33
EVQLVESGGGLVQAGGSLRLSCAASGRAADTYAMGWFRQSPGSRR
EFVTGISWTGSGTYYADSVKGRFTISRDTAKNTVYLQMNSLKPED
TAVYYCAAGSDYSDSHYDKSYEYDYWGQGTQVTVSS
SIL-2F5 FR1
SEQ ID NO: 34
EVQLVESGGGLVQAGGSLRLSCAAS
SIL-2F5 CDR1
SEQ ID NO: 35
GRAADTYA
SIL-2F5 FR2
SEQ ID NO: 36
MGWFRQSPGSRREFVTG
SIL-2F5 CDR2
SEQ ID NO: 37
ISWTGSGT
SIL-2F5 FR3
SEQ ID NO: 38
YYADSVKGRFTISRDTAKNTVYLQMNSLKPEDTAVYYC
SIL-2F5 CDR3
SEQ ID NO: 39
AAGSDYSDSHYDKSYEYDY
SIL-2F5 FR4
SEQ ID NO: 40
WGQGTQVTVSS
full length SIL-3B6 sdAb
SEQ ID NO: 41
EVQLVESGGGLVQPGGSLRLSCAASGSIFDDHAIGWFRQAPGKER
EGVSSIMTSDDSTHYADSVKGRFTVSRDKAKNTVYLQMNDLKPED
TAVYYCAATDVFGLQYSSFLKSYEYDYWGQGTQVTVSS
SIL-3B6 FR1
SEQ ID NO: 42
EVQLVESGGGLVQPGGSLRLSCAAS
SIL-3B6 CDR1
SEQ ID NO: 43
GSIFDDHA
SIL-3B6 FR2
SEQ ID NO: 44
IGWFRQAPGKEREGVSS
SIL-3B6 CDR2
SEQ ID NO: 45
IMTSDDST
SIL-3B6 FR3
SEQ ID NO: 46
HYADSVKGRFTVSRDKAKNTVYLQMNDLKPEDTAVYYC
SIL-3B6 CDR3
SEQ ID NO: 47
AATDVFGLQYSSFLKSYEYDY
SIL-3B6 FR4
SEQ ID NO: 48
WGQGTQVTVSS
full length SIL-3B7 sdAb
SEQ ID NO: 49
EVQLVESGGGLVQAGGSLRLSCAASARTFSNYAMGWFRQAPGKER
EFVAGISWSGGYTSYADSVKGRFTISRDNAKNTVALEMNSLKPED
TAVYYCAAEGVIDELPIKTMRYEYDYWGQGTQVTVSS
SIL-3B7 FR1
SEQ ID NO: 50
EVQLVESGGGLVQAGGSLRLSCAAS
SIL-3B7 CDR1
SEQ ID NO: 51
ARTFSNYA
SIL-3B7 FR2
SEQ ID NO: 52
MGWFRQAPGKEREFVAG
SIL-3B7 CDR2
SEQ ID NO: 53
ISWSGGYT
SIL-3B7 FR3
SEQ ID NO: 54
SYADSVKGRFTISRDNAKNTVALEMNSLKPEDTAVYYC
SIL-3B7 CDR3
SEQ ID NO: 55
AAEGVIDELPIKTMRYEYDY
SIL-3B7 FR4
SEQ ID NO: 56
WGQGTQVTVSS
full length SIL-3E7 sdAb
SEQ ID NO: 57
EVQLVESGGGLVQAGGSLRLSCAASARTFSNYALGWFRQVPGKER
EFVAGITWSGDNTYYADSVKDRFTISRDNAKNTVWLQMNSLRPED
TAVYICAAEAVLEDKVAVSTMRYEYDYWGQGTQVTVSS
SIL-3E7 FR1
SEQ ID NO: 58
EVQLVESGGGLVQAGGSLRLSCAAS
SIL-3E7 CDR1
SEQ ID NO: 59
ARTFSNYA
SIL-3E7 FR2
SEQ ID NO: 60
LGWFRQVPGKEREFVAG
SIL-3E7 CDR2
SEQ ID NO: 61
ITWSGDNT
SIL-3E7 FR3
SEQ ID NO: 62
YYADSVKDRFTISRDNAKNTVWLQMNSLRPEDTAVYIC
SIL-3E7 CDR3
SEQ ID NO: 63
AAEAVLEDKVAVSTMRYEYDY
SIL-3E7 FR4
SEQ ID NO: 64
WGQGTQVTVSS

TABLE 25
Table summarizing the herein disclosed sequences:

| SEQ ID NO: | sdAb | Fragment |
|---|---|---|
| 1 | SIL-2D1 | full length sdAb |
| 2 | SIL-2D1 | FR1 |
| 3 | SIL-2D1 | CDR1 |
| 4 | SIL-2D1 | FR2 |
| 5 | SIL-2D1 | CDR2 |
| 6 | SIL-2D1 | FR3 |
| 7 | SIL-2D1 | CDR3 |
| 8 | SIL-2D1 | FR4 |
| 9 | SIL-2D8 | full length sdAb |
| 10 | SIL-2D8 | FR1 |
| 11 | SIL-2D8 | CDR1 |
| 12 | SIL-2D8 | FR2 |
| 13 | SIL-2D8 | CDR2 |
| 14 | SIL-2D8 | FR3 |
| 15 | SIL-2D8 | CDR3 |
| 16 | SIL-2D8 | FR4 |
| 17 | SIL-2E4 | full length sdAb |
| 18 | SIL-2E4 | FR1 |
| 19 | SIL-2E4 | CDR1 |
| 20 | SIL-2E4 | FR2 |
| 21 | SIL-2E4 | CDR2 |
| 22 | SIL-2E4 | FR3 |
| 23 | SIL-2E4 | CDR3 |
| 24 | SIL-2E4 | FR4 |
| 25 | SIL-2E7 | full length sdAb |
| 26 | SIL-2E7 | FR1 |
| 27 | SIL-2E7 | CDR1 |
| 28 | SIL-2E7 | FR2 |
| 29 | SIL-2E7 | CDR2 |
| 30 | SIL-2E7 | FR3 |
| 31 | SIL-2E7 | CDR3 |
| 32 | SIL-2E7 | FR4 |
| 33 | SIL-2F5 | full length sdAb |
| 34 | SIL-2F5 | FR1 |
| 35 | SIL-2F5 | CDR1 |
| 36 | SIL-2F5 | FR2 |
| 37 | SIL-2F5 | CDR2 |
| 38 | SIL-2F5 | FR3 |
| 39 | SIL-2F5 | CDR3 |
| 40 | SIL-2F5 | FR4 |
| 41 | SIL-3B6 | full length sdAb |
| 42 | SIL-3B6 | FR1 |
| 43 | SIL-3B6 | CDR1 |
| 44 | SIL-3B6 | FR2 |
| 45 | SIL-3B6 | CDR2 |
| 46 | SIL-3B6 | FR3 |
| 47 | SIL-3B6 | CDR3 |
| 48 | SIL-3B6 | FR4 |
| 49 | SIL-3B7 | full length sdAb |
| 50 | SIL-3B7 | FR1 |
| 51 | SIL-3B7 | CDR1 |
| 52 | SIL-3B7 | FR2 |
| 53 | SIL-3B7 | CDR2 |
| 54 | SIL-3B7 | FR3 |
| 55 | SIL-3B7 | CDR3 |
| 56 | SIL-3B7 | FR4 |
| 57 | SIL-3E7 | full length sdAb |
| 58 | SIL-3E7 | FR1 |
| 59 | SIL-3E7 | CDR1 |
| 60 | SIL-3E7 | FR2 |
| 61 | SIL-3E7 | CDR2 |
| 62 | SIL-3E7 | FR3 |
| 63 | SIL-3E7 | CDR3 |
| 64 | SIL-3E7 | FR4 |

1. An anti-interleukin 6 (IL6) single-domain antibody (sdAb) comprising an on-rate constant (kₒₙ) of about 1.1×10\hat{ }5 s-M⁻¹-3×10\hat{ }6 s⁻¹M⁻¹ and an off-rate constant (k_{off}) of about 2.5×10\hat{ }−4 s⁻¹-2×10\hat{ }−3 s⁻¹, preferably wherein said sdAb comprises an IC50 of about 1 nM or more than about 1 nM, and preferably wherein said sdAb is capable of detecting and/or quantifying IL6 at a concentration of about 45 fM or less to about 4.5 pM or more.

2. The anti-IL6 sdAb of claim 1, wherein the sdAb comprises a first complementary determining region (CDR1), a second CDR (CDR2) and third CDR (CDR3), wherein:
(i) CDR1 comprises SEQ ID NO: 3 or a variant thereof having up to two amino acid substitutions, CDR2 comprises SEQ ID NO: 5 or a variant thereof having up to two amino acid substitutions, CDR3 comprises SEQ ID NO: 7 or a variant thereof having up to five amino acid substitutions; or
(ii) CDR1 comprises SEQ ID NO: 11 or a variant thereof having up to two amino acid substitutions, CDR2 comprises SEQ ID NO: 13 or a variant thereof having up to two amino acid substitutions, CDR3 comprises SEQ ID NO: 15 or a variant thereof having up to five amino acid substitutions; or
(iii) CDR1 comprises SEQ ID NO: 19 or a variant thereof having up to two amino acid substitutions, CDR2 comprises SEQ ID NO: 21 or a variant thereof having up to two amino acid substitutions, CDR3 comprises SEQ ID NO: 23 or a variant thereof having up to five amino acid substitutions; or
(iv) CDR1 comprises SEQ ID NO: 27 or a variant thereof having up to two amino acid substitutions, CDR2 comprises SEQ ID NO: 29 or a variant thereof having up to two amino acid substitutions, CDR3 comprises SEQ ID NO: 31 or a variant thereof having up to five amino acid substitutions; or
(v) CDR1 comprises SEQ ID NO: 35 or a variant thereof having up to two amino acid substitutions, CDR2 comprises SEQ ID NO: 37 or a variant thereof having up to two amino acid substitutions, CDR3 comprises SEQ ID NO: 39 or a variant thereof having up to five amino acid substitutions; or
(vi) CDR1 comprises SEQ ID NO: 43 or a variant thereof having up to two amino acid substitutions, CDR2 comprises SEQ ID NO: 45 or a variant thereof having up to two amino acid substitutions, CDR3 comprises SEQ ID NO: 47 or a variant thereof having up to five amino acid substitutions; or
(vii) CDR1 comprises SEQ ID NO: 51 or a variant thereof having up to two amino acid substitutions, CDR2 comprises SEQ ID NO: 53 or a variant thereof having up to two amino acid substitutions, CDR3 comprises SEQ ID NO: 55 or a variant thereof having up to five amino acid substitutions; or
(viii) CDR1 comprises SEQ ID NO: 59 or a variant thereof having up to two amino acid substitutions, CDR2 comprises SEQ ID NO: 61 or a variant thereof having up to two amino acid substitutions, CDR3 comprises SEQ ID NO: 63 or a variant thereof having up to five amino acid substitutions.

3. The anti-IL6 sdAb of claim 1 or 2, wherein the sdAb comprises a first framework region (FR1), a second framework region (FR2) and third framework region (FR3), wherein:
(i) FR1 comprises SEQ ID NO: 2 or a variant thereof having up to six amino acid substitutions, FR2 comprises SEQ ID NO: 4 or a variant thereof having up to four amino acid substitutions, FR3 comprises SEQ ID NO: 6 or a variant thereof having up to ten amino acid substitutions, and FR4 comprises SEQ ID NO: 8 or a variant thereof having up to three amino acid substitutions; or
(ii) FR1 comprises SEQ ID NO: 10 or a variant thereof having up to six amino acid substitutions, FR2 comprises SEQ ID NO: 12 or a variant thereof having up to four amino acid substitutions, FR3 comprises SEQ ID NO: 14 or a variant thereof having up to ten amino acid substitutions, and FR4 comprises SEQ ID NO: 16 or a variant thereof having up to three amino acid substitutions; or
(iii) FR1 comprises SEQ ID NO: 18 or a variant thereof having up to six amino acid substitutions, FR2 comprises SEQ ID NO: 20 or a variant thereof having up to four amino acid substitutions, FR3 comprises SEQ ID NO: 22 or a variant thereof having up to ten amino acid substitutions, and FR4 comprises SEQ ID NO: 24 or a variant thereof having up to three amino acid substitutions; or
(iv) FR1 comprises SEQ ID NO: 26 or a variant thereof having up to six amino acid substitutions, FR2 comprises SEQ ID NO: 28 or a variant thereof having up to four amino acid substitutions, FR3 comprises SEQ ID NO: 30 or a variant thereof having up to ten amino acid substitutions, and FR4 comprises SEQ ID NO: 32 or a variant thereof having up to three amino acid substitutions; or
(v) FR1 comprises SEQ ID NO: 34 or a variant thereof having up to six amino acid substitutions, FR2 comprises SEQ ID NO: 36 or a variant thereof having up to four amino acid substitutions, FR3 comprises SEQ ID NO: 38 or a variant thereof having up to ten amino acid substitutions, and FR4 comprises SEQ ID NO: 40 or a variant thereof having up to three amino acid substitutions; or
(vi) FR1 comprises SEQ ID NO: 42 or a variant thereof having up to six amino acid substitutions, FR2 comprises SEQ ID NO: 44 or a variant thereof having up to four amino acid substitutions, FR3 comprises SEQ ID NO: 46 or a variant thereof having up to ten amino acid substitutions, and FR4 comprises SEQ ID NO: 48 or a variant thereof having up to three amino acid substitutions; or
(vii) FR1 comprises SEQ ID NO: 50 or a variant thereof having up to six amino acid substitutions, FR2 comprises SEQ ID NO: 52 or a variant thereof having up to four amino acid substitutions, FR3 comprises SEQ ID NO: 54 or a variant thereof having up to ten amino acid substitutions, and FR4 comprises SEQ ID NO: 56 or a variant thereof having up to three amino acid substitutions; or
(viii) FR1 comprises SEQ ID NO: 58 or a variant thereof having up to six amino acid substitutions, FR2 comprises SEQ ID NO: 60 or a variant thereof having up to four amino acid substitutions, FR3 comprises SEQ ID NO: 62 or a variant thereof having up to ten amino acid substitutions, and FR4 comprises SEQ ID NO: 64 or a variant thereof having up to three amino acid substitutions.

4. The anti-IL6 sdAb of any one of claims 1 to 3, wherein the sdAb comprises any one of
(i) SEQ ID NO: 1 or a variant thereof having at least about 90% sequence identity to SEQ ID NO: 1; or
(ii) SEQ ID NO: 9 or a variant thereof having at least about 90% sequence identity to SEQ ID NO: 9; or
(iii) SEQ ID NO: 17 or a variant thereof having at least about 90% sequence identity to SEQ ID NO: 17; or
(iv) SEQ ID NO: 25 or a variant thereof having at least about 90% sequence identity to SEQ ID NO: 25; or
(v) SEQ ID NO: 33 or a variant thereof having at least about 90% sequence identity to SEQ ID NO: 33; or
(vi) SEQ ID NO: 41 or a variant thereof having at least about 90% sequence identity to SEQ ID NO: 41; or
(vii) SEQ ID NO: 49 or a variant thereof having at least about 90% sequence identity to SEQ ID NO: 49; or
(viii) SEQ ID NO: 57 or a variant thereof having at least about 90% sequence identity to SEQ ID NO: 57.

5. The anti-IL6 sdAb of any one of claims 1-4, wherein the anti-IL6 sdAb comprises an on-rate constant (kₒₙ) of about 0.15×10\hat{ }6 s⁻¹M⁻¹-2.25×10\hat{ }6 s⁻¹M⁻¹ and an off-rate constant (k_{off}) of about 0.0002 s⁻¹-0.002 s⁻¹.

6. The anti-IL6 sdAb of any one of claims 1-4, wherein the anti-IL6 sdAb comprises an on-rate constant (kₒₙ) of about 1.86×10\hat{ }5 s⁻¹M⁻¹-1.82×10\hat{ }6 s⁻¹M⁻¹ and an off-rate constant (k_{off}) of about 0.000308 s⁻¹-0.00161 s⁻¹ and optionally a K_{D} of about 3×10\hat{ }−10 M-6.8×10\hat{ }−9 M.

7. The anti-IL6 sdAb of any one of claims 1-4, wherein the anti-IL6 sdAb comprises an on-rate constant (kₒₙ) of about 1.20×10\hat{ }6 s⁻¹M⁻¹-1.30×10\hat{ }7 s⁻¹M⁻¹ and an off-rate constant (k_{off}) of about 1.20×10\hat{ }−6 s⁻¹-7.40×10\hat{ }−2 s⁻¹ and optionally a K_{D} of about 2.80×10\hat{ }−13 M-1.40×10\hat{ }−8 M.

8. The anti-IL6 sdAb of any one of claims 1-4, wherein the anti-IL6 sdAb comprises an on-rate constant (kₒₙ) of about 1.86×10\hat{ }5 s⁻¹M⁻¹-2.21×10\hat{ }6 s-M⁻¹ 1.12×10's⁻¹M⁻¹-1.69×10\hat{ }4 s⁻¹M⁻¹ and an off-rate constant (k_{off}) of about 3.08×10\hat{ }−4 s⁻¹-1.61×10\hat{ }−3 s⁻¹±1.86×10\hat{ }−6 s⁻¹-2.94×10\hat{ }−5 s⁻¹ and optionally a K_{D} of about 0.30 M-6.80 M, optionally wherein the values are obtained by a SPR Biacore binding kinetic measurement, preferably wherein the sdAb is defined in any one of SEQ ID NOs: 1, 9, 17, 33, 41, 49, 57.

9. The anti-IL6 sdAb of any one of claims 1-4, wherein the anti-IL6 sdAb comprises an on-rate constant (kₒₙ) of about 6.11×10\hat{ }5 s⁻¹M⁻¹-2.33×10\hat{ }6 s-M⁻¹ 2.60×10\hat{ }4 s⁻¹M⁻¹-3.10×10\hat{ }6 s⁻¹M⁻¹ and an off-rate constant (k_{off}) of about 1.25×10\hat{ }−4 s⁻¹-3.57×10\hat{ }−2 s⁻¹±6.11×10\hat{ }−5 s⁻¹-2.32×10\hat{ }−2 s⁻¹ and optionally a K_{D} of about 2.81×10\hat{ }−1 M-2.66×10\hat{ }1 M, optionally wherein the values are obtained by an Octet BLI binding kinetic measurement, preferably wherein the sdAb is defined in any one of SEQ ID NOs: 1, 9, 17, 25, 33, 41, 49, 57.

10. The anti-IL6 sdAb of any one of claims 1-9, wherein the anti-IL6 sdAb continuously and reversibly binds IL-6.

11. The anti-IL6 sdAb of any one of claims 1-10, wherein the anti-IL6 sdAb binds a second or further IL6 after dissociating from a first IL6.

12. The anti-IL6 sdAb of any one of claims 1-11, wherein the anti-IL6 sdAb inhibits the biological function of IL6.

13. The anti-IL6 sdAb of any one of claims 1-11, wherein the anti-IL6 sdAb does not inhibit the biological function of IL6.

14. The anti-IL6 sdAb of claim 12 or 13, wherein the biological function of IL6 is IL6 signalling or the IL6 signalling pathway.

15. The anti-IL6 sdAb of any one of claims 1-14, wherein the anti-IL6 sdAb is conjugated to a biosensor electrode, such as a working electrode.

16. The anti-IL6 sdAb of claim 15, wherein the anti-IL6 sdAb is conjugated to a biosensor electrode via a linker, such as a nucleic acid.

17. The anti-IL6 sdAb of claim 15 or 16, wherein the biosensor comprises one or more electrode, such as a working electrode and/or a counter electrode and/or a reference electrode, and a substrate.

18. The anti-IL6 sdAb of any one of claims 15-17, wherein the biosensor comprises at least a working electrode and a substrate.

19. The anti-IL6 sdAb of any one of claims 15-18, wherein the biosensor comprises an electrode comprising a conductive material such as carbon, metal, or metallic compound.

20. The anti-IL6 sdAb of any one of claims 1-19, wherein the anti-IL6 sdAb further comprises a detection label or tag.

21. The anti-IL6 sdAb of any one of claims 1-19, wherein the anti-IL6 sdAb does not comprise a detection label or tag.

22. A nucleic acid encoding the anti-IL6 sdAb of any one of claims 1-21.

23. A vector comprising the nucleic acid of claim 22.

24. The vector of claim 23, wherein the vector is an expression vector.

25. A method for detecting and/or quantifying IL6 in a sample, the method comprising:
(i) contacting a sample with the anti-IL6 sdAb of any one of claims 1-21, and
(ii) detecting binding of the anti-IL6 sdAb.

26. An in situ method for detecting and/or quantifying IL6 in a subject, the method comprising
(i) contacting a subject's biological fluid with the anti-IL6 sdAb of any one of claims 1-21; and
(ii) detecting binding of the anti-IL6 sdAb.

27. The method of claim 25 or 26, further comprising detecting the binding of the anti-IL6 sdAb over time.

28. The method of any one of claims 25-27, further comprising detecting the repeated binding of the anti-IL6 sdAb over time.

29. The method of any one of claims 25-28, wherein binding of the anti-IL6 sdAb is detected by a biosensor.

30. The method of any one of claims 25-29, wherein the sample comprises a biological fluid, such as a bodily fluid, for example interstitial fluid (ISF), blood, urine, stool, serum, tear fluid, sweat or saliva; or wherein the biological fluid is a bodily fluid, for example interstitial fluid (ISF), blood, urine, stool, serum, tear fluid, sweat or saliva.

31. The method of any one of claims 25-30, wherein the sample comprises human interstitial fluid (ISF), blood, urine, stool, serum, tear, sweat or saliva, or wherein the biological fluid is a human bodily fluid, for example human interstitial fluid (ISF), blood, urine, stool, serum, tear fluid, sweat or saliva.

32. The method of any one of claims 25 and 27-29, wherein the sample comprises a sample from a biological production system such as a culture sample.

33. The anti-IL6 sdAb of any one of claims 1-21 for use as a medicament.

34. The anti-IL6 sdAb of any one of claims 1-21 for use in in vivo diagnosis.

35. The anti-IL6 sdAb of any one of claims 1-21 for use in the treatment of a disease associated with an inflammation, optionally a systemic inflammation.

36. A method of treatment a disease associated with an inflammation, optionally a systemic inflammation comprising a step of administering the anti-IL6 sdAb of any one of claims 1-21 to a subject in need thereof.

37. The anti-IL6 sdAb for use of claim 35, or the method of claim 36, wherein the disease is associated with an increased production or release of inflammatory markers.

38. The anti-IL6 sdAb for use of claim 35 or 37, or the method of claim 36 or 37, wherein the disease is associated with an increased production or release of IL6.

39. The anti-IL6 sdAb for use of any one of claims 35 or 37-38, or the method of any one of claims 36-38, wherein the disease is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

40. The anti-IL6 sdAb of any one of claims 1-21 for use in a method of in vivo diagnosis of a disease associated with an inflammation, optionally a systemic inflammation, the method comprising:
(i) contacting a subject with the anti-IL6 sdAb of any one of claims 1-21, and
(ii) detecting the binding of the anti-IL6 sdAb.

41. A method of in vivo diagnosis of a disease associated with an inflammation, optionally a systemic inflammation, the method comprising:
(i) contacting a subject with the anti-IL6 sdAb of any one of claims 1-21, and
(ii) detecting binding of the anti-IL6 sdAb.

42. An ex vivo or in vitro method of monitoring the level of IL6 comprising:
(i) contacting a sample of a subject with the anti-IL6 sdAb of any one of claims 1-21, and
(ii) detecting binding of the anti-IL6 sdAb.

43. A method of monitoring the level of IL6 in a subject comprising:
(i) contacting the subject with the anti-IL6 sdAb of any one of claims 1-21, and
(ii) detecting binding of the anti-IL6 sdAb.

44. The anti-IL6 sdAb for use of any one of claims 35 or 37-40, or the method of any one of claims 36-39 or 41-43, wherein binding of the anti-IL6 sdAb is repeatedly detected over time.

45. An apparatus comprising:
(i) the anti-IL6 sdAb of any one of claims 1-21, and
(ii) a contacting unit for contacting a sample or a subject with the anti-IL6 sdAb, and
(iii) a detecting unit for detecting and/or quantifying binding of the anti-IL6 sdAb, and optionally
(iv) a dispensing unit for dispensing a drug, which optionally is the anti-IL6 sdAb of any one of claims 1-21.

46. The apparatus of claim 45 for use in the treatment or in vivo diagnosis of a disease associated with an inflammation, optionally a systemic inflammation.

47. The apparatus of claim 45 or the apparatus for use of claim 46, wherein the detecting unit for detecting and/or quantifying binding of the anti-IL6 sdAb comprises a biosensor.

48. The apparatus or the apparatus for use of claim 47, wherein the biosensor comprises one or more electrode, such as a working electrode and/or a counter electrode and/or a reference electrode, and a substrate, optionally wherein the sdAb is conjugated to the working electrode.

49. The apparatus or the apparatus for use of claim 47 or 48, wherein the biosensor comprises at least a working electrode and a substrate.

50. The apparatus or the apparatus for use of any one of claims 47-49, wherein the biosensor comprises an electrode comprising a conductive material such as carbon, a metal or metallic compound.

51. The apparatus or the apparatus for use of any one of claims 48-50, wherein the anti-IL6 sdAb is layered on the one or more electrode or conjugated to the one or more electrode, preferably the working electrode and optionally, wherein the anti-IL6 sdAb is conjugated to said electrode via a linker.

52. The anti-IL6 sdAb for use of claim 35 or 40, or the method of claim 36 or 41, or the apparatus for use of any one of claims 46-51, wherein the disease is associated with an increased production or release of inflammatory markers.

53. The anti-IL6 sdAb for use of any one of claims 35, 40 or 52, or the method of any one of claims 36, 41 or 52, or the apparatus for use of any one of claims 46-52, wherein the disease is associated with an increased production or release of IL6.

54. The anti-IL6 sdAb for use of any one of claims 35, 40 or 52-53, or the method of any one of claims 36, 41 or 52-53, or the apparatus of any one of claims 46-53, wherein the disease is any one of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

55. A method of producing an anti-IL6 sdAb comprising:
(i) contacting a subject with a recombinant human IL6, and
(ii) isolating a sdAb which successfully binds to IL6, and
(iii) selecting for an on-rate constant (kₒₙ) of about 6.3×10\hat{ }4 s⁻¹M⁻¹-1.3×10\hat{ }7 s⁻¹M⁻¹ and an off-rate constant (k_{off}) of about 1.2×10\hat{ }−6 s⁻¹-0.31 s⁻¹.

56. The method of claim 55, wherein the method comprises selecting for an on-rate constant (kₒₙ) of about 0.15×10\hat{ }6 s⁻¹M⁻¹-2.25×10\hat{ }6 s⁻¹M⁻¹ and an off-rate constant (k_{off}) of about 0.0002 s⁻¹-0.002 s⁻¹.

57. The method of claim 55 or 56, wherein the method further comprises a step of selecting for continuous and reversible binding to IL6.

58. The method of any one of claims 55-57, wherein the method further comprises a step of selecting for binding to a second or further IL6 after dissociation from a first IL6.

59. The method of any one of claims 55-58, wherein the method further comprises a step of selecting for inhibiting the biological function of IL6 or not inhibiting the biological function of IL6.

60. The method of claim 59, wherein the biological function of IL6 is IL6 signalling or the IL6 signalling pathway.

61. An anti-IL6 sdAb produced by the method of any one of claims 55-60.

62. A biosensor comprising the anti-IL6 sdAb of any one of claims 1-21.

63. The biosensor of claim 62, wherein the biosensor comprises one or more biosensor electrodes.

64. The biosensor of claim 62 or 63, wherein the biosensor comprises a working electrode and a substrate

65. The biosensor of claim 64, wherein the biosensor further comprises a counter electrode and/or a reference electrode.

66. The biosensor of any one of claims 62-65, wherein the anti-IL6 sdAb is conjugated to a biosensor electrode, optionally to a working electrode.

67. The biosensor of claim 66, wherein the anti-IL6 sdAb is conjugated to a biosensor electrode, optionally to a working electrode, via a linker, such as a nucleic acid linker, amino acid/peptide linker, peptide nucleic acid linker, polymer linker, polyethylenglycol (PEG) linker, protein-tag linker, or conventional chemistry technique linker.

68. The biosensor of any one of claims 64-67, wherein the working electrode comprises a conductive material, such as carbon, metal or metallic compound.

69. The biosensor of any one of claims 62-68, wherein the biosensor, the anti-IL6 sdAb or the linker comprises a detection label, a tag, or red-ox reporters/indicators.

70. The method of any one of claims 25-32, 36-39 or 41-44, or the anti-IL6 sdAb for use of any one of claims 35, 37-40 or 44, wherein binding of the anti-IL6 sdAb to IL6 is detected and/or quantified by electrochemical ELISA.


SEQUENCE LISTING (68 sequences)

SEQ ID NO: 1 - protein, 124 aa, synthetic construct
  source 1..124: note = full length SIL-2D1 sdAb
EVQLVESGGGSVQPGGSLRLSCAASGFTLADESSGWFRQAPGKEREGVSCISITDGITYYADSVKGRFTISRDNAKNTLYLQMNSLKPEDTAVYYCATGNPYCSGHAGTEYDDWGQGTQVTVSS

SEQ ID NO: 2 - protein, 25 aa, synthetic construct
  source 1..25: note = SIL-2D1 FR1
EVQLVESGGGSVQPGGSLRLSCAAS

SEQ ID NO: 3 - protein, 8 aa, synthetic construct
  source 1..8: note = SIL-2D1 CDR1
GFTLADES

SEQ ID NO: 4 - protein, 17 aa, synthetic construct
  source 1..17: note = SIL-2D1 FR2
SGWFRQAPGKEREGVSC

SEQ ID NO: 5 - protein, 8 aa, synthetic construct
  source 1..8: note = SIL-2D1 CDR2
ISITDGIT

SEQ ID NO: 6 - protein, 38 aa, synthetic construct
  source 1..38: note = SIL-2D1 FR3
YYADSVKGRFTISRDNAKNTLYLQMNSLKPEDTAVYYC

SEQ ID NO: 7 - protein, 17 aa, synthetic construct
  source 1..17: note = SIL-2D1 CDR3
ATGNPYCSGHAGTEYDD

SEQ ID NO: 8 - protein, 11 aa, synthetic construct
  source 1..11: note = SIL-2D1 FR4
WGQGTQVTVSS

SEQ ID NO: 9 - protein, 126 aa, synthetic construct
  source 1..126: note = full length SIL-2D8 sdAb
EVQLVESGGGLVQAGGSLRLSCTASGHNADTYAMGWFRQAPGKEREFVTGISWTGVGTYIADSVKGRFTISRDNAKNTVYLQMNSLKPEDTAVYRCAAGSDYSDSHYDKSYEYDYWGQGTQVTVSS

SEQ ID NO: 10 - protein, 25 aa, synthetic construct
  source 1..25: note = SIL-2D8 FR1
EVQLVESGGGLVQAGGSLRLSCTAS

SEQ ID NO: 11 - protein, 8 aa, synthetic construct
  source 1..8: note = SIL-2D8 CDR1
GHNADTYA

SEQ ID NO: 12 - protein, 17 aa, synthetic construct
  source 1..17: note = SIL-2D8 FR2
MGWFRQAPGKEREFVTG

SEQ ID NO: 13 - protein, 8 aa, synthetic construct
  source 1..8: note = SIL-2D8 CDR2
ISWTGVGT

SEQ ID NO: 14 - protein, 38 aa, synthetic construct
  source 1..38: note = SIL-2D8 FR3
YIADSVKGRFTISRDNAKNTVYLQMNSLKPEDTAVYRC

SEQ ID NO: 15 - protein, 19 aa, synthetic construct
  source 1..19: note = SIL-2D8 CDR3
AAGSDYSDSHYDKSYEYDY

SEQ ID NO: 16 - protein, 11 aa, synthetic construct
  source 1..11: note = SIL-2D8 FR4
WGQGTQVTVSS

SEQ ID NO: 17 - protein, 128 aa, synthetic construct
  source 1..128: note = full length SIL-2E4 sdAb
EVQLVESGGGLVQPGGSLRLSCAASGSIFDDHAIGWFRQAPGKEREGVSSIMTSDDSTHYADSVKGRFTISRDKAKNTVYLQMNNLKPEDTAVYYCAATDVFGLQYSSFLKSYEYDYWGQGTQVTVSS

SEQ ID NO: 18 - protein, 25 aa, synthetic construct [sequence omitted]

SEQ ID NO: 19 - protein, 8 aa, synthetic construct
  source 1..8: note = SIL-2E4 CDR1
GSIFDDHA

SEQ ID NO: 20 - protein, 17 aa, synthetic construct
  source 1..17: note = SIL-2E4 FR2
IGWFRQAPGKEREGVSS

SEQ ID NO: 21 - protein, 8 aa, synthetic construct
  source 1..8: note = SIL-2E4 CDR2
IMTSDDST

SEQ ID NO: 22 - protein, 38 aa, synthetic construct
  source 1..38: note = SIL-2E4 FR3
HYADSVKGRFTISRDKAKNTVYLQMNNLKPEDTAVYYC

SEQ ID NO: 23 - protein, 21 aa, synthetic construct
  source 1..21: note = SIL-2E4 CDR3
AATDVFGLQYSSFLKSYEYDY

SEQ ID NO: 24 - protein, 11 aa, synthetic construct
  source 1..11: note = SIL-2E4 FR4
WGQGTQVTVSS

SEQ ID NO: 25 - protein, 125 aa, synthetic construct
  source 1..125: note = full length SIL-2E7 sdAb
EVQLVESGGGLVQAGGSLRLSCAASGRTFSKNTMGWFRQAPGKEREFVAAISWSVGSTYYGDSVNGRFTISRDDAKNTVYLQMNSLKPEDTAIYYCAADSSHYMLHPELREYDYWGQGTQVTVSS

SEQ ID NO: 26 - protein, 25 aa, synthetic construct
  source 1..25: note = SIL-2E7 FR1
EVQLVESGGGLVQAGGSLRLSCAAS

SEQ ID NO: 27 - protein, 8 aa, synthetic construct
  source 1..8: note = SIL-2E7 CDR1
GRTFSKNT

SEQ ID NO: 28 - protein, 17 aa, synthetic construct
  source 1..17: note = SIL-2E7 FR2
MGWFRQAPGKEREFVAA

SEQ ID NO: 29 - protein, 8 aa, synthetic construct
  source 1..8: note = SIL-2E7 CDR2
ISWSVGST

SEQ ID NO: 30 - protein, 38 aa, synthetic construct
  source 1..38: note = SIL-2E7 FR3
YYGDSVNGRFTISRDDAKNTVYLQMNSLKPEDTAIYYC

SEQ ID NO: 31 - protein, 18 aa, synthetic construct
  source 1..18: note = SIL-2E7 CDR3
AADSSHYMLHPELREYDY

SEQ ID NO: 32 - protein, 11 aa, synthetic construct
  source 1..11: note = SIL-2E7 FR4
WGQGTQVTVSS

SEQ ID NO: 33 - protein, 126 aa, synthetic construct
  source 1..126: note = full length SIL-2F5 sdAb
EVQLVESGGGLVQAGGSLRLSCAASGRAADTYAMGWFRQSPGSRREFVTGISWTGSGTYYADSVKGRFTISRDTAKNTVYLQMNSLKPEDTAVYYCAAGSDYSDSHYDKSYEYDYWGQGTQVTVSS

SEQ ID NO: 34 - protein, 25 aa, synthetic construct
  source 1..25: note = SIL-2F5 FR1
EVQLVESGGGLVQAGGSLRLSCAAS

SEQ ID NO: 35 - protein, 8 aa, synthetic construct
  source 1..8: note = SIL-2F5 CDR1
GRAADTYA

SEQ ID NO: 36 - protein, 17 aa, synthetic construct
  source 1..17: note = SIL-2F5 FR2
MGWFRQSPGSRREFVTG

SEQ ID NO: 37 - protein, 8 aa, synthetic construct
  source 1..8: note = SIL-2F5 CDR2
ISWTGSGT

SEQ ID NO: 38 - protein, 38 aa, synthetic construct
  source 1..38: note = SIL-2F5 FR3
YYADSVKGRFTISRDTAKNTVYLQMNSLKPEDTAVYYC

SEQ ID NO: 39 - protein, 19 aa, synthetic construct
  source 1..19: note = SIL-2F5 CDR3
AAGSDYSDSHYDKSYEYDY

SEQ ID NO: 40 - protein, 11 aa, synthetic construct
  source 1..11: note = SIL-2F5 FR4
WGQGTQVTVSS

SEQ ID NO: 41 - protein, 128 aa, synthetic construct
  source 1..128: note = full length SIL-3B6 sdAb
EVQLVESGGGLVQPGGSLRLSCAASGSIFDDHAIGWFRQAPGKEREGVSSIMTSDDSTHYADSVKGRFTVSRDKAKNTVYLQMNDLKPEDTAVYYCAATDVFGLQYSSFLKSYEYDYWGQGTQVTVSS

SEQ ID NO: 42 - protein, 25 aa, synthetic construct [sequence omitted]

SEQ ID NO: 43 - protein, 8 aa, synthetic construct
  source 1..8: note = SIL-3B6 CDR1
GSIFDDHA

SEQ ID NO: 44 - protein, 17 aa, synthetic construct
  source 1..17: note = SIL-3B6 FR2
IGWFRQAPGKEREGVSS

SEQ ID NO: 45 - protein, 8 aa, synthetic construct
  source 1..8: note = SIL-3B6 CDR2
IMTSDDST

SEQ ID NO: 46 - protein, 38 aa, synthetic construct
  source 1..38: note = SIL-3B6 FR3
HYADSVKGRFTVSRDKAKNTVYLQMNDLKPEDTAVYYC

SEQ ID NO: 47 - protein, 21 aa, synthetic construct
  source 1..21: note = SIL-3B6 CDR3
AATDVFGLQYSSFLKSYEYDY

SEQ ID NO: 48 - protein, 11 aa, synthetic construct
  source 1..11: note = SIL-3B6 FR4
WGQGTQVTVSS

SEQ ID NO: 49 - protein, 127 aa, synthetic construct
  source 1..127: note = full length SIL-3B7 sdAb
EVQLVESGGGLVQAGGSLRLSCAASARTFSNYAMGWFRQAPGKEREFVAGISWSGGYTSYADSVKGRFTISRDNAKNTVALEMNSLKPEDTAVYYCAAEGVIDELPIKTMRYEYDYWGQGTQVTVSS

SEQ ID NO: 50 - protein, 25 aa, synthetic construct
  source 1..25: note = SIL-3B7 FR1
EVQLVESGGGLVQAGGSLRLSCAAS

SEQ ID NO: 51 - protein, 8 aa, synthetic construct
  source 1..8: note = SIL-3B7 CDR1
ARTFSNYA

SEQ ID NO: 52 - protein, 17 aa, synthetic construct
  source 1..17: note = SIL-3B7 FR2
MGWFRQAPGKEREFVAG

SEQ ID NO: 53 - protein, 8 aa, synthetic construct
  source 1..8: note = SIL-3B7 CDR2
ISWSGGYT

SEQ ID NO: 54 - protein, 38 aa, synthetic construct
  source 1..38: note = SIL-3B7 FR3
SYADSVKGRFTISRDNAKNTVALEMNSLKPEDTAVYYC

SEQ ID NO: 55 - protein, 20 aa, synthetic construct
  source 1..20: note = SIL-3B7 CDR3
AAEGVIDELPIKTMRYEYDY

SEQ ID NO: 56 - protein, 11 aa, synthetic construct
  source 1..11: note = SIL-3B7 FR4
WGQGTQVTVSS

SEQ ID NO: 57 - protein, 128 aa, synthetic construct
  source 1..128: note = full length SIL-3E7 sdAb
EVQLVESGGGLVQAGGSLRLSCAASARTFSNYALGWFRQVPGKEREFVAGITWSGDNTYYADSVKDRFTISRDNAKNTVWLQMNSLRPEDTAVYICAAEAVLEDKVAVSTMRYEYDYWGQGTQVTVSS

SEQ ID NO: 58 - protein, 25 aa, synthetic construct
  source 1..25: note = SIL-3E7 FR1
EVQLVESGGGLVQAGGSLRLSCAAS

SEQ ID NO: 59 - protein, 8 aa, synthetic construct
  source 1..8: note = SIL-3E7 CDR1
ARTFSNYA

SEQ ID NO: 60 - protein, 17 aa, synthetic construct
  source 1..17: note = SIL-3E7 FR2
LGWFRQVPGKEREFVAG

SEQ ID NO: 61 - protein, 8 aa, synthetic construct
  source 1..8: note = SIL-3E7 CDR2
ITWSGDNT

SEQ ID NO: 62 - protein, 38 aa, synthetic construct
  source 1..38: note = SIL-3E7 FR3
YYADSVKDRFTISRDNAKNTVWLQMNSLRPEDTAVYIC

SEQ ID NO: 63 - protein, 21 aa, synthetic construct
  source 1..21: note = SIL-3E7 CDR3
AAEAVLEDKVAVSTMRYEYDY

SEQ ID NO: 64 - protein, 11 aa, synthetic construct
  source 1..11: note = SIL-3E7 FR4
WGQGTQVTVSS

SEQ ID NO: 65 - protein, 14 aa, synthetic construct
AAGSDYSDSHYDKS

SEQ ID NO: 66 - protein, 14 aa, synthetic construct
AAEAVLEDKVVVST

SEQ ID NO: 67 - protein, 10 aa, synthetic construct
AATDVFGLQY

SEQ ID NO: 68 - protein, 8 aa, synthetic construct
AAEGVIDE